(19) United States
(12) Patent Application Publication
(10) Pub. No.: US 2025/0313627 A1
(43) Pub. Date: Oct. 9, 2025

(54) RECEPTOR TYROSINE KINASE-LIKE ORPHAN RECEPTOR 1 (ROR1)-SPECIFIC VHH ANTIBODIES AND MULTISPECIFIC ANTIBODIES THEREOF AS IMMUNE CELL ENGAGERS

(71) Applicant: Fuse Biotherapeutics Inc., Woburn, MA (US)

(72) Inventors: Xueyuan Zhou, Belmont, MA (US); Brian Rabinovich, Winchester, MA (US); Jeffrey Takimoto, Cerritos, CA (US)

(73) Assignee: Fuse Biotherapeutics Inc., Woburn, MA (US)

(21) Appl. No.: 18/881,640

(22) PCT Filed: Jul. 20, 2023
(86) PCT No.: PCT/US2023/070587

§ 371 Date: Jan. 6, 2025

Related U.S. Application Data
(60) Provisional application No. 63/390,781, filed on Jul. 20, 2022.
(60) Provisional application No. 63/415,039, filed on Oct. 11, 2022.

(51) Int. Cl. C07K 16/28; A61K 39/00; A61P 35/00

(52) U.S. Cl. CPC C07K 16/2803; A61P 35/00; C07K 16/2809; A61K 2039/505; C07K 2317/31; C07K 2317/52; C07K 2317/565; C07K 2317/567

(57) ABSTRACT

ROR1-specific antigen-binders, and multispecific antibodies are provided, which contains one or more ROR1-specific antigen-binding sites and at least one antigen-specific binding site for an activation receptor (such as CD3) on an immune cell, wherein various configurations are presented of new VHH-based anti-ROR1 sequences in relation to the antigen-specific binding site for the immune cell activation receptor, as well as to a scaffolding segment forming a constant region of the antibodies. These multispecific antibodies have been demonstrated to bind to ROR1-positive cancer cells, induce immune cell-mediated cytotoxicity against ROR1-positive target cells, and to inhibit growth of tumor size in animals.

### CROSS-REFERENCE TO RELATED APPLICATIONS

This application includes a claim of priority under to U.S. provisional patent application No. 63/390,781, filed Jul. 20, 2022, and 63/415,039, filed Oct. 11, 2022, the entirety of both is hereby incorporated by reference.

### REFERENCE TO SEQUENCE LISTING

This application contains a Sequence Listing submitted as a computer readable form named “096034_000001WOPT_SequenceListing.xml”, having a size in bytes of 223,342 bytes, and created on Jul. 19, 2023. The information contained in this computer readable form is hereby incorporated by reference in its entirety.

### FIELD OF INVENTION

This invention relates to multispecific antibodies for eliciting killing of tumor cells by engaging immune effector cells and redirecting their cytotoxicity against ROR1-expressing tumor cells.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

ROR1 is a transmembrane receptor tyrosine kinase broadly expressed on the cell surface of malignant B cells in chronic lymphocytic leukemia (CLL) and mantle cell lymphoma (MCL), as well as being expressed in an array of major solid tumor cancer categories including but not limited to subsets of carcinomas such as triple negative breast cancer (TNBC), ovarian cancer, lung cancer, and melanoma. ROR1 has an extracellular domain (ECD) composed of 3 domains: a membrane-distal immunoglobulin (Ig)-like domain, an intervening frizzled (Fz) domain, and a membrane-proximal kringle (Kr) domain, together comprising about 375 extracellular amino acids. It plays an important role during normal embryogenesis in the development of the central nervous system, heart, lung, and skeletal systems, but its expression in adult tissues is severely restricted, with expression limited to low-level expression in the pancreas, parathyroid, gut, adipocytes and pre-B cells. Since ROR1 expression is tightly regulated in normal adult tissues, high levels have been noted in both hematological and solid tumors.

Therefore, it is an objective of the present invention to provide compounds and therapeutics with multispecific (including bispecific) activity for retargeting cytolytic immune effectors including pro-inflammatory T helper type 1 cells and cytotoxic T lymphocytes toward tumor-associated antigen-expressing cells (e.g., ROR1-expressing cells).

### SUMMARY OF THE INVENTION

The following embodiments and aspects thereof are described and illustrated in conjunction with compositions and methods which are meant to be exemplary and illustrative, not limiting in scope.

Various embodiments provide for a polypeptide, comprising: a polypeptide having a complementarity-determining region (CDR) 1, a polypeptide having a CDR2, and a polypeptide having a CDR3 selected from Table 8A or Table 8B, wherein CDR1, CDR2 and CDR3 are selected from the same row in Table 8A or Table 8B.

Various embodiments provide for a variant of the polypeptide having the polypeptide having the CDR1, the polypeptide having the CDR2, and the polypeptide having the CDR3, wherein the variant of the polypeptide having the CDR1 comprises one or more deletions, additions or substitutions of an amino acid residue in the polypeptide having the CDR1, wherein the variant of the polypeptide having the CDR2 comprises one or more deletions, additions or substitutions of an amino acid residue in the polypeptide having the CDR2, and wherein the variant of the polypeptide having the CDR3 comprises one or more deletions, additions or substitutions of an amino acid residue in the polypeptide having the CDR3, wherein CDR1, CDR2 and CDR3 are selected from the same row in Table 8A or Table 8B.

In various embodiments, the polypeptide can comprise: a polypeptide having a CDR1 of SEQ ID NO: 4, a polypeptide having a CDR2 of SEQ ID NO:77, and a polypeptide having a CDR3 of SEQ ID NO: 6; OR a polypeptide having a CDR1 of SEQ ID NO:73, a polypeptide having a CDR2 of SEQ ID NO:77, and a polypeptide having a CDR3 of SEQ ID NO:84; OR a polypeptide having a CDR1 of SEQ ID NO:73, a polypeptide having a CDR2 of SEQ ID NO:77, and a polypeptide having a CDR3 of SEQ ID NO:85; OR a polypeptide having a CDR1 of SEQ ID NO:73, a polypeptide having a CDR2 of SEQ ID NO:5, and a polypeptide having a CDR3 of SEQ ID NO:84; OR a polypeptide having a CDR1 of SEQ ID NO:73, a polypeptide having a CDR2 of SEQ ID NO:5, and a polypeptide having a CDR3 of SEQ ID NO:85.

In various embodiments, the polypeptide can further comprise framework region (FWR) 1, framework region 2, framework region 3, and framework region 4 selected from Table 8B, and wherein FWR1, FWR2, FWR3, and FWR4 are selected from the same row in Table 8B.

In various embodiments, the polypeptide can be selected from Table 7, or a variant of the polypeptide selected from Table 7, wherein the variant can comprise one or more deletions, additions or substitutions of an amino acid residues of the polypeptide. In various embodiments, the variant can comprise up to 5 deletions, additions or substitutions of an amino acid residues of the polypeptide. In various embodiments, the variant is at least 95% identical to the polypeptide selected from Table 7.

In various embodiments, the polypeptide can comprise a polypeptide having SEQ ID NO:16, SEQ ID NO:39, SEQ ID NO:40, or SEQ ID NO:60.

Various embodiments provide for a polypeptide, comprising: a polypeptide having SEQ ID NO: 1 (complementarity-determining region (CDR) 1 of 2A11), a polypeptide having SEQ ID NO:2 (CDR2 of 2A11), a polypeptide having SEQ ID NO:3 (CDR3 of 2A11), or a combination thereof, OR a variant of the polypeptide having SEQ ID NO:1 (CDR1 of 2A11), a variant of the polypeptide having SEQ ID NO:2 (CDR2 of 2A11), a variant of the polypeptide having SEQ ID NO:3 (CDR3 of 2A11), or a combination thereof, wherein the variant of the polypeptide having SEQ ID NO:1 comprises one or more deletions, additions or substitutions of an amino acid residue in the polypeptide having SEQ ID NO:1, wherein the variant of the polypeptide having SEQ ID NO:2 comprises one or more deletions, additions or substitutions of an amino acid residue in the polypeptide having SEQ ID NO:2, wherein the variant of the polypeptide having SEQ ID NO:3 comprises one or more deletions, additions or substitutions of an amino acid residue in the polypeptide having SEQ ID NO:3, and wherein: the variant of the polypeptide having SEQ ID NO:1 and the variant of the polypeptide having SEQ ID NO:3 do not replace cysteine residues in the polypeptide having SEQ ID NO:1 and the polypeptide having SEQ ID NO:3, or the variant of the polypeptide having SEQ ID NO:1 and the variant of the polypeptide having SEQ ID NO:3 replaces one or both of the cysteine residues in the polypeptide having SEQ ID NO:1 and/or one or both of the cysteine residues in the polypeptide having SEQ ID NO: 3 with an amino acid that contains a cross-linking functional group.

In various embodiments, the polypeptide can comprise a polypeptide having SEQ ID NO: 7.

Various embodiments provide for a polypeptide, comprising: a polypeptide having SEQ ID NO: 4 (CDR1 of 5A1), a polypeptide having SEQ ID NO:5 (CDR2 of 5A1), a polypeptide having SEQ ID NO: 6 (CDR3 of 5A1), or a combination thereof, OR a variant of the polypeptide having SEQ ID NO:4 (CDR1 of 5A1), a variant of the polypeptide having SEQ ID NO:5 (CDR2 of 5A1), a variant of the polypeptide having SEQ ID NO:6 (CDR3 of 2A11), or a combination thereof, wherein the variant of the polypeptide having SEQ ID NO:4 comprises one or more deletions, additions or substitutions of an amino acid residue in the polypeptide having SEQ ID NO:4, wherein the variant of the polypeptide having SEQ ID NO:5 comprises one or more deletions, additions or substitutions of an amino acid residue in the polypeptide having SEQ ID NO: 5, and wherein the variant of the polypeptide having SEQ ID NO:6 comprises one or more deletions, additions or substitutions of an amino acid residue in the polypeptide having SEQ ID NO:6, and wherein: the variant of the polypeptide having SEQ ID NO:4 and the variant of the polypeptide having SEQ ID NO:6 do not replace cysteine residues in SEQ ID NO:4 and SEQ ID NO:6, or the variant of the polypeptide having SEQ ID NO:4 and the variant of the polypeptide having SEQ ID NO:6 replaces one or both of the cysteine residues in the polypeptide having SEQ ID NO:4 and/or one or both of the cysteine residues in the polypeptide having SEQ ID NO:6 with an amino acid that contains a cross-linking functional group.

In various embodiments, the polypeptide can comprise a polypeptide having SEQ ID NO:8; OR the polypeptide can comprise a polypeptide having: SEQ ID NO:11, SEQ ID NO:12, SEQ ID NO:13, SEQ ID NO:14, or SEQ ID NO: 15.

Various embodiments provide for a polynucleotide encoding any one of the polypeptides of the present invention as described herein.

In various embodiments, the polynucleotide can comprise a polynucleotide having: SEQ ID NO: 9, OR SEQ ID NO: 10.

In various embodiments, the polynucleotide can comprise a polynucleotide encoding a polypeptide selected from Table 7.

Various embodiments provide for a vector comprising any one of the polynucleotides of the present invention as described herein.

Various embodiments provide for an isolated cell comprising any one of the vectors of the present invention as described herein.

Various embodiments provide for a protein comprising any one of the polypeptides as described herein, and a fragment crystallizable region (Fc) of an antibody.

Various embodiments provide for a multispecific antibody construct, comprising: one or more first polypeptides, each independently any one of the polypeptides of the invention described herein; and a second polypeptide capable of binding an activation receptor and/or a costimulatory receptor expressed on an immune cell. In various embodiments, the activation receptor can comprise cluster of differentiation (CD) 3, CD16, γ9 TCR, δ2 TCR or δ1 TCR, and the co-stimulatory/co-activation receptor can comprise cluster of differentiation (CD) 137, CD28, DNAM-1, NKp46, NKG2D, NKp30, CD2, ICOS, OX40, CD40L, or CD40.

In various embodiments, the multispecific antibody can further comprise a fragment crystallizable region (Fc) of an antibody or a human serum albumin (HSA).

In various embodiments, the multispecific antibody can comprise two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide having SEQ ID NO:7 or 8.

In various embodiments, the multispecific antibody can comprise two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide selected from Table 7.

In various embodiments, the multispecific antibody can comprise two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide having a sequence as set forth in SEQ ID NO: 16, 39, 40 and 60.

In various embodiments, the multispecific antibody can further comprise one or more linkers, and at least one of the linkers is between the Fc or the HSA and at least one of the first polypeptide, between the Fc or the HSA and the second polypeptide, between any two of the more first polypeptides, or between at least one of the one or more first polypeptides and the second polypeptide.

In various embodiments, the multispecific antibody can be bispecific antibody.

Various embodiments of the invention provide for a method of killing cancer cells in a subject in need thereof, comprising: administering any one of the multispecific antibodies of the present invention as described herein to the subject in need thereof. In various embodiments, the cancer cells express receptor tyrosine kinase-like orphan receptor 1 (ROR1). In various embodiments, the cancer cells are cancer cells of the lung, bronchus, non-Hodgkin lymphoma, leukemia, pancreas, breast, prostate, colon, rectum, bladder, skin, kidney, mouth, tongue, pharynx, ovary, oral cavity, head and neck, thyroid, myeloid leukemia, mantle cell lymphoma, multiple myeloma, or combinations thereof.

Various embodiments of the invention provide for a method of treating cancer in a subject in need thereof, comprising administering any one of the multispecific antibodies of the present invention as described herein to the subject in need thereof. In various embodiments, the cancer cells express receptor tyrosine kinase-like orphan receptor 1 (ROR1). In various embodiments, the cancer cells are cancer cells of the lung, bronchus, non-Hodgkin lymphoma, leukemia, pancreas, breast, prostate, colon, rectum, bladder, skin, kidney, mouth, tongue, pharynx, ovary, oral cavity, head and neck, thyroid, myeloid leukemia, mantle cell lymphoma, multiple myeloma, or combinations thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1E depict schematics of multiple formats of anti-ROR1/CD3 bispecific antibodies, also termed T cell engagers (TCEs), illustrating protein designs of TCE fusion proteins comprising a knob-in-hole (KIH) heterodimeric IgG1 Fc scaffold with (1) a CD3-specific fragment antigen-binding region (Fab) on the N-terminal end of a knob chain (shown in panels 1A, 1B, 1D and 1E) or a hole chain (shown in 1C) of the KIH, and (2) one or more VHH or single-chain variable fragment (scFV) specific for a tumor-associated antigen (TAA) (e.g., ROR1) in one or more positions. The examples shown illustrate the TAA binder linked to (a) the N-terminal end of the hole chain in tandem (1A), (b) the N-terminal end of the hole chain and on the C-terminal end of the CD3-specific (anti-CD3) Fab light chain (1B), (c) the N-terminal end of the knob chain and on the C-terminal end of the CD3-specific (anti-CD3) Fab light chain (1C), (d) the N and C-terminal end of the hole chain (1D) and (e) the N-terminal end of the hole chain only (1E).

FIGS. 2A-2B show the production yield (2A) and the melting temperature (Tm) (2B) of exemplary fusion proteins (denoted as a FUSE ID, “FUSE-XXX”).

FIGS. 3A-3D shows the apparent binding affinity of exemplary TCEs for cell membrane expressed CD3 in human T cells and in non-human primate (NHP), Cynomolgus T cells.

FIGS. 4A-4E depict the apparent binding affinity of exemplary TCEs for cell membrane expressed ROR1 using a series of engineered MDA-MB-231 variant cell lines expressing different cell surface densities of the ROR1. FIGS. 4F-4K depict the capacity of exemplary TCEs specific for ROR1 to bind the membrane distal ROR1 Ig domain.

FIGS. 5A-5D depict the binding affinity (KD) for ROR1, determined via Bio-layer Interferometry (BLI), of TCEs: FUSE-211 (in FIG. 5A), FUSE-393 (in FIG. 5B), and FUSE-394 (in FIG. 5C), with their calculated binding affinity shown in FIG. 5D.

FIGS. 5E-5G depict the binding affinity for ROR1 of a 2A11 VHH-Fc fusion protein (denoted as FUSE-112, in FIG. 5E) and of a 2A11 VHH mutant-Fc fusion protein (denoted as FUSE-453, in FIG. 5F), with their calculated binding affinity shown in FIG. 5G, which demonstrated the impact of replacing the cysteine residues in CDR1 and CDR3 of the ROR1-specific VHH.

FIGS. 5H-5J depict the binding affinity for ROR1 of a 5A1 VHH-Fc fusion protein (denoted as FUSE-179, in FIG. 5H) and of a 5A1 VHH mutant-Fc fusion protein (denoted as FUSE-454, in FIG. 5I), with their calculated binding affinity shown in FIG. 5J, which demonstrated the impact of replacing the cysteine residues in CDR1 and CDR3 of the ROR1-specific VHH.

FIGS. 6A-6D illustrate TCEs induction of human peripheral blood mononuclear cells (PBMC)-mediated, ROR1-dependent cytotoxicity (characterized as percentage killed) of MDA-MB-231 tumor cells, wherein the tested TCEs include FUSE-211, FUSE-393, and FUSE-394 and the human PBMC were from donor 1 (FIG. 6A), donor 2 (FIG. 6B), or donor 3 (FIG. 6C). FIG. 6D summarizes the calculated EC₅₀ of each TCE for inducing a killing percentage of 50% of the tumor cells.

FIGS. 6E-6H illustrate TCEs induction of IFN-γ release from human PBMC (characterized as concentration of IFN-γ in cell culture supernatant) in the presence of MDA-MB-231 tumor cells. The tested TCEs include FUSE-211, FUSE-393, and FUSE-394, and the human PBMC were from donor 1 (FIG. 6E), donor 2 (FIG. 6F), or donor 3 (FIG. 6G). FIG. 6H summarizes the calculated EC₅₀ of each TCE for inducing 50% of a maximum IFN-γ release.

FIGS. 6I-6O illustrate the capacity of FUSE-211, FUSE-393, and FUSE-394 to mediate reduced ROR1-dependent IFN-γ release from human PBMC (average of three donors) or Pan-T cells while maintaining cytotoxic efficacy (maximum killing of MDA-MB-231), or “decoupling of cytotoxicity from cytokine release” compared to a FUSE-277, a prototypical highly potent TCE that serves as the “non-decoupling” control TCE.

FIGS. 6P-6V depict ROR1 dependent killing, induction of cytokine release and calculated decoupling ratios following 24, 48 and 72 hour co-cultures of PBMC effectors with ROR1+ MDA-MB-231 tumor targets.

FIGS. 7A-7C illustrates TCEs induction of human PBMC-mediated, ROR1-independent cytotoxicity of T47-D tumor cells (which do not express ROR1), characterized as IFN-γ concentration in the co-culture supernatant. Panels A-C show the results wherein the human PBMC were from donor 1 (panel A), donor 2 (panel B), and donor 3 (panel C), respectively.

FIGS. 8A-8E and 8G-8K depict TCEs induction of human PBMC-mediated, ROR1-dependent cytotoxicity and cytokine release, respectively, as a function of the cell surface density of ROR1. Data was generated using a series of cell lines of different origins expressing different numbers of ROR1 molecules on their cell surfaces. The cell lines are shown in a descending order of cell surface ROR1 density, from high to low. That is, NCCIT (FIG. 8A,G), MDA-MB-231 (FIG. 8B,H), NCI-H1975 (FIG. 8C,I), DU-145 (FIG. 8D,J) and ROR1-negative T-47D (FIG. 8E,K). FIGS. 8F and 8L summarize the potencies for cytotoxicity and cytokine release induced by each of the TCEs towards a series of cell lines with different cell-surface ROR1 densities.

FIG. 8M illustrates the calculated decoupling ratio for each TCE across the cell line series.

FIGS. 9A-9E and 9G-9K depict TCEs induction of human PBMC-mediated, ROR1-dependent cytotoxicity and cytokine release, respectively, as a function of the cell surface density of ROR1 using a series of engineered MDA-MB-231 variant cell lines expressing different cell surface densities of the ROR1. The surface densities of ROR1 on the engineered MDA-MB-231 were as follows: ˜2,000,000 molecules per cell (MDA++; FIG. 9A, 9G), ˜500,000 molecules per cell (MDA+; FIG. 9B, 9H), ˜35,000 molecules per cell (MDA; FIG. 9C, 9D), ˜17,000 molecules per cell (MDA-low; FIG. 9D, 9J), and 0 molecule per cell (MDA-KO; FIG. 9E, 9K). FIGS. 9F and 9L summarize the potencies for cytotoxicity and cytokine release induced by each of the TCEs towards MDA-MB-231 cells with different cell-surface ROR1 densities. FIG. 9M represents the calculated decoupling ratios (cytotoxicity from cytokine release) for each TCE and FIG. 9N is a regression analysis between the magnitude of IFNγ versus ROR1 cell surface density of a non-decoupling TCE control versus an exemplary decoupling TCE.

FIGS. 10A and 10B depict TCE-induced expression of activation markers, CD69 and CD25, respectively, on T cells following co-culture of PBMCs with ROR1-expressing tumor cells. FIG. 10C summarizes the potency by which each TCE induces CD69 or CD25, defined as the EC₅₀-binding. FIG. 10D and FIG. 10F are bar charts illustrating the decoupling of cytotoxicity from the induction of the cell surface biomarkers of T cell activation, CD69 and CD25. FIG. 10E and FIG. 10G are bar charts illustrating the decoupling of the induction CD69 and CD25 from cytokine release.

FIGS. 11A-11C depict the pharmacokinetics (PK) of exemplary TCEs in a single dose of either 5 mg/kg (FIG. 11A) or 0.5 mg/kg (FIG. 11B) administered to wild type C57BL/6 mice over a 10-day period. FIG. 11C depicts the PK parameters for each test article.

FIGS. 12A-12E depict a schematics of two humanized mouse xenograft tumor models (FIGS. 12A and 12C). In both cases, the highly aggressive human ROR1⁺ teratoma, NCCIT was injected into immune incompetent mice. Tumor growth over time for the first model is shown in FIG. 12B and second model in FIGS. 12D and 12E. In both cases, the test articles consisted of FUSE-394 and the non-decoupled control TCE, FUSE-399.

FIGS. 13A-13D depict the capacity of an exemplary TCE to induce T cell mediated killing of ROR1-positive tumor cells (MDA-MB-231; FIG. 13A), ROR1 negative tumor cells (T-47D; FIG. 13B) and a mixture of ROR1-positive tumor cells and ROR1 negative tumor cells (FIG. 13C and FIG. 13D). For the latter, killing of the ROR1 positive tumor is shown in FIG. 13C. Killing of the ROR1 negative tumor is termed “bystander killing”, the comparison of which to a non-decoupling TCE is shown in FIG. 13D. FIG. 13E depicts IFNγ production by the same T cells used to assess “bystander killing” as a function of exemplary TCE concentration.

FIGS. 14A-14B depict the (a) percent killing of ROR1-positive tumor cells (MDA-MB-231) as a function of T cell numbers (FIG. 14A) in the context of exemplary TCEs and (b) TCE mediated “serial killing” calculated as the average number of TCE induced ROR1-positive tumor cells killed per T cell in a 24-bour period (FIG. 14B).

FIG. 15 depicts the apparent binding affinity (EC50-binding) of the ROR1 specific VHH-Fc incorporating either the non humanized VHH clone 5A1 (FUSE179; black circle), the humanized clone 5A1-FA9-HK1 (FUSE559; black square) or the humanized clone 5A1-HK1 (FUSE524; black triangle) was calculated.

FIGS. 16A-B depict the affinity (KD) of the ROR1 specific VHH-Fc incorporating cither the non humanized VHH clone 5A1 (FUSE179; FIG. 16A) or the humanized clone 5A1-FA9-HK1 (FUSE559; FIG. 16B) was calculated.

FIGS. 17A-17B depict the apparent binding affinity (EC50-binding) of the two bispecific antibodies (bsAbs).

FIGS. 18A-B depict capacity of several ROR1×CD3 specific bsAbs to induce (1; FIG. 18A) T cell mediated killing of ROR1+ MDA-MB-231 tumor cells and (2; FIG. 18B) T cell mediated release of IFNγ when co-mixed with ROR1+ MDA-MB-231 tumor cells.

FIGS. 19A-19I depict capacity of several ROR1×CD3 specific bsAbs to induce (1; FIGS. 19A-D) PBMC mediated killing of four different tumor cell lines and (2; FIGS. 19E-H), PBMC mediated release of IFNγ when co-mixed with the aforementioned tumor cells.

FIGS. 20A-20F depict capacity of several ROR1×CD3 specific bsAbs to induce (1; FIG. 20A) PBMC mediated killing of ROR1+ MDA-MB-231 tumor cells and (2; FIG. 20B), PBMC mediated release of IFNγ when co-mixed with ROR1+ MDA-MB-231 tumor cells, (3. FIG. 20C) upregulation of cell surface CD69 expression on T cells within PBMC when co-mixed with ROR1+ MDA-MB-231 tumor cells, (4. FIG. 20D) upregulation of cell surface PD-1 expression on T cells within PBMC when co-mixed with ROR1+ MDA-MB-231 tumor cells, and (5. FIG. 20E) upregulation of cell surface TIGIT expression on T cells within PBMC when co-mixed with ROR1+ MDA-MB-231 tumor cells.

FIGS. 21A-C depict capacity of FUSE399 (black reverse triangle) and FUSE608 (black circle) to induce CD8+ T cell mediated killing of a constant number (5000 cells) of ROR1+ MDA-MB-231 cells across a range of CD8+ T cell numbers from 313 to 40,000.

FIGS. 22A-22D depict whether FUSE608 induced bystander killing of ROR1 negative tumors co-mixed with ROR1 positive tumors.

FIGS. 23A-23B depict the capacity of FUSE608 and the non-decoupled benchmark ROR1×CD3 bsAb, FUSE399, to mediate tumor growth inhibition (TGI) in a mouse tumor xenograft model.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see e.g., D. Lane, Antibodies: A Laboratory Manual 2^{nd} ed. (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23 (9): 1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “about” when used in reference to numerical ranges, cutoffs, or specific values is used to indicate that the recited values may vary by up to as much as 10% from the listed value. Thus, the term “about” is used to encompass variations of ±10% or less, and where specifically indicated or claimed as such, variations of ±5% or less, variations of ±1% or less, variations of ±0.5% or less, or variations of ±0.1% or less from the specified value.

“Antibody” refers to all isotypes of immunoglobulins (IgG, IgA, IgE, IgM, IgD, and IgY) including various monomeric, polymeric and chimeric forms, unless otherwise specified. Specifically encompassed by the term “antibody” are VHH antibodies or nanobody antibodies that consist of antigen binding fragment(s) of heavy chain only antibodies fused to an Fc, where the VHH are preferably derived from camelids, polyclonal antibodies, monoclonal antibodies (mAbs), and antibody-like polypeptides, such as chimeric antibodies and humanized antibodies.

“Antigen-binding fragments” or “bispecific antigen-binding fragments” are any proteinaceous structure that may exhibit binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation, diabodies, Fab, F(ab′)2, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CHI domains, or a monovalent antibody, bivalent fragments comprising two Fab fragments linked by a disulfide bridge at the hinge region, a Fd fragment consisting essentially of the V_{H} and C_{H1} domains; a Fv fragment consisting essentially of the V_{L} and V_{H} domains of a single an of an antibody, a dAb fragment (e.g., Ward et al., Nature 341, 544-546 (1989)), which consists essentially of a Va domain and also called domain antibodies (e.g., Holt et al; Trends Biotechnol. 2003 November; 21(11):484-90); camelid VHH or nanobodies (e.g., Revets et al; Expert Opin Biol Ther. 2005 January; 5(1):111-24); an isolated complementarity determining region (CDR), and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds (e.g., a receptor-ligand pair, for example, a PD1-Fc fusion would target PDL1 and PDL2). Antigen-binding fragments may be recombinantly produced or produced by enzymatic or chemical cleavage of intact antibodies. The phrase “an antibody or antigen-binding fragment thereof” may be used to denote that a given antigen-binding fragment incorporates one or more amino acid segments of the antibody referred to in the phrase.

“VHH” refers to the single variable domain on a heavy chain. It may also be termed as a nanobody. The VHH (or nanobody) includes three CDR domains that make up the majority of the paratope or antigen binding fragment of heavy chain only antibodies (HcAb). Generally, HcAb is naturally produced by camelids and sharks.

“Single-chain variable fragment” or “scFv” refers to a fusion protein of the variable regions of the heavy (V_{H}) and light chains (V_{L}) of immunoglobulins, connected with a short flexible linker peptide (generally about ten to 25 amino acids long).

In pharmacokinetics study, half-life (t½) generally refers to the time that the drug concentration needs to decrease by 50%. “T½ alpha” refers to the half-life of distribution phase, whereas “T½ beta” refers to the half-life of elimination phases. By some descriptions, the alpha half-life is the rate of decline in plasma concentrations due to the process of drug redistribution from the central to the peripheral compartment, and the beta half-life is the rate of decline due to the process of drug elimination due to metabolism. During the distribution phase, changes in the concentration of drug in plasma reflect primarily movement of drug from the circulation to internal compartments, rather than loss from, the body. However, once the drug in the plasma and tissues has reached equilibrium, the decline of plasma concentration is driven by elimination of the drug from the body, called the elimination phase (late phase).

“ROR1” (Receptor Tyrosine Kinase-Like Orphan Receptor 1) refers to the 106-kDa member of the receptor tyrosine kinase family having a UniProt Accession Number Q01973 (human) and Q9Z139 (mouse).

The term “CD3” refers to the human CD3 protein multi-subunit complex. The CD3 protein multi-subunit complex is composed to 6 distinctive polypeptide chains. These include a CD3γ chain (e.g., SwissProt P09693), a CD3δ chain (e.g., SwissProt P04234), two CD3ε chains (SwissProt P07766), and one CD3ζ chain homodimer (e.g., SwissProt 20963), and which is associated with the T cell receptor α and β chain. CD3 is further clustered with the T cell receptor (TCR) or TCR/CD3 complex, which represents the key activation receptor expressed on T cells. Thus, crosslinking of CD3 or the TCR result in similar signaling pathways and activation of T cells. The term “CD3” includes any CD3 variant, isoform and species homolog which is naturally expressed by cells (including T cells) or can be expressed on cells transfected with genes or cDNA encoding those polypeptides, unless noted.

The term “linker” with respect to amino acid linker in a polypeptide can be a short peptide, such as a dimer of two amino acids, a tri-mer of three amino acids, or a peptide selected from the group consisting of T, PT, MPT, S, GS, GGS, GGGS (SEQ ID NO:150), and (GGGGX_{λ} (SEQ ID NO:151) n wherein X_{λ} is Q, A, E or S and n=1-5 or an integer larger than 5. In some embodiments, the amino acid linker has the amino acid sequence of (GGGGS (SEQ ID NO:152), where n is an integer between 1 and 5, thereby an amino acid linker of 25 amino acids or shorter in length.

“Immunospecifically” when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Phrases such as “anti-[antigen] antibody” (e.g., anti-ROR1 antibody) or “[antigen]-specific antibody” (e.g., ROR1-specific antibody” are meant to convey that the recited antibody specifically binds the recited antigen.

“Isolated” means a biological component (such as an antibody) has been substantially separated, produced apart from, or purified away from other biological components of the organism in which the component naturally occurs, i.e., other chromosomal and extrachromosomal DNA and RNA, and proteins. Antibodies that have been “isolated” thus include antibodies purified by standard purification methods. “Isolated antibodies” can be part of a composition and still be isolated if such composition is not part of the native environment of the antibody. The term also embraces antibodies prepared by recombinant expression in a host cell as well as chemically synthesized antibodies.

### ROR1-Specific Antibodies and Antigen-Binding Fragments

Disclosed herein include antibodies (e.g., isolated antibodies), or antigen-binding fragments thereof, that immunospecifically bind to ROR1. The inventors have identified human ROR1-specific VHH produced in camels in response to stimulation by human ROR1, (thereby termed as VHH derived from Bactrian camels). These human ROR1-stimulated, camel-derived VHH include:
2A11 (a polypeptide comprising a polypeptide having an amino acid sequence of QVQLQESGGGSVPAGGSLRLSCAASGSTYSANCMGWFRQAPGKEREEVASMSIRSGRTYYSDSVK GRFTISQDGSKNTLYLQLNSLKAEDTALYYCAAAYGGSRCVYNYRGQGTQVTVSS, SEQ ID NO:7), and
5A1 (a polypeptide comprising a polypeptide having an amino acid sequence of QVQLQESGGGSVQAGGSLKLSCTASGYTNRLKCMGWFRQAPGKEREEIATISTGTGNTYYADSVK GRFTFSQDKVKNTVYLQMNTLKPDDTGMYYCAADVRPDGTTCHYNSGGQGTQVTVSS, SEQ ID NO: 8).

An isolated anti-ROR1 (or ROR1-specific) antibody, or ROR1-binding fragment thereof, can comprise a VHH which comprises a polypeptide having an amino acid sequence that is at least 85%, 90%, 91%, 92%, 93%, 94%, 95%, 96%, 97%, 98%, or 99% identical to the amino acid sequence of SEQ ID NO:7. In some embodiments, a VHH antibody (or nanobody) is provided, which can comprise, or consist of, the polypeptide having an amino acid sequence of SEQ ID NO:7.

In some embodiments, a polypeptide is provided comprising one or more complementarity-determining regions (CDRs) for binding human ROR1, and these CDRs are identified/present in 2A11, wherein CDR1 of 2A11 consists of a polypeptide having an amino acid sequence of GSTYSANC (SEQ ID NO: 1), CDR2 of 2A11 consists of a polypeptide having an amino acid sequence of MSIRSGRTY (SEQ ID NO: 2), and CDR3 of 2A11 consists of a polypeptide having an amino acid sequence of AYGGSRCVYNY (SEQ ID NO:3).

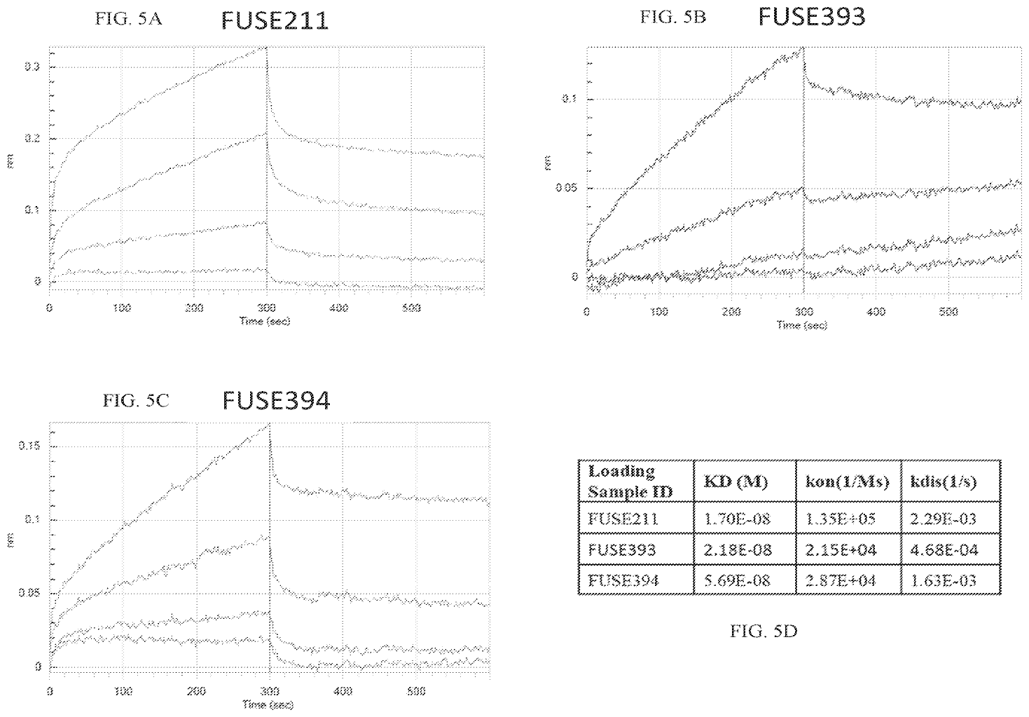

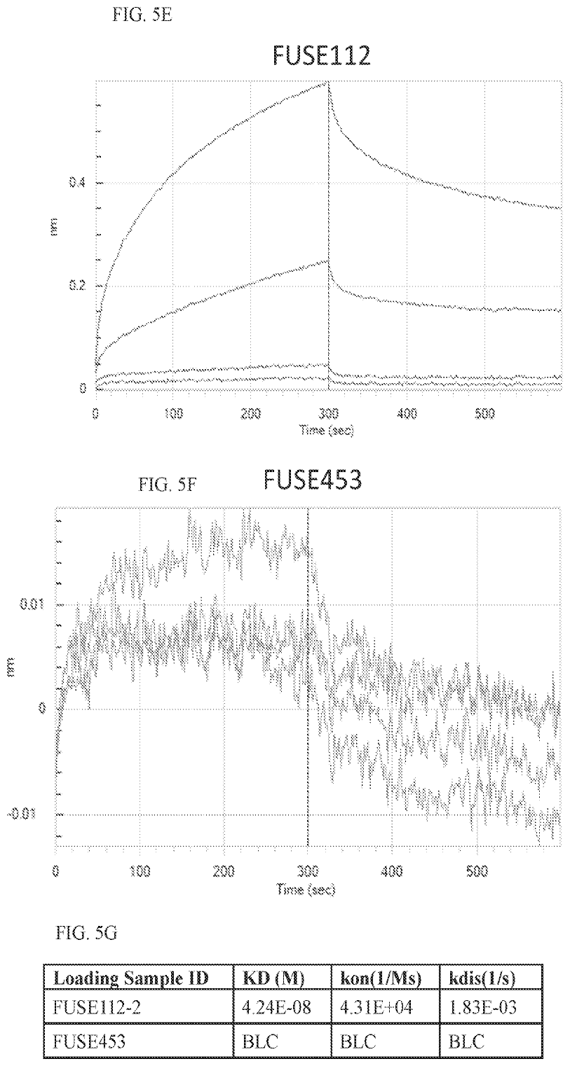

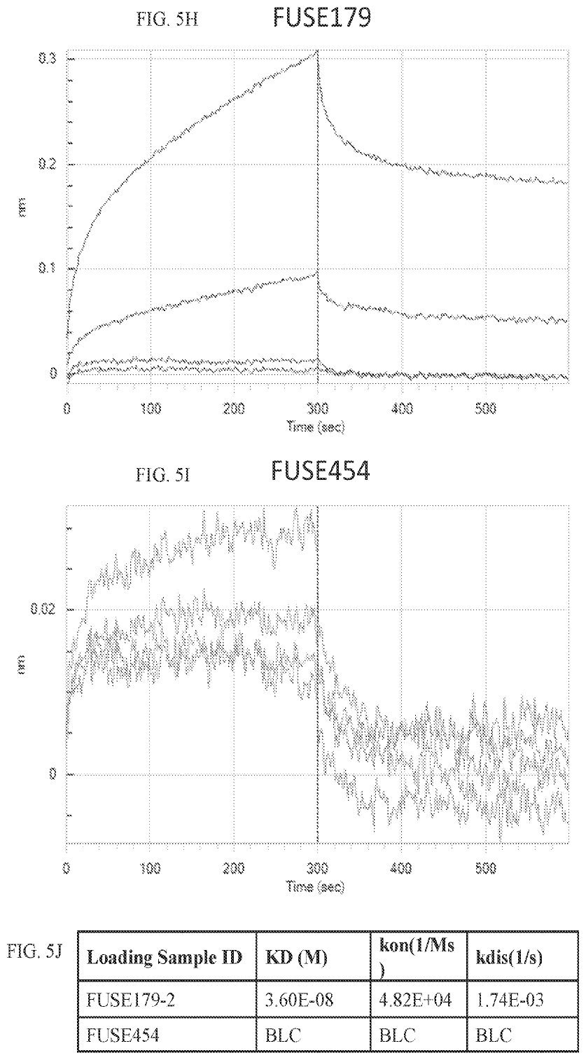

In some embodiments, a polypeptide is provided comprising one or more of a variant of the polypeptide having an amino acid sequence of SEQ ID NO:1, a variant of the polypeptide having an amino acid sequence of SEQ ID NO:2, and a variant of the polypeptide having an amino acid sequence of SEQ ID NO: 3, wherein each of the variants comprises one or more deletions, additions, or substitutions of an amino acid residue compared to respective “wild-type” polypeptides having the amino acid sequence of SEQ ID NOs: 1, 2, or 3, respectively. In various embodiments, the substitutions are conservative substitutions. Preferably, a variant of the polypeptide having an amino acid sequence of SEQ ID NO:1, a variant of the polypeptide having an amino acid sequence of SEQ ID NO:2, and/or a variant of the polypeptide having an amino acid sequence of SEQ ID NO:3 are capable of binding human ROR1, more preferably still capable of immunospecifically binding human ROR1, or possess a binding affinity to ROR1 that is at least 90%, 80%, 70%, 60%, or 50% compared to respective “wild-type” CDR polypeptides present in 2A11. Preferably, one or both of the cysteine residues in the CDRs of 2A11 (i.e., cysteine residues in SEQ ID NO:1 and in SEQ ID NO: 3) is unchanged, in a polypeptide that is a variant of 2A11 or in a polypeptide that is a variant of the polypeptide having an amino acid sequence of SEQ ID NO:1, 2, or 3. Hence, in some embodiments, a polypeptide comprising one or more of a variant of the polypeptide having an amino acid sequence of SEQ ID NO: 1, a variant of the polypeptide having an amino acid sequence of SEQ ID NO:2, and a variant of the polypeptide having an amino acid sequence of SEQ ID NO:3, still maintains the cysteine residues as those in the polypeptide having an amino acid sequence of SEQ ID NO:1 and in the polypeptide having an amino acid sequence of SEQ ID NO:3. Alternatively, a polypeptide can be a variant of 2A11, wherein one or both of the cysteine residues in the CDRs of 2A11 is replaced with a natural or non-natural amino acid that contains a cross-linking functional group, so as to support formation of inter-/intra-molecular bonds; examples of these amino acids including but not limited to p-benzoyl-L-phenylalanine (Bpa), azide, and haloalkane. Preserving the capability to form disulfide and/or intramolecular bonds at those residue locations where cysteine is present in the CDR of 2A11 is indicated to be important, as data in FIG. 5G demonstrates that replacing the cysteine residues with valine significantly weakens the binding affinity of 2A11 to antigen.

In some embodiments, the amino acids in the non-CDR portion of 2A11 are modified, so as to generate a humanized version of 2A11 (comprising the CDRs of SEQ ID NOs: 1, 2, and 3 and a framework/non-CDR portion that is humanized), so as to better suit applications in human subjects. Therefore, various embodiments provide a humanized, anti-ROR1 antibody or fragment thereof.

In additional embodiments, an anti-ROR1 (or ROR1-specific) heavy chain antibody is provided, which comprises (1) an antigen binding portion being a VHH comprising a polypeptide having an amino acid sequence of SEQ ID NO:7, or being a VHH comprising the polypeptides having amino acid sequences of SEQ ID NOs: 1, 2, and 3, and (2) a Fc-domain of an Ig heavy chain. Preferably, the heavy chain antibody further comprises a hinge domain of an Ig heavy chain, so that the VHH is linked to the hinge and the Fc-domains of the Ig heavy chain. The disclosed anti-ROR1 heavy chain antibody, or an antigen-binding fragment thereof, include all isotypes, IgA, IgD, IgE, IgG and IgM, and synthetic multimers of the four-chain immunoglobulin (Ig) structure, as well as the IgY isotype generally found in hen or turkey serum and hen or turkey egg yolk. In some embodiments, the anti-ROR1 heavy chain antibody comprises (1) an antigen binding portion being a VHH comprising a polypeptide having the amino acid sequence of SEQ ID NO:7, or a VHH comprising CDRs having amino acid sequences of SEQ ID NOs: 1, 2, and 3, and (2) the hinge and Fc-domains of an IgG heavy chain, wherein the IgG can be any of the subclasses, IgG1, IgG2, IgG3, and IgG4.

An isolated anti-ROR1 (or ROR1-specific) antibody, or ROR1-binding fragment thereof, can alternatively comprise a VHH comprising a polypeptide having an amino acid sequence that is at least 85%, 90%, 91%, 92%, 93%, 94%, 95%, 96%, 97%, 98%, or 99% identical to the amino acid sequence of SEQ ID NO: 7. In some embodiments, a VHH antibody (or nanobody) is provided, which can comprise, or consist of, a polypeptide having the amino acid sequence of SEQ ID NO:7.

In some embodiments, a polypeptide is provided comprising one or more complementarity-determining regions (CDRs) of 5A1, wherein CDR1 of 5A1 consists of a polypeptide having the amino acid sequence of GYTNRLKC (SEQ ID NO:4), CDR2 of 5A1 consists of a polypeptide having the amino acid sequence of ISTGTGNTY (SEQ ID NO:5), and CDR3 of 5A1 consists of a polypeptide having the amino acid sequence of DVRPDGTTCHYNS (SEQ ID NO:6).

In some embodiments, a polypeptide is provided comprising one or more of a variant of the polypeptide having the amino acid sequence of SEQ ID NO:4, a variant of the polypeptide having the amino acid sequence of SEQ ID NO:5, and a variant of the polypeptide having the amino acid sequence of SEQ ID NO: 6, wherein each of the variants comprises one or more deletions, additions, or substitutions of an amino acid residue compared to respective “wild-type” polypeptide having the amino acid sequences of SEQ ID NOs: 4, 5, or 6. In various embodiments, the substitutions are conservative substitutions. Preferably, a variant of the polypeptide having the amino acid sequence of SEQ ID NO:4, a variant of the polypeptide having the amino acid sequence of SEQ ID NO:5, and/or a variant of the polypeptide having the amino acid sequence of SEQ ID NO:6 are capable of binding human ROR1, more preferably still capable of immunospecifically binding human ROR1, or possess a binding affinity to human ROR1 that is at least 90%, 80%, 70%, 60%, or 50% compared to respective “wild-type” CDRs present in 5A1. Preferably, one or both of the cysteine residues in the CDRs of 5A1 (i.e., cysteine residues in SEQ ID NO:4 and in SEQ ID NO:6) are unchanged, in a polypeptide that is a variant of 5A1 or in a polypeptide that is a variant of the polypeptide having an amino acid sequence of SEQ ID NO:4, 5, or 6. Hence, in some embodiments, a polypeptide comprising one or more of a variant of SEQ ID NO:4, a variant of SEQ ID NO:5, and a variant of SEQ ID NO:6 still maintains the cysteine residues as those in SEQ ID NO:4 and SEQ ID NO:6. Alternatively, a polypeptide can be a variant of 5A1, wherein one or both of the cysteine residues in the CDRs of 5A1 is replaced with a natural or non-natural amino acid that contains a cross-linking functional group, so as to support formation of inter-/intra-molecular bonds; examples of these amino acids including but not limited to p-benzoyl-L-phenylalanine (Bpa), azide, and haloalkane. Preserving the capability to form disulfide and/or intramolecular bonds at those residue locations is indicated to be important, as data in FIG. 5F demonstrates that replacing the cysteine residues with valine significantly weakens the binding affinity of 5A1 to antigen.

In some embodiments, the amino acids in the non-CDR portion of 5A1 are modified, so as to generate a humanized version of 5A1 (comprising the CDRs whose amino acid sequences are SEQ ID NOs: 4, 5, 6, and a framework/non-CDR portion that is humanized), so as to better suit applications in human subjects. Exemplary humanized VHH derived from 5A1 include:
5A1-H1 (a polypeptide having an amino acid sequence of EVQLLESGGGLVQPGGSLRLSCAASGYTNRLKCMGWFRQAPGKERELASISTGTGNTYYADSVKG RFTISRDNSKNTLYLQMNSLKAEDTAVYYCAADVRPDGTTCHYNSRGQGTLVTVSS, SEQ ID NO: 11),
5A1-H2 (a polypeptide having an amino acid sequence of QVQLQESGGGLVQPGGSLRLSCTASGYTNRLKCMGWVRQAPGKEREEVATISTGTGNTYYADSVK GRFTISRDNSKNTLYLQMNSLRAEDTAMYYCAADVRPDGTTCHYNSGGQGTQVTVSS, SEQ ID NO: 12),
5A1-H3 (a polypeptide having an amino acid sequence of EVQLLESGGGLVQPGGSLRLSCAASGYTNRLKCMGWFRQAPGKEREEVSTISTGTGNTYYADSVK GRFTISQDKSKNTLYLRMNSLRAEDTALYYCAADVRPDGTTCHYNSGGQGTQVTVSS, SEQ ID NO: 13),
5A1-H4 (a polypeptide having an amino acid sequence of EVQLLESGGGLVPRGGSLRLSCTASGYTNRLKCMGWFRQAPGKEREEIATISTGTGNTYYADSVKG RFTISRDNSRNTLYLQMKTLRAEDTAVYYCAADVRPDGTTCHYNSWGQGTQVTVSS, SEQ ID NO: 14), and
5A1-H5 (a polypeptide having an amino acid sequence of EVOLVESGGGLVQPGGSLRLSCTASGYTNRLKCMGWFRQAPGKEREEIATISTGTGNTYYADSVKG RFTFSRDNSKNTLYLQMNSLRAEDTAVYYCAADVRPDGTTCHYNSGGQGTQVTVSS, SEQ ID NO: 15).

Additional exemplary humanized VHH are shown in Table 7.

In additional embodiments, an anti-ROR1 (or ROR1-specific) heavy chain antibody is provided, which comprises (1) an antigen binding portion (1) being a VHH comprising a polypeptide having the amino acid sequence of SEQ ID NO:8, or (ii) being a VHH comprising CDRs which are polypeptides having amino acid sequences of SEQ ID NOs: 4, 5, and 6, or (iii) being a VHH comprising a polypeptide having the amino acid sequence of any one of SEQ ID NO:11-15, or (iv) comprising one or more of (i), (ii) and (iii), and (2) a Fc-domain of an Ig heavy chain. Preferably, the heavy chain antibody further comprises a hinge domain of an Ig heavy chain, so that the VHH is linked to the hinge and the Fc-domains of the Ig heavy chain. The disclosed anti-ROR1 heavy chain antibody, or an antigen-binding fragment thereof, include all isotypes, IgA, IgD, IgE, IgG and IgM, and synthetic multimers of the four-chain immunoglobulin (Ig) structure, as well as the IgY isotype generally found in hen or turkey serum and hen or turkey egg yolk. In some embodiments, the anti-ROR1 heavy chain antibody comprises (1) an antigen binding portion being a VHH having the amino acid sequence of SEQ ID NO:8, or being a VHH comprising CDRs whose amino acid sequences are SEQ ID NOs: 4, 5, and 6, or being a VHH having the amino acid sequence of any one of SEQ ID NOs: 11-15, and (2) the binge and Fc-domains of an IgG heavy chain, wherein the IgG can be any of the subclasses, IgG1, IgG2, IgG3, and IgG4.

Polypeptides are also provided comprising (1) two or more repeats of a polypeptide having an amino acid sequence of SEQ ID NO:7, (2) two or more repeats of a polypeptide having an amino acid sequence of SEQ ID NO:8, (3) one or more repeats of a polypeptide having an amino acid sequence of SEQ ID NO: 7 and one or more repeats of a polypeptide having an amino acid sequence of SEQ ID NO:8, (4) two or more repeats of a polypeptide having an amino acid sequence of any one of SEQ ID NOs: 11-15, or (5) one or more repeats of a first polypeptide having an amino acid sequence of one of SEQ ID NOs: 11-15 and one or more repeats of a second polypeptide having an amino acid sequence of one of SEQ ID NOs: 11-15, where the first and the second polypeptides have different amino acid sequences. In some embodiments, a polypeptide comprises a tandem sequence of a first VHH and a second VHH, wherein the first VHH and the second VHH can be the same or different. The amino acid sequence of the first VHH and the amino acid sequence of the second VHH can be individually selected from any one of SEQ ID NOs: 7 and 8, or selected from any one of SEQ ID NOs: 11-15. In one aspect, the first VHH and the second VHH are the same and both have the amino acid sequence of SEQ ID NO:7; in another aspect, the first VHH and the second VHH are the same and both have the amino acid sequence of SEQ ID NO:8; in yet another aspect, the first VHH and the second VHH have the amino acid sequence of SEQ ID NO:7 and/or a variant of the polypeptide having an amino acid sequence of SEQ ID NO:7; in yet another aspect, the first VHH and the second VHH have the amino acid sequence of SEQ ID NO:8 and/or a variant of the polypeptide having the amino acid sequence of SEQ ID NO: 8; or in an alternative aspect, the first VHH may have the amino acid sequence of SEQ ID NO:7 or may be a variant of the polypeptide having the amino acid sequence of SEQ ID NO:7, and the second VHH may have the amino acid sequence of SEQ ID NO:8 or may be a variant of the polypeptide having the amino acid sequence of SEQ ID NO:8. In further embodiments, the polypeptide comprising the tandem sequence further comprises a linker, wherein the first VHH and the second VHH are connected with a linker. The linker may be on the N-terminal end of the VHH closer to the N-terminus of the Fc, or alternatively on the C-terminal end. In various embodiments the one or more is 1, 2, 3, 4, 5 or 6. In various embodiments the two or more is 2, 3, 4, 5 or 6.

Various embodiments provide for a polypeptide, comprising: a polypeptide having a complementarity-determining region (CDR) 1, a polypeptide having a CDR2, and a polypeptide having a CDR3 selected from Table 8A, wherein CDR1, CDR2 and CDR3 are selected from the same row in Table 8A.

Various embodiments provide for a polypeptide, comprising: a polypeptide having a complementarity-determining region (CDR) 1, a polypeptide having a CDR2, and a polypeptide having a CDR3 selected from Table 8B, wherein CDR1, CDR2 and CDR3 are selected from the same row in Table 8B.

Various embodiments provide for a polypeptide, comprising: a variant of the polypeptide having the polypeptide having the CDR1, the polypeptide having the CDR2, and the polypeptide having the CDR3 selected from Table 8A, wherein the variant of the polypeptide having the CDR1 comprises one or more deletions, additions or substitutions of an amino acid residue in the polypeptide having the CDR1, wherein the variant of the polypeptide having the CDR2 comprises one or more deletions, additions or substitutions of an amino acid residue in the polypeptide having the CDR2, and wherein the variant of the polypeptide having the CDR3 comprises one or more deletions, additions or substitutions of an amino acid residue in the polypeptide having the CDR3, wherein CDR1, CDR2 and CDR3 are selected from the same row in Table 8A.

Various embodiments provide for a polypeptide, comprising: a variant of the polypeptide having the polypeptide having the CDR1, the polypeptide having the CDR2, and the polypeptide having the CDR3 selected from Table 8B, wherein the variant of the polypeptide having the CDR1 comprises one or more deletions, additions or substitutions of an amino acid residue in the polypeptide having the CDR1, wherein the variant of the polypeptide having the CDR2 comprises one or more deletions, additions or substitutions of an amino acid residue in the polypeptide having the CDR2, and wherein the variant of the polypeptide having the CDR3 comprises one or more deletions, additions or substitutions of an amino acid residue in the polypeptide having the CDR3, wherein CDR1, CDR2 and CDR3 are selected from the same row in Table 8B.

Various embodiments provide for a polypeptide, comprising: a polypeptide having a CDR1 of SEQ ID NO:4, a polypeptide having a CDR2 of SEQ ID NO:77, and a polypeptide having a CDR3 of SEQ ID NO: 6.

Various embodiments provide for a polypeptide, comprising: a polypeptide having a CDR1 of SEQ ID NO:73, a polypeptide having a CDR2 of SEQ ID NO:77, and a polypeptide having a CDR3 of SEQ ID NO:84.

Various embodiments provide for a polypeptide, comprising: a polypeptide having a CDR1 of SEQ ID NO:73, a polypeptide having a CDR2 of SEQ ID NO:77, and a polypeptide having a CDR3 of SEQ ID NO:85.

Various embodiments provide for a polypeptide, comprising: a polypeptide having a CDR1 of SEQ ID NO:73, a polypeptide having a CDR2 of SEQ ID NO:5, and a polypeptide having a CDR3 of SEQ ID NO: 84.

Various embodiments provide for a polypeptide, comprising: a polypeptide having a CDR1 of SEQ ID NO:73, a polypeptide having a CDR2 of SEQ ID NO:5, and a polypeptide having a CDR3 of SEQ ID NO: 85.

In various embodiments, the substitutions are conservative substitutions. Preferably, variants of the CDR1, CDR2 or CDR3 are capable of binding human ROR1, more preferably still capable of immunospecifically binding human ROR1, or possess a binding affinity to ROR1 that is at least 90%, 80%, 70%, 60%, or 50% compared to respective CDR polypeptides.

In various embodiments, the polypeptide further comprises framework region (FWR) 1, framework region 2, framework region 3, and framework region 4 selected from Table 8B, and wherein FWR1, FWR2, FWR3, and FWR4 are selected from the same row in Table 8B.

In various embodiments, the polypeptide having CDR1, CDR2, CDR3, further comprises a FWR1, FWR2, FWR3 and FWR4 from a human IgG. In various embodiments, the polypeptide having a variant of CDR1, a variant of CDR2, a variant of CDR3, further comprises a FWR1, FWR2, FWR3 and FWR4 from a human IgG.

In various embodiments, the polypeptide is a VHH polypeptide selected from Table 7. In various embodiments, the polypeptide is a variant of a VHH polypeptide selected from Table 7, wherein the variant comprises one or more deletions, additions or substitutions of an amino acid residues of the polypeptide. In various embodiments, the variant comprises up to 5 deletions, additions or substitutions of an amino acid residues of the polypeptide. In various embodiments, the variant comprises up to 1, 2 3 or 4 deletions, additions or substitutions of an amino acid residues of the polypeptide.

In various embodiments, the variant is at least 95% identical to the polypeptide selected from Table 7. In various embodiments, the variant is at least 96%, 97%, 98%, or 99% identical to the polypeptide selected from Table 7. In various embodiments, the variant is at least 99.5%, 99.6%, 99.7%, 99.8%, or 99.9% identical to its corresponding polypeptide selected from Table 7.

In various embodiments, the polypeptide is a VHH polypeptide having an amino acid sequence of SEQ ID NO:16, SEQ ID NO:60, SEQ ID NO:39, or SEQ ID NO:40. In various embodiments, the polypeptide is a variant of a VHH polypeptide having an amino acid sequence of SEQ ID NO:16, SEQ ID NO: 60, SEQ ID NO:39, or SEQ ID NO:40, wherein the variant comprises one or more deletions, additions or substitutions of an amino acid residues of the polypeptide. In various embodiments, the variant comprises up to 5 deletions, additions or substitutions of an amino acid residues of the polypeptide. In various embodiments, the variant comprises up to 1, 2 3 or 4 deletions, additions or substitutions of an amino acid residues of the polypeptide.

In various embodiments, the variant is at least 95% identical to the VHH polypeptide having an amino acid sequence of SEQ ID NO:16, SEQ ID NO:60, SEQ ID NO:39, or SEQ ID NO:40. In various embodiments, the variant is at least 96%, 97%, 98%, or 99% identical to the VHH polypeptide having an amino acid sequence of SEQ ID NO:16, SEQ ID NO:60, SEQ ID NO:39, or SEQ ID NO:40. In various embodiments, the variant is at least 99.5%, 99.6%, 99.7%, 99.8%, or 99.9% identical to its VHH polypeptide having an amino acid sequence of SEQ ID NO:16, SEQ ID NO:60, SEQ ID NO:39, or SEQ ID NO:40.

In additional embodiments, an anti-ROR1 (or ROR1-specific) heavy chain antibody is provided, which comprises (1) an antigen binding portion (i) being a VHH comprising a polypeptide having an amino acid sequence selected from Table 7, or (ii) being a VHH comprising CDRs which are polypeptides having amino acid sequences selected from Table 8A or Table 8B, or (iii) being a VHH comprising a polypeptide having the amino acid sequence of any one of SEQ ID NO:16, 39, 40 or 60, or (iv) comprising one or more of (i), (ii) and (iii), and (2) a Fc-domain of an Ig heavy chain. Preferably, the heavy chain antibody further comprises a hinge domain of an Ig heavy chain, so that the VHH is linked to the hinge and the Fc-domains of the Ig heavy chain. The disclosed anti-ROR1 heavy chain antibody, or an antigen-binding fragment thereof, include all isotypes, IgA, IgD, IgE, IgG and IgM, and synthetic multimers of the four-chain immunoglobulin (Ig) structure, as well as the IgY isotype generally found in hen or turkey serum and hen or turkey egg yolk. In some embodiments, the anti-ROR1 heavy chain antibody comprises (1) an antigen binding portion being a VHH having the amino acid sequence selected from Table 7, or being a VHH comprising CDRs whose amino acid sequences are selected from the same row of Table 8A or the same row of Table 8B, or being a VHH having the amino acid sequence of any one of SEQ ID NOs: 16, 39, 40 or 60, and (2) the hinge and Fc-domains of an IgG heavy chain, wherein the IgG can be any of the subclasses, IgG1, IgG2, IgG3, and IgG4.

Polypeptides are also provided comprising (1) two or more repeats of a polypeptide having an amino acid sequence selected from Table 7, (2) one or more repeats of a first polypeptide having an amino acid sequence selected from Table 7 and one or more repeats of a second polypeptide having an amino acid sequence selected from Table 7, where the first and the second polypeptides have different amino acid sequences, (3) two or more repeats of a polypeptide having an amino acid sequence of any one of SEQ ID NOs: 16, 39, 40 or 60, or (4) one or more repeats of a first polypeptide having an amino acid sequence of one of SEQ ID NOs: 16, 39, 40 or 60 and one or more repeats of a second polypeptide having an amino acid sequence of one of SEQ ID NOs: 16, 39, 40 or 60, where the first and the second polypeptides have different amino acid sequences. In some embodiments, a polypeptide comprises a tandem sequence of a first VHH and a second VHH, wherein the first VHH and the second VHH can be the same or different. The amino acid sequence of the first VHH and the amino acid sequence of the second VHH can be individually selected from Table 7, or selected from any one of SEQ ID NOs: 16, 39, 40 or 60.

In one aspect, the first VHH and the second VHH are the same and both have the same amino acid sequence selected from Table 7; in another aspect, the first VHH and the second VHH have are the same and both have the same amino acid sequence selected from Table 7 and/or a variant thereof. In further embodiments, the polypeptide comprising the tandem sequence further comprises a linker, wherein the first VHH and the second VHH are connected with a linker. The linker may be on the N-terminal end of the VHH closer to the N-terminus of the Fc, or alternatively on the C-terminal end. In various embodiments the one or more is 1, 2, 3, 4, 5 or 6. In various embodiments the two or more is 2, 3, 4, 5 or 6.

The isolated antibody, or antigen-binding fragment thereof, can preferably bind to a region of the ROR1 extracellular domain, and therefore bind to cells with surface expression of ROR1.

The anti-ROR1 VHH (nanobody), heavy chain antibodies, and/or antigen-binding fragments thereof, or polypeptides containing the anti-ROR1 VHH or CDRs thereof, as disclosed above, may be derived from any species by recombinant means. For example, the VHH, heavy chain antibodies or antigen-binding fragments may be camelid, mouse, rat, goat, horse, swine, bovine, chicken, rabbit, donkey, human, or chimeric versions thereof. Particularly, the VHH, heavy chain antibodies or antigen-binding fragments may be camelid or humanized version thereof. For use in administration to humans, non-human derived antibodies or antigen-binding fragments may be genetically or structurally altered to be less antigenic upon administration to the human patient.

Also provided are polynucleotide sequences encoding the disclosed anti-ROR1 VHH, the disclosed anti-ROR1 heavy chain antibodies, and/or the disclosed polypeptides containing the anti-ROR1 VHH or CDRs thereof. For example, vectors encoding the sequence of 2A11, 5A1, or humanized versions of 2A11 or 5A1 can be expressed in various transfection-compatible cell types, not limited to camel cells or human cells. Also, vectors encoding any one of the sequences noted in Table 7 can be expressed in various transfection-compatible cell types, not limited to camel cells or human cells. Vectors comprising the polynucleotides are also provided. The vectors can be expression vectors, such as recombinant expression vectors. The expression vector may contain one or more additional sequences such as, but not limited, to regulatory sequences (e.g., promoter, enhancer), selection markers, and polyadenylation signals. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors. Also provided are cells expressing, and capable of expressing, the disclosed vectors. These cells may be mammalian cells (such as 293F cells, CHO cells), insect cells (such as Sf7 cells), yeast cells, plant cells, or bacteria cells (such as E. coli). The disclosed antibodies may also be produced by hybridoma cells.

### Multispecific Antibodies and Multispecific Antigen-Binding Fragments

Also disclosed herein are multispecific (including bispecific or trispecific) antibodies, or multispecific antigen-binding fragments thereof, which bind to at least ROR1 and CD3 (anti-ROR1/anti-CD3 antibodies), or bind to at least ROR1 and an activation receptor expressed on an immune cell (including but not limited to CD3, CD16, γ9 TCR, δ2 TCR or δ1 TCR, NKp46, and NKG2D). In various embodiments, isolated bispecific antibodies, or bispecific antigen-binding fragments thereof, are provided which bind to ROR1 and CD3 (anti-ROR1/anti-CD3 bispecific antibodies).

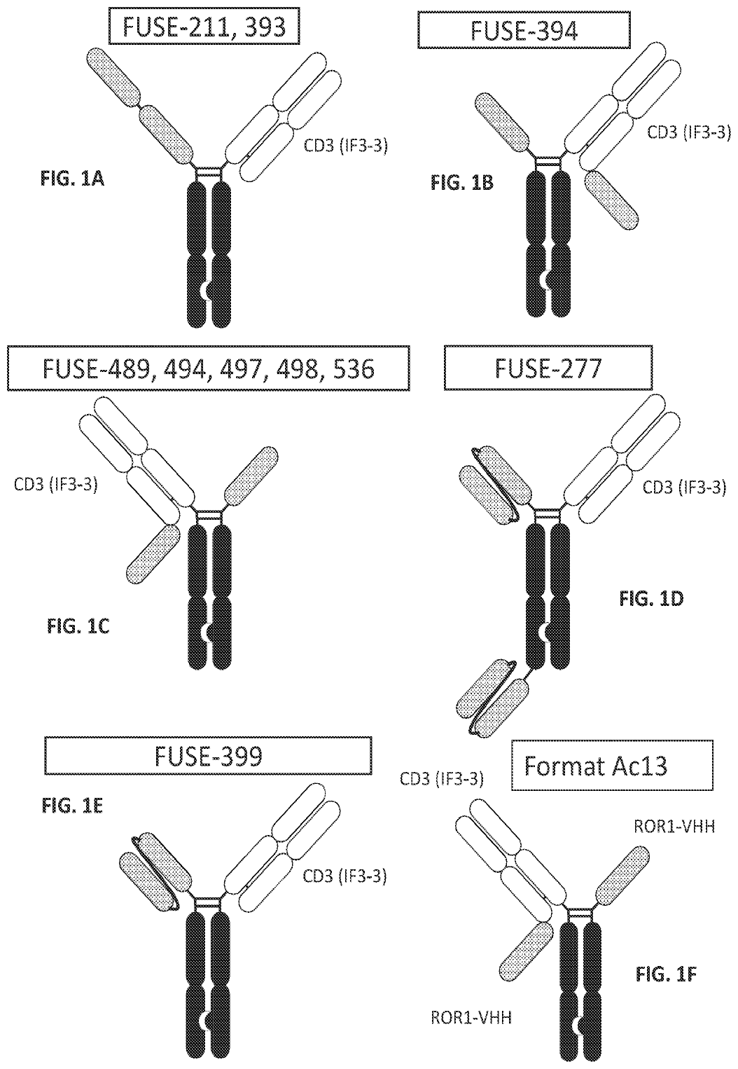

In various embodiments, the anti-ROR1/anti-CD3 antibodies have at least a first antigen-binding site that immunospecifically binds ROR1 (ROR1 arm) and a second antigen-binding site that immunospecifically binds CD3 (CD3 arm). Exemplary anti-ROR1/anti-CD3 antibodies of these embodiments are shown in FIGS. 1A and 1E.

In some embodiments, the anti-ROR1/anti-CD3 antibodies have at least a first arm that is ROR1-specific and a second arm that is bispecific, wherein the first arm that is ROR1-specific comprises a first antigen-binding site that immunospecifically binds ROR1, and the second arm that is bispecific comprises an antigen-biding site that immunospecifically binds CD3 and a second antigen-binding site that immunospecifically binds ROR1. Exemplary anti-ROR1/anti-CD3 antibodies of these embodiments are shown in FIGS. 1B and 1C.

In various embodiments, the anti-ROR1/anti-NKG2D antibodies have at least a first antigen-binding site that immunospecifically binds ROR1 (ROR1 arm) and a second antigen-binding site that immunospecifically binds NKG2D (NKG2D arm).

In some embodiments, the anti-ROR1/anti-NKG2D antibodies have at least a first arm that is ROR1-specific and a second arm that is bispecific, wherein the first arm that is ROR1-specific comprises a first antigen-binding site that immunospecifically binds ROR1, and the second arm that is bispecific comprises an antigen-biding site that immunospecifically binds NKG2D and a second antigen-binding site that immunospecifically binds ROR1.

In various embodiments, the anti-ROR1/anti-CD16 antibodies have at least a first antigen-binding site that immunospecifically binds ROR1 (ROR1 arm) and a second antigen-binding site that immunospecifically binds CD16 (CD16 arm).

In some embodiments, the anti-ROR1/anti-CD16 antibodies have at least a first arm that is ROR1-specific and a second arm that is bispecific, wherein the first arm that is ROR1-specific comprises a first antigen-binding site that immunospecifically binds ROR1, and the second arm that is bispecific comprises an antigen-biding site that immunospecifically binds CD16 and a second antigen-binding site that immunospecifically binds ROR1.

In various embodiments, the anti-ROR1/anti-NKp46 antibodies have at least a first antigen-binding site that immunospecifically binds ROR1 (ROR1 arm) and a second antigen-binding site that immunospecifically binds NKp46 (NKp46 arm).

In some embodiments, the anti-ROR1/anti-NKp46 antibodies have at least a first arm that is ROR1-specific and a second arm that is bispecific, wherein the first arm that is ROR1-specific comprises a first antigen-binding site that immunospecifically binds ROR1, and the second arm that is bispecific comprises an antigen-biding site that immunospecifically binds NKp46 and a second antigen-binding site that immunospecifically binds ROR1.

In further embodiments, isolated multispecific antibodies, or multispecific antigen-binding fragments thereof, are provided, which contains at least one ROR1-binding site, at least one CD3-binding site, and a Fc-domain of an Ig heavy chain, and optionally a hinge domain of the Ig heavy chain. The Ig heavy chain can be any one of isotypes IgA, IgD, IgE, IgG and IgM, and synthetic multimers of the four-chain immunoglobulin (Ig) structure, as well as the IgY isotype generally found in hen or turkey serum and hen or turkey egg yolk. Preferably, the Ig heavy chain is a IgG heavy chain, which can be any of the subclasses, IgG1, IgG2, IgG3, and IgG4.

Hence, in some embodiments, anti-ROR1/anti-CD3 antibodies have at least (1) a first antigen-binding site specific for ROR1, (2) a second antigen-binding site at least specific for CD3, or a bispecific antigen-binding site for ROR1 and CD3, and (3) the hinge and Fc domains of two or more chains of Ig, which can be dimerized or multimerized, wherein at least one heaving chain of the Ig may further comprise an antigen-binding site specific for ROR1, preferably on the C-terminus of the Ig heavy chain.

Hence, in other embodiments, anti-ROR1/anti-NKG2D antibodies have at least (1) a first antigen-binding site specific for ROR1, (2) a second antigen-binding site at least specific for NKG2D, or a bispecific antigen-binding site for ROR1 and NKG2D, and (3) the hinge and Fc domains of two or more chains of Ig, which can be dimerized or multimerized, wherein at least one heaving chain of the Ig may further comprise an antigen-binding site specific for ROR1, preferably on the C-terminus of the Ig heavy chain.

Hence, in other embodiments, anti-ROR1/anti-CD16 antibodies have at least (1) a first antigen-binding site specific for ROR1, (2) a second antigen-binding site at least specific for CD16, or a bispecific antigen-binding site for ROR1 and CD16, and (3) the hinge and Fe domains of two or more chains of Ig, which can be dimerized or multimerized, wherein at least one heaving chain of the Ig may further comprise an antigen-binding site specific for ROR1, preferably on the C-terminus of the Ig heavy chain.

Hence, in other embodiments, anti-ROR1/anti-NKp46 antibodies have at least (1) a first antigen-binding site specific for ROR1, (2) a second antigen-binding site at least specific for NKp46, or a bispecific antigen-binding site for ROR1 and NKp46, and (3) the hinge and Fc domains of two or more chains of Ig, which can be dimerized or multimerized, wherein at least one heaving chain of the Ig may further comprise an antigen-binding site specific for ROR1, preferably on the C-terminus of the Ig heavy chain.

Alternatively, isolated multispecific antibodies, or multispecific antigen-binding fragments thereof, are provided, which contains at least one ROR1-binding site, at least one CD3-binding site, and a scaffold protein such as human serum albumin. Alternatively, isolated multispecific antibodies, or multispecific antigen-binding fragments thereof, are provided, which contains at least one ROR1-binding site, at least one NKG2D-binding site, and a scaffold protein such as human serum albumin. Alternatively, isolated multispecific antibodies, or multispecific antigen-binding fragments thereof, are provided, which contains at least one ROR1-binding site, at least one CD16-binding site, and a scaffold protein such as human serum albumin. Alternatively, isolated multispecific antibodies, or multispecific antigen-binding fragments thereof, are provided, which contains at least one ROR1-binding site, at least one NKp46-binding site, and a scaffold protein such as human serum albumin.

The isolated multispecific antibodies, or multispecific antigen-binding fragments thereof, can comprise:
a) a first antigen-binding site that immunospecifically binds ROR1, the first antigen-binding site comprising one or more of:
2A11 (the peptide having an amino acid sequence of SEQ ID NO:7),
a variant of 2A11 disclosed above,
5A1 (the peptide having an amino acid sequence of SEQ ID NO:8),
a variant of 5A1 disclosed above (such as a polypeptide having an amino acid sequence of any one of SEQ ID NOs: 11-15),
three, two, or at least one of the CDR1 (having an amino acid sequence of SEQ ID NO:1), CDR2 (having an amino acid sequence of SEQ ID NO:2), and CDR3 (having an amino acid sequence of SEQ ID NO:3) of 2A11,
one or more variants of the three, two, or at least one of the CDR1 (having an amino acid sequence of SEQ ID NO: 1), CDR2 (having an amino acid sequence of SEQ ID NO:2), and CDR3 (having an amino acid sequence of SEQ ID NO:3) of 2A11,
three, two, or at least one of the CDR1 (having an amino acid sequence of SEQ ID NO:4), CDR2 (having an amino acid sequence of SEQ ID NO:5), and CDR3 (having an amino acid sequence of SEQ ID NO:6) of 5A1, and
one or more variants of the three, two, or at least one of the CDR1 (having an amino acid sequence of SEQ ID NO: 4), CDR2 (having an amino acid sequence of SEQ ID NO:5), and CDR3 (having an amino acid sequence of SEQ ID NO:6) of 5A1; and
b) a second antigen-binding site that immunospecifically binds CD3, CD16, NKp46, or NKG2D.

In various embodiments, the second antigen-binding site immunospecifically binds to CD3. In various embodiments, the antigen-binding site that immunospecifically binds to NKG2D.

In other embodiments, the isolated multispecific antibodies, or multispecific antigen-binding fragments thereof, can comprise:
a1) a first antigen-binding site comprising a polypeptide selected from Table 7, OR
a2) a first antigen-binding site comprising CDR1, CDR2, and CDR3 selected from Table &A, OR
a3) a first antigen-binding site comprising CDR1, CDR2, and CDR3 selected from Table 8B; and
b) a second antigen-binding site that immunospecifically binds to CD3, CD16, NKp46, or NKG2D.

In various embodiments, the second antigen-binding site immunospecifically binds to CD3. In various embodiments, the antigen-binding site that immunospecifically binds to NKG2D.

In other embodiments, the isolated multispecific antibodies, or multispecific antigen-binding fragments thereof, can comprise:
a1) a first antigen-binding site comprising a variant of CDR1, a variant of CDR2, and a variant of CDR3 selected from Table 8A, OR
a2) a first antigen-binding site comprising a variant of CDR1, a variant of CDR2, and a variant of CDR3 selected from Table 8B; and
b) a second antigen-binding site that immunospecifically binds to CD3, CD16, NKp46, or NKG2D.

In various embodiments, the multispecific antibody comprises two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide selected from Table 7. In various embodiments the two or more is 2, 3, 4, 5 or 6.

In various embodiments, the multispecific antibody comprises two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide having a sequence as set forth in SEQ ID NO:11-15. In various embodiments the two or more is 2, 3, 4, 5 or 6.

In various embodiments, the multispecific antibody comprises two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide having a sequence as set forth in SEQ ID NO:11, 14-70. In various embodiments the two or more is 2, 3, 4, 5 or 6.

Variants of CDR1, CDR2, and CDR3 are as discussed herein. In various embodiments, the first antigen-binding site further comprises a FWR1, FWR2, FWR3 and FWR4 selected from Table &B. In various embodiments, the first antigen-binding site further comprises a FWR1, FWR2, FWR3 and FWR4 from a human IgG. In various embodiments, the second antigen-binding site immunospecifically binds to CD3. In various embodiments, the antigen-binding site that immunospecifically binds to NKG2D.

Suitable antigen-binding sites can be in the form of a VHH, scFv, Fab, (Fab′)₂, one or more CDRs, or a fusion (tandem sequence optionally connected via a linker) of one or more of the VHH, scFv, Fab, (Fab′)₂, or CDRs. Suitable antigen-binding sites that immunospecifically bind to ROR1 include any of the above disclosed anti-ROR1 antibodies or ROR1-binding fragments thereof. Suitable antigen-binding sites that immunospecifically bind to CD3 can be derived from CD3 antibodies disclosed in one or more publications including but not limited to U.S. Pat. No. 8,236,308, U.S. Patent App. Pub. Nos. 2010/0260668, 2013/0018174, 2012/0321626, 2013/0060011, 2013/0058936, 2013/0078249, and 2013/0058937.

In some embodiments, the antigen-binding site of the multispecific antibodies disclosed herein is connected to an Ig heavy chain constant region that contains a mutation. In some aspects, “knob-in-hole” (KiH) mutations are present in the CH3 domains of two arms (or chains) of Ig heavy chain constant regions, so that the two can heterodimerize. This structural feature in the polypeptide arms allows for assembly of two half antibodies (e.g., Fc heterodimer; and VH-CH and VL-CL domains). For example, a heteromultimer (including a heterodimer) may comprise a first polypeptide and a second polypeptide each comprising a CH3 domain, wherein the polypeptides meet at an engineered interface within the CH3 domain, and the first polypeptide contains an engineered protuberance (“knob”) in the interface with at least one contact residue replaced with an import residue having a larger side chain volume than the original residue, and the second polypeptide contains an engineered cavity (“hole”) in the interface with at least one contact residue replaced with an import residue having a smaller side chain volume than the original residue. In some embodiments, the engineered interface of a heteromultimer includes at least two protuberance-into-cavity mutant pairs. Volumes and accessible surface areas of each amino acid are described in A. A. Zamyatnin, Prog. Biophys. Mol. Biol. 24:107-123, 1972 and C. Chothia, J. Mol. Biol. 105:1-14, 1975. For example, import residues for the formation of a protuberance can be arginine I, phenylalanine (F), tyrosine (Y) and tryptophan (W); and preferably the original residue for the formation of the protuberance has a small side chain volume, such as alanine, asparagine, aspartic acid, glycine, serine, threonine or valine. As another example, import residues for the formation of a cavity can be alanine (A), serine(S), threonine (T) and valine (V); and preferably the original residue for the formation of the cavity has a large side chain volume, such as tyrosine, arginine, phenylalanine or tryptophan. For example, a T366W mutation in CH3 domain for the “knob”/protuberance chain, and a T366S/L368A/Y407V mutation in CH3 domain for the “hole”/cavity chain. Additionally, the KiH configuration may be coupled further mutations to permit S—S disulfide linkage between the two chains.

In some embodiments, the two or more arms (or chains) of immunoglobulin heavy chain constant regions (e.g., Fc polypeptides) can contain another symmetric-to-asymmetric steric complementarity design (e.g., HA-TF, ZW1), a charge-to-charge swap interaction (DD-KK), a charge-to-steric complementarity swap plus additional long-range electrostatic interaction (e.g., EW-RVT), or an isotype strand swap design (e.g., strand-exchange engineered domain (SEED)), or Xmab, 7.8.60, Electrostatic Steering, A107, or Duobody, so as to form heterodimeris/heteromultimers. Further description of these configurations and exemplary mutations/residues are seen in, for example, Front Immunol. 2016; 7:394.

Further embodiments provide that a multispecific antibody may further comprise one or more linkers, such as amino acid linkers. In some embodiments, at least one linker is between the Fc or the HSA and at least one of the antigen-binding sites. In some embodiments, at least one linker is positioned between two antigen-binding sites.

In some embodiments, a multispecific antibody comprises (1) a “knob-in-hole” IgG Fc domains with (2) a CD3-specific Fab on the N-terminus of the “knob” chain (or the “hole” chain) of the IgG Fc domain and (3) two ROR1-specific VHH fused in tandem, optionally connected via a linker (VHH-linker-VHH), on the N-terminus of the “hold” chain (or the “knob chain, respectively) of the IgG Fc domain. The two ROR1-specific VHH fused in tandem herein may be two 2A11, two 5A1, or one 2A11 and one 5A1, two variants of 2A11, or two variants of 5A1. Exemplary multispecific antibodies of these embodiments include fusion proteins of FUSE-211, and FUSE-393, whose amino acid sequences are shown in Table 1. In some embodiments, the Fc domain is selected from Table 10A.

In some embodiments, a multispecific antibody comprises (1) a “knob-in-hole” IgG Fc domains with (2) a CD3-specific Fab (comprising one constant and one variable domain of each of the CD3-specific heavy and the CD3-specific light chain) on the N-terminus of the “knob” chain (or the “hole” chain) of the IgG Fc domain, (3) a first ROR1-specific VHH fused to the N-terminus of the “hole” chain (or the “knob” chain, respectively), and (4) a second ROR1-specific VHH fused to the C-terminus of the CD3-specific light chain. The first and second ROR1-specific VHH can be identical or different, independently selected from 2A11, SAL, or a variant of 2A11 or a variant of 5A1. Exemplary multispecific antibodies of these embodiments include fusion protein FUSE-394, whose amino acid sequence is shown in Table 1. In some embodiments, the Fc domain is selected from Table 10A.

EVQLVESGGGLVQPGGSLRLSCAASGFTFSTYAMNWVRQAPGKGLEWVARIRSKYNNYAT YYADSVKDRFTISRDDSKNTLYLQMNSLRAEDTAVYYCARHGNFGNSYVSWFAYWGQGTMVTVSSASTKGP SVFPLAPSSKSTSGGTAALGCLVKDYFPEPVTVSWNSGALTSGVHTFPAVLQSSGLYSLSSVVTVPSSSLGTQTYI CNVNHKPSNTKVDKKV (SEQ ID NO:208) is an exemplary heavy chain of an anti-CD3 Fab, and SEQ ID NO: 155 is an exemplary light chain of an anti-CD3 Fab. Tin various embodiments, the anti-CD3 Fab is constant and sometimes present on the knob Fc and sometimes present on the hole Fc.

In some embodiments, a multispecific antibody comprises (1) a “knob-in-hole” IgG Fc domains with (2) a CD3-specific Fab (comprising one constant and one variable domain of each of the CD3-specific heavy and the CD3-specific light chain) fused to the N-terminal of the “knob” chain (or the “hole” chain) of the IgG Fc domain, (3) two ROR1-specific VHH fused in tandem to the N-terminus of the “hole” chain (or the “knob” chain, respectively) of the IgG Fc domain, and (4) a third specific VHH fused to the C-terminus of the CD3-specific light chain. The two, fused-in-tandem ROR1-specific VHH and the third ROR1-specific VHH may be identical or different, each independently selected from 2A11, 5A1, or a variant of 2A11 or a variant of 5A1. In other embodiments, the first and second ROR1-specific VHH can be identical or different, independently selected from Table 7. In some embodiments, the Fc domain is selected from Table 10A.

In some embodiments, a multispecific antibody comprises (1) a “knob-in-hole” IgG Fc domains with (2) a CD3-specific Fab fused to the N-terminus of the “knob” chain (or the “hole” chain) of the IgG Fc domains. (3) a first ROR1-specific VHH fused to the N-terminus of the “hole” chain (or the “key” chain, respectively) of the IgG Fc domains, and (4) a second ROR1-specific VHH fused to the N-terminus of the CD3-specific light chain of the Fab. The first and second ROR1-specific VHH can be identical or different, independently selected from 2A11, 5A1, or a variant of 2A11 or a variant of 5A1. In other embodiments, the first and second ROR1-specific VHH can be identical or different, independently selected from Table 7. In some embodiments, the Fc domain is selected from Table 10A.

In some embodiments, a multispecific antibody comprises (1) a “knob-in-hole” IgG Fc domains with (2) a CD3-specific Fab fused to the N-terminus of the “knob” chain (or the “hole” chain) of the IgG Fc domains, (3) a first ROR1-specific scFV fused to the N-terminus of the “hole” chain (or the “knob” chain, respectively) of the IgG Fc domains, and (4) a second ROR1-specific scFV fused to the C-terminus of the “hole” (or the “knob” chain, respectively) of the IgG Fc domains. The first and second ROR1-specific scFv can be identical or different. In some embodiments, the Fc domain is selected from Table 10A.

In some embodiments, a multispecific antibody comprises (1) a “knob-in-hole” IgG Fc domains with (2) a CD3-specific Fab fused to the N-terminus of the “hole” chain (or the “knob” chain) of the IgG Fc domains, (3) a first ROR1-specific VHH fused to the N-terminus of the “knob” chain (or the “hole” chain) of the IgG Fc domains, and (4) a second ROR1-specific VHH fused to the C-terminus of the CD3-specific light chain of the Fab. The first and second ROR1-specific VHH can be identical or different, independently selected from 2A11, 5A1, or a variant of 2A11 or a variant of 5A1. In other embodiments, the first and second ROR1-specific VHH can be identical or different, independently selected from Table 7. In some embodiments, the Fc domain is selected from Table 10A.

In various embodiments, the disclosed multispecific antibodies (e.g., at least with immunospecificity towards ROR1 and CD3) can bind to ROR1 with an apparent binding affinity that generally increases (i.e., corresponding to a binding EC₅₀—a concentration of the antibody that results in 50% of maximum binding to a certain population of ROR1-expressing target cells—that generally decreases) with an increase in the cell surface density of ROR1 of the target cells. That is, the multispecific antibodies can in various embodiments have a greater binding avidity towards ROR1-expressing cells with a higher expression level of ROR1 than towards cells with a lower or zero expression level of ROR1.

In various embodiments, a system comprising the disclosed multispecific antibodies (e.g., at least with immunospecificity towards ROR1 and CD3) and a population of CD3-positive immune effector cells or a CD3-positive fraction of mononuclear cells or a population of mononuclear cells comprising a CD3-positive fraction can induce cytotoxicity against ROR1-positive cells, i.e., ROR1-expressing tumor cells.

In some embodiments, the multispecific antibodies comprising an antigen-binding site that immunospecifically binds CD3 can bind CD3-epsilon on primary human T cells and/or primary cynomolgus T cells. In some embodiments, the multispecific antibodies comprising an antigen-binding site that immunospecifically binds CD3 activates cytotoxicity of primary human CD3+ T cells and/or primary cynomolgus CD3+ T cells. In general, CD8+ T cells are cytotoxic whereas CD4+ T cells primary secrete cytokines to “help” CD8+ T cells. In some instances, CD4+ T cells have also been reported to be cytotoxic (David Oh, et al., Immunity, Volume 54, Issue 12, 14 Dec. 2021, Pages 2701-2711). Cytotoxicity generally is associated with killing of target cells via the perforin/Granzyme pathway that activates Caspase 3 and subsequent target cell apoptosis. Other mechanisms of target killing include but are not limited to activation of target cell express Fas, TNF-R, and DR4. TCEs directly induce cytotoxicity and may also indirectly induce the other said mechanisms of target killing (Sandra Ross et al., PLoS One, 2017 Aug. 24; 12(8):e0183390).

In some embodiments, the disclosed multispecific antibodies can induce cytotoxicity against ROR1-positive cells, i.e., ROR1-expressing tumor cells, in the presence of a population of CD3-positive immune effector cells, a CD3-positive fraction of mononuclear cells, or a population of mononuclear cells comprising a CD3-positive fraction. In some embodiments, the disclosed multispecific antibodies can induce a greater cytotoxicity against ROR1-positive cells that have a higher expression level of ROR1 than against ROR1-negative cells or ROR1-positive cells that have a lower expression level of ROR1, when in the presence of a population of CD3-positive immune effector cells, a CD3-positive fraction of mononuclear cells, or a population of mononuclear cells comprising a CD3-positive fraction.

Further embodiments provide that in place of, or in addition to, having CD3-specificity, a multispecific antibody of the invention may have a specificity against another activation receptor and/or costimulatory receptor and/or co-activation receptor typically expressed on an immune cell, besides having a specificity against ROR1. For example, an activation receptor can be expressed on a T cell, such that the multispecific antibody may be a T cell engager that immunospecifically binds to the activation receptor on the T cell and activates the cytotoxicity or a cytokine expression property of the T cell. Alternatively, an activation receptor can be expressed on a natural killer (NK) cell, such that the multispecific antibody may be a NK cell engager that immunospecifically binds to the activation receptor on the NK cell and activates the cytotoxicity of the NK cell. Additionally, an activation receptor may be expressed on multiple immune cells such as T cells, NK cells and Dendritic cells (DC). Hence, the multispecific antibodies may be trifunctional immune cell engagers, or trispecific antibodies, which bind to two or more activation receptors (such as two or more of CD3, CD16, γ9 TCR, δ2 TCR or δ1 TCR, NKp46, CD137 and CD40), thereby inducing activity of the immune cells, and also bind to ROR1-positive cells, thereby directing the immune cell activity towards the ROR-positive cells. In various embodiments the two or more is 2, 3, 4, 5 or 6.

Exemplary activation receptors that can be immunospecifically bound by an antigen-binding site of the multispecific antibodies disclosed herein include, but are not limited to, CD16, γ9 TCR, δ2 TCR or δ1 TCR, NKp46, CD137, CD40 or NKG2D. Exemplary antigen-binding fragments against said activation and/or costimulatory and/or co-activation receptors include scFv, VH, VL, VHH, Fab, etc., described in various publications including U.S. Pat. No. 9,035,026 and Gauthier et al., 2019, Cell 177, 1701-1713. Additional exemplary antigen-binding antibodies and/or fragments against said activation and/or costimulatory and/or co-activation receptors include those noted in Table 11. Of those listed as the antigen-binding antibodies, their VHH, Fab regions, or single-chain variable fragments (scFv) can be used as the antigen-binding site of the multispecific antibodies disclosed herein.

Additional embodiments provide ROR1-specific antibody-drug conjugates (ADCs), which comprises an anti-ROR1 antibody, or an ROR1-binding fragment thereof, disclosed herein, and a drug unit that is conjugated or bound to the anti-ROR1 antibody or ROR1-binding fragment thereof. Optionally, a linker unit is positioned between the anti-ROR1 antibody or ROR1-binding fragment thereof and the drug unit, and the linker unit may be an amino acid linker or a chemical moiety linker. Exemplary drug units can be cytotoxic agents such as antitumor drugs or chemotherapy agents, including small molecules as well as siRNA.

### Methods of Killing Cancer Cells and Methods of Treating Cancer

Disclosed herein are methods of treating a subject having cancer, the method comprising administering to the subject a therapeutically effective amount of a multispecific antibody, or a multispecific antigen-binding fragment thereof, disclosed herein.

Also provided are methods of killing cancer cells, inhibiting the growth or proliferation of the cancer cells or other disease cells that express ROR1, and/or activating cytotoxicity of an immune effector cell towards the cancer cells, which comprise administering an effective amount of a multispecific antibody, or a multispecific antigen-binding fragment thereof, disclosed herein, or contacting the effective amount of the multispecific antibody (or multispecific antigen-binding fragment thereof) disclosed herein with the cancer cells or diseased cells in the presence of the immune effector cell, to kill or inhibit the growth or proliferation of cancer cells or ROR1-expressing disease cells, wherein the immune effector cell expresses an activation receptor, and the multispecific antibody or multispecific antigen-binding fragment thereof is capable of immunospecifically binding to the activation receptor and to ROR1.

In some embodiments, the cancer is a ROR1-expressing cancer, such as lung cancer, hematological cancer, breast cancer, prostate cancer, pancreatic cancer, colon cancer, ovarian cancer, renal cancer, uterine cancer, bladder cancer, kidney cancer, melanoma, thyroid cancer, myeloid leukemia, mantle cell lymphoma, or multiple myeloma. The ROR1-expressing cancer can be a lung cancer, such as non-small cell lung cancer (NSCLC) or small cell lung cancer (SCLC). The ROR1-expressing cancer can be a hematological cancer, such as acute myeloid leukemia (AML), myelodysplastic syndrome (MDS, low or high risk), acute lymphocytic leukemia (ALL, including all subtypes), diffuse large B-cell lymphoma (DLBCL), chronic myeloid leukemia (CML), or blastic plasmacytoid dendritic cell neoplasm (DPDCN). The ROR1-expressing cancer can be breast cancer. The ROR1-expressing cancer can be prostate cancer. The ROR1-expressing cancer can be pancreatic cancer. The ROR1-expressing cancer can be colon cancer. The ROR1-expressing cancer can be ovarian cancer. The ROR1-expressing cancer can be renal cancer. The ROR1-expressing cancer can be uterine cancer. The ROR1-expressing cancer can be melanoma.

In some embodiments, the multispecific antibody or multispecific antigen-binding fragment thereof can be administered to the subject as a pharmaceutical composition. The pharmaceutical compositions provided herein can comprise: a) an effective amount of a multispecific antibody or multispecific antigen-binding fragment thereof, and b) a pharmaceutically acceptable carrier, which may be inert or physiologically active. As used herein, the term “pharmaceutically acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable carriers include: (1) Dulbecco's phosphate buffered saline, pH about 7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as TWEEN 20®.

In additional embodiments, the multispecific antibody, multispecific antigen-binding fragment, or composition comprising the same may also contain a further therapeutic agent/therapy, as necessary for the particular disorder being treated. Preferably, the multispecific antibody or multispecific antigen-binding fragment thereof and the further therapeutic agent have complementary activities that do not adversely affect each other. In some embodiments, the further therapeutic agent/therapy is a chemotherapeutic agent or a radiation therapy. Combined administration of the disclosed multispecific antibodies or multispecific antigen-binding fragments thereof and the other therapeutic agent may be simultaneous, separate or sequential, in any order. For simultaneous administration, the agents may be administered as one composition or as separate compositions, as appropriate.

The dose of the multispecific antibody, multispecific antigen-binding fragment, or composition comprising the same depends on the desired effect, the duration of the treatment, and the route of administration used. For example, a therapeutically effective amount of a multispecific antibody disclosed herein includes one or more doses, wherein a dose is in the range of about 10-50 mg, 50-100 mg, 100-150 mg, 150-200 mg, 100-200 mg, 200-300 mg, 300-400 mg, 400-500 mg, 500-600 mg, 600-700 mg, 700-800 mg, 800-900 mg, 900-1000 mg, 1000-1100 mg, 1100-1200 mg, 1200-1300 mg, 1300-1400 mg, 1400-1500 mg, 1500-1600 mg, 1600-1700 mg, 1700-1800 mg, 1800-1900 mg, 1900-2000 mg, 2000-2100 mg, 2100-2200 mg, 2200-2300 mg, 2300-2400 mg, 2400-2500 mg, 2500-2600 mg, 2600-2700 mg, 2700-2800 mg, 2800-2900 mg or 2900-3000 mg. In another embodiment, a therapeutically effective amount of a multispecific antibody disclosed herein includes one or more doses, wherein a dose is in the range of 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 10-50 mg/kg, 50-100 mg/kg, 100-150 mg/kg, 150-200 mg/kg, 100-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg or 2900-3000 mg/kg. In some embodiments, a therapeutically effective amount includes two, three, or more doses administered on a daily, weekly, biweekly, monthly, quarterly, or yearly frequency; or continued when evaluation of the severity of the disease shows improvement, compared to before the last dose, until the disease is successfully treated or the subject shows no symptoms or signs of the disease.

TABLE 1
Amino acid sequences of exemplary fusion proteins. Polypeptide 1 comprises
the ‘hole’ of the KiH Fe; Polypeptide 2 comprises the ‘knob’ of the KiH Fc;
and Polypeptide 3 comprises the light chain. The VHH may be included in
Polypeptide 1, polypeptide 2 and/or polypeptide 3 with a linker between them.
In this table the anti-CD3 domain is on the N-terminal of the knob Fc
or hole Fc.

| | Polypeptide 1 | Polypeptide 2 | Polypeptide 3 |
|---|---|---|---|
| FUSE-211 | QVQLQESGGGSVPAGGSLRL | EVQLVESGGGLVQPGGSLRL | QAVVTQEPSLTVSPGGTVTL |
| | SCAASGSTYSANCMGWFRQA | SCAASGFTFSTYAMNWVRQA | TCRSSTGAVTTSNYANWVQQ |
| | PGKEREEVASMSIRSGRTYY | PGKGLEWVARIRSKYNNYAT | KPGQAPRGLIGGINKRAPGT |
| | SDSVKGRFTISQDGSKNTLY | YYADSVKDRFTISRDDSKNT | PARFSGSLLGGKAALTLSGV |
| | LQLNSLKAEDTALYYCAAAY | LYLQMNSLRAEDTAVYYCAR | QPEDEAEYYCALWYSNLWVF |
| | GGSRCVYNYRGQGTQVTVSS | HGNFGNSYVSWFAYWGQGTM | GGGTKLTVLGQPKAAPSVTL |
| | GGGGSGGGGSGGGGSQVQLQ | VTVSSASTKGPSVFPLAPSS | FPPSSEELQANKATLVCLIS |
| | ESGGGSVPAGGSLRLSCAAS | KSTSGGTAALGCLVKDYFPE | DFYPGAVTVAWKADSSPVKA |
| | GSTYSANCMGWFRQAPGKER | PVTVSWNSGALTSGVHTFPA | GVETTTPSKQSNNKYAASSY |
| | EEVASMSIRSGRTYYSDSVK | VLQSSGLYSLSSVVTVPSSS | LSLTPEQWKSHRSYSCQVTH |
| | GRFTISQDGSKNTLYLQLNS | LGTQTYICNVNHKPSNTKVD | EGSTVEKTVAPTECS |
| | LKAEDTALYYCAAAYGGSRC | KKVEPKSCDKTHTCPPCPAP | (SEQ ID NO: 155) |
| | VYNYRGQGTQVTVSSEPKSS | EAAGGPSVFLFPPKPKDTLM | |
| | DKTHTCPPCPAPEAAGGPSV | ISRTPEVTCVVVDVSHEDPE | |
| | FLFPPKPKDTLMISRTPEVT | VKFNWYVDGVEVHNAKTKPR | |
| | CVVVDVSHEDPEVKFNWYVD | EEQYNSTYRVVSVLTVLHQD | |
| | GVEVHNAKTKPREEQYNSTY | WLNGKEYKCKVSNKALPAPI | |
| | RVVSVLTVLHQDWLNGKEYK | EKTISKAKGQPREPQVYTLP | |
| | CKVSNKALPAPIEKTISKAK | PSREEMTKNQVSLWCLVKGF | |
| | GQPREPQVYTLPPSREEMTK | YPSDIAVEWESNGQPENNYK | |
| | NQVSLSCAVKGFYPSDIAVE | TTPPVLDSDGSFFLYSKLTV | |
| | WESNGQPENNYKTTPPVLDS | DKSRWQQGNVFSCSVMHEAL | |
| | DGSFFLVSKLTVDKSRWQQG | HNHYTQKSLSLSPGK | |
| | NVFSCSVMHEALHNRFTQKS | (SEQ ID NO: 154) | |
| | LSLSPGK | | |
| | (SEQ ID NO: 157) | | |
| FUSE-393 | QVQLQESGGGSVQAGGSLKL | EVQLVESGGGLVQPGGSLRL | QAVVTQEPSLTVSPGGTVTL |
| | SCTASGYTNRLKCMGWFRQA | SCAASGFTFSTYAMNWVRQA | TCRSSTGAVTTSNYANWVQQ |
| | PGKEREEIATISTGTGNTYY | PGKGLEWVARIRSKYNNYAT | KPGQAPRGLIGGTNKRAPGT |
| | ADSVKGRFTFSQDKVKNTVY | YYADSVKDRFTISRDDSKNT | PARFSGSLLGGKAALTLSGV |
| | LQMNTLKPDDTGMYYCAADV | LYLQMNSLRAEDTAVYYCAR | QPEDEAEYYCALWYSNLWVF |
| | RPDGTTCHYNSGGQGTQVTV | HGNFGNSYVSWFAYWGQGTM | GGGTKLTVLGQPKAAPSVTL |
| | SSGGGGSGGGGSGGGGSQVQ | VTVSSASTKGPSVFPLAPSS | FPPSSEELQANKATLVCLIS |
| | LQESGGGSVQAGGSLKLSCT | KSTSGGTAALGCLVKDYFPE | DFYPGAVTVAWKADSSPVKA |
| | ASGYTNRLKCMGWFRQAPGK | PVTVSWNSGALTSGVHTFPA | GVETTTPSKQSNNKYAASSY |
| | EREEIATISTGTGNTYYADS | VLQSSGLYSLSSVVTVPSSS | LSLTPEQWKSHRSYSCQVTH |
| | VKGRFTFSQDKVKNTVYLQM | LGTQTYICNVNHKPSNTKVD | EGSTVEKTVAPTECS |
| | NTLKPDDTGMYYCAADVRPD | KKVEPKSCDKTHTCPPCPAP | (SEQ ID NO: 155) |
| | GTTCHYNSGGQGTQVTVSSE | EAAGGPSVFLFPPKPKDTLM | |
| | PKSSDKTHTCPPCPAPEAAG | ISRTPEVTCVVVDVSHEDPE | |
| | GPSVFLFPPKPKDTLMISRT | VKFNWYVDGVEVHNAKTKPR | |
| | PEVTCVVVDVSHEDPEVKFN | EEQYNSTYRVVSVLTVLHQD | |
| | WYVDGVEVHNAKTKPREEQY | WLNGKEYKCKVSNKALPAPI | |
| | NSTYRVVSVLTVLHQDWLNG | EKTISKAKGQPREPQVYTLP | |
| | KEYKCKVSNKALPAPIEKTI | PSREEMTKNQVSLWCLVKGF | |
| | SKAKGQPREPQVYTLPPSRE | YPSDIAVEWESNGQPENNYK | |
| | EMTKNQVSLSCAVKGFYPSD | TTPPVLDSDGSFFLYSKLTV | |
| | IAVEWESNGQPENNYKTTPP | DKSRWQQGNVFSCSVMHEAL | |
| | VLDSDGSFFLVSKLTVDKSR | HNHYTQKSLSLSPGK | |
| | WQQGNVFSCSVMHEALHNRF | (SEQ ID NO: 154) | |
| | TQKSLSLSPGK | | |
| | (SEQ ID NO: 158) | | |
| FUSE-394 | QVQLQESGGGSVQAGGSLKL | EVQLVESGGGLVQPGGSLRL | QAVVTQEPSLTVSPGGTVTL |
| | SCTASGYTNRLKCMGWFRQA | SCAASGFTFSTYAMNWVRQA | TCRSSTGAVTTSNYANWVQQ |
| | PGKEREEIATISTGTGNTYY | PGKGLEWVARIRSKYNNYAT | KPGQAPRGLIGGTNKRAPGT |
| | ADSVKGRFTFSQDKVKNTVY | YYADSVKDRFTISRDDSKNT | PARFSGSLLGGKAALTLSGV |
| | LQMNTLKPDDTGMYYCAADV | LYLQMNSLRAEDTAVYYCAR | QPEDEAEYYCALWYSNLWVF |
| | RPDGTTCHYNSGGQGTQVTV | HGNFGNSYVSWFAYWGQGTM | GGGTKLTVLGQPKAAPSVTL |
| | SSEPKSSDKTHTCPPCPAPE | VTVSSASTKGPSVFPLAPSS | FPPSSEELQANKATLVCLIS |
| | AAGGPSVFLFPPKPKDTLMI | KSTSGGTAALGCLVKDYFPE | DFYPGAVTVAWKADSSPVKA |
| | SRTPEVTCVVVDVSHEDPEV | PVTVSWNSGALTSGVHTFPA | GVETTTPSKQSNNKYAASSY |
| | KFNWYVDGVEVHNAKTKPRE | VLQSSGLYSLSSVVTVPSSS | LSLTPEQWKSHRSYSCQVTH |
| | EQYNSTYRVVSVLTVLHQDW | LGTQTYICNVNHKPSNTKVD | EGSTVEKTVAPTECSGGGGS |
| | LNGKEYKCKVSNKALPAPIE | KKVEPKSCDKTHTCPPCPAP | GGGGSGGGGSGGGGSQVQLQ |
| | KTISKAKGQPREPQVYTLPP | EAAGGPSVFLFPPKPKDTLM | ESGGGSVQAGGSLKLSCTAS |
| | SREEMTKNQVSLSCAVKGFY | ISRTPEVTCVVVDVSHEDPE | GYTNRLKCMGWFRQAPGKER |
| | PSDIAVEWESNGQPENNYKT | VKFNWYVDGVEVHNAKTKPR | EEIATISTGTGNTYYADSVK |
| | TPPVLDSDGSFFLVSKLTVD | EEQYNSTYRVVSVLTVLHQD | GRFTFSQDKVKNTVYLQMNT |
| | KSRWQQGNVFSCSVMHEALH | WLNGKEYKCKVSNKALPAPI | LKPDDTGMYYCAADVRPDGT |
| | NRFTQKSLSLSPGK | EKTISKAKGQPREPQVYTLP | TCHYNSGGQGTQVTVSS |
| | (SEQ ID NO: 159) | PSREEMTKNQVSLWCLVKGF | (SEQ ID NO: 156) |
| | | YPSDIAVEWESNGQPENNYK | |
| | | TTPPVLDSDGSFFLYSKLTV | |
| | | DKSRWQQGNVFSCSVMHEAL | |
| | | HNHYTQKSLSLSPGK | |
| | | (SEQ ID NO: 154) | |
| FUSE-399 | ELVMTQTPSSTSGAVGGTVT | EVQLVESGGGLVQPGGSLRL | QAVVTQEPSLTVSPGGTVTL |
| | INCQASQSIDSNLAWFQQKP | SCAASGFTFSTYAMNWVRQA | TCRSSTGAVTTSNYANWVQQ |
| | GQPPTLLIYRASNLASGVPS | PGKGLEWVARIRSKYNNYAT | KPGQAPRGLIGGTNKRAPGT |
| | RFSGSRSGTEYTLTISGVQR | YYADSVKDRFTISRDDSKNT | PARFSGSLLGGKAALTLSGV |
| | EDAATYYCLGGVGNVSYRTS | LYLQMNSLRAEDTAVYYCAR | QPEDEAEYYCALWYSNLWVF |
| | FGGGTEVVVKGGGGSGGGGS | HGNFGNSYVSWFAYWGQGTM | GGGTKLTVLGQPKAAPSVTL |
| | GGGGSGGGGSQSVKESEGDL | VTVSSASTKGPSVFPLAPSS | FPPSSEELQANKATLVCLIS |
| | VTPAGNLTLTCTASGSDIND | KSTSGGTAALGCLVKDYFPE | DFYPGAVTVAWKADSSPVKA |
| | YPISWVRQAPGKGLEWIGFI | PVTVSWNSGALTSGVHTFPA | GVETTTPSKQSNNKYAASSY |
| | NSGGSTWYASWVKGRFTISR | VLQSSGLYSLSSVVTVPSSS | LSLTPEQWKSHRSYSCQVTH |
| | TSTTVDLKMTSLTTDDTATY | LGTQTYICNVNHKPSNTKVD | EGSTVEKTVAPTECS |
| | FCARGYSTYYCDFNIWGPGT | KKVEPKSCDKTHTCPPCPAP | (SEQ ID NO: 155) |
| | LVTISSEPKSSDKTHTCPPC | EAAGGPSVFLFPPKPKDTLM | |
| | PAPEAAGGPSVFLFPPKPKD | ISRTPEVTCVVVDVSHEDPE | |
| | TLMISRTPEVTCVVVDVSHE | VKFNWYVDGVEVHNAKTKPR | |
| | DPEVKFNWYVDGVEVHNAKT | EEQYNSTYRVVSVLTVLHQD | |
| | KPREEQYNSTYRVVSVLTVL | WLNGKEYKCKVSNKALPAPI | |
| | HQDWLNGKEYKCKVSNKALP | EKTISKAKGQPREPQVYTLP | |
| | APIEKTISKAKGQPREPQVY | PSREEMTKNQVSLWCLVKGF | |
| | TLPPSREEMTKNQVSLSCAV | YPSDIAVEWESNGQPENNYK | |
| | KGFYPSDIAVEWESNGQPEN | TTPPVLDSDGSFFLYSKLTV | |
| | NYKTTPPVLDSDGSFFLVSK | DKSRWQQGNVFSCSVMHEAL | |
| | LTVDKSRWQQGNVFSCSVMH | HNHYTQKSLSLSPGK | |
| | EALHNRFTQKSLSLSPGK | (SEQ ID NO: 154) | |
| | (SEQ ID NO: 160) | | |
| FUSE-489 | EVQLVESGGGLVQPGGSLRL | QVQLQESGGGLVQPGGSLRL | QAVVTQEPSLTVSPGGTVTL |
| | SCAASGFTFSTYAMNWVRQA | SCTASGYTNRLKCMGWVRQA | TCRSSTGAVTTSNYANWVQQ |
| | PGKGLEWVARIRSKYNNYAT | PGKEREEVATISTGTGNTYY | KPGQAPRGLIGGINKRAPGT |
| | YYADSVKDRFTISRDDSKNT | ADSVKGRFTISRDNSKNTLY | PARFSGSLLGGKAALTLSGV |
| | LYLQMNSLRAEDTAVYYCAR | LQMNSLRAEDTAMYYCAADV | QPEDEAEYYCALWYSNLWVF |
| | HGNFGNSYVSWFAYWGQGTM | RPDGTTCHYNSGGQGTQVTV | GGGTKLTVLGQPKAAPSVTL |
| | VTVSSASTKGPSVFPLAPSS | SSEPKSSDKTHTCPPCPAPE | FPPSSEELQANKATLVCLIS |
| | KSTSGGTAALGCLVKDYFPE | AAGGPSVFLFPPKPKDTLMI | DFYPGAVTVAWKADSSPVKA |
| | PVTVSWNSGALTSGVHTFPA | SRTPEVTCVVVDVSHEDPEV | GVETTTPSKQSNNKYAASSY |
| | VLQSSGLYSLSSVVTVPSSS | KFNWYVDGVEVHNAKTKPRE | LSLTPEQWKSHRSYSCQVTH |
| | LGTQTYICNVNHKPSNTKVD | EQYNSTYRVVSVLIVLHQDW | EGSTVEKTVAPTECSGGGGS |
| | KKVEPKSCDKTHTCPPCPAP | INGKEYKCKVSNKALPAPIE | GGGGSGGGGSGGGGSQVQLQ |
| | EAAGGPSVFLFPPKPKDTLM | KTISKAKGQPREPQVYTLPP | ESGGGLVQPGGSLRLSCTAS |
| | ISRTPEVTCVVVDVSHEDPE | SREEMTKNQVSLWCLVKGFY | GYTNRLKCMGWVRQAPGKER |
| | VKFNWYVDGVEVHNAKTKPR | PSDIAVEWESNGQPENNYKT | EEVATISTGTGNTYYADSVK |
| | EEQYNSTYRVVSVLTVLHQD | TPPVLDSDGSFFLYSKLTVD | GRFTISRDNSKNTLYLQMNS |
| | WLNGKEYKCKVSNKALPAPI | KSRWQQGNVFSCSVMHEALH | LRAEDTAMYYCAADVRPDGT |
| | EKTISKAKGQPREPQVYTLP | NHYTQKSLSLSPGK | TCHYNSGGQGTQVTVSS |
| | PSREEMTKNQVSLSCAVKGF | (SEQ ID NO: 161) | (SEQ ID NO: 162) |
| | YPSDIAVEWESNGQPENNYK | | |
| | TTPPVLDSDGSFFLVSKLTV | | |
| | DKSRWQQGNVFSCSVMHEAL | | |
| | HNRFTQKSLSLSPGK | | |
| | (SEQ ID NO: 153) | | |
| FUSE-494 | EVQLVESGGGLVQPGGSLRL | EVQLLESGGGLVQPGGSLRL | QAVVTQEPSLTVSPGGTVTL |
| | SCAASGFTFSTYAMNWVRQA | SCAASGYTNRLKCMGWFRQA | TCRSSTGAVTTSNYANWVQQ |
| | PGKGLEWVARIRSKYNNYAT | PGKEREEVSTISTGTGNTYY | KPGQAPRGLIGGTNKRAPGT |
| | YYADSVKDRFTISRDDSKNT | ADSVKGRFTISQDKSKNTLY | PARFSGSLLGGKAALTLSGV |
| | LYLQMNSLRAEDTAVYYCAR | LRMNSLRAEDTALYYCAADV | QPEDEAEYYCALWYSNLWVF |
| | HGNFGNSYVSWFAYWGQGTM | RPDGTTCHYNSGGQGTQVTV | GGGTKLTVLGQPKAAPSVTL |
| | VTVSSASTKGPSVFPLAPSS | SSEPKSSDKTHTCPPCPAPE | FPPSSEELQANKATLVCLIS |
| | KSTSGGTAALGCLVKDYFPE | AAGGPSVFLFPPKPKDTLMI | DFYPGAVTVAWKADSSPVKA |
| | PVTVSWNSGALTSGVHTFPA | SRTPEVTCVVVDVSHEDPEV | GVETTTPSKQSNNKYAASSY |
| | VLQSSGLYSLSSVVTVPSSS | KFNWYVDGVEVHNAKTKPRE | LSLTPEQWKSHRSYSCQVTH |
| | LGTQTYICNVNHKPSNTKVD | EQYNSTYRVVSVLTVLHQDW | EGSTVEKTVAPTECSGGGGS |
| | KKVEPKSCDKTHTCPPCPAP | LNGKEYKCKVSNKALPAPIE | GGGGSGGGGSGGGGSEVQLL |
| | EAAGGPSVFLFPPKPKDTLM | KTISKAKGQPREPQVYTLPP | ESGGGLVQPGGSLRLSCAAS |
| | ISRTPEVTCVVVDVSHEDPE | SREEMTKNQVSLWCLVKGFY | GYTNRLKCMGWFRQAPGKER |
| | VKFNWYVDGVEVHNAKTKPR | PSDIAVEWESNGQPENNYKT | EEVSTISTGTGNTYYADSVK |
| | EEQYNSTYRVVSVLTVLHQD | TPPVLDSDGSFFLYSKLTVD | GRFTISQDKSKNTLYLRMNS |
| | WLNGKEYKCKVSNKALPAPI | KSRWQQGNVFSCSVMHEALH | LRAEDTALYYCAADVRPDGT |
| | EKTISKAKGQPREPQVYTLP | NHYTQKSLSLSPGK | TCHYNSGGQGTQVTVSS |
| | PSREEMTKNQVSLSCAVKGF | (SEQ ID NO: 163) | (SEQ ID NO: 164) |
| | YPSDIAVEWESNGQPENNYK | | |
| | TTPPVLDSDGSFFLVSKLTV | | |
| | DKSRWQQGNVFSCSVMHEAL | | |
| | HNRFTQKSLSLSPGK | | |
| | (SEQ ID NO: 153) | | |
| FUSE-497 | EVQLVESGGGLVQPGGSLRL | EVQLLESGGGLVPRGGSLRL | QAVVTQEPSLTVSPGGTVTL |
| | SCAASGFTFSTYAMNWVRQA | SCTASGYTNRLKCMGWFRQA | TCRSSTGAVTTSNYANWVQQ |
| | PGKGLEWVARIRSKYNNYAT | PGKEREEIATISTGTGNTYY | KPGQAPRGLIGGTNKRAPGT |
| | YYADSVKDRFTISRDDSKNT | ADSVKGRFTISRDNSRNTLY | PARFSGSLLGGKAALTLSGV |
| | LYLQMNSLRAEDTAVYYCAR | LQMKTLRAEDTAVYYCAADV | QPEDEAEYYCALWYSNLWVF |
| | HGNFGNSYVSWFAYWGQGTM | RPDGTTCHYNSWGQGTQVTV | GGGTKLTVLGQPKAAPSVTL |
| | VTVSSASTKGPSVFPLAPSS | SSEPKSSDKTHTCPPCPAPE | FPPSSEELQANKATLVCLIS |
| | KSTSGGTAALGCLVKDYFPE | AAGGPSVFLFPPKPKDTLMI | DFYPGAVTVAWKADSSPVKA |
| | PVTVSWNSGALTSGVHTFPA | SRTPEVTCVVVDVSHEDPEV | GVETTTPSKQSNNKYAASSY |
| | VLQSSGLYSLSSVVTVPSSS | KFNWYVDGVEVHNAKTKPRE | LSLTPEQWKSHRSYSCQVTH |
| | LGTQTYICNVNHKPSNTKVD | EQYNSTYRVVSVLTVLHQDW | EGSTVEKTVAPTECSGGGGS |
| | KKVEPKSCDKTHTCPPCPAP | LNGKEYKCKVSNKALPAPIE | GGGGSGGGGSGGGGSEVQLL |
| | EAAGGPSVFLFPPKPKDTLM | KTISKAKGQPREPQVYTLPP | ESGGGLVPRGGSLRLSCTAS |
| | ISRTPEVTCVVVDVSHEDPE | SREEMTKNQVSLWCLVKGFY | GYTNRLKCMGWFRQAPGKER |
| | VKFNWYVDGVEVHNAKTKPR | PSDIAVEWESNGQPENNYKT | EEIATISTGTGNTYYADSVK |
| | EEQYNSTYRVVSVLIVLHQD | TPPVLDSDGSFFLYSKLTVD | GRFTISRDNSRNTLYLQMKT |
| | WLNGKEYKCKVSNKALPAPI | KSRWQQGNVFSCSVMHEALH | LRAEDTAVYYCAADVRPDGT |
| | EKTISKAKGQPREPQVYTLP | NHYTQKSLSLSPGK | TCHYNSWGQGTQVTVSS |
| | PSREEMTKNQVSLSCAVKGF | (SEQ ID NO: 165) | (SEQ ID NO: 166) |
| | YPSDIAVEWESNGQPENNYK | | |
| | TTPPVLDSDGSFFLVSKLTV | | |
| | DKSRWQQGNVFSCSVMHEAL | | |
| | HNRFTQKSLSLSPGK | | |
| | (SEQ ID NO: 153) | | |
| FUSE-498 | EVQLVESGGGLVQPGGSLRL | EVQLVESGGGLVQPGGSLRL | QAVVTQEPSLTVSPGGTVTL |
| | SCAASGFTFSTYAMNWVRQA | SCTASGYTNRLKCMGWFRQA | TCRSSTGAVTTSNYANWVQQ |
| | PGKGLEWVARIRSKYNNYAT | PGKEREEIATISTGTGNTYY | KPGQAPRGLIGGINKRAPGT |
| | YYADSVKDRFTISRDDSKNT | ADSVKGRFTFSRDNSKNTLY | PARFSGSLLGGKAALTLSGV |
| | LYLQMNSLRAEDTAVYYCAR | LQMNSLRAEDTAVYYCAADV | QPEDEAEYYCALWYSNLWVF |
| | HGNFGNSYVSWFAYWGQGTM | RPDGITCHYNSGGQGTQVTV | GGGTKLTVLGQPKAAPSVTL |
| | VTVSSASTKGPSVFPLAPSS | SSEPKSSDKTHTCPPCPAPE | FPPSSEELQANKATLVCLIS |
| | KSTSGGTAALGCLVKDYFPE | AAGGPSVFLFPPKPKDTLMI | DFYPGAVTVAWKADSSPVKA |
| | PVTVSWNSGALTSGVHTFPA | SRTPEVTCVVVDVSHEDPEV | GVETTTPSKQSNNKYAASSY |
| | VLQSSGLYSLSSVVTVPSSS | KFNWYVDGVEVHNAKTKPRE | LSLTPEQWKSHRSYSCQVTH |
| | LGTQTYICNVNHKPSNTKVD | EQYNSTYRVVSVLTVLHQDW | EGSTVEKTVAPTECSGGGGS |
| | KKVEPKSCDKTHTCPPCPAP | LNGKEYKCKVSNKALPAPIE | GGGGSGGGGSGGGGSEVQLV |
| | EAAGGPSVFLFPPKPKDTLM | KTISKAKGQPREPQVYTLPP | ESGGGLVQPGGSLRLSCTAS |
| | ISRTPEVTCVVVDVSHEDPE | SREEMTKNQVSLWCLVKGFY | GYTNRLKCMGWFRQAPGKER |
| | VKFNWYVDGVEVHNAKTKPR | PSDIAVEWESNGQPENNYKT | EEIATISTGTGNTYYADSVK |
| | EEQYNSTYRVVSVLTVLHQD | TPPVLDSDGSFFLYSKLTVD | GRFTFSRDNSKNTLYLQMNS |
| | WLNGKEYKCKVSNKALPAPI | KSRWQQGNVFSCSVMHEALH | LRAEDTAVYYCAADVRPDGT |
| | EKTISKAKGQPREPQVYTLP | NHYTQKSLSLSPGK | TCHYNSGGQGTQVTVSS |
| | PSREEMTKNQVSLSCAVKGF | (SEQ ID NO: 167) | (SEQ ID NO: 168) |
| | YPSDIAVEWESNGQPENNYK | | |
| | TTPPVLDSDGSFFLVSKLTV | | |
| | DKSRWQQGNVFSCSVMHEAL | | |
| | HNRFTQKSLSLSPGK | | |
| | (SEQ ID NO: 153) | | |
| FUSE-536 | EVQLVESGGGLVQPGGSLRL | EVQLLESGGGLVQPGGSLRL | QAVVTQEPSLTVSPGGTVTL |
| | SCAASGFTFSTYAMNWVRQA | SCAASGYTNRLKCMGWFRQA | TCRSSTGAVTTSNYANWVQQ |
| | PGKGLEWVARIRSKYNNYAT | PGKERELASISTGTGNTYYA | KPGQAPRGLIGGTNKRAPGT |
| | YYADSVKDRFTISRDDSKNT | DSVKGRFTISRDNSKNTLYL | PARFSGSLLGGKAALTLSGV |
| | LYLQMNSLRAEDTAVYYCAR | QMNSLKAEDTAVYYCAADVR | QPEDEAEYYCALWYSNLWVF |
| | HGNFGNSYVSWFAYWGQGTM | PDGTTCHYNSRGQGTLVTVS | GGGTKLTVLGQPKAAPSVTL |
| | VTVSSASTKGPSVFPLAPSS | SEPKSSDKTHTCPPCPAPEA | FPPSSEELQANKATLVCLIS |
| | KSTSGGTAALGCLVKDYFPE | AGGPSVFLFPPKPKDTLMIS | DFYPGAVTVAWKADSSPVKA |
| | PVTVSWNSGALTSGVHTFPA | RTPEVTCVVVDVSHEDPEVK | GVETTTPSKQSNNKYAASSY |
| | VLQSSGLYSLSSVVTVPSSS | ENWYVDGVEVHNAKTKPREE | LSLTPEQWKSHRSYSCQVTH |
| | LGTQTYICNVNHKPSNTKVD | QYNSTYRVVSVLTVLHQDWL | EGSTVEKTVAPTECSGGGGS |
| | KKVEPKSCDKTHTCPPCPAP | NGKEYKCKVSNKALPAPIEK | GGGGSGGGGSGGGGSEVQLL |
| | EAAGGPSVFLFPPKPKDTLM | TISKAKGQPREPQVYTLPPS | ESGGGLVQPGGSLRLSCAAS |
| | ISRTPEVTCVVVDVSHEDPE | REEMTKNQVSLWCLVKGFYP | GYTNRLKCMGWFRQAPGKER |
| | VKFNWYVDGVEVHNAKTKPR | SDIAVEWESNGQPENNYKTT | ELASISTGTGNTYYADSVKG |
| | EEQYNSTYRVVSVLTVLHQD | PPVLDSDGSFFLYSKLTVDK | RFTISRDNSKNTLYLQMNSL |
| | WLNGKEYKCKVSNKALPAPI | SRWQQGNVFSCSVMHEALHN | KAEDTAVYYCAADVRPDGTT |
| | EKTISKAKGQPREPQVYTLP | HYTQKSLSLSPGK | CHYNSRGQGTLVTVSS |
| | PSREEMTKNQVSLSCAVKGF | (SEQ ID NO: 169) | (SEQ ID NO: 170) |
| | YPSDIAVEWESNGQPENNYK | | |
| | TTPPVLDSDGSFFLVSKLTV | | |
| | DKSRWQQGNVFSCSVMHEAL | | |
| | HNRFTQKSLSLSPGK | | |
| | (SEQ ID NO: 153) | | |

TABLE 2
Polynucleotide sequences encoding the fusion proteins corresponding to those in Table 1.

| | Encoding Polypeptide 1 | Encoding Polypeptide 2 | Encoding Polypeptide 3 |
|---|---|---|---|
| FUSE-211 | CAGGTGCAGCTGCAGGAGAG | GAGGTGCAGCTGGTGGAGAG | CAGGCTGTGGTGACCCAGGA |
| | CGGCGGCGGATCAGTGCCAG | CGGGGGGGGACTGGTGCAGC | GCCCAGCCTGACCGTGAGCC |
| | CCGGAGGAAGCCTGAGACTG | CAGGAGGAAGCCTGAGACTG | CCGGAGGAACAGTGACCCTG |
| | AGCTGCGCCGCCAGCGGCAG | AGCTGTGCCGCCAGCGGGTT | ACCTGCAGAAGCAGCACCGG |
| | CACATACAGCGCCAACTGCA | TACATTTAGCACATATGCCA | CGCCGTGACAACCTCAAACT |
| | TGGGCTGGTTCAGACAGGCT | TGAACTGGGTGAGACAGGCC | ACGCCAACTGGGTGCAGCAG |
| | CCCGGAAAAGAAAGAGAAGA | CCCGGAAAAGGACTGGAGTG | AAACCTGGACAGGCCCCCAG |
| | AGTGGCCAGCATGAGCATCA | GGTGGCTAGAATCAGAAGCA | AGGACTGATCGGCGGAACCA |
| | GAAGCGGCCGGACCTACTAC | AGTACAACAACTACGCCACC | ACAAGAGAGCCCCCGGCACC |
| | AGTGATAGTGTGAAGGGCAG | TACTACGCCGACAGTGTGAA | CCCGCCAGATTCAGCGGAAG |
| | ATTCACCATTAGCCAGGACG | GGACAGATTCACCATTAGCA | TCTGCTGGGCGGCAAAGCCG |
| | GCAGCAAGAACACACTGTAC | GAGATGATAGCAAGAACACA | CCCTGACCCTGAGCGGAGTG |
| | CTGCAGCTGAACAGCCTGAA | CTGTACCTGCAGATGAATAG | CAGCCTGAGGATGAAGCCGA |
| | AGCCGAAGACACCGCCCTGT | TCTGAGAGCCGAGGACACAG | GTACTACTGCGCTCTGTGGT |
| | ACTACTGCGCCGCCGCCTAC | CCGTGTACTACTGTGCCAGG | ACAGCAACCTGTGGGTGTTC |
| | GGCGGATCTAGGTGTGTGTA | CACGGAAACTTCGGGAACAG | GGAGGAGGCACCAAGCTGAC |
| | CAACTACAGAGGCCAGGGCA | CTACGTGAGCTGGTTCGCCT | CGTGCTGGGTCAGCCCAAGG |
| | CCCAGGTGACCGTGAGCAGC | ACTGGGGCCAGGGCACCATG | CTGCCCCCTCGGTCACTCTG |
| | GGTGGTGGCGGATCAGGTGG | GTGACCGTGAGCTCCGCTAG | TTCCCGCCCTCCTCTGAGGA |
| | GGGAGGCTCTGGTGGAGGCG | CACCAAGGGCCCATCGGTCT | GCTTCAAGCCAACAAGGCCA |
| | GTAGTCAGGTGCAGCTGCAG | TCCCCCTGGCACCCTCCTCC | CACTGGTGTGTCTCATAAGT |
| | GAGAGCGGCGGCGGATCAGT | AAGAGCACCTCTGGGGGCAC | GACTTCTACCCGGGAGCCGT |
| | GCCAGCCGGAGGAAGCCTGA | AGCGGCCCTGGGCTGCCTGG | GACAGTGGCCTGGAAGGCAG |
| | GACTGAGCTGCGCCGCCAGC | TCAAGGACTACTTCCCCGAA | ATAGCAGCCCCGTCAAGGCG |
| | GGCAGCACATACAGCGCCAA | CCGGTGACGGTGTCGTGGAA | GGAGTGGAGACCACCACACC |
| | CTGCATGGGCTGGTTCAGAC | CTCAGGCGCCCTGACCAGCG | CTCCAAACAAAGCAACAACA |
| | AGGCTCCCGGAAAAGAAAGA | GCGTGCACACCTTCCCGGCT | AGTACGCGGCCAGCAGCTAC |
| | GAAGAAGTGGCCAGCATGAG | GTCCTACAGTCCTCAGGACT | CTGAGCCTGACGCCTGAGCA |
| | CATCAGAAGCGGCCGGACCT | CTACTCCCTCAGCAGCGTGG | GTGGAAGTCCCACAGGAGCT |
| | ACTACAGTGATAGTGTGAAG | TGACCGTGCCCTCCAGCAGC | ACAGTTGCCAGGTCACGCAT |
| | GGCAGATTCACCATTAGCCA | TTGGGCACCCAGACCTACAT | GAAGGGAGCACCGTGGAGAA |
| | GGACGGCAGCAAGAACACAC | CTGCAACGTGAATCACAAGC | GACAGTGGCCCCTACAGAAT |
| | TGTACCTGCAGCTGAACAGC | CCAGCAACACCAAGGTGGAC | GTTCA |
| | CTGAAAGCCGAAGACACCGC | AAGAAAGTTGAGCCCAAATC | (SEQ ID NO: 173) |
| | CCTGTACTACTGCGCCGCCG | TTGTGACAAAACTCACACAT | |
| | CCTACGGCGGATCTAGGTGT | GCCCACCGTGCCCAGCACCT | |
| | GTGTACAACTACAGAGGCCA | GAAGCCGCAGGGGGACCGTC | |
| | GGGCACCCAGGTGACCGTGA | AGTCTTCCTCTTCCCCCCAA | |
| | GCAGCGAGCCCAAATCTAGC | AACCCAAGGACACCCTCATG | |
| | GACAAAACTCACACATGCCC | ATCTCCCGGACCCCCGAGGT | |
| | ACCGTGCCCAGCACCTGAAG | CACATGCGTGGTGGTGGACG | |
| | CCGCAGGGGGACCGTCAGTC | TGAGCCACGAAGACCCTGAG | |
| | TTCCTCTTCCCCCCAAAACC | GTCAAGTTCAACTGGTACGT | |
| | CAAGGACACCCTCATGATCT | GGACGGCGTGGAGGTGCATA | |
| | CCCGGACCCCCGAGGTCACA | ATGCCAAGACAAAGCCGCGG | |
| | TGCGTGGTGGTGGACGTGAG | GAGGAGCAGTACAACAGCAC | |
| | CCACGAAGACCCTGAGGTCA | GTACCGTGTGGTCAGCGTCC | |
| | AGTTCAACTGGTACGTGGAC | TCACCGTCCTGCACCAGGAC | |
| | GGCGTGGAGGTGCATAATGC | TGGCTGAATGGCAAGGAGTA | |
| | CAAGACAAAGCCGCGGGAGG | CAAGTGCAAGGTCTCCAACA | |
| | AGCAGTACAACAGCACGTAC | AAGCCCTCCCAGCCCCCATC | |
| | CGTGTGGTCAGCGTCCTCAC | GAGAAAACCATCTCCAAAGC | |
| | CGTCCTGCACCAGGACTGGC | CAAAGGGCAGCCCCGAGAAC | |
| | TGAATGGCAAGGAGTACAAG | CACAGGTGTACACCCTGCCC | |
| | TGCAAGGTCTCCAACAAAGC | CCATCCCGGGAGGAGATGAC | |
| | CCTCCCAGCCCCCATCGAGA | CAAGAACCAGGTCAGCCTGT | |
| | AAACCATCTCCAAAGCCAAA | GGTGCCTGGTCAAAGGCTTC | |
| | GGGCAGCCCCGAGAACCACA | TATCCCAGCGACATCGCCGT | |
| | GGTGTACACCCTGCCCCCAT | GGAGTGGGAGAGCAATGGGC | |
| | CCCGGGAGGAGATGACCAAG | AGCCGGAGAACAACTACAAG | |
| | AACCAGGTCAGCCTGAGCTG | ACCACGCCTCCCGTGCTGGA | |
| | CGCCGTCAAAGGCTICTATC | CTCCGACGGCTCCTTCTTCC | |
| | CCAGCGACATCGCCGTGGAG | TCTACAGCAAGCTCACCGTG | |
| | TGGGAGAGCAATGGGCAGCC | GACAAGAGCAGGTGGCAGCA | |
| | GGAGAACAACTACAAGACCA | GGGGAACGTCTTCTCATGCT | |
| | CGCCTCCCGTGCTGGACTCC | CCGTGATGCATGAGGCTCTG | |
| | GACGGCTCCTTCTTCCTCGT | CACAACCACTACACGCAGAA | |
| | GAGCAAGCTCACCGTGGACA | GAGCCTCTCCCTGTCTCCGG | |
| | AGAGCAGGTGGCAGCAGGGG | GTAAA | |
| | AACGTCTTCTCATGCTCCGT | (SEQ ID NO: 172) | |
| | GATGCATGAGGCTCTGCACA | | |
| | ACCGGTTCACGCAGAAGAGC | | |
| | CTCTCCCTGTCTCCGGGTAA | | |
| | A | | |
| | (SEQ ID NO: 175) | | |
| FUSE-393 | CAGGTGCAGCTGCAGGAGAG | GAGGTGCAGCTGGTGGAGAG | CAGGCTGTGGTGACCCAGGA |
| | CGGCGGCGGATCAGTGCAGG | CGGGGGGGGACTGGTGCAGC | GCCCAGCCTGACCGTGAGCC |
| | CCGGAGGAAGCCTGAAGCTG | CAGGAGGAAGCCTGAGACTG | CCGGAGGAACAGTGACCCTG |
| | AGCTGCACAGCCAGCGGCTA | AGCTGTGCCGCCAGCGGGTT | ACCTGCAGAAGCAGCACCGG |
| | CACAAACAGACTGAAATGCA | TACATTTAGCACATATGCCA | CGCCGTGACAACCTCAAACT |
| | TGGGCTGGTTCAGACAGGCC | TGAACTGGGTGAGACAGGCC | ACGCCAACTGGGTGCAGCAG |
| | CCCGGAAAAGAGAGAGAAGA | CCCGGAAAAGGACTGGAGTG | AAACCTGGACAGGCCCCCAG |
| | GATCGCCACCATCAGCACCG | GGTGGCTAGAATCAGAAGCA | AGGACTGATCGGCGGAACCA |
| | GCACAGGAAACACATACTAC | AGTACAACAACTACGCCACC | ACAAGAGAGCCCCCGGCACC |
| | GCCGACAGCGTGAAAGGCAG | TACTACGCCGACAGTGTGAA | CCCGCCAGATTCAGCGGAAG |
| | ATTCACCTTCAGCCAGGACA | GGACAGATTCACCATTAGCA | TCTGCTGGGCGGCAAAGCCG |
| | AAGTGAAGAACACCGTGTAC | GAGATGATAGCAAGAACACA | CCCTGACCCTGAGCGGAGTG |
| | CTGCAGATGAACACACTGAA | CTGTACCTGCAGATGAATAG | CAGCCTGAGGATGAAGCCGA |
| | GCCCGACGACACCGGCATGT | TCTGAGAGCCGAGGACACAG | GTACTACTGCGCTCTGTGGT |
| | ACTACTGCGCCGCCGACGTG | CCGTGTACTACTGTGCCAGG | ACAGCAACCTGTGGGTGTTC |
| | AGGCCTGACGGAACAACCTG | CACGGAAACTTCGGGAACAG | GGAGGAGGCACCAAGCTGAC |
| | CCACTACAACAGCGGAGGAC | CTACGTGAGCTGGTTCGCCT | CGTGCTGGGTCAGCCCAAGG |
| | AGGGAACCCAGGTGACCGTG | ACTGGGGCCAGGGCACCATG | CTGCCCCCTCGGTCACTCTG |
| | TCCAGCGGTGGTGGCGGATC | GTGACCGTGAGCTCCGCTAG | TTCCCGCCCTCCTCTGAGGA |
| | AGGTGGGGGAGGCTCTGGTG | CACCAAGGGCCCATCGGTCT | GCTTCAAGCCAACAAGGCCA |
| | GAGGCGGTAGTCAGGTGCAG | TCCCCCTGGCACCCTCCTCC | CACTGGTGTGTCTCATAAGT |
| | CTGCAGGAGAGCGGCGGCGG | AAGAGCACCTCTGGGGGCAC | GACTICTACCCGGGAGCCGT |
| | ATCAGTGCAGGCCGGAGGAA | AGCGGCCCTGGGCTGCCTGG | GACAGTGGCCTGGAAGGCAG |
| | GCCTGAAGCTGAGCTGCACA | TCAAGGACTACTTCCCCGAA | ATAGCAGCCCCGTCAAGGCG |
| | GCCAGCGGCTACACAAACAG | CCGGTGACGGTGTCGTGGAA | GGAGTGGAGACCACCACACC |
| | ACTGAAATGCATGGGCTGGT | CTCAGGCGCCCTGACCAGCG | CTCCAAACAAAGCAACAACA |
| | TCAGACAGGCCCCCGGAAAA | GCGTGCACACCTTCCCGGCT | AGTACGCGGCCAGCAGCTAC |
| | GAGAGAGAAGAGATCGCCAC | GTCCTACAGTCCTCAGGACT | CTGAGCCTGACGCCTGAGCA |
| | CATCAGCACCGGCACAGGAA | CTACTCCCTCAGCAGCGTGG | GTGGAAGTCCCACAGGAGCT |
| | ACACATACTACGCCGACAGC | TGACCGTGCCCTCCAGCAGC | ACAGTTGCCAGGTCACGCAT |
| | GTGAAAGGCAGATICACCTT | TTGGGCACCCAGACCTACAT | GAAGGGAGCACCGTGGAGAA |
| | CAGCCAGGACAAAGTGAAGA | CTGCAACGTGAATCACAAGC | GACAGTGGCCCCTACAGAAT |
| | ACACCGTGTACCTGCAGATG | CCAGCAACACCAAGGTGGAC | GTTCA |
| | AACACACTGAAGCCCGACGA | AAGAAAGTTGAGCCCAAATC | (SEQ ID NO: 173) |
| | CACCGGCATGTACTACTGCG | TTGTGACAAAACTCACACAT | |
| | CCGCCGACGTGAGGCCTGAC | GCCCACCGTGCCCAGCACCT | |
| | GGAACAACCTGCCACTACAA | GAAGCCGCAGGGGGACCGTC | |
| | CAGCGGAGGACAGGGAACCC | AGTCTTCCTCTTCCCCCCAA | |
| | AGGTGACCGTGTCCAGCGAG | AACCCAAGGACACCCTCATG | |
| | CCCAAATCTAGCGACAAAAC | ATCTCCCGGACCCCCGAGGT | |
| | TCACACATGCCCACCGTGCC | CACATGCGTGGTGGTGGACG | |
| | CAGCACCTGAAGCCGCAGGG | TGAGCCACGAAGACCCTGAG | |
| | GGACCGTCAGTCTTCCTCTT | GTCAAGTTCAACTGGTACGT | |
| | CCCCCCAAAACCCAAGGACA | GGACGGCGTGGAGGTGCATA | |
| | CCCTCATGATCTCCCGGACC | ATGCCAAGACAAAGCCGCGG | |
| | CCCGAGGTCACATGCGTGGT | GAGGAGCAGTACAACAGCAC | |
| | GGTGGACGTGAGCCACGAAG | GTACCGTGTGGTCAGCGTCC | |
| | ACCCTGAGGTCAAGTTCAAC | TCACCGTCCTGCACCAGGAC | |
| | TGGTACGTGGACGGCGTGGA | TGGCTGAATGGCAAGGAGTA | |
| | GGTGCATAATGCCAAGACAA | CAAGTGCAAGGTCTCCAACA | |
| | AGCCGCGGGAGGAGCAGTAC | AAGCCCTCCCAGCCCCCATC | |
| | AACAGCACGTACCGTGTGGT | GAGAAAACCATCTCCAAAGC | |
| | CAGCGTCCTCACCGTCCTGC | CAAAGGGCAGCCCCGAGAAC | |
| | ACCAGGACTGGCTGAATGGC | CACAGGTGTACACCCTGCCC | |
| | AAGGAGTACAAGTGCAAGGT | CCATCCCGGGAGGAGATGAC | |
| | CTCCAACAAAGCCCTCCCAG | CAAGAACCAGGTCAGCCTGT | |
| | CCCCCATCGAGAAAACCATC | GGTGCCTGGTCAAAGGCTTC | |
| | TCCAAAGCCAAAGGGCAGCC | TATCCCAGCGACATCGCCGT | |
| | CCGAGAACCACAGGTGTACA | GGAGTGGGAGAGCAATGGGC | |
| | CCCTGCCCCCATCCCGGGAG | AGCCGGAGAACAACTACAAG | |
| | GAGATGACCAAGAACCAGGT | ACCACGCCTCCCGTGCTGGA | |
| | CAGCCTGAGCTGCGCCGTCA | CTCCGACGGCTCCTTCTTCC | |
| | AAGGCTTCTATCCCAGCGAC | TCTACAGCAAGCTCACCGTG | |
| | ATCGCCGTGGAGTGGGAGAG | GACAAGAGCAGGTGGCAGCA | |
| | CAATGGGCAGCCGGAGAACA | GGGGAACGTCTTCTCATGCT | |
| | ACTACAAGACCACGCCTCCC | CCGTGATGCATGAGGCTCTG | |
| | GTGCTGGACTCCGACGGCTC | CACAACCACTACACGCAGAA | |
| | CTTCTTCCTCGTGAGCAAGC | GAGCCTCTCCCTGTCTCCGG | |
| | TCACCGTGGACAAGAGCAGG | GTAAA | |
| | TGGCAGCAGGGGAACGTCTT | (SEQ ID NO: 172) | |
| | CTCATGCTCCGTGATGCATG | | |
| | AGGCTCTGCACAACCGGTTC | | |
| | ACGCAGAAGAGCCTCTCCCT | | |
| | GTCTCCGGGTAAA | | |
| | (SEQ ID NO: 176) | | |
| FUSE-394 | CAGGTGCAGCTGCAGGAGTC | GAGGTGCAGCTGGTGGAGAG | CAGGCTGTGGTGACCCAGGA |
| | CGGGGCGGAAGCGTGCAGGC | CGGGGGGGGACTGGTGCAGC | GCCCAGCCTGACCGTGAGCC |
| | AGGAGGAAGCCTGAAGCTGA | CAGGAGGAAGCCTGAGACTG | CCGGAGGAACAGTGACCCTG |
| | GCTGCACAGCCTCCGGCTAC | AGCTGTGCCGCCAGCGGGTT | ACCTGCAGAAGCAGCACCGG |
| | ACAAACAGGCTGAAATGCAT | TACATTTAGCACATATGCCA | CGCCGTGACAACCTCAAACT |
| | GGGCTGGTTCAGGCAGGCAC | TGAACTGGGTGAGACAGGCC | ACGCCAACTGGGTGCAGCAG |
| | CCGGCAAAGAGAGAGAGGAG | CCCGGAAAAGGACTGGAGTG | AAACCTGGACAGGCCCCCAG |
| | ATTGCTACAATCAGCACCGG | GGTGGCTAGAATCAGAAGCA | AGGACTGATCGGCGGAACCA |
| | AACCGGAAATACCTACTACG | AGTACAACAACTACGCCACC | ACAAGAGAGCCCCCGGCACC |
| | CCGACTCCGTGAAAGGCAGG | TACTACGCCGACAGTGTGAA | CCCGCCAGATTCAGCGGAAG |
| | TTCACATTCAGCCAGGACAA | GGACAGATTCACCATTAGCA | TCTGCTGGGCGGCAAAGCCG |
| | AGTGAAGAACACCGTGTACC | GAGATGATAGCAAGAACACA | CCCTGACCCTGAGCGGAGTG |
| | TGCAGATGAACACCCTGAAG | CTGTACCTGCAGATGAATAG | CAGCCTGAGGATGAAGCCGA |
| | CCCGACGACACCGGCATGTA | TCTGAGAGCCGAGGACACAG | GTACTACTGCGCTCTGTGGT |
| | CTACTGCGCCGCCGACGTGA | CCGTGTACTACTGTGCCAGG | ACAGCAACCTGTGGGTGTTC |
| | GGCCCGATGGAACCACCTGC | CACGGAAACTTCGGGAACAG | GGAGGAGGCACCAAGCTGAC |
| | CACTACAACTCCGGAGGACA | CTACGTGAGCTGGTTCGCCT | CGTGCTGGGTCAGCCCAAGG |
| | GGGAACCCAGGTGACCGTGT | ACTGGGGCCAGGGCACCATG | CTGCCCCCTCGGTCACTCTG |
| | CCTCCGAGCCCAAATCTAGC | GTGACCGTGAGCTCCGCTAG | TTCCCGCCCTCCTCTGAGGA |
| | GACAAAACTCACACATGCCC | CACCAAGGGCCCATCGGTCT | GCTTCAAGCCAACAAGGCCA |
| | ACCGTGCCCAGCACCTGAAG | TCCCCCTGGCACCCTCCTCC | CACTGGTGTGTCTCATAAGT |
| | CCGCAGGGGGACCGTCAGTC | AAGAGCACCTCTGGGGGCAC | GACTTCTACCCGGGAGCCGT |
| | TTCCTCTTCCCCCCAAAACC | AGCGGCCCTGGGCTGCCTGG | GACAGTGGCCTGGAAGGCAG |
| | CAAGGACACCCTCATGATCT | TCAAGGACTACTTCCCCGAA | ATAGCAGCCCCGTCAAGGCG |
| | CCCGGACCCCCGAGGTCACA | CCGGTGACGGTGTCGTGGAA | GGAGTGGAGACCACCACACC |
| | TGCGTGGTGGTGGACGTGAG | CTCAGGCGCCCTGACCAGCG | CTCCAAACAAAGCAACAACA |
| | CCACGAAGACCCTGAGGTCA | GCGTGCACACCTTCCCGGCT | AGTACGCGGCCAGCAGCTAC |
| | AGTTCAACTGGTACGTGGAC | GTCCTACAGTCCTCAGGACT | CTGAGCCTGACGCCTGAGCA |
| | GGCGTGGAGGTGCATAATGC | CTACTCCCTCAGCAGCGTGG | GTGGAAGTCCCACAGGAGCT |
| | CAAGACAAAGCCGCGGGAGG | TGACCGTGCCCTCCAGCAGC | ACAGTTGCCAGGTCACGCAT |
| | AGCAGTACAACAGCACGTAC | TTGGGCACCCAGACCTACAT | GAAGGGAGCACCGTGGAGAA |
| | CGTGTGGTCAGCGTCCTCAC | CTGCAACGTGAATCACAAGC | GACAGTGGCCCCTACAGAAT |
| | CGTCCTGCACCAGGACTGGC | CCAGCAACACCAAGGTGGAC | GTTCAGGTGGCGGAGGGTCT |
| | TGAATGGCAAGGAGTACAAG | AAGAAAGTTGAGCCCAAATC | GGTGGTGGAGGATCAGGGGG |
| | TGCAAGGTCTCCAACAAAGC | TTGTGACAAAACTCACACAT | TGGAGGTTCAGGAGGGGGGG |
| | CCTCCCAGCCCCCATCGAGA | GCCCACCGTGCCCAGCACCT | AAGTCAGGTGCAGCTGCAGG |
| | AAACCATCTCCAAAGCCAAA | GAAGCCGCAGGGGGACCGTC | AATCAGGCGGAGGAAGCGTG |
| | GGGCAGCCCCGAGAACCACA | AGTCTTCCTCTTCCCCCCAA | CAGGCTGGAGGATCTCTGAA |
| | GGTGTACACCCTGCCCCCAT | AACCCAAGGACACCCTCATG | GCTGAGCTGCACAGCCAGCG |
| | CCCGGGAGGAGATGACCAAG | ATCTCCCGGACCCCCGAGGT | GCTACACAAACAGACTGAAG |
| | AACCAGGTCAGCCTGAGCTG | CACATGCGTGGTGGTGGACG | TGCATGGGCTGGTTCAGACA |
| | CGCCGTCAAAGGCTTCTATC | TGAGCCACGAAGACCCTGAG | GGCCCCCGGAAAAGAGAGAG |
| | CCAGCGACATCGCCGTGGAG | GTCAAGTTCAACTGGTACGT | AGGAGATIGCTACAATTAGC |
| | TGGGAGAGCAATGGGCAGCC | GGACGGCGTGGAGGTGCATA | ACAGGAACCGGCAACACCTA |
| | GGAGAACAACTACAAGACCA | ATGCCAAGACAAAGCCGCGG | CTACGCCGACAGTGTGAAAG |
| | CGCCTCCCGTGCTGGACTCC | GAGGAGCAGTACAACAGCAC | GCAGATTCACATTCAGCCAG |
| | GACGGCTCCTTCTTCCTCGT | GTACCGTGTGGTCAGCGTCC | GACAAAGTGAAGAACACCGT |
| | GAGCAAGCTCACCGTGGACA | TCACCGTCCTGCACCAGGAC | GTACCTGCAGATGAACACCC |
| | AGAGCAGGTGGCAGCAGGGG | TGGCTGAATGGCAAGGAGTA | TGAAGCCCGACGACACCGGA |
| | AACGTCTTCTCATGCTCCGT | CAAGTGCAAGGTCTCCAACA | ATGTACTACTGCGCCGCCGA |
| | GATGCATGAGGCTCTGCACA | AAGCCCTCCCAGCCCCCATC | CGTGAGACCCGATGGAACAA |
| | ACCGGTTCACGCAGAAGAGC | GAGAAAACCATCTCCAAAGC | CATGCCACTACAACAGCGGA |
| | CTCTCCCTGTCTCCGGGTAA | CAAAGGGCAGCCCCGAGAAC | GGACAGGGAACCCAGGTGAC |
| | A | CACAGGTGTACACCCTGCCC | CGTGTCTAGC |
| | (SEQ ID NO: 177) | CCATCCCGGGAGGAGATGAC | (SEQ ID NO: 174) |
| | | CAAGAACCAGGTCAGCCTGT | |
| | | GGTGCCTGGTCAAAGGCTTC | |
| | | TATCCCAGCGACATCGCCGT | |
| | | GGAGTGGGAGAGCAATGGGC | |
| | | AGCCGGAGAACAACTACAAG | |
| | | ACCACGCCTCCCGTGCTGGA | |
| | | CTCCGACGGCTCCTTCTTCC | |
| | | TCTACAGCAAGCTCACCGTG | |
| | | GACAAGAGCAGGTGGCAGCA | |
| | | GGGGAACGTCTTCTCATGCT | |
| | | CCGTGATGCATGAGGCTCTG | |
| | | CACAACCACTACACGCAGAA | |
| | | GAGCCTCTCCCTGTCTCCGG | |
| | | GTAAA | |
| | | (SEQ ID NO: 172) | |
| FUSE-399 | GAGCTGGTGATGACACAGAC | GAGGTGCAGCTGGTGGAGAG | CAGGCTGTGGTGACCCAGGA |
| | ACCCAGCAGCACCTCCGGCG | CGGGGGGGGACTGGTGCAGC | GCCCAGCCTGACCGTGAGCC |
| | CCGTGGGAGGAACAGTGACA | CAGGAGGAAGCCTGAGACTG | CCGGAGGAACAGTGACCCTG |
| | ATTAACTGTCAGGCCAGCCA | AGCTGTGCCGCCAGCGGGTT | ACCTGCAGAAGCAGCACCGG |
| | GAGCATTGATAGCAACCTGG | TACATTTAGCACATATGCCA | CGCCGTGACAACCTCAAACT |
| | CCTGGTTCCAGCAGAAGCCC | TGAACTGGGTGAGACAGGCC | ACGCCAACTGGGTGCAGCAG |
| | GGCCAGCCCCCCACCCTGCT | CCCGGAAAAGGACTGGAGTG | AAACCTGGACAGGCCCCCAG |
| | GATCTACAGAGCTAGTAACC | GGTGGCTAGAATCAGAAGCA | AGGACTGATCGGCGGAACCA |
| | TGGCCAGCGGAGTGCCCAGC | AGTACAACAACTACGCCACC | ACAAGAGAGCCCCCGGCACC |
| | AGATTTTCCGGAAGCAGAAG | TACTACGCCGACAGTGTGAA | CCCGCCAGATTCAGCGGAAG |
| | CGGCACAGAATACACCCTGA | GGACAGATTCACCATTAGCA | TCTGCTGGGGGGCAAAGCCG |
| | CCATCTCCGGCGTGCAGAGA | GAGATGATAGCAAGAACACA | CCCTGACCCTGAGCGGAGTG |
| | GAAGACGCCGCCACCTACTA | CTGTACCTGCAGATGAATAG | CAGCCTGAGGATGAAGCCGA |
| | CTGCCTGGGCGGGGTGGGCA | TCTGAGAGCCGAGGACACAG | GTACTACTGCGCTCTGTGGT |
| | ATGTGAGTTACAGAACATCC | CCGTGTACTACTGTGCCAGG | ACAGCAACCTGTGGGTGTTC |
| | TTTGGCGGAGGAACCGAGGT | CACGGAAACTTCGGGAACAG | GGAGGAGGCACCAAGCTGAC |
| | GGTGGTGAAGGGTGGCGGAG | CTACGTGAGCTGGTTCGCCT | CGTGCTGGGTCAGCCCAAGG |
| | GGTCTGGTGGTGGAGGATCA | ACTGGGGCCAGGGCACCATG | CTGCCCCCTCGGTCACTCTG |
| | GGGGGTGGAGGTTCAGGAGG | GTGACCGTGAGCTCCGCTAG | TTCCCGCCCTCCTCTGAGGA |
| | CGGGGGAAGTCAGAGCGTGA | CACCAAGGGCCCATCGGTCT | GCTTCAAGCCAACAAGGCCA |
| | AGGAGAGCGAGGGCGACCTG | TCCCCCTGGCACCCTCCTCC | CACTGGTGTGTCTCATAAGT |
| | GTGACCCCCGCTGGAAACCT | AAGAGCACCTCTGGGGGCAC | GACTTCTACCCGGGAGCCGT |
| | GACCCTGACCTGCACAGCCA | AGCGGCCCTGGGCTGCCTGG | GACAGTGGCCTGGAAGGCAG |
| | GCGGCAGCGACATCAACGAC | TCAAGGACTACTTCCCCGAA | ATAGCAGCCCCGTCAAGGCG |
| | TACCCCATCAGCTGGGTGAG | CCGGTGACGGTGTCGTGGAA | GGAGTGGAGACCACCACACC |
| | ACAGGCCCCCGGAAAGGGAC | CTCAGGCGCCCTGACCAGCG | CTCCAAACAAAGCAACAACA |
| | TGGAATGGATTGGATTCATT | GCGTGCACACCTTCCCGGCT | AGTACGCGGCCAGCAGCTAC |
| | AACAGCGGAGGAAGCACCTG | GTCCTACAGTCCTCAGGACT | CTGAGCCTGACGCCTGAGCA |
| | GTACGCCAGCTGGGTGAAAG | CTACTCCCTCAGCAGCGTGG | GTGGAAGTCCCACAGGAGCT |
| | GAAGATTTACCATCAGCCGC | TGACCGTGCCCTCCAGCAGC | ACAGTTGCCAGGTCACGCAT |
| | ACCAGCACCACTGTGGACCT | TTGGGCACCCAGACCTACAT | GAAGGGAGCACCGTGGAGAA |
| | GAAAATGACAAGCCTGACTA | CTGCAACGTGAATCACAAGC | GACAGTGGCCCCTACAGAAT |
| | CCGACGACACAGCCACCTAC | CCAGCAACACCAAGGTGGAC | GTTCA |
| | TTCTGCGCCAGAGGCTATAG | AAGAAAGTTGAGCCCAAATC | (SEQ ID NO: 173) |
| | CACCTACTACTGCGACTTTA | TTGTGACAAAACTCACACAT | |
| | ACATCTGGGGCCCCGGCACC | GCCCACCGTGCCCAGCACCT | |
| | CTGGTGACCATTAGCTCCGA | GAAGCCGCAGGGGGACCGTC | |
| | GCCCAAATCTAGCGACAAAA | AGTCTTCCTCTTCCCCCCAA | |
| | CTCACACATGCCCACCGTGC | AACCCAAGGACACCCTCATG | |
| | CCAGCACCTGAAGCCGCAGG | ATCTCCCGGACCCCCGAGGT | |
| | GGGACCGTCAGTCTTCCTCT | CACATGCGTGGTGGTGGACG | |
| | TCCCCCCAAAACCCAAGGAC | TGAGCCACGAAGACCCTGAG | |
| | ACCCTCATGATCTCCCGGAC | GTCAAGTTCAACTGGTACGT | |
| | CCCCGAGGTCACATGCGTGG | GGACGGCGTGGAGGTGCATA | |
| | TGGTGGACGTGAGCCACGAA | ATGCCAAGACAAAGCCGCGG | |
| | GACCCTGAGGTCAAGTTCAA | GAGGAGCAGTACAACAGCAC | |
| | CTGGTACGTGGACGGCGTGG | GTACCGTGTGGTCAGCGTCC | |
| | AGGTGCATAATGCCAAGACA | TCACCGTCCTGCACCAGGAC | |
| | AAGCCGCGGGAGGAGCAGTA | TGGCTGAATGGCAAGGAGTA | |
| | CAACAGCACGTACCGTGTGG | CAAGTGCAAGGTCTCCAACA | |
| | TCAGCGTCCTCACCGTCCTG | AAGCCCTCCCAGCCCCCATC | |
| | CACCAGGACTGGCTGAATGG | GAGAAAACCATCTCCAAAGC | |
| | CAAGGAGTACAAGTGCAAGG | CAAAGGGCAGCCCCGAGAAC | |
| | TCTCCAACAAAGCCCTCCCA | CACAGGTGTACACCCTGCCC | |
| | GCCCCCATCGAGAAAACCAT | CCATCCCGGGAGGAGATGAC | |
| | CTCCAAAGCCAAAGGGCAGC | CAAGAACCAGGTCAGCCTGT | |
| | CCCGAGAACCACAGGTGTAC | GGTGCCTGGTCAAAGGCTTC | |
| | ACCCTGCCCCCATCCCGGGA | TATCCCAGCGACATCGCCGT | |
| | GGAGATGACCAAGAACCAGG | GGAGTGGGAGAGCAATGGGC | |
| | TCAGCCTGAGCTGCGCCGTC | AGCCGGAGAACAACTACAAG | |
| | AAAGGCTTCTATCCCAGCGA | ACCACGCCTCCCGTGCTGGA | |
| | CATCGCCGTGGAGTGGGAGA | CTCCGACGGCTCCTTCTTCC | |
| | GCAATGGGCAGCCGGAGAAC | TCTACAGCAAGCTCACCGTG | |
| | AACTACAAGACCACGCCTCC | GACAAGAGCAGGTGGCAGCA | |
| | CGTGCTGGACTCCGACGGCT | GGGGAACGTCTTCTCATGCT | |
| | CCTTCTTCCTCGTGAGCAAG | CCGTGATGCATGAGGCTCTG | |
| | CTCACCGTGGACAAGAGCAG | CACAACCACTACACGCAGAA | |
| | GTGGCAGCAGGGGAACGTCT | GAGCCTCTCCCTGTCTCCGG | |
| | TCTCATGCTCCGTGATGCAT | GTAAA | |
| | GAGGCTCTGCACAACCGGTT | (SEQ ID NO: 172) | |
| | CACGCAGAAGAGCCTCTCCC | | |
| | TGTCTCCGGGTAAA | | |
| | (SEQ ID NO: 178) | | |
| FUSE-489 | GAGGTGCAGCTGGTGGAGTC | CAGGTCCAGCTCCAGGAAAG | CAGGCTGTGGTGACCCAGGA |
| | CGGAGGAGGACTGGTGCAGC | CGGCGGCGGCCTCGTGCAGC | GCCCAGCCTGACCGTGAGCC |
| | CTGGAGGAAGCCTGAGGCTG | CTGGAGGAAGTTTGAGGCTC | CCGGAGGAACAGTGACCCTG |
| | TCCTGCGCTGCTTCCGGATT | TCCTGCACAGCCTCCGGCTA | ACCTGCAGAAGCAGCACCGG |
| | TACATTTAGCACATATGCCA | CACCAACAGACTCAAATGCA | CGCCGTGACAACCTCAAACT |
| | TGAACTGGGTGAGGCAGGCC | TGGGCTGGGTCAGACAGGCC | ACGCCAACTGGGTGCAGCAG |
| | CCTGGAAAAGGTCTGGAATG | CCCGGAAAAGAAAGGGAGGA | AAACCTGGACAGGCCCCCAG |
| | GGTGGCTAGGATTCGGTCCA | GGTGGCTACTATTTCCACCG | AGGACTGATCGGCGGAACCA |
| | AGTACAACAATTACGCCACC | GAACAGGAAACACCTACTAC | ACAAGAGAGCCCCCGGCACC |
| | TACTATGCCGACAGTGTCAA | GCCGACAGTGTGAAGGGCAG | CCCGCCAGATTCAGCGGAAG |
| | GGACAGGTITACCATCTCCA | GTTCACCATCTCCAGGGACA | TCTGCTGGGCGGCAAAGCCG |
| | GGGACGATTCAAAGAACACC | ATTCTAAGAACACACTGTAC | CCCTGACCCTGAGCGGAGTG |
| | CTGTACCTGCAGATGAACTC | TTGCAGATGAACAGCTTGAG | CAGCCTGAGGATGAAGCCGA |
| | CCTGAGGGCCGAGGATACCG | GGCCGAGGACACCGCCATGT | GTACTACTGCGCTCTGTGGT |
| | CTGTGTATTACTGTGCCCGG | ACTACTGCGCCGCCGACGTG | ACAGCAACCTGTGGGTGTTC |
| | CACGGAAATTTCGGGAACTC | AGGCCCGACGGAACAACCTG | GGAGGAGGCACCAAGCTGAC |
| | CTACGTGTCCTGGTTCGCAT | CCACTACAACAGCGGAGGCC | CGTGCTGGGTCAGCCCAAGG |
| | ACTGGGGCCAGGGAACAATG | AGGGAACCCAGGTGACCGTG | CTGCCCCCTCGGTCACTCTG |
| | GTGACTGTGTCCTCCGCTAG | TCCTCCGAGCCCAAATCTAG | TTCCCGCCCTCCTCTGAGGA |
| | CACCAAGGGCCCATCGGTCT | CGACAAAACTCACACATGCC | GCTTCAAGCCAACAAGGCCA |
| | TCCCCCTGGCACCCTCCTCC | CACCGTGCCCAGCACCTGAA | CACTGGTGTGTCTCATAAGT |
| | AAGAGCACCTCTGGGGGCAC | GCCGCCGGGGGACCGTCAGT | GACTTCTACCCGGGAGCCGT |
| | AGCGGCCCTGGGCTGCCTGG | CTTCCTCTTCCCCCCAAAAC | GACAGTGGCCTGGAAGGCAG |
| | TCAAGGACTACTTCCCCGAA | CCAAGGACACCCTCATGATC | ATAGCAGCCCCGTCAAGGCG |
| | CCGGTGACGGTGTCGTGGAA | TCCCGGACCCCCGAGGTCAC | GGAGTGGAGACCACCACACC |
| | CTCAGGCGCCCTGACCAGCG | ATGCGTGGTGGTGGACGTGA | CTCCAAACAAAGCAACAACA |
| | GCGTGCACACCTTCCCGGCT | GCCACGAAGACCCTGAGGTC | AGTACGCGGCCAGCAGCTAC |
| | GTCCTACAGTCCTCAGGACT | AAGTTCAACTGGTACGTGGA | CTGAGCCTGACGCCTGAGCA |
| | CTACTCCCTCAGCAGCGTGG | CGGCGTGGAGGTGCATAATG | GTGGAAGTCCCACAGGAGCT |
| | TGACCGTGCCCTCCAGCAGC | CCAAGACAAAGCCGCGGGAG | ACAGTTGCCAGGTCACGCAT |
| | TTGGGCACCCAGACCTACAT | GAGCAGTACAACAGCACGTA | GAAGGGAGCACCGTGGAGAA |
| | CTGCAACGTGAATCACAAGC | CCGTGTGGTCAGCGTCCTCA | GACAGTGGCCCCTACAGAAT |
| | CCAGCAACACCAAGGTGGAC | CCGTCCTGCACCAGGACTGG | GTTCAGGTGGCGGAGGGTCT |
| | AAGAAAGTIGAGCCCAAATC | CTGAATGGCAAGGAGTACAA | GGTGGTGGAGGATCAGGGGG |
| | TTGTGACAAAACTCACACAT | GTGCAAGGTCTCCAACAAAG | TGGAGGTTCAGGAGGCGGGG |
| | GCCCACCGTGCCCAGCACCT | CCCTCCCAGCCCCCATCGAG | GAAGTCAGGTCCAGCTCCAG |
| | GAAGCCGCAGGGGGACCGTC | AAAACCATCTCCAAAGCCAA | GAAAGCGGCGGCGGCCTCGT |
| | AGTCTTCCTCTTCCCCCCAA | AGGGCAGCCCCGAGAACCAC | GCAGCCTGGAGGAAGTTTGA |
| | AACCCAAGGACACCCTCATG | AGGTGTACACCCTGCCCCCA | GGCTCTCCTGCACAGCCTCC |
| | ATCTCCCGGACCCCCGAGGT | TCCCGGGAGGAGATGACCAA | GGCTACACCAACAGACTCAA |
| | CACATGCGTGGTGGTGGACG | GAACCAGGTCAGCCTGTGGT | ATGCATGGGCTGGGTCAGAC |
| | TGAGCCACGAAGACCCTGAG | GCCTGGTCAAAGGCTTCTAT | AGGCCCCCGGAAAAGAAAGG |
| | GTCAAGTTCAACTGGTACGT | CCCAGCGACATCGCCGTGGA | GAGGAGGTGGCTACTATTTC |
| | GGACGGCGTGGAGGTGCATA | GTGGGAGAGCAATGGGCAGC | CACCGGAACAGGAAACACCT |
| | ATGCCAAGACAAAGCCGCGG | CGGAGAACAACTACAAGACC | ACTACGCCGACAGTGTGAAG |
| | GAGGAGCAGTACAACAGCAC | ACGCCTCCCGTGCTGGACTC | GGCAGGTTCACCATCTCCAG |
| | GTACCGTGTGGTCAGCGTCC | CGACGGCTCCTTCTTCCTCT | GGACAATTCTAAGAACACAC |
| | TCACCGTCCTGCACCAGGAC | ACAGCAAGCTCACCGTGGAC | TGTACTTGCAGATGAACAGC |
| | TGGCTGAATGGCAAGGAGTA | AAGAGCAGGTGGCAGCAGGG | TTGAGGGCCGAGGACACCGC |
| | CAAGTGCAAGGTCTCCAACA | GAACGTCTTCTCATGCTCCG | CATGTACTACTGCGCCGCCG |
| | AAGCCCTCCCAGCCCCCATC | TGATGCATGAGGCTCTGCAC | ACGTGAGGCCCGACGGAACA |
| | GAGAAAACCATCTCCAAAGC | AACCACTACACGCAGAAGAG | ACCTGCCACTACAACAGCGG |
| | CAAAGGGCAGCCCCGAGAAC | CCTCTCCCTGTCTCCGGGTA | AGGCCAGGGAACCCAGGTGA |
| | CACAGGTGTACACCCTGCCC | AA | CCGTGTCCTCC |
| | CCATCCCGGGAGGAGATGAC | (SEQ ID NO: 179) | (SEQ ID NO: 180) |
| | CAAGAACCAGGTCAGCCTGA | | |
| | GCTGCGCGGTCAAAGGCTTC | | |
| | TATCCCAGCGACATCGCCGT | | |
| | GGAGTGGGAGAGCAATGGGC | | |
| | AGCCGGAGAACAACTACAAG | | |
| | ACCACGCCTCCCGTGCTGGA | | |
| | CTCCGACGGCTCCTTCTTCC | | |
| | TCGTCAGCAAGCTCACCGTG | | |
| | GACAAGAGCAGGTGGCAGCA | | |
| | GGGGAACGTCTTCTCATGCT | | |
| | CCGTGATGCATGAGGCTCTG | | |
| | CACAACAGATTTACGCAGAA | | |
| | GAGCCTCTCCCTGTCTCCGG | | |
| | GTAAA | | |
| | (SEQ ID NO: 171) | | |
| FUSE-494 | GAGGTGCAGCTGGTGGAGTC | GAGGTGCAGTIGTTGGAGAG | CAGGCTGTGGTGACCCAGGA |
| | CGGAGGAGGACTGGTGCAGC | CGGGGGGGGGCTGGTGCAGC | GCCCAGCCTGACCGTGAGCC |
| | CTGGAGGAAGCCTGAGGCTG | CAGGAGGAAGTCTGAGGTTG | CCGGAGGAACAGTGACCCTG |
| | TCCTGCGCTGCTTCCGGATT | TCCTGCGCAGCATCCGGATA | ACCTGCAGAAGCAGCACCGG |
| | TACATTTAGCACATATGCCA | TACAAACAGGTTGAAGTGTA | CGCCGTGACAACCTCAAACT |
| | TGAACTGGGTGAGGCAGGCC | TGGGGTGGTTTAGGCAGGCA | ACGCCAACTGGGTGCAGCAG |
| | CCTGGAAAAGGTCTGGAATG | CCTGGCAAGGAGAGGGAGGA | AAACCTGGACAGGCCCCCAG |
| | GGTGGCTAGGATTCGGTCCA | GGTGAGTACAATCAGCACCG | AGGACTGATCGGCGGAACCA |
| | AGTACAACAATTACGCCACC | GAACCGGAAACACCTATTAC | ACAAGAGAGCCCCCGGCACC |
| | TACTATGCCGACAGTGTCAA | GCCGACAGTGTCAAGGGCAG | CCCGCCAGATTCAGCGGAAG |
| | GGACAGGTTTACCATCTCCA | GTTCACAATCTCCCAGGACA | TCTGCTGGGCGGCAAAGCCG |
| | GGGACGATTCAAAGAACACC | AGAGCAAGAACACACTGTAC | CCCTGACCCTGAGCGGAGTG |
| | CTGTACCTGCAGATGAACTC | CTGAGGATGAACAGCCTGAG | CAGCCTGAGGATGAAGCCGA |
| | CCTGAGGGCCGAGGATACCG | GGCAGAAGATACTGCTCTGT | GTACTACTGCGCTCTGTGGT |
| | CTGTGTATTACTGTGCCCGG | ATTATTGCGCCGCCGACGTT | ACAGCAACCTGTGGGTGTTC |
| | CACGGAAATTTCGGGAACTC | AGGCCCGATGGAACAACCTG | GGAGGAGGCACCAAGCTGAC |
| | CTACGTGTCCTGGTTCGCAT | CCACTACAACTCCGGAGGAC | CGTGCTGGGTCAGCCCAAGG |
| | ACTGGGGCCAGGGAACAATG | AGGGAACCCAGGTCACCGTG | CTGCCCCCTCGGTCACTCTG |
| | GTGACTGTGTCCTCCGCTAG | AGCTCCGAGCCCAAATCTAG | TTCCCGCCCTCCTCTGAGGA |
| | CACCAAGGGCCCATCGGTCT | CGACAAAACTCACACATGCC | GCTTCAAGCCAACAAGGCCA |
| | TCCCCCTGGCACCCTCCTCC | CACCGTGCCCAGCACCTGAA | CACTGGTGTGTCTCATAAGT |
| | AAGAGCACCTCTGGGGGCAC | GCCGCCGGGGGACCGTCAGT | GACTTCTACCCGGGAGCCGT |
| | AGCGGCCCTGGGCTGCCTGG | CTTCCTCTTCCCCCCAAAAC | GACAGTGGCCTGGAAGGCAG |
| | TCAAGGACTACTTCCCCGAA | CCAAGGACACCCTCATGATC | ATAGCAGCCCCGTCAAGGCG |
| | CCGGTGACGGTGTCGTGGAA | TCCCGGACCCCCGAGGTCAC | GGAGTGGAGACCACCACACC |
| | CTCAGGCGCCCTGACCAGCG | ATGCGTGGTGGTGGACGTGA | CTCCAAACAAAGCAACAACA |
| | GCGTGCACACCTTCCCGGCT | GCCACGAAGACCCTGAGGTC | AGTACGCGGCCAGCAGCTAC |
| | GTCCTACAGTCCTCAGGACT | AAGTTCAACTGGTACGTGGA | CTGAGCCTGACGCCTGAGCA |
| | CTACTCCCTCAGCAGCGTGG | CGGCGTGGAGGTGCATAATG | GTGGAAGTCCCACAGGAGCT |
| | TGACCGTGCCCTCCAGCAGC | CCAAGACAAAGCCGCGGGAG | ACAGTIGCCAGGTCACGCAT |
| | TTGGGCACCCAGACCTACAT | GAGCAGTACAACAGCACGTA | GAAGGGAGCACCGTGGAGAA |
| | CTGCAACGTGAATCACAAGC | CCGTGTGGTCAGCGTCCTCA | GACAGTGGCCCCTACAGAAT |
| | CCAGCAACACCAAGGTGGAC | CCGTCCTGCACCAGGACTGG | GTTCAGGTGGCGGAGGGTCT |
| | AAGAAAGTTGAGCCCAAATC | CTGAATGGCAAGGAGTACAA | GGTGGTGGAGGATCAGGGGG |
| | TTGTGACAAAACTCACACAT | GTGCAAGGTCTCCAACAAAG | TGGAGGTTCAGGAGGCGGGG |
| | GCCCACCGTGCCCAGCACCT | CCCTCCCAGCCCCCATCGAG | GAAGTGAGGTGCAGTTGTTG |
| | GAAGCCGCAGGGGGACCGTC | AAAACCATCTCCAAAGCCAA | GAGAGCGGGGGGGGGCTGGT |
| | AGTCTTCCTCTTCCCCCCAA | AGGGCAGCCCCGAGAACCAC | GCAGCCAGGAGGAAGTCTGA |
| | AACCCAAGGACACCCTCATG | AGGTGTACACCCTGCCCCCA | GGTTGTCCTGCGCAGCATCC |
| | ATCTCCCGGACCCCCGAGGT | TCCCGGGAGGAGATGACCAA | GGATATACAAACAGGTTGAA |
| | CACATGCGTGGTGGTGGACG | GAACCAGGTCAGCCTGTGGT | GTGTATGGGGTGGTTTAGGC |
| | TGAGCCACGAAGACCCTGAG | GCCTGGTCAAAGGCTTCTAT | AGGCACCTGGCAAGGAGAGG |
| | GTCAAGTTCAACTGGTACGT | CCCAGCGACATCGCCGTGGA | GAGGAGGTGAGTACAATCAG |
| | GGACGGCGTGGAGGTGCATA | GTGGGAGAGCAATGGGCAGC | CACCGGAACCGGAAACACCT |
| | ATGCCAAGACAAAGCCGCGG | CGGAGAACAACTACAAGACC | ATTACGCCGACAGTGTCAAG |
| | GAGGAGCAGTACAACAGCAC | ACGCCTCCCGTGCTGGACTC | GGCAGGTTCACAATCTCCCA |
| | GTACCGTGTGGTCAGCGTCC | CGACGGCTCCTTCTTCCTCT | GGACAAGAGCAAGAACACAC |
| | TCACCGTCCTGCACCAGGAC | ACAGCAAGCTCACCGTGGAC | TGTACCTGAGGATGAACAGC |
| | TGGCTGAATGGCAAGGAGTA | AAGAGCAGGTGGCAGCAGGG | CTGAGGGCAGAAGATACTGC |
| | CAAGTGCAAGGTCTCCAACA | GAACGTCTTCTCATGCTCCG | TCTGTATTATTGCGCCGCCG |
| | AAGCCCTCCCAGCCCCCATC | TGATGCATGAGGCTCTGCAC | ACGTTAGGCCCGATGGAACA |
| | GAGAAAACCATCTCCAAAGC | AACCACTACACGCAGAAGAG | ACCTGCCACTACAACTCCGG |
| | CAAAGGGCAGCCCCGAGAAC | CCTCTCCCTGTCTCCGGGTA | AGGACAGGGAACCCAGGTCA |
| | CACAGGTGTACACCCTGCCC | AA | CCGTGAGCTCC |
| | CCATCCCGGGAGGAGATGAC | (SEQ ID NO: 181) | (SEQ ID NO: 182) |
| | CAAGAACCAGGTCAGCCTGA | | |
| | GCTGCGCGGTCAAAGGCTTC | | |
| | TATCCCAGCGACATCGCCGT | | |
| | GGAGTGGGAGAGCAATGGGC | | |
| | AGCCGGAGAACAACTACAAG | | |
| | ACCACGCCTCCCGTGCTGGA | | |
| | CTCCGACGGCTCCTTCTTCC | | |
| | TCGTCAGCAAGCTCACCGTG | | |
| | GACAAGAGCAGGTGGCAGCA | | |
| | GGGGAACGTCTTCTCATGCT | | |
| | CCGTGATGCATGAGGCTCTG | | |
| | CACAACAGATITACGCAGAA | | |
| | GAGCCTCTCCCTGTCTCCGG | | |
| | GTAAA | | |
| | (SEQ ID NO: 171) | | |
| FUSE-497 | GAGGTGCAGCTGGTGGAGTC | GAGGTGCAGTTGTTGGAGAG | CAGGCTGTGGTGACCCAGGA |
| | CGGAGGAGGACTGGTGCAGC | CGGGGGGGGGCTGGTGCCCA | GCCCAGCCTGACCGTGAGCC |
| | CTGGAGGAAGCCTGAGGCTG | GAGGAGGAAGTTTGAGGTTG | CCGGAGGAACAGTGACCCTG |
| | TCCTGCGCTGCTTCCGGATT | TCCTGCACAGCAAGCGGGTA | ACCTGCAGAAGCAGCACCGG |
| | TACATTTAGCACATATGCCA | CACAAACAGGCTGAAGTGTA | CGCCGTGACAACCTCAAACT |
| | TGAACTGGGTGAGGCAGGCC | TGGGGTGGTTTAGGCAGGCA | ACGCCAACTGGGTGCAGCAG |
| | CCTGGAAAAGGTCTGGAATG | CCTGGCAAGGAGAGGGAGGA | AAACCTGGACAGGCCCCCAG |
| | GGTGGCTAGGATTCGGTCCA | GATCGCAACAATCAGCACAG | AGGACTGATCGGCGGAACCA |
| | AGTACAACAATTACGCCACC | GAACAGGCAACACCTACTAT | ACAAGAGAGCCCCCGGCACC |
| | TACTATGCCGACAGTGTCAA | GCCGACAGTGTGAAGGGCAG | CCCGCCAGATTCAGCGGAAG |
| | GGACAGGTTTACCATCTCCA | GTTCACAATCTCCCGTGACA | TCTGCTGGGCGGCAAAGCCG |
| | GGGACGATTCAAAGAACACC | ACAGCAGGAACACCCTGTAC | CCCTGACCCTGAGCGGAGTG |
| | CTGTACCTGCAGATGAACTC | CTGCAGATGAAAACTTTACG | CAGCCTGAGGATGAAGCCGA |
| | CCTGAGGGCCGAGGATACCG | TGCCGAAGATACTGCTGTGT | GTACTACTGCGCTCTGTGGT |
| | CTGTGTATTACTGTGCCCGG | ACTATTGCGCCGCCGACGTT | ACAGCAACCTGTGGGTGTTC |
| | CACGGAAATTTCGGGAACTC | AGGCCCGATGGAACAACCTG | GGAGGAGGCACCAAGCTGAC |
| | CTACGTGTCCTGGTTCGCAT | CCACTACAACTCCTGGGGAC | CGTGCTGGGTCAGCCCAAGG |
| | ACTGGGGCCAGGGAACAATG | AGGGAACCCAGGTCACCGTG | CTGCCCCCTCGGTCACTCTG |
| | GTGACTGTGTCCTCCGCTAG | AGCTCCGAGCCCAAATCTAG | TTCCCGCCCTCCTCTGAGGA |
| | CACCAAGGGCCCATCGGTCT | CGACAAAACTCACACATGCC | GCTTCAAGCCAACAAGGCCA |
| | TCCCCCTGGCACCCTCCTCC | CACCGTGCCCAGCACCTGAA | CACTGGTGTGTCTCATAAGT |
| | AAGAGCACCTCTGGGGGCAC | GCCGCCGGGGGACCGTCAGT | GACTTCTACCCGGGAGCCGT |
| | AGCGGCCCTGGGCTGCCTGG | CTTCCTCTTCCCCCCAAAAC | GACAGTGGCCTGGAAGGCAG |
| | TCAAGGACTACTTCCCCGAA | CCAAGGACACCCTCATGATC | ATAGCAGCCCCGTCAAGGCG |
| | CCGGTGACGGTGTCGTGGAA | TCCCGGACCCCCGAGGTCAC | GGAGTGGAGACCACCACACC |
| | CTCAGGCGCCCTGACCAGCG | ATGCGTGGTGGTGGACGTGA | CTCCAAACAAAGCAACAACA |
| | GCGTGCACACCTTCCCGGCT | GCCACGAAGACCCTGAGGTC | AGTACGCGGCCAGCAGCTAC |
| | GTCCTACAGTCCTCAGGACT | AAGTTCAACTGGTACGTGGA | CTGAGCCTGACGCCTGAGCA |
| | CTACTCCCTCAGCAGCGTGG | CGGCGTGGAGGTGCATAATG | GTGGAAGTCCCACAGGAGCT |
| | TGACCGTGCCCTCCAGCAGC | CCAAGACAAAGCCGCGGGAG | ACAGTTGCCAGGTCACGCAT |
| | TTGGGCACCCAGACCTACAT | GAGCAGTACAACAGCACGTA | GAAGGGAGCACCGTGGAGAA |
| | CTGCAACGTGAATCACAAGC | CCGTGTGGTCAGCGTCCTCA | GACAGTGGCCCCTACAGAAT |
| | CCAGCAACACCAAGGTGGAC | CCGTCCTGCACCAGGACTGG | GTTCAGGTGGCGGAGGGTCT |
| | AAGAAAGTTGAGCCCAAATC | CTGAATGGCAAGGAGTACAA | GGTGGTGGAGGATCAGGGGG |
| | TTGTGACAAAACTCACACAT | GTGCAAGGTCTCCAACAAAG | TGGAGGTTCAGGAGGCGGGG |
| | GCCCACCGTGCCCAGCACCT | CCCTCCCAGCCCCCATCGAG | GAAGTGAGGTGCAGTTGTTG |
| | GAAGCCGCAGGGGGACCGTC | AAAACCATCTCCAAAGCCAA | GAGAGCGGGGGGGGGCTGGT |
| | AGTCTTCCTCTTCCCCCCAA | AGGGCAGCCCCGAGAACCAC | GCCCAGAGGAGGAAGTTTGA |
| | AACCCAAGGACACCCTCATG | AGGTGTACACCCTGCCCCCA | GGTTGTCCTGCACAGCAAGC |
| | ATCTCCCGGACCCCCGAGGT | TCCCGGGAGGAGATGACCAA | GGGTACACAAACAGGCTGAA |
| | CACATGCGTGGTGGTGGACG | GAACCAGGTCAGCCTGTGGT | GTGTATGGGGGGTTTAGGCA |
| | TGAGCCACGAAGACCCTGAG | GCCTGGTCAAAGGCTTCTAT | GGCACCTGGCAAGGAGAGGG |
| | GTCAAGTTCAACTGGTACGT | CCCAGCGACATCGCCGTGGA | AGGAGATCGCAACAATCAGC |
| | GGACGGCGTGGAGGTGCATA | GTGGGAGAGCAATGGGCAGC | ACAGGAACAGGCAACACCTA |
| | ATGCCAAGACAAAGCCGCGG | CGGAGAACAACTACAAGACC | CTATGCCGACAGTGTGAAGG |
| | GAGGAGCAGTACAACAGCAC | ACGCCTCCCGTGCTGGACTC | GCAGGTTCACAATCTCCCGT |
| | GTACCGTGTGGTCAGCGTCC | CGACGGCTCCTTCTTCCTCT | GACAACAGCAGGAACACCCT |
| | TCACCGTCCTGCACCAGGAC | ACAGCAAGCTCACCGTGGAC | GTACCTGCAGATGAAAACTT |
| | TGGCTGAATGGCAAGGAGTA | AAGAGCAGGTGGCAGCAGGG | TACGTGCCGAAGATACTGCT |
| | CAAGTGCAAGGTCTCCAACA | GAACGTCTTCTCATGCTCCG | GTGTACTATTGCGCCGCCGA |
| | AAGCCCTCCCAGCCCCCATC | TGATGCATGAGGCTCTGCAC | CGTTAGGCCCGATGGAACAA |
| | GAGAAAACCATCTCCAAAGC | AACCACTACACGCAGAAGAG | CCTGCCACTACAACTCCTGG |
| | CAAAGGGCAGCCCCGAGAAC | CCTCTCCCTGTCTCCGGGTA | GGACAGGGAACCCAGGTCAC |
| | CACAGGTGTACACCCTGCCC | AA | CGTGAGCTCC |
| | CCATCCCGGGAGGAGATGAC | (SEQ ID NO: 183) | (SEQ ID NO: 184) |
| | CAAGAACCAGGTCAGCCTGA | | |
| | GCTGCGCGGTCAAAGGCTTC | | |
| | TATCCCAGCGACATCGCCGT | | |
| | GGAGTGGGAGAGCAATGGGC | | |
| | AGCCGGAGAACAACTACAAG | | |
| | ACCACGCCTCCCGTGCTGGA | | |
| | CTCCGACGGCTCCTTCTTCC | | |
| | TCGTCAGCAAGCTCACCGTG | | |
| | GACAAGAGCAGGTGGCAGCA | | |
| | GGGGAACGTCTTCTCATGCT | | |
| | CCGTGATGCATGAGGCTCTG | | |
| | CACAACAGATTTACGCAGAA | | |
| | GAGCCTCTCCCTGTCTCCGG | | |
| | GTAAA | | |
| | (SEQ ID NO: 171) | | |
| FUSE-498 | GAGGTGCAGCTGGTGGAGTC | GAGGTGCAGTTGGTGGAGAG | CAGGCTGTGGTGACCCAGGA |
| | CGGAGGAGGACTGGTGCAGC | CGGGGGGGGGCTGGTGCAGC | GCCCAGCCTGACCGTGAGCC |
| | CTGGAGGAAGCCTGAGGCTG | CTGGAGGAAGTTTGAGGTTG | CCGGAGGAACAGTGACCCTG |
| | TCCTGCGCTGCTTCCGGATT | AGCTGTACAGCAAGCGGGTA | ACCTGCAGAAGCAGCACCGG |
| | TACATTTAGCACATATGCCA | CACAAACAGGTTGAAGTGTA | CGCCGTGACAACCTCAAACT |
| | TGAACTGGGTGAGGCAGGCC | TGGGGTGGTTCAGGCAGGCA | ACGCCAACTGGGTGCAGCAG |
| | CCTGGAAAAGGTCTGGAATG | CCTGGAAAGGAGAGGGAGGA | AAACCTGGACAGGCCCCCAG |
| | GGTGGCTAGGATTCGGTCCA | GATCGCAACCATTICCACAG | AGGACTGATCGGCGGAACCA |
| | AGTACAACAATTACGCCACC | GAACAGGCAACACCTACTAC | ACAAGAGAGCCCCCGGCACC |
| | TACTATGCCGACAGTGTCAA | GCCGACAGTGTGAAGGGCAG | CCCGCCAGATTCAGCGGAAG |
| | GGACAGGTTTACCATCTCCA | GTTCACATTCTCCAGGGACA | TCTGCTGGGCGGCAAAGCCG |
| | GGGACGATTCAAAGAACACC | ACTCCAAGAACACACTGTAC | CCCTGACCCTGAGCGGAGTG |
| | CTGTACCTGCAGATGAACTC | TTGCAGATGAACAGCCTGAG | CAGCCTGAGGATGAAGCCGA |
| | CCTGAGGGCCGAGGATACCG | GGCCGAAGATACTGCTGTGT | GTACTACTGCGCTCTGTGGT |
| | CTGTGTATTACTGTGCCCGG | ACTACTGCGCCGCCGACGTT | ACAGCAACCTGTGGGTGTTC |
| | CACGGAAATTTCGGGAACTC | AGGCCCGACGGAACAACATG | GGAGGAGGCACCAAGCTGAC |
| | CTACGTGTCCTGGTTCGCAT | CCACTACAACTCCGGTGGCC | CGTGCTGGGTCAGCCCAAGG |
| | ACTGGGGCCAGGGAACAATG | AGGGCACCCAGGTCACCGTG | CTGCCCCCTCGGTCACTCTG |
| | GTGACTGTGTCCTCCGCTAG | TCCTCCGAGCCCAAATCTAG | TTCCCGCCCTCCTCTGAGGA |
| | CACCAAGGGCCCATCGGTCT | CGACAAAACTCACACATGCC | GCTTCAAGCCAACAAGGCCA |
| | TCCCCCTGGCACCCTCCTCC | CACCGTGCCCAGCACCTGAA | CACTGGTGTGTCTCATAAGT |
| | AAGAGCACCTCTGGGGGCAC | GCCGCCGGGGGACCGTCAGT | GACTTCTACCCGGGAGCCGT |
| | AGCGGCCCTGGGCTGCCTGG | CTTCCTCTTCCCCCCAAAAC | GACAGTGGCCTGGAAGGCAG |
| | TCAAGGACTACTTCCCCGAA | CCAAGGACACCCTCATGATC | ATAGCAGCCCCGTCAAGGCG |
| | CCGGTGACGGTGTCGTGGAA | TCCCGGACCCCCGAGGTCAC | GGAGTGGAGACCACCACACC |
| | CTCAGGCGCCCTGACCAGCG | ATGCGTGGTGGTGGACGTGA | CTCCAAACAAAGCAACAACA |
| | GCGTGCACACCTTCCCGGCT | GCCACGAAGACCCTGAGGTC | AGTACGCGGCCAGCAGCTAC |
| | GTCCTACAGTCCTCAGGACT | AAGTTCAACTGGTACGTGGA | CTGAGCCTGACGCCTGAGCA |
| | CTACTCCCTCAGCAGCGTGG | CGGCGTGGAGGTGCATAATG | GTGGAAGTCCCACAGGAGCT |
| | TGACCGTGCCCTCCAGCAGC | CCAAGACAAAGCCGCGGGAG | ACAGTTGCCAGGTCACGCAT |
| | TTGGGCACCCAGACCTACAT | GAGCAGTACAACAGCACGTA | GAAGGGAGCACCGTGGAGAA |
| | CTGCAACGTGAATCACAAGC | CCGTGTGGTCAGCGTCCTCA | GACAGTGGCCCCTACAGAAT |
| | CCAGCAACACCAAGGTGGAC | CCGTCCTGCACCAGGACTGG | GTTCAGGTGGCGGAGGGTCT |
| | AAGAAAGTTGAGCCCAAATC | CTGAATGGCAAGGAGTACAA | GGTGGTGGAGGATCAGGGGG |
| | TTGTGACAAAACTCACACAT | GTGCAAGGTCTCCAACAAAG | TGGAGGTTCAGGAGGGGGGG |
| | GCCCACCGTGCCCAGCACCT | CCCTCCCAGCCCCCATCGAG | AAGTGAGGTGCAGTTGGTGG |
| | GAAGCCGCAGGGGGACCGTC | AAAACCATCTCCAAAGCCAA | AGAGCGGGGGGGGGCTGGTG |
| | AGTCTTCCTCTTCCCCCCAA | AGGGCAGCCCCGAGAACCAC | CAGCCTGGAGGAAGTTTGAG |
| | AACCCAAGGACACCCTCATG | AGGTGTACACCCTGCCCCCA | GTTGAGCTGTACAGCAAGCG |
| | ATCTCCCGGACCCCCGAGGT | TCCCGGGAGGAGATGACCAA | GGTACACAAACAGGTTGAAG |
| | CACATGCGTGGTGGTGGACG | GAACCAGGTCAGCCTGTGGT | TGTATGGGGGGTTCAGGCAG |
| | TGAGCCACGAAGACCCTGAG | GCCTGGTCAAAGGCTTCTAT | GCACCTGGAAAGGAGAGGGA |
| | GTCAAGTTCAACTGGTACGT | CCCAGCGACATCGCCGTGGA | GGAGATCGCAACCATTICCA |
| | GGACGGCGTGGAGGTGCATA | GTGGGAGAGCAATGGGCAGC | CAGGAACAGGCAACACCTAC |
| | ATGCCAAGACAAAGCCGCGG | CGGAGAACAACTACAAGACC | TACGCCGACAGTGTGAAGGG |
| | GAGGAGCAGTACAACAGCAC | ACGCCTCCCGTGCTGGACTC | CAGGTTCACATTCTCCAGGG |
| | GTACCGTGTGGTCAGCGTCC | CGACGGCTCCTTCTTCCTCT | ACAACTCCAAGAACACACTG |
| | TCACCGTCCTGCACCAGGAC | ACAGCAAGCTCACCGTGGAC | TACTTGCAGATGAACAGCCT |
| | TGGCTGAATGGCAAGGAGTA | AAGAGCAGGTGGCAGCAGGG | GAGGGCCGAAGATACTGCTG |
| | CAAGTGCAAGGTCTCCAACA | GAACGTCTTCTCATGCTCCG | TGTACTACTGCGCCGCCGAC |
| | AAGCCCTCCCAGCCCCCATC | TGATGCATGAGGCTCTGCAC | GTTAGGCCCGACGGAACAAC |
| | GAGAAAACCATCTCCAAAGC | AACCACTACACGCAGAAGAG | ATGCCACTACAACTCCGGTG |
| | CAAAGGGCAGCCCCGAGAAC | CCTCTCCCTGTCTCCGGGTA | GCCAGGGCACCCAGGTCACC |
| | CACAGGTGTACACCCTGCCC | AA | GTGTCCTCC |
| | CCATCCCGGGAGGAGATGAC | (SEQ ID NO: 185) | (SEQ ID NO: 186) |
| | CAAGAACCAGGTCAGCCTGA | | |
| | GCTGCGCGGTCAAAGGCTTC | | |
| | TATCCCAGCGACATCGCCGT | | |
| | GGAGTGGGAGAGCAATGGGC | | |
| | AGCCGGAGAACAACTACAAG | | |
| | ACCACGCCTCCCGTGCTGGA | | |
| | CTCCGACGGCTCCTTCTTCC | | |
| | TCGTCAGCAAGCTCACCGTG | | |
| | GACAAGAGCAGGTGGCAGCA | | |
| | GGGGAACGTCTTCTCATGCT | | |
| | CCGTGATGCATGAGGCTCTG | | |
| | CACAACAGATTTACGCAGAA | | |
| | GAGCCTCTCCCTGTCTCCGG | | |
| | GTAAA | | |
| | (SEQ ID NO: 171) | | |
| FUSE-536 | GAGGTGCAGCTGGTGGAGTC | GAGGTGCAGCTGCTGGAGTC | CAGGCTGTGGTGACCCAGGA |
| | CGGAGGAGGACTGGTGCAGC | CGGAGGAGGACTGGTGCAGC | GCCCAGCCTGACCGTGAGCC |
| | CTGGAGGAAGCCTGAGGCTG | CTGGAGGAAGCCTGAGGCTG | CCGGAGGAACAGTGACCCTG |
| | TCCTGCGCTGCTTCCGGATT | TCCTGCGCTGCTTCCGGATA | ACCTGCAGAAGCAGCACCGG |
| | TACATTTAGCACATATGCCA | TACCAACAGGCTGAAGTGTA | CGCCGTGACAACCTCAAACT |
| | TGAACTGGGTGAGGCAGGCC | TGGGATGGTTTAGGCAGGCC | ACGCCAACTGGGTGCAGCAG |
| | CCTGGAAAAGGTCTGGAATG | CCCGGAAAAGAGAGGGAGCT | AAACCTGGACAGGCCCCCAG |
| | GGTGGCTAGGATTCGGTCCA | GGCTTCCATCAGCACCGGAA | AGGACTGATCGGCGGAACCA |
| | AGTACAACAATTACGCCACC | CTGGAAACACCTACTACGCC | ACAAGAGAGCCCCCGGCACC |
| | TACTATGCCGACAGTGTCAA | GACTCCGTGAAGGGAAGGTT | CCCGCCAGATTCAGCGGAAG |
| | GGACAGGTTTACCATCTCCA | CACAATCTCCAGAGACAACA | TCTGCTGGGCGGCAAAGCCG |
| | GGGACGATTCAAAGAACACC | GCAAGAACACACTCTACCTC | CCCTGACCCTGAGCGGAGTG |
| | CTGTACCTGCAGATGAACTC | CAGATGAACTCCCTGAAAGC | CAGCCTGAGGATGAAGCCGA |
| | CCTGAGGGCCGAGGATACCG | CGAGGACACCGCCGTGTATT | GTACTACTGCGCTCTGTGGT |
| | CTGTGTATTACTGTGCCCGG | ACTGCGCTGCTGACGTGAGG | ACAGCAACCTGTGGGTGTTC |
| | CACGGAAATTTCGGGAACTC | CCCGATGGAACCACCTGCCA | GGAGGAGGCACCAAGCTGAC |
| | CTACGTGTCCTGGTTCGCAT | CTATAACAGCCGGGGACAGG | CGTGCTGGGTCAGCCCAAGG |
| | ACTGGGGCCAGGGAACAATG | GAACCCTGGTCACCGTGTCC | CTGCCCCCTCGGTCACTCTG |
| | GTGACTGTGTCCTCCGCTAG | TCCGAGCCTAAATCCTCCGA | TTCCCGCCCTCCTCTGAGGA |
| | CACCAAGGGCCCATCGGTCT | CAAAACACACACATGCCCCC | GCTTCAAGCCAACAAGGCCA |
| | TCCCCCTGGCACCCTCCTCC | CCTGCCCTGCACCTGAGGCT | CACTGGTGTGTCTCATAAGT |
| | AAGAGCACCTCTGGGGGCAC | GCTGGAGGACCTTCCGTGTT | GACTTCTACCCGGGAGCCGT |
| | AGCGGCCCTGGGCTGCCTGG | TCTGTTCCCCCCTAAACCCA | GACAGTGGCCTGGAAGGCAG |
| | TCAAGGACTACTTCCCCGAA | AGGACACCCTCATGATTTCC | ATAGCAGCCCCGTCAAGGCG |
| | CCGGTGACGGTGTCGTGGAA | CGCACCCCCGAAGTGACCTG | GGAGTGGAGACCACCACACC |
| | CTCAGGCGCCCTGACCAGCG | CGTGGTGGTGGACGTGTCTC | CTCCAAACAAAGCAACAACA |
| | GCGTGCACACCTTCCCGGCT | ATGAAGACCCCGAGGTGAAG | AGTACGCGGCCAGCAGCTAC |
| | GTCCTACAGTCCTCAGGACT | TTCAATTGGTACGTGGACGG | CTGAGCCTGACGCCTGAGCA |
| | CTACTCCCTCAGCAGCGTGG | CGTGGAGGTGCACAATGCAA | GTGGAAGTCCCACAGGAGCT |
| | TGACCGTGCCCTCCAGCAGC | AAACCAAACCCAGGGAGGAG | ACAGTTGCCAGGTCACGCAT |
| | TTGGGCACCCAGACCTACAT | CAGTATAACTCCACATATAG | GAAGGGAGCACCGTGGAGAA |
| | CTGCAACGTGAATCACAAGC | GGTGGTGTCCGTGCTGACCG | GACAGTGGCCCCTACAGAAT |
| | CCAGCAACACCAAGGTGGAC | TGCTGCACCAGGACTGGCTG | GTTCAGGTGGCGGAGGGTCT |
| | AAGAAAGTTGAGCCCAAATC | AATGGAAAGGAGTACAAATG | GGTGGTGGAGGATCCGGGGG |
| | TTGTGACAAAACTCACACAT | CAAGGTGTCCAACAAAGCAC | AGGAGGTTCAGGAGGCGGGG |
| | GCCCACCGTGCCCAGCACCT | TGCCAGCCCCCATTGAGAAA | GATCCGAGGTGCAGCTGCTG |
| | GAAGCCGCAGGGGGACCGTC | ACCATCTCCAAAGCCAAGGG | GAGTCCGGAGGAGGACTGGT |
| | AGTCTTCCTCTTCCCCCCAA | CCAGCCCAGAGAACCACAGG | GCAGCCTGGAGGAAGCCTGA |
| | AACCCAAGGACACCCTCATG | TGTACACACTGCCCCCCTCA | GGCTGTCCTGCGCTGCTTCC |
| | ATCTCCCGGACCCCCGAGGT | AGAGAAGAAATGACCAAGAA | GGATATACCAACAGGCTGAA |
| | CACATGCGTGGTGGTGGACG | CCAGGTGAGCCTCTGGTGCC | GTGTATGGGATGGTTTAGGC |
| | TGAGCCACGAAGACCCTGAG | TCGTGAAGGGATTCTACCCC | AGGCCCCCGGAAAAGAGAGG |
| | GTCAAGTTCAACTGGTACGT | TCCGACATCGCCGTGGAATG | GAGCTGGCTTCCATCAGCAC |
| | GGACGGCGTGGAGGTGCATA | GGAATCAAACGGCCAGCCTG | CGGAACTGGAAACACCTACT |
| | ATGCCAAGACAAAGCCGCGG | AGAACAACTACAAAACCACC | ACGCCGACTCCGTGAAGGGA |
| | GAGGAGCAGTACAACAGCAC | CCCCCCGTGCTCGACTCAGA | AGGTTCACAATCTCCAGAGA |
| | GTACCGTGTGGTCAGCGTCC | TGGATCTTTTTTCCTGTATT | CAACAGCAAGAACACACTCT |
| | TCACCGTCCTGCACCAGGAC | CCAAGCTGACTGTGGACAAG | ACCTCCAGATGAACTCCCTG |
| | TGGCTGAATGGCAAGGAGTA | TCTAGGTGGCAGCAGGGCAA | AAAGCCGAGGACACCGCCGT |
| | CAAGTGCAAGGTCTCCAACA | CGTCTTCAGCTGTAGCGTGA | GTATTACTGCGCTGCTGACG |
| | AAGCCCTCCCAGCCCCCATC | TGCACGAGGCCCTGCATAAC | TGAGGCCCGATGGAACCACC |
| | GAGAAAACCATCTCCAAAGC | CACTACACCCAGAAGAGTCT | TGCCACTATAACAGCCGGGG |
| | CAAAGGGCAGCCCCGAGAAC | GTCCCTGAGCCCCGGAAAG | ACAGGGAACCCTGGTCACCG |
| | CACAGGTGTACACCCTGCCC | (SEQ ID NO: 187) | TGTCCTCC |
| | CCATCCCGGGAGGAGATGAC | | (SEQ ID NO: 188) |
| | CAAGAACCAGGTCAGCCTGA | | |
| | GCTGCGCGGTCAAAGGCTTC | | |
| | TATCCCAGCGACATCGCCGT | | |
| | GGAGTGGGAGAGCAATGGGC | | |
| | AGCCGGAGAACAACTACAAG | | |
| | ACCACGCCTCCCGTGCTGGA | | |
| | CTCCGACGGCTCCTTCTTCC | | |
| | TCGTCAGCAAGCTCACCGTG | | |
| | GACAAGAGCAGGTGGCAGCA | | |
| | GGGGAACGTCTICTCATGCT | | |
| | CCGTGATGCATGAGGCTCTG | | |
| | CACAACAGATTTACGCAGAA | | |
| | GAGCCTCTCCCTGTCTCCGG | | |
| | GTAAA | | |
| | (SEQ ID NO: 171) | | |

TABLE 3
Amino acid sequences of exemplary VHH.

| | |
|---|---|
| 2A11 | QVQLQESGGGSVPAGGSLRLSCAASGSTYSANCMG |
| | WFRQAPGKEREEVASMSIRSGRTYYSDSVKGRFTI |
| | SQDGSKNTLYLQLNSLKAEDTALYYCAAAYGGSRC |
| | VYNYRGQGTQVTVSS (SEQ ID NO: 7) |
| 5A1 | QVQLQESGGGSVQAGGSLKLSCTASGYTNRLKCMG |
| | WFRQAPGKEREEIATISTGTGNTYYADSVKGRFTF |
| | SQDKVKNTVYLQMNTLKPDDTGMYYCAADVRPDGT |
| | TCHYNSGGQGTQVTVSS (SEQ ID NO: 8) |
| 5A1-H1 | EVQLLESGGGLVQPGGSLRLSCAASGYTNRLKCMG |
| | WFRQAPGKERELASISTGTGNTYYADSVKGRFTIS |
| | RDNSKNTLYLQMNSLKAEDTAVYYCAADVRPDGTT |
| | CHYNSRGQGTLVTVSS (SEQ ID NO: 11) |
| 5A1-H2 | QVQLQESGGGLVQPGGSLRLSCTASGYTNRLKCMG |
| | WVRQAPGKEREEVATISTGTGNTYYADSVKGRFTI |
| | SRDNSKNTLYLQMNSLRAEDTAMYYCAADVRPDGT |
| | TCHYNSGGQGTQVTVSS (SEQ ID NO: 12) |
| 5A1-H3 | EVQLLESGGGLVQPGGSLRLSCAASGYTNRLKCMG |
| | WFRQAPGKEREEVSTISTGTGNTYYADSVKGRFTI |
| | SQDKSKNTLYLRMNSLRAEDTALYYCAADVRPDGT |
| | TCHYNSGGQGTQVTVSS (SEQ ID NO: 13) |
| 5A1-H4 | EVQLLESGGGLVPRGGSLRLSCTASGYTNRLKCMG |
| | WFRQAPGKEREEIATISTGTGNTYYADSVKGRFTI |
| | SRDNSRNTLYLQMKTLRAEDTAVYYCAADVRPDGT |
| | TCHYNSWGQGTQVTVSS (SEQ ID NO: 14) |
| 5A1-H5 | EVQLVESGGGLVQPGGSLRLSCTASGYTNRLKCMG |
| | WFRQAPGKEREEIATISTGTGNTYYADSVKGRFTF |
| | SRDNSKNTLYLQMNSLRAEDTAVYYCAADVRPDGT |
| | TCHYNSGGQGTQVTVSS (SEQ ID NO: 15) |
| 2A11-CV | QVQLQESGGGSVPAGGSLRLSCAASGSTYSANvMG |
| | WFRQAPGKEREEVASMSIRSGRTYYSDSVKGRFTI |
| | SQDGSKNTLYLQLNSLKAEDTALYYCAAAGGSRvV |
| | YNYRGQGTQVTVSS (SEQ ID NO: 189) |
| 5A1-CV | QVQLQESGGGSVQAGGSLKLSCTASGYTNRLKvMG |
| | WFRQAPGKEREEIATISTGTGNTYYADSVKGRFTF |
| | SQDKVKNTVYLQMNTLKPDDTGMYYCAADVRPDGT |
| | TvHYNSGGQGTQVTVSS (SEQ ID NO: 190) |

TABLE 4
Polynucleotide sequences encoding the VHH
shown in Table 3.

| | |
|---|---|
| 2A11 | CAGGTCCAACTCCAAGAGAGCGGCGGCGGCTCCGT |
| | CCCAGCTGGAGGATCACTCAGACTCAGCTGCGCCG |
| | CCAGCGGCTCCACC11TACAGCGCTAACTGCATGG |
| | GCTGGTTCAGACAAGCCCCCGGCAAAGAGAGAGAA |
| | GAGGTGGCTTCCATGTCAATCAGAAGCGGCCGTAC |
| | CTACTACAGTGATTCCGTGAAAGGCAGATTCACAA |
| | TCAGCCAGGACGGAAGCAAGAACACCCTGTACCTG |
| | CAGCTGAACAGCCTGAAAGCCGAGGACACCGCCCT |
| | GTACTACTGCGCCGCCGCCTACGGGGGCTCTAGGT |
| | GTGTGTACAACTACAGAGGCCAGGGCACACAAGTC |
| | ACCGTCTCTAGC (SEQ ID NO: 9) |
| 5A1 | CAGGTCCAGCTCCAGGAAAGCGGCGGCGGCTCCGT |
| | CCAGGCAGGAGGAAGTCTCAAACTCTCCTGCACAG |
| | CCTCCGGCTACACCAACAGACTCAAATGCATGGGC |
| | TGGTTCAGACAGGCACCCGGAAAAGAGAGGGAAGA |
| | GATCGCTACCATCTCCACCGGCACCGGCAACACCT |
| | ACTACGCCGACTCCGTCAAAGGCAGGITCACATTC |
| | AGCCAGGACAAGGTGAAGAACACAGTGTACCTGCA |
| | GATGAACACACTGAAACCCGACGACACAGGCATGT |
| | ACTACTGCGCCGCCGACGTTAGGCCCGATGGAACC |
| | ACCTGCCACTACAACTCCGGAGGACAGGGAACCCA |
| | GGTCACCGTGAGCTCC (SEQ ID NO: 10) |
| 5A1-H1 | GAGGTGCAGCTGCTGGAGTCCGGAGGAGGACTGGT |
| | GCAGCCTGGAGGAAGCCTGAGGCTGTCCTGCGCTG |
| | CTTCCGGATATACCAACAGGCTGAAGTGTATGGGA |
| | TGGTTTAGGCAGGCCCCCGGAAAAGAGAGGGAGCT |
| | GGCTTCCATCAGCACCGGAACTGGAAACACCTACT |
| | ACGCCGACTCCGTGAAGGGAAGGTTCACAATCTCC |
| | AGAGACAACAGCAAGAACACACTCTACCTCCAGAT |
| | GAACTCCCTGAAAGCCGAGGACACCGCCGTGTATT |
| | ACTGCGCTGCTGACGTGAGGCCCGATGGAACCACC |
| | TGCCACTATAACAGCCGGGGACAGGGAACCCTGGT |
| | CACCGTGTCCTCC (SEQ ID NO: 191) |
| 5A1-H2 | CAGGTCCAGCTCCAGGAAAGCGGCGGCGGCCTCGT |
| | GCAGCCTGGAGGAAGTTTGAGGCTCTCCTGCACAG |
| | CCTCCGGCTACACCAACAGACTCAAATGCATGGGC |
| | TGGGTCAGACAGGCCCCCGGAAAAGAAAGGGAGGA |
| | GGTGGCTACTATTTCCACCGGAACAGGAAACACCT |
| | ACTACGCCGACAGTGTGAAGGGCAGGTTCACCATC |
| | TCCAGGGACAATTCTAAGAACACACTGTACTTGCA |
| | GATGAACAGCTTGAGGGCCGAGGACACCGCCATGT |
| | ACTACTGCGCCGCCGACGTGAGGCCCGACGGAACA |
| | ACCTGCCACTACAACAGCGGAGGCCAGGGAACCCA |
| | GGTGACCGTGTCCTCC (SEQ ID NO: 192) |
| 5A1-H3 | GAGGTGCAGTTGTTGGAGAGCGGGGGGGGGCTGGT |
| | GCAGCCAGGAGGAAGTCTGAGGTTGTOCTGCGCAG |
| | CATCCGGATATACAAACAGGTTGAAGTGTATGGGG |
| | TGGTTTAGGCAGGCACCTGGCAAGGAGAGGGAGGA |
| | GGTGAGTACAATCAGCACCGGAACCGGAAACACCT |
| | ATTACGCCGACAGTGTCAAGGGCAGGTTCACAATC |
| | TCCCAGGACAAGAGCAAGAACACACTGTACCTGAG |
| | GATGAACAGCCTGAGGGCAGAAGATACTGCTCTGT |
| | ATTATTGCGCCGCCGACGTTAGGCCCGATGGAACA |
| | ACCTGCCACTACAACTCCGGAGGACAGGGAACCCA |
| | GGTCACCGTGAGCTCC (SEQ ID NO: 193) |
| 5A1-H4 | GAGGTGCAGTTGTTGGAGAGCGGGGGGGGGCTGGT |
| | GCCCAGAGGAGGAAGTTTGAGGTTGTCCTGCACAG |
| | CAAGCGGGTACACAAACAGGCTGAAGTGTATGGGG |
| | TGGTTTAGGCAGGCACCTGGCAAGGAGAGGGAGGA |
| | GATCGCAACAATCAGCACAGGAACAGGCAACACCT |
| | ACTATGCCGACAGTGTGAAGGGCAGGTTCACAATC |
| | TCCCGTGACAACAGCAGGAACACCCTGTACCTGCA |
| | GATGAAAACTTTACGTGCCGAAGATACTGCTGTGT |
| | ACTATTGCGCCGCCGACGTTAGGCCCGATGGAACA |
| | ACCTGCCACTACAACTCCTGGGGACAGGGAACCCA |
| | GGTCACCGTGAGCTCC (SEQ ID NO: 194) |
| 5A1-H5 | GAGGTGCAGTTGGTGGAGAGCGGGGGGGGGCTGGT |
| | GCAGCCTGGAGGAAGTTTGAGGTTGAGCTGTACAG |
| | CAAGCGGGTACACAAACAGGTTGAAGTGTATGGGG |
| | TGGTTCAGGCAGGCACCTGGAAAGGAGAGGGAGGA |
| | GATCGCAACCATTICCACAGGAACAGGCAACACCT |
| | ACTACGCCGACAGTGTGAAGGGCAGGTTCACATTC |
| | TCCAGGGACAACTCCAAGAACACACTGTACTTGCA |
| | GATGAACAGCCTGAGGGCCGAAGATACTGCTGTGT |
| | ACTACTGCGCCGCCGACGTTAGGCCCGACGGAACA |
| | ACATGCCACTACAACTCCGGTGGCCAGGGCACCCA |
| | GGTCACCGTGTCCTCC (SEQ ID NO: 195) |
| 2A11-CV | CAGGTGCAGCTGCAGGAGTCCGGAGGAGGAAGCGT |
| | GCCTGCTGGAGGAAGCCTGAGGCTGTCCTGCGCTG |
| | CTTCCGGAAGCACCTATTCCGCTAACGTCATGGGC |
| | TGGTTTAGGCAGGCCCCTGGAAAAGAGAGGGAGGA |
| | AGTGGCTAGTATGAGCATCAGGAGCGGCAGGACCT |
| | ACTACTCTGACTCCGTCAAGGGAAGATTCACAATC |
| | AGCCAGGACGGAAGCAAGAACACACTGTACCTGCA |
| | GCTGAACTCTCTGAAGGCCGAGGACACCGCACTGT |
| | ACTACTGCGCTGCTGCTTACGGCGGTTCCAGAGTG |
| | GTGTACAACTACAGGGGCCAGGGAACACAGGTCAC |
| | CGTGTCCTCC (SEQ ID NO: 196) |
| 5A1-CV | CAGGTGCAGCTGCAGGAGTCCGGAGGAGGAAGCGT |
| | GCAGGCTGGAGGAAGCCTGAAACTCTCCTGCACAG |
| | CCTCCGGATATACAAACAGGCTCAAAGTGATGGGC |
| | TGGTTCAGGCAGGCCCCTGGAAAAGAAAGGGAGGA |
| | GATTGCAACAATCTCCACCGGCACAGGAAACACAT |
| | ACTACGCCGACTCTGTCAAGGGCCGGTTCACATTC |
| | TCCCAGGACAAGGTGAAGAACACAGTGTACCTCCA |
| | GATGAACACCCTGAAGCCCGACGACACAGGCATGT |
| | ACTACTGCGCAGCCGACGTGAGGCCTGATGGAACC |
| | ACCGTGCATTACAACAGCGGAGGCCAGGGAACACA |
| | GGTGACAGTGAGTTCC (SEQ ID NO: 197) |

TABLE 5
Yield of expressions as demonstrated
in Examples for some fusion proteins.

| | Yield |
|---|---|
| FUSE-211 | 360 mg/L |
| FUSE-393 | 458 mg/L |
| FUSE-394 | 455 mg/L |
| FUSE-489 | 253 mg/L |
| FUSE-497 | 213 mg/L |
| FUSE-498 | 117 mg/L |

TABLE 6
Melting temperature (Tm) of exemplary fusion proteins.

| | Tm |
|---|---|
| FUSE-210 | 70.5° C. |
| FUSE-211 | 70.4° C. |
| FUSE-394 | 70.6° C. |

TABLE 7
Additional exemplary VHH sequences used in in accordance with
various embodiments of the invention. All sequences are humanized
sequences except for SEQ ID NO: 7 and SEQ ID NO: 8.

| Framework | | |
|---|---|---|
| region | VHH | |
| FUSE-112 | camel | QVQLQESGGGSVPAGGSLRLSCAASGSTYSANCMGWFRQAPGKEREEVASMSIRSGRTYYSDSVKGRFTIS |
| | | QDGSKNTLYLQLNSLKAEDTALYYCAAAYGGSRCVYNYRGQGTQVTVSS |
| | | (SEQ ID NO: 7) |
| FUSE-179 | camel | QVQLQESGGGSVQAGGSLKLSCTASGYTNRLKCMGWFRQAPGKEREEIATISTGTGNTYYADSVKGRFTFS |
| | | QDKVKNTVYLQMNTLKPDDTGMYYCAADVRPDGTTCHYNSGGQGTQVTVSS |
| | | (SEQ ID NO: 8) |
| FUSE-488 | camel | QVQLQESGGGSVQAGGSLKLSCTASGYTNRLKCMGWFRQAPGKEREEIATISTGTGNTYYADSVKGRFTFS |
| | | QDKVKNTVYLQMNTLKPDDTGMYYCAADVRPDGTTCHYNSGGQGTQVTVSS |
| | | (SEQ ID NO: 8) |
| FUSE-497 | GenBank | EVQLLESGGGLVPRGGSLRLSCTASGYTNRLKCMGWFRQAPGKEREEIATISTGTGNTYYADSVKGRFTISR |
| | AWH66715 | DNSRNTLYLQMKTLRAEDTAVYYCAADVRPDGTTCHYNSWGQGTQVTVSS |
| | | (SEQ ID NO: 14) |
| FUSE-498 | Fusion Abs | EVQLVESGGGLVQPGGSLRLSCTASGYTNRLKCMGWFRQAPGKEREEIATISTGTGNTYYADSVKGRFTFSR |
| | | DNSKNTLYLQMNSLRAEDTAVYYCAADVRPDGTICHYNSGGQGTQVTVSS |
| | | (SEQ ID NO: 15) |
| FUSE-524 | IGHV3-23 | EVQLLESGGGLVQPGGSLRLSCAASGYTNRLKCMGWFRQAPGKERELASISTGTGNTYYADSVKGRFTISRD |
| | | NSKNTLYLQMNSLKAEDTAVYYCAADVRPDGTTCHYNSRGQGTLVTVSS |
| | | (SEQ ID NO: 11) |
| FUSE-525 | IGHV3-23 | EVQLLESGGGLVQPGGSLRLSCAASGYTNRLKCMGWFRQAPGKERELvASISTGTGNTYYADSVKGRFTISR |
| | | DNSKNTLYLQMNSLKAEDTAVYYCAADVRPDGTTCHYNSRGQGTLVTVSS |
| | | (SEQ ID NO: 16) |
| FUSE-537 | IGHV3-23 | EVQLLESGGGLVQPGGSLRLSCAASGSTYSANCMGWFRQAPGKERELASMSIRSGRTYYADSVKGRFTISRD |
| | | NSKNTLYLQMNSLKAEDTAVYYCAAAYGGSRCVYNYRGQGTLVTVSS |
| | | (SEQ ID NO: 17) |
| FUSE-559 | GenBank | EVQLLESGGGLVQPGGSLRLSCTASGYTNRLKCMGWFRQAPGKEREEIASISTGTGNTYYADSVKGRFTISR |
| | AWH66715 | DKSRNTLYLQMNTLRAEDTAVYYCAADVRPHGTTCHYNSRGQGTQVTVSS |
| | | (SEQ ID NO: 18) |
| FUSE-560 | IGHV3-23 | EVQLLESGGGLVQPGGSLRLSCAASGSTYSANCMGWFRQAPGKERELASMSIRSGRTYYADSVKGRFTISRD |
| | | SSKNTLYLQMNSLKAEDTAVYYCAAAYGGSRCVYNYRGQGTLVTVSS |
| | | (SEQ ID NO: 19) |
| FUSE-561 | IGHV3-23 | EVQLLESGGGLVQPGGSLRLSCAASGSTYSANCMGWFRQAPGKERELASMSIRSGRTYYADSVKGRFTISRD |
| | | SSKNTLYLQMNSLRAEDTAVYYCAAAYGGSRCVYNYRGQGTLVTVSS |
| | | (SEQ ID NO: 20) |
| FUSE-562 | IGHV3-23 | EVQLLESGGGLVQPGGSLRLSCAASGSTYSANCMGWFRQAPGKERELASMSIRSGRTYYSDSVKGRFTISRD |
| | | GSKNTLYLQMNSLKAEDTAVYYCAAAYGGSRCVYNYRGQGTLVTVSS |
| | | (SEQ ID NO: 21) |
| FUSE-563 | Fusion Abs | EVQLLESGGGLVQPGGSLKLSCTASGYTNRLKCMGWFRQAPGKEREEIATISTGTGNTYYADSVKGRFTISQ |
| | | DRSKNTLYLQMNSLRAEDTAVYYCAADVRPDGTTCHYNSWGQGTLVTVSS |
| | | (SEQ ID NO: 22) |
| FUSE-569 | Fusion Abs | QVQLVESGGGLVQPGGSLRLSCSASGYTNRLKCMGWFRQAPGKERELASISTGTGNTYYADSVKGRFTISR |
| | | DNSKNTLYLQMNSLRADDTAVYYCAADVRPDGTTCHYNSWGQGTQVTVSS |
| | | (SEQ ID NO: 23) |
| FUSE-570 | Fusion Abs | QVQLVESGGGLVQPGGSLRLSCSASGYTNRLKCMGWFRQAPGKEREEIATISSGTGNTYYADSVKGRFTFSR |
| | | DNSKNTLYLQMNSLRAEDTGMYYCAADVRPDGTTCHYNSGGQGTQVTVSS |
| | | (SEQ ID NO: 24) |
| FUSE-589 | IGHV3-23 | EVQLLESGGGLVQPGGSLRLSCAASGSTYSANCMGWFRQAPGKERELASMSIRSGRTYYADSVKGRFTISQD |
| | | GSKNTLYLQMNSLKAEDTAVYYCAAAYGGSRCVYNYRGQGTLVTVSS |
| | | (SEQ ID NO: 25) |
| FUSE-590 | GenBank | EVQLLESGGGLVQRGGSLRLSCTASGYTNRLKCMGWFRQAPGKEREEIASISTGTGNTYYSDSVKGRFTISR |
| | AWH66715 | DKSRNTLYLQMKSLRAEDTAVYYCAADVRPRGTTCHYNSRGQGTQVTVSS |
| | | (SEQ ID NO: 26) |
| FUSE-591 | GenBank | EVQLLESGGGLVPRGGSLRLSCAASGYTNRLKCMGWFRQAPGKEREEIATISTGTGNTYYADSVKGRFTISQ |
| | AWH66715 | DKSRNTLYLQMNSLKAEDTAVYYCAADVRPKGTTCHYNKRGQGTQVTVSS |
| | | (SEQ ID NO: 27) |
| FUSE-592 | GenBank | EVQLLESGGGLVPRGGSLRLSCAASGYTNRLKCMGWFRQAPGKEREEVASISTGTGNTYYADSVKGRFTISQ |
| | AWH66715 | DKSRNTLYLQLNSLRAEDTAVYYCAADVRPRGTTCHYNSRGQGTQVTVSS |
| | | (SEQ ID NO: 28) |
| FUSE-593 | GenBank | EVQLLESGGGLVQRGGSLRLSCAASGYTNRLKCMGWFRQAPGKEREEIASISTGTGNTYYSDSVKGRFTISR |
| | AWH66715 | DKSRNTLYLQLNSLKAEDTAVYYCAADVRPKGTTCHYNSRGQGTQVTVSS |
| | | (SEQ ID NO: 29) |
| FUSE-594 | GenBank | EVQLLESGGGLVPRGGSLRLSCTASGYTNRLKCMGWFRQAPGKEREEIASISTGTGNTYYADSVKGRFTISQ |
| | AWH66715 | DKSRNTLYLQMNSLKAEDTAVYYCAADVRPRGTTCHYNSRGQGTQVTVSS |
| | | (SEQ ID NO: 30) |
| FUSE-595 | GenBank | EVQLLESGGGLVQRGGSLRLSCTASGYTNRLKCMGWFRQAPGKEREEIATISTGTGNTYYADSVKGRFTISR |
| | AWH66715 | DKSRNTLYLQMKSLKAEDTAVYYCAADVRPKGTTCHYNSRGQGTQVTVSS |
| | | (SEQ ID NO: 31) |
| FUSE-596 | GenBank | EVQLLESGGGLVPRGGSLRLSCTASGYTNRLKCMGWFRQAPGKEREEIASISTGTGNTYYSDSVKGRFTISRD |
| | AWH66715 | KSRNTLYLKLKSLRAEDTAVYYCAADVRPDGTTCHYNSRGQGTQVTVSS |
| | | (SEQ ID NO: 32) |
| FUSE-597 | IGHV3-23 | EVQLLESGGGLVQPGGSLRLSCAASGYTNRLKCMGWFRQAPGKERELASISTGTGNTYY ADSVKGRFTISRD |
| | | GSKNTLYLQMNSLRAEDTAVYYCAADVRPDGTTCHYNSRGQGTLVTVSS |
| | | (SEQ ID NO: 33) |
| FUSE-598 | IGHV3-23 | EVQLLESGGGLVQPGGSLRLSCAASGYTNRLKCMGWFRQAPGKERELASISTGTGNTYYADSVKGRFTISQ |
| | | DGSKNTLYLQMNSLRAEDTAVYYCAADVRPDGTTCHYNSRGQGTLVTVSS |
| | | (SEQ ID NO: 34) |
| FUSE-654 | GenBank | EVQLLESGGGLVPRGGSLKLSCTASGYTNRLKCMGWFRQAPGKEREEIASISTGTGNTYYADSVKGRFTISQ |
| | AWH66715 | DKSRNTLYLQMNSLKAEDTAVYYCAADVRPRGTTCHYNSRGQGTQVTVSS |
| | | (SEQ ID NO: 35) |
| FUSE-655 | GenBank | EVQLLESGGGLVPRGGSLRLSCTASGYTNRLKCMGWFRQAPGKEREEIASISTGTGNTYYADSVKGRFTISQ |
| | AWH66715 | DKSRNTLYLQM&SLKAEDTAVYYCAADVRPRGTTCHYNSRGQGTQVTVSS |
| | | (SEQ ID NO: 36) |
| FUSE-661 | GenBank | EVQLLESGGGLVQPGGSLRLSCAASGSQRSANCMGWFRQAPGKDRELASMSIRSGRTYYADSVKGRFTISR |
| | AWH66715 | DSSKNTLYLQMSSLKAEDTAVYYCAAAYGGSRCVYNYRGQGTLVTVSS |
| | | (SEQ ID NO: 37) |
| FUSE-662 | GenBank | EVQLLESGGGLVQPGGSLRLSCAASGVGYSANCMGWFRQAPGKDRELASMSIRSGRTYYADSVKGRFTISR |
| | AWH66715 | DSSKNTLYLQMSSLKAEDTAVYYCAAAYGGSRCVYNYRGQGTLVTVSS |
| | | (SEQ ID NO: 38) |
| F22158101 | GenBank | EVQLLESGGGLVQPGGSLRLSCAASGYTNRIKCMGWFRQAPGKEREEVASISTGTGNTYYADSVKGRFTISQ |
| hSA1-BI- | AWH66715 | DKSKNTLYLQMNSLKAEDTAVYYCAADVRPDGFTCHYNSRGQGTLVTVSS |
| 001 | | (SEQ ID NO: 39) |
| F22158102 | GenBank | EVQLLESGGGLVQPGGSLRLSCAASGYTNRIKCMGWFRQAPGKEREEVASISTGTGNTYYADSVKGRFTISQ |
| hSA1-BI- | AWH66715 | DKSKNTLYLQMNSLKAEDTAVYYCAADVRPDGTTCHYNKRGQGTLVTVSS |
| 002 | | (SEQ ID NO: 40) |
| F22158103 | GenBank | EVQLLESGGGLVQPGGSLRLSCAASGYTNRLKCMGWFRQAPGKEREEVASISTGTGNAYYADSVKGRFTIS |
| h5A1-BI- | AWH66715 | QDKSKNTLYLQMNSLKAEDTAVYYCAADVRPDGTTCHYNKRGQGTLVTVSS |
| 003 | | (SEQ ID NO: 41) |
| F22158104 | GenBank | EVQLLESGGGLVQPGGSLRLSCAASGYTNRIKCMGWFRQAPGKEREEVASISTGTGNAYYADSVKGRFTISQ |
| h5A1-BI- | AWH66715 | DKSKNTLYLQMNSLKAEDTAVYYCAADVRPDGTTCHYNKRGQGTLVTVSS |
| 004 | | (SEQ ID NO: 42) |
| F22158105 | GenBank | EVQLLESGGGLVQPGGSLRLSCAASGYTNRLKCMGWFRQAPGKEREEVASISTGTGNTYYADSVKGRFTIS |
| h5AI-BI- | AWH66715 | QDKSKNTLYLQMNSLKAEDTAVYYCAADVRPDGFTCHYNKRGQGTLVTVSS |
| 005 | | (SEQ ID NO: 43) |
| F22158106 | GenBank | EVQLLESGGGLVQPGGSLRLSCAASGYTNRIKCMGWFRQAPGKEREEVASISTGTGNTYYADSVKGRFTISQ |
| h5A1-BI- | AWH66715 | DKSKNTLYLQMNSLKAEDTAVYYCAADVRPDGRTCHYNSRGQGTLVTVSS |
| 006 | | (SEQ ID NO: 44) |
| F22158107 | GenBank | EVQLLESGGGLVQPGGSLRLSCAASGYTNRLKCMGWFRQAPGKEREEVASISTGTGNAYYADSVKGRFTIS |
| hSAI-BI- | AWH66715 | QDKSKNTLYLQMNSLKAEDTAVYYCAADVRPDGRTCHYNSRGQGTLVTVSS |
| 007 | | (SEQ ID NO: 45) |
| F22158108 | GenBank | EVQLLESGGGLVQPGGSLRLSCAASGYTNRIKCMGWFRQAPGKEREEVASISTGTGNAYYADSVKGRFTISQ |
| h5A1-BI- | AWH66715 | DKSKNTLYLQMNSLKAEDTAVYYCAADVRPDGRTCHYNSRGQGTLVTVSS |
| 008 | | (SEQ ID NO: 46) |
| F22158109 | GenBank | EVQLLESGGGLVQPGGSLRLSCAASGYTNRIKCMGWFRQAPGKEREEVASISTGTGNSYYADSVKGRFTISQ |
| h5A1-BI- | AWH66715 | DKSKNTLYLQMNSLKAEDTAVYYCAADVRPDGRTCHYNKRGQGTLVTVSS |
| 009 | | (SEQ ID NO: 47) |
| F22158110 | GenBank | EVQLLESGGGLVQPGGSLRLSCAASGYTNRLKCMGWFRQAPGKEREEVASISTGTGNTYYADSVKGRFTIS |
| hSAI-BI- | AWH66715 | QDKSKNTLYLQMNSLKAEDTAVYYCAADVRPDGRTCHYNKRGQGTLVTVSS |
| 010 | | (SEQ ID NO: 48) |
| F22158111 | GenBank | EVQLLESGGGLVQPGGSLRLSCAASGYTNRIKCMGWFRQAPGKEREEVASISTGTGNTYYADSVKGRFTISQ |
| hSA1-BI- | AWH66715 | DKSKNTLYLQMNSLKAEDTAVYYCAADVRPFGTTCHYNSRGQGTLVTVSS |
| 011 | | (SEQ ID NO: 49) |
| F22158112 | GenBank | EVQLLESGGGLVQPGGSLRLSCAASGYTNRLKCMGWFRQAPGKEREEVASISTGTGNTYYADSVKGRFTIS |
| hSAI-BI- | AWH66715 | QDKSKNTLYLQMNSLKAEDTAVYYCAADVRPFGRTCHYNSRGQGTLVTVSS |
| 012 | | (SEQ ID NO: 50) |
| F22158113 | GenBank | EVQLLESGGGLVQPGGSLRLSCAASGYTNRLKCMGWFRQAPGKEREEVASISTGTGNTYYADSVKGRFTIS |
| hSA1-BI- | AWH66715 | QDKSKNTLYLQMNSLKAEDTAVYYCAADVRPFGTTCHYNKRGQGTLVTVSS |
| 013 | | (SEQ ID NO: 51) |
| F22158114 | GenBank | EVQLLESGGGLVQPGGSLRLSCAASGYTNRIKCMGWFRQAPGKEREEVASISTGTGNTYYADSVKGRFTISQ |
| RSA1-BI- | AWH66715 | DKSKNTLYLQMNSLKAEDTAVYYCAADVRPFGRTCHYNSRGQGTLVTVSS |
| 014 | | (SEQ ID NO: 52) |
| F22158115 | GenBank | EVQLLESGGGLVQPGGSLRLSCAASGYTNRIKCMGWFRQAPGKEREEVASISTGTGNTYYADSVKGRFTISQ |
| h5A1-BI- | AWH66715 | DKSKNTLYLQMNSLKAEDTAVYYCAADVRPFGTTCHYNKRGQGTLVTVSS |
| 015 | | (SEQ ID NO: 53) |
| F22158116 | GenBank | EVQLLESGGGLVQPGGSLRLSCAASGYTNRLKCMGWFRQAPGKEREEVASISTGTGNAYYADSVKGRFTIS |
| h5A1-BI- | AWH66715 | QDKSKNTLYLQMNSLKAEDTAVYYCAADVRPHGTTCHYNSRGQGTLVTVSS |
| 016 | | (SEQ ID NO: 54) |
| F22158117 | GenBank | EVQLLESGGGLVQPGGSLRLSCAASGYTNRIKCMGWFRQAPGKEREEVASISTGTGNAYYADSVKGRFTISQ |
| hSAI-BI- | AWH66715 | DKSKNTLYLQMNSLKAEDTAVYYCAADVRPHGTTCHYNSRGQGTLVTVSS |
| 017 | | (SEQ ID NO: 55) |
| F22158118 | GenBank | EVQLLESGGGLVQPGGSLRLSCAASGYTNRLKCMGWFRQAPGKEREEVASISTGTGNAYYADSVKGRFTIS |
| hSAI-BI- | AWH66715 | QDKSKNTLYLQMNSLKAEDTAVYYCAADVRPHGRTCHYNSRGQGTLVTVSS |
| 018 | | (SEQ ID NO: 56) |
| F22158119 | GenBank | EVQLLESGGGLVQPGGSLRLSCAASGYTNRIKCMGWFRQAPGKEREEVASISTGTGNAYYADSVKGRFTISQ |
| h5A1-BI- | AWH66715 | DKSKNTLYLQMNSLKAEDTAVYYCAADVRPHGRTCHYNSRGQGTLVTVSS |
| 019 | | (SEQ ID NO: 57) |
| F22158120 | GenBank | EVQLLESGGGLVQPGGSLRLSCAASGYTNRLKCMGWFRQAPGKEREEVASISTGTGNAYYADSVKGRFTIS |
| hSA1-BI- | AWH66715 | QDKSKNTLYLQMNSLKAEDTAVYYCAADVRPHGTTCHYNKRGQGTLVTVSS |
| 020 | | (SEQ ID NO: 58) |
| F22158121 | GenBank | EVQLLESGGGLVQPGGSLRLSCAASGYTNRIKCMGWFRQAPGKEREEVASISTGTGNAYYADSVKGRFTISQ |
| h5A1-BI- | AWH66715 | DKSKNTLYLQMNSLKAEDTAVYYCAADVRPHGTTCHYNKRGQGTLVTVSS |
| 021 | | (SEQ ID NO: 59) |
| BI-HU9- | IGHV3-23 | EVQLLESGGGLVQPGGSLRLSCAASGYTNRLKCMGWFRQAPGKERELVASISTGTGNTYYADSVKGRFTISR |
| HK RSA1 | | DNSKNTLYLQMNSLKAEDTAVYYCAADVRPDGTTCHYNSRGQGTLVTVSS |
| | | (SEQ ID NO: 16) |
| BI-HU9- | IGHV3-23 | EVQLLESGGGLVQPGGSLRLSCAASGYTNRLKCMGWFRQAPGKEREEVASISTGTGNTYYADSVKGRFTIS |
| HK-1 h5A1 | | QDKSKNTLYLQMNSLKAEDTAVYYCAADVRPDGTTCHYNSRGQGTLVTVSS |
| | | (SEQ ID NO: 60) |
| BI-HU9- | GenBank | EVQLLESGGGLVPRGGSLRLSCTASGYTNRLKCMGWFRQAPGKEREEIATISTGTGNTYYADSVKGRFTISR |
| VH9 h5A1 | AWH66715 | DNSRNTLYLQMKTLRAEDTAVYYCAADVRPDGTTCHYNSWGQGTQVTVSS |
| | | (SEQ ID NO: 14) |
| BI-HU9- | GenBank | EVQLLESGGGLVPRGGSLRLSCTASGYTNRLKCMGWFRQAPGKEREEIATISTGTGNTYYADSVKGRFTISQ |
| VH9-1 | AWH66715 | DKSRNTLYLQMKTLRAEDTAVYYCAADVRPDGTTCHYNSWGQGTQVTVSS |
| h5A1 | | (SEQ ID NO: 61) |
| BI-HU9- | IGHV3-23 | EVQLLESGGGLVQPGGSLRLSCAASGFTFRLKCMGWFRQAPGKGLEEVATISTGTGNTYYADSVKGRFTISR |
| VHH11 | | DNSKNTLYLQMNSLRAEDTAVYYCAADVRPDGTTCHYNSWGQGTTVTVSS |
| h5A1 Fc | | (SEQ ID NO: 62) |
| BI-HU9- | IGHV3-23 | EVQLLESGGGLVQPGGSLRLSCAASGYTNRLKCMGWFRQAPGKGLEEVATISTGTGNTYYADSVKGRFTIS |
| VHH12 | | RDNSKNTLYLQMNSLRAEDTAVYYCAADVRPDGTTCHYNSWGQGTTVTVSS |
| h5A1 Fc | | (SEQ ID NO: 63) |
| BI-HU9- | IGHV3-23 | EVQLLESGGGLVQPGGSLRLSCAASGYTNRLKCMGWFRQAPGKGLEEVATISTGTGNTYYADSVKGRFTIS |
| VHH13 | | QDKSKNTLYLQMNSLRAEDTAVYYCAADVRPDGTTCHYNSWGQGTTVTVSS |
| h5A1 | | (SEQ ID NO: 64) |
| BI-HU9- | IGHV3-30 | QVQLVESGGGVVQPGGSLRLSCAASGFTFRLKCMGWFRQAPGKGLEEVATISTGTGNTYYADSVKGRFTIS |
| VHH21 | | RDNSKNTLYLQMNSLRAEDTAVYYCAADVRPDGTTCHYNSWGQGTTVTVSS |
| hSA1 | | (SEQ ID NO: 65) |
| BI-HU9- | IGHV3-30 | QVQLVESGGGVVQPGGSLRLSCAASGYTNRLKCMGWFRQAPGKGLEEVATISTGTGNTYYADSVKGRFTIS |
| VHH22 | | RDNSKNTLYLQMNSLRAEDTAVYYCAADVRPDGTTCHYNSWGQGTTVTVSS |
| h5A1 | | (SEQ ID NO: 66) |
| BI-HU9- | IGHV3-30 | QVQLVESGGGVVQPGGSLRLSCAASGYTNRLKCMGWFRQAPGKGLEEVATISTGTGNTYYADSVKGRFTIS |
| VHH23 | | QDKSKNTLYLQMNSLRAEDTAVYYCAADVRPDGTTCHYNSWGQGTTVTVSS |
| h5A1 | | (SEQ ID NO: 67) |
| BI-HU9- | IGHV3-66 | EVQLVESGGGLVQPGGSLRLSCAASGFTVRLKCMGWFRQAPGKGLEEVATISTGTGNTYYADSVKGRFTIS |
| VHH31 | | RDNSKNTLYLQMNSLRAEDTAVYYCAADVRPDGTTCHYNSWGQGTTVTVSS |
| h5A1 | | (SEQ ID NO: 68) |
| BI-HU9- | IGHV3-66 | EVQLVESGGGLVQPGGSLRLSCAASGYTNRLKCMGWFRQAPGKGLEEVATISTGTGNTYYADSVKGRFTIS |
| VHH32 | | RDNSKNTLYLQMNSLRAEDTAVYYCAADVRPDGTTCHYNSWGQGTTVTVSS |
| h5A1 | | (SEQ ID NO: 69) |
| BI-HU9- | IGHV3-66 | EVQLVESGGGLVQPGGSLRLSCAASGYTNRLKCMGWFRQAPGKGLEEVATISTGTGNTYYADSVKGRFTIS |
| VHH33 | | QDKSKNTLYLQMNSLRAEDTAVYYCAADVRPDGTTCHYNSWGQGTTVTVSS |
| h5A1 | | (SEQ ID NO: 70) |

TABLE 8A
CDRs

| | CDR1 | CDR2 | CDR3 |
|---|---|---|---|
| FUSE-112 | GSTYSANC | SIRSGR | AYGGSRCVYNY |
| | (SEQ ID NO: 1) | (SEQ ID NO: 76) | (SEQ ID NO: 3) |
| FUSE-179 | GYTNRLKC | STGTGN | DVRPDGTTCHYNS |
| | (SEQ ID NO: 4) | (SEQ ID NO: 77) | (SEQ ID NO: 6) |
| FUSE-488 | GYTNRLKC | STGTGN | DVRPDGTTCHYNS |
| | (SEQ ID NO: 4) | (SEQ ID NO: 77) | (SEQ ID NO: 6) |
| FUSE-497 | GYTNRLKC | STGTGN | DVRPDGTTCHYNS |
| | (SEQ ID NO: 4) | (SEQ ID NO: 77) | (SEQ ID NO: 6) |
| FUSE-498 | GYTNRLKC | STGTGN | DVRPDGTTCHYNS |
| | (SEQ ID NO: 4) | (SEQ ID NO: 77) | (SEQ ID NO: 6) |
| FUSE-524 | GYTNRLKC | STGTGN | DVRPDGTTCHYNS |
| | (SEQ ID NO: 4) | (SEQ ID NO: 77) | (SEQ ID NO: 6) |
| FUSE-525 | GYTNRLKC | STGTGN | DVRPDGTTCHYNS |
| | (SEQ ID NO: 4) | (SEQ ID NO: 77) | (SEQ ID NO: 6) |
| FUSE-537 | GSTYSANC | IRSGR | AYGGSRCVYNY |
| | (SEQ ID NO: 1) | (SEQ ID NO: 78) | (SEQ ID NO: 3) |
| FUSE-559 | GYTNRLKC | STGTGN | DVRPHGTTCHYNS |
| | (SEQ ID NO: 4) | (SEQ ID NO: 77) | (SEQ ID NO: 80) |
| FUSE-560 | GSTYSANC | IRSGR | AYGGSRCVYNY |
| | (SEQ ID NO: 1) | (SEQ ID NO: 78) | (SEQ ID NO: 3) |
| FUSE-561 | GSTYSANC | IRSGR | AYGGSRCVYNY |
| | (SEQ ID NO: 1) | (SEQ ID NO: 78) | (SEQ ID NO: 3) |
| FUSE-562 | GSTYSANC | IRSGR | AYGGSRCVYNY |
| | (SEQ ID NO: 1) | (SEQ ID NO: 78) | (SEQ ID NO: 3) |
| FUSE-563 | GYTNRLKC | STGTGN | DVRPDGTTCHYNS |
| | (SEQ ID NO: 4) | (SEQ ID NO: 77) | (SEQ ID NO: 6) |
| FUSE-569 | GYTNRLKC | STGTGN | DVRPDGTTCHYNS |
| | (SEQ ID NO: 4) | (SEQ ID NO: 77) | (SEQ ID NO: 6) |
| FUSE-570 | GYTNRLKC | SSGTGN | DVRPDGTTCHYNS |
| | (SEQ ID NO: 4) | (SEQ ID NO: 79) | (SEQ ID NO: 6) |
| FUSE-589 | GSTYSANC | IRSGR | AYGGSRCVYNY |
| | (SEQ ID NO: 1) | (SEQ ID NO: 78) | (SEQ ID NO: 3) |
| FUSE-590 | GYTNRLKC | STGTGN | DVRPRGTTCHYNS |
| | (SEQ ID NO: 4) | (SEQ ID NO: 77) | (SEQ ID NO: 81) |
| FUSE-591 | GYTNRLKC | STGTGN | DVRPKGTTCHYNK |
| | (SEQ ID NO: 4) | (SEQ ID NO: 77) | (SEQ ID NO: 82) |
| FUSE-592 | GYTNRLKC | STGTGN | DVRPRGTTCHYNS |
| | (SEQ ID NO: 4) | (SEQ ID NO: 77) | (SEQ ID NO: 81) |
| FUSE-593 | GYTNRLKC | STGTGN | DVRPKGTTCHYNS |
| | (SEQ ID NO: 4) | (SEQ ID NO: 77) | (SEQ ID NO: 83) |
| FUSE-594 | GYTNRLKC | STGTGN | DVRPRGTTCHYNS |
| | (SEQ ID NO: 4) | (SEQ ID NO: 77) | (SEQ ID NO: 81) |
| FUSE-595 | GYTNRLKC | STGTGN | DVRPKGTTCHYNS |
| | (SEQ ID NO: 4) | (SEQ ID NO: 77) | (SEQ ID NO: 83) |
| FUSE-596 | GYTNRLKC | STGTGN | DVRPDGTTCHYNS |
| | (SEQ ID NO: 4) | (SEQ ID NO: 77) | (SEQ ID NO: 6) |
| FUSE-597 | GYTNRLKC | STGTGN | DVRPDGTTCHYNS |
| | (SEQ ID NO: 4) | (SEQ ID NO: 77) | (SEQ ID NO: 6) |
| FUSE-598 | GYTNRLKC | STGTGN | DVRPDGTTCHYNS |
| | (SEQ ID NO: 4) | (SEQ ID NO: 77) | (SEQ ID NO: 6) |
| FUSE-654 | GYTNRLKC | STGTGN | DVRPRGTTCHYNS |
| | (SEQ ID NO: 4) | (SEQ ID NO: 77) | (SEQ ID NO: 81) |
| FUSE-655 | GYTNRLKC | STGTGN | DVRPRGTTCHYNS |
| | (SEQ ID NO: 4) | (SEQ ID NO: 77) | (SEQ ID NO: 81) |
| FUSE-661 | GSQRSANC | IRSGR | AYGGSRCVYNY |
| | (SEQ ID NO: 71) | (SEQ ID NO: 78) | (SEQ ID NO: 3) |
| FUSE-662 | GVGYSANC | IRSGR | AYGGSRCVYNY |
| | (SEQ ID NO: 72) | (SEQ ID NO: 78) | (SEQ ID NO: 3) |
| F22158101 h5A1- | GYTNRIKC | STGTGN | DVRPDGFTCHYNS |
| BI-001 | (SEQ ID NO: 73) | (SEQ ID NO: 77) | (SEQ ID NO: 84) |
| F22158102 h5A1- | GYTNRIKC | STGTGN | DVRPDGTTCHYNK |
| BI-002 | (SEQ ID NO: 73) | (SEQ ID NO: 77) | (SEQ ID NO: 85) |
| F22158103 h5A1- | GYTNRLKC | STGTGN | DVRPDGTTCHYNK |
| BI-003 | (SEQ ID NO: 4) | (SEQ ID NO: 77) | (SEQ ID NO: 85) |
| F22158104 h5A1- | GYTNRIKC | STGTGN | DVRPDGTTCHYNK |
| BI-004 | (SEQ ID NO: 73) | (SEQ ID NO: 77) | (SEQ ID NO: 85) |
| F22158105 h5A1- | GYTNRLKC | STGTGN | DVRPDGFTCHYNK |
| BI-005 | (SEQ ID NO: 4) | (SEQ ID NO: 77) | (SEQ ID NO: 86) |
| F22158106 h5A1- | GYTNRIKC | STGTGN | DVRPDGRTCHYNS |
| BI-006 | (SEQ ID NO: 73) | (SEQ ID NO: 77) | (SEQ ID NO: 87) |
| F22158107 h5A1- | GYTNRLKC | STGTGN | DVRPDGRTCHYNS |
| BI-007 | (SEQ ID NO: 4) | (SEQ ID NO: 77) | (SEQ ID NO: 87) |
| F22158108 h5A1- | GYTNRIKC | STGTGN | DVRPDGRTCHYNS |
| BI-008 | (SEQ ID NO: 73) | (SEQ ID NO: 77) | (SEQ ID NO: 87) |
| F22158109 h5A1- | GYTNRIKC | STGTGN | DVRPDGRTCHYNK |
| BI-009 | (SEQ ID NO: 73) | (SEQ ID NO: 77) | (SEQ ID NO: 88) |
| F22158110 h5A1- | GYTNRLKC | STGTGN | DVRPDGRTCHYNK |
| BI-010 | (SEQ ID NO: 4) | (SEQ ID NO: 77) | (SEQ ID NO: 88) |
| F22158111 h5A1- | GYTNRIKC | STGTGN | DVRPFGTTCHYNS |
| BI-011 | (SEQ ID NO: 73) | (SEQ ID NO: 77) | (SEQ ID NO: 89) |
| F22158112 h5A1- | GYTNRLKC | STGTGN | DVRPFGRTCHYNS |
| BI-012 | (SEQ ID NO: 4) | (SEQ ID NO: 77) | (SEQ ID NO: 90) |
| F22158113 h5A1- | GYTNRLKC | STGTGN | DVRPFGTTCHYNK |
| BI-013 | (SEQ ID NO: 4) | (SEQ ID NO: 77) | (SEQ ID NO: 91) |
| F22158114 h5A1- | GYTNRIKC | STGTGN | DVRPFGRTCHYNS |
| BI-014 | (SEQ ID NO: 73) | (SEQ ID NO: 77) | (SEQ ID NO: 90) |
| F22158115 h5A1- | GYTNRIKC (SEQ | STGTGN | DVRPFGTTCHYNK |
| BI-015 | ID NO: 73) | (SEQ ID NO: 77) | (SEQ ID NO: 91) |
| F22158116 h5A1- | GYTNRLKC | STGTGN | DVRPHGTTCHYNS |
| BI-016 | (SEQ ID NO: 4) | (SEQ ID NO: 77) | (SEQ ID NO: 80) |
| F22158117 h5A1- | GYTNRIKC | STGTGN | DVRPHGTTCHYNS |
| BI-017 | (SEQ ID NO: 73) | (SEQ ID NO: 77) | (SEQ ID NO: 80) |
| F22158118 h5A1- | GYTNRLKC | STGTGN | DVRPHGRTCHYNS |
| BI-018 | (SEQ ID NO: 4) | (SEQ ID NO: 77) | (SEQ ID NO: 92) |
| F22158119 hSA1- | GYTNRIKC | STGTGN | DVRPHGRTCHYNS |
| BI-019 | (SEQ ID NO: 73) | (SEQ ID NO: 77) | (SEQ ID NO: 92) |
| F22158120 h5A1- | GYTNRLKC | STGTGN | DVRPHGTTCHYNK |
| BI-020 | (SEQ ID NO: 4) | (SEQ ID NO: 77) | (SEQ ID NO: 93) |
| F22158121 h5A1- | GYTNRIKC | STGTGN | DVRPHGTTCHYNK |
| BI-021 | (SEQ ID NO: 73) | (SEQ ID NO: 77) | (SEQ ID NO: 93) |
| BI-HU9-HK h5A1 | GYTNRLKC | STGTGN | DVRPDGTTCHYNS |
| | (SEQ ID NO: 4) | (SEQ ID NO: 77) | (SEQ ID NO: 6) |
| BI-HU9-HK-1 | GYTNRLKC | STGTGN | DVRPDGTTCHYNS |
| h5A1 | (SEQ ID NO: 4) | (SEQ ID NO: 77) | (SEQ ID NO: 6) |
| BI-HU9-VH9 | GYTNRLKC | STGTGN | DVRPDGTTCHYNS |
| h5A1 | (SEQ ID NO: 4) | (SEQ ID NO: 77) | (SEQ ID NO: 6) |
| BI-HU9-VH9-1 | GYTNRLKC | STGTGN | DVRPDGTTCHYNS |
| h5A1 | (SEQ ID NO: 4) | (SEQ ID NO: 77) | (SEQ ID NO: 6) |
| BI-HU9-VHH11 | GFTFRLKC | STGTGN | DVRPDGTTCHYNS |
| h5A1 Fc | (SEQ ID NO: 74) | (SEQ ID NO: 77) | (SEQ ID NO: 6) |
| BI-HU9-VHH12 | GYTNRLKC | STGTGN | DVRPDGTTCHYNS |
| h5A1 Fc | (SEQ ID NO: 4) | (SEQ ID NO: 77) | (SEQ ID NO: 6) |
| BI-HU9-VHH13 | GYTNRLKC | STGTGN | DVRPDGTTCHYNS |
| h5A1 | (SEQ ID NO: 4) | (SEQ ID NO: 77) | (SEQ ID NO: 6) |
| BI-HU9-VHH21 | GFTFRLKC | STGTGN | DVRPDGTTCHYNS |
| b5A1 | (SEQ ID NO: 74) | (SEQ ID NO: 77) | (SEQ ID NO: 6) |
| BI-HU9-VHH22 | GYTNRLKC | STGTGN | DVRPDGTTCHYNS |
| h5A1 | (SEQ ID NO: 4) | (SEQ ID NO: 77) | (SEQ ID NO: 6) |
| BI-HU9-VHH23 | GYTNRLKC | STGTGN | DVRPDGTTCHYNS |
| h5A1 | (SEQ ID NO: 4) | (SEQ ID NO: 77) | (SEQ ID NO: 6) |
| BI-HU9 VHH31 | GFTVRLKC | STGTGN | DVRPDGTTCHYNS |
| h5A1 | (SEQ ID NO: 75) | (SEQ ID NO: 77) | (SEQ ID NO: 6) |
| BI-HU9-VHH32 | GYTNRLKC | STGTGN | DVRPDGTTCHYNS |
| h5A1 | (SEQ ID NO: 4) | (SEQ ID NO: 77) | (SEQ ID NO: 6) |
| BI-HU9-VHH33 | GYTNRLKC | STGTGN | DVRPDGTTCHYNS |
| h5A1 | (SEQ ID NO: 4) | (SEQ ID NO: 77) | (SEQ ID NO: 6) |

TABLE 8B
Framework regions and some alternative CDRs

| | FW1 | CDRI | FW2 | CDR2 | FW3 | CDR3 | FW4 |
|---|---|---|---|---|---|---|---|
| FUSE-112 | QVQLQESGGG | GSTYSANC | MGWFRQAPG | MSIRSGRTY | YSDSVKGRFTI | AYGGSRCVYN | RGQGTQVTVS |
| | SVPAGGSLRLS | (SEQ ID NO: 1) | KEREEVAS | (SEQ ID NO: 2) | SQDGSKNTLY | Y | S |
| | CAAS | | (SEQ ID | | LQLNSLKAED | (SEQ ID NO: 3) | (SEQ ID |
| | (SEQ ID NO: 97) | | NO: 111) | | TALYYCAA | | NO: 144) |
| | | | | | (SEQ ID | | |
| | | | | | NO: 118) | | |
| FUSE-179 | QVQLQESGGG | GYTNRLKC | MGWFRQAPG | ISTGTGNTY | YADSVKGRFT | DVRPDGTTCH | GGQGTQVTVS |
| | SVQAGGSLKL | (SEQ ID NO: 4) | KEREEIAT | (SEQ ID NO: 5) | FSQDKVKNTV | YNS (SEQ ID | S |
| | SCTAS | | (SEQ ID | | YLQMNTLKPD | NO: 6) | (SEQ ID |
| | (SEQ ID NO: 98) | | NO: 112) | | DTGMYYCAA | | NO: 145) |
| | | | | | (SEQ ID | | |
| | | | | | NO: 119) | | |
| FUSE-488 | QVQLQESGGG | GYTNRLKC | MGWFRQAPG | ISTGTGNTY | YADSVKGRFT | DVRPDGTTCH | GGQGTQVTVS |
| | SVQAGGSLKL | (SEQ ID NO: 4) | KEREEIAT | (SEQ ID NO: 5) | FSQDKVKNTV | YNS (SEQ ID | S |
| | SCTAS | | (SEQ ID | | YLQMNTLKPD | NO: 6) | (SEQ ID |
| | (SEQ ID NO: 98) | | NO: 112) | | DTGMYYCAA | | NO: 145) |
| | | | | | (SEQ ID | | |
| | | | | | NO: 119) | | |
| FUSE-497 | EVQLLESGGG | GYTNRLKC | MGWFRQAPG | ISTGTGNTY | YADSVKGRFTI | DVRPDGTTCH | WGQGTQVTV |
| | LVPRGGSLRLS | (SEQ ID NO: 4) | KEREEIAT | (SEQ ID NO: 5) | SRDNSRNTLY | YNS (SEQ ID | SS |
| | CTAS | | (SEQ ID | | LQMKTLRAED | NO: 6) | (SEQ ID |
| | (SEQ ID NO: 99) | | NO: 112) | | TAVYYCAA | | NO: 146) |
| | | | | | (SEQ ID | | |
| | | | | | NO: 120) | | |
| FUSE-498 | EVQLVESGGG | GYTNRLKC | MGWFRQAPG | ISTGTGNTY | YADSVKGRFT | DVRPDGTTCH | GGQGTQVTVS |
| | LVQPGGSLRLS | (SEQ ID NO: 4) | KEREEIAT | (SEQ ID NO: 5) | ISRDNSRNTLY | YNS (SEQ ID | S |
| | CTAS | | (SEQ ID | | LQMKTLRAED | NO: 6) | (SEQ ID |
| | (SEQ ID | | NO: 112) | | TAVYYCAA | | NO: 145) |
| | NO: 100) | | | | (SEQ ID | | |
| | | | | | NO: 120) | | |
| FUSE-524 | EVQLLESGGG | GYTNRLKC | MGWFRQAPG | ISTGTGNTY | YADSVKGRFTI | DVRPDGTTCH | RGQGTLVTVS |
| | LVQPGGSLRLS | (SEQ ID NO: 4) | KERELAS | (SEQ ID NO: 5) | SRDNSKNTLY | YNS (SEQ ID | S |
| | CAAS | | (SEQ ID | | LQMNSLKAED | NO: 6) | (SEQ ID |
| | (SEQ ID | | NO: 113) | | TAVYYCAA | | NO: 147) |
| | NO: 101) | | | | (SEQ ID | | |
| | | | | | NO: 121) | | |
| FUSE-525 | EVQLLESGGG | GYTNRLKC | MGWFRQAPG | ISTGTGNTY | YADSVKGRFTI | DVRPDGTTCH | RGQGTLVTVS |
| | LVQPGGSLRLS | (SEQ ID NO: 4) | KERELVAS | (SEQ ID NO: 5) | SRDNSKNTLY | YNS (SEQ ID | S |
| | CAAS | | (SEQ ID | | LQMNSLKAED | NO: 6) | (SEQ ID |
| | (SEQ ID | | NO: 114) | | TAVYYCAA | | NO: 147) |
| | NO: 101) | | | | (SEQ ID | | |
| | | | | | NO: 121) | | |
| FUSE-537 | EVQLLESGGG | GSTYSANC | MGWFRQAPG | MSIRSGRTY | YADSVKGRFTI | AYGGSRCVYN | RGQGTLVTVS |
| | LVQPGGSLRLS | (SEQ ID NO: 1) | KERELAS | (SEQ ID NO: 2) | SRDNSKNTLY | Y | S |
| | CAAS | | (SEQ ID | | LQMNSLKAED | (SEQ ID NO: 3) | (SEQ ID |
| | (SEQ ID | | NO: 113) | | TAVYYCAA | | NO: 147) |
| | NO: 101) | | | | (SEQ ID | | |
| | | | | | NO: 121) | | |
| FUSE-559 | EVQLLESGGG | GYTNRLKC | MGWFRQAPG | ISTGTGNTY | YADSVKGRFTI | DVRPHGTTCH | RGQGTQVTVS |
| | LVQPGGSLRLS | (SEQ ID NO: 4) | KEREEIAS | (SEQ ID NO: 5) | SRDKSRNTLY | YNS | S |
| | CTAS | | (SEQ ID | | LQMNTLRAED | (SEQ ID NO: 80) | (SEQ ID |
| | (SEQ ID | | NO: 115) | | TAVYYCAA | | NO: 144) |
| | NO: 102) | | | | (SEQ ID | | |
| | | | | | NO: 122) | | |
| FUSE-560 | EVQLLESGGG | GSTYSANC | MGWFRQAPG | MSIRSGRTY | YADSVKGRFTI | AYGGSRCVYN | RGQGTLVTVS |
| | LVQPGGSLRLS | (SEQ ID NO: 1) | KERELAS | (SEQ ID NO: 2) | SRDSSKNTLYL | Y | S |
| | CAAS | | (SEQ ID | | QMNSLKAEDT | (SEQ ID NO: 3) | (SEQ ID |
| | (SEQ ID | | NO: 113) | | AVYYCAA | | NO: 147) |
| | NO: 101) | | | | (SEQ ID | | |
| | | | | | NO: 123) | | |
| FUSE-561 | EVQLLESGGG | GSTYSANC | MGWFRQAPG | MSIRSGRTY | YADSVKGRFTI | AYGGSRCVYN | RGQGTLVTVS |
| | LVQPGGSLRLS | (SEQ ID NO: 1) | KERELAS | (SEQ ID NO: 2) | SRDSSKNTLYL | Y | S |
| | CAAS | | (SEQ ID | | QMNSLRAEDT | (SEQ ID NO: 3) | (SEQ ID |
| | (SEQ ID | | NO: 113) | | AVYYCAA | | NO: 147) |
| | NO: 101) | | | | (SEQ ID | | |
| | | | | | NO: 124) | | |
| FUSE-562 | EVQLLESGGG | GSTYSANC | MGWFRQAPG | MSIRSGRTY | YSDSVKGRFTI | AYGGSRCVYN | RGQGTLVTVS |
| | LVQPGGSLRLS | (SEQ ID NO: 1) | KERELAS | (SEQ ID NO: 2) | SRDGSKNTLY | Y | S |
| | CAAS | | (SEQ ID | | LQMNSLKAED | (SEQ ID NO: 3) | (SEQ ID |
| | (SEQ ID | | NO: 113) | | TAVYYCAA | | NO: 147) |
| | NO: 101) | | | | (SEQ ID | | |
| | | | | | NO: 125) | | |
| FUSE-563 | EVQLLESGGG | GYTNRLKC | MGWFRQAPG | ISTGTGNTY | YADSVKGRFTI | DVRPDGTTCH | WGQGTLVTVS |
| | LVQPGGSLKL | (SEQ ID NO: 4) | KEREEIAT | (SEQ ID NO: 5) | SQDRSKNTLY | YNS (SEQ ID | S |
| | SCTAS | | (SEQ ID | | LQMNSLRAED | NO: 6) | (SEQ ID |
| | (SEQ ID | | NO: 112) | | TAVYYCAA | | NO: 148) |
| | NO: 103) | | | | (SEQ ID | | |
| | | | | | NO: 126) | | |
| FUSE-569 | QVQLVESGGG | GYTNRLKC | MGWFRQAPG | ISTGTGNTY | YADSVKGRFTI | DVRPDGTTCH | WGQGTQVTV |
| | LVQPGGSLRLS | (SEQ ID NO: 4) | KERELAS | (SEQ ID NO: 5) | SRDNSKNTLY | YNS (SEQ ID | SS |
| | CSAS | | (SEQ ID | | LQMNSLRADD | NO: 6) | (SEQ ID |
| | (SEQ ID | | NO: 113) | | TAVYYCAA | | NO: 146) |
| | NO: 104) | | | | (SEQ ID | | |
| | | | | | NO: 127) | | |
| FUSE-570 | QVQLVESGGG | GYTNRLKC | MGWFRQAPG | ISSGTGNTY | YADSVKGRFT | DVRPDGTTCH | GGQGTQVTVS |
| | LVQPGGSLRLS | (SEQ ID NO: 4) | KEREEIAT | (SEQ ID NO: 94) | FSRDNSKNTL | YNS (SEQ ID | S |
| | CSAS | | (SEQ ID | | YLQMNSLRAE | NO: 6) | (SEQ ID |
| | (SEQ ID | | NO: 112) | | DTGMYYCAA | | NO: 145) |
| | NO: 104) | | | | (SEQ ID | | |
| | | | | | NO: 128) | | |
| FUSE-589 | EVQLLESGGG | GSTYSANC | MGWFRQAPG | MSIRSGRTY | YADSVKGRFTI | AYGGSRCVYN | RGQGTLVTVS |
| | LVQPGGSLRLS | (SEQ ID NO: 1) | KERELAS | (SEQ ID NO: 2) | SQDGSKNTLY | Y | S |
| | CAAS | | (SEQ ID | | LQMNSLKAED | (SEQ ID NO: 3) | (SEQ ID |
| | (SEQ ID | | NO: 113) | | TAVYYCAA | | NO: 147) |
| | NO: 101) | | | | (SEQ ID | | |
| | | | | | NO: 129) | | |
| FUSE-590 | EVQLLESGGG | GYTNRLKC | MGWFRQAPG | ISTGTGNTY | YSDSVKGRFTI | DVRPRGTTCH | RGQGTQVTVS |
| | LVQRGGSLRL | (SEQ ID NO: 4) | KEREEIAS | (SEQ ID NO: 5) | SRDKSRNTLY | YNS | S |
| | SCTAS | | (SEQ ID | | LQMKSLRAED | (SEQ ID NO: 81) | (SEQ ID |
| | (SEQ ID | | NO: 115) | | TAVYYCAA | | NO: 144) |
| | NO: 105) | | | | (SEQ ID | | |
| | | | | | NO: 130) | | |
| FUSE-591 | EVQLLESGGG | GYTNRLKC | MGWFRQAPG | ISTGTGNTY | YADSVKGRFTI | DVRPRGTTCH | RGQGTQVTVS |
| | LVPRGGSLRLS | (SEQ ID NO: 4) | KEREEIAT | (SEQ ID NO: 5) | SQDKSRNTLY | YNS(SEQ ID | S |
| | CAAS | | (SEQ ID | | LQMNSLKAED | NO: 81) | (SEQ ID |
| | (SEQ ID | | NO: 112) | | TAVYYCAA | | NO: 144) |
| | NO: 106) | | | | (SEQ ID | | |
| | | | | | NO: 131) | | |
| FUSE-592 | EVQLLESGGG | GYTNRLKC | MGWFRQAPG | ISTGTGNTY | YADSVKGRFTI | DVRPRGTTCH | RGQGTQVTVS |
| | LVPRGGSLRLS | (SEQ ID NO: 4) | KEREEVAS | (SEQ ID NO: 5) | SQDKSRNTLY | YNS | S |
| | CAAS | | (SEQ ID | | LQLNSLRAED | (SEQ ID NO: 81) | (SEQ ID |
| | (SEQ ID | | NO: 111) | | TAVYYCAA | | NO: 144) |
| | NO: 106) | | | | (SEQ ID | | |
| | | | | | NO: 132) | | |
| FUSE-593 | EVQLLESGGG | GYTNRLKC | MGWFRQAPG | ISTGTGNTY | YSDSVKGRFTI | DVRPKGTTCH | RGQGTQVTVS |
| | LVQRGGSLRL | (SEQ ID NO: 4) | KEREEIAS | (SEQ ID NO: 5) | SRDKSRNTLY | YNS | S |
| | SCAAS | | (SEQ ID | | LQLNSLKAED | (SEQ ID NO: 83) | (SEQ ID |
| | (SEQ ID | | NO: 115) | | TAVYYCAA | | NO: 144) |
| | NO: 107) | | | | (SEQ ID | | |
| | | | | | NO: 133) | | |
| FUSE-594 | EVQLLESGGG | GYTNRLKC | MGWFRQAPG | ISTGTGNTY | YADSVKGRFTI | DVRPRGTTCH | RGQGTQVTVS |
| | LVPRGGSLRLS | (SEQ ID NO: 4) | KEREEIAS | (SEQ ID NO: 5) | SQDKSRNTLY | YNS | S |
| | CTAS | | (SEQ ID | | LQMNSLKAED | (SEQ ID NO: 81) | (SEQ ID |
| | (SEQ ID NO: 99) | | NO: 115) | | TAVYYCAA | | NO: 144) |
| | | | | | (SEQ ID | | |
| | | | | | NO: 131) | | |
| FUSE-595 | EVQLLESGGG | GYTNRLKC | MGWFRQAPG | ISTGTGNTY | YADSVKGRFTI | DVRPKGTTCH | RGQGTQVTVS |
| | LVQRGGSLRL | (SEQ ID NO: 4) | KEREEIAT | (SEQ ID NO: 5) | SRDKSRNTLY | YNS | S |
| | SCTAS | | (SEQ ID | | LQMKSLKAED | (SEQ ID NO: 83) | (SEQ ID |
| | (SEQ ID | | NO: 112) | | TAVYYCAA | | NO: 144) |
| | NO: 105) | | | | (SEQ ID | | |
| | | | | | NO: 134) | | |
| FUSE-596 | EVQLLESGGG | GYTNRLKC | MGWFRQAPG | ISTGTGNTY | YSDSVKGRFTI | DVRPDGTTCH | RGQGTQVTVS |
| | LVPRGGSLRLS | (SEQ ID NO: 4) | KEREEIAS | (SEQ ID NO: 5) | SRDKSRNTLY | YNS (SEQ ID | S |
| | CTAS | | (SEQ ID | | LKLKSLRAED | NO: 6) | (SEQ ID |
| | (SEQ ID NO: 99) | | NO: 115) | | TAVYYCAA | | NO: 144) |
| | | | | | (SEQ ID | | |
| | | | | | NO: 135) | | |
| FUSE-597 | EVQLLESGGG | GYTNRLKC | MGWFRQAPG | ISTGTGNTY | YADSVKGRFTI | DVRPDGTTCH | RGQGTLVTVS |
| | LVQPGGSLRLS | (SEQ ID NO: 4) | KERELAS | (SEQ ID NO: 5) | SRDGSKNTLY | YNS (SEQ ID | S |
| | CAAS | | (SEQ ID | | LQMNSLRAED | NO: 6) | (SEQ ID |
| | (SEQ ID | | NO: 113) | | TAVYYCAA | | NO: 147) |
| | NO: 101) | | | | (SEQ ID | | |
| | | | | | NO: 136) | | |
| FUSE-598 | EVQLLESGGG | GYTNRLKC | MGWFRQAPG | ISTGTGNTY | YADSVKGRFTI | DVRPDGTTCH | RGQGTLVTVS |
| | LVQPGGSLRLS | (SEQ ID NO: 4) | KERELAS | (SEQ ID NO: 5) | SQDGSKNTLY | YNS (SEQ ID | S |
| | CAAS | | (SEQ ID | | LQMNSLRAED | NO: 6) | (SEQ ID |
| | (SEQ ID | | NO: 113) | | TAVYYCAA | | NO: 147) |
| | NO: 101) | | | | (SEQ ID | | |
| | | | | | NO: 137) | | |
| FUSE-654 | EVQLLESGGG | GYTNRLKC | MGWFRQAPG | ISTGTGNTY | YADSVKGRFTI | DVRPRGTTCH | RGQGTQVTVS |
| | LVPRGGSLKLS | (SEQ ID NO: 4) | KEREEIAS | (SEQ ID NO: 5) | SQDKSRNTLY | YNS | S |
| | CTAS | | (SEQ ID | | LQMNSLKAED | (SEQ ID NO: 81) | (SEQ ID |
| | (SEQ ID | | NO: 115) | | TAVYYCAA | | NO: 144) |
| | NO: 108) | | | | (SEQ ID | | |
| | | | | | NO: 131) | | |
| FUSE-655 | EVQLLESGGG | GYTNRLKC | MGWFRQAPG | ISTGTGNTY | YADSVKGRFTI | DVRPRGTTCH | RGQGTQVTVS |
| | LVPRGGSLRLS | (SEQ ID NO: 4) | KEREEIAS | (SEQ ID NO: 5) | SQDKSRNTLY | YNS | S |
| | CTAS | | (SEQ ID | | LQMsSLKAED | (SEQ ID NO: 81) | (SEQ ID |
| | (SEQ ID NO: 99) | | NO: 115) | | TAVYYCAA | | NO: 144) |
| | | | | | (SEQ ID | | |
| | | | | | NO: 138) | | |
| FUSE-661 | EVQLLESGGG | GSQRSANC | MGWFRQAPG | MSIRSGRTY | YADSVKGRFTI | AYGGSRCVYN | RGQGTLVTVS |
| | LVQPGGSLRLS | (SEQ ID NO: 71) | KDRELAS | (SEQ ID NO: 2) | SRDSSKNTLYL | Y | S |
| | CAAS | | (SEQ ID | | QMSSLKAEDT | (SEQ ID NO: 3) | (SEQ ID |
| | (SEQ ID | | NO: 116) | | AVYYCAA | | NO: 147) |
| | NO: 101) | | | | (SEQ ID | | |
| | | | | | NO: 139) | | |
| FUSE-662 | EVQLLESGGG | GVGYSANC | MGWFRQAPG | MSIRSGRTY | YADSVKGRFTI | AYGGSRCVYN | RGQGTLVTVS |
| | LVQPGGSLRLS | (SEQ ID NO: 72) | KDRELAS | (SEQ ID NO: 2) | SRDSSKNTLYL | Y | S |
| | CAAS | | (SEQ ID | | QMSSLKAEDT | (SEQ ID NO: 3) | (SEQ ID |
| | (SEQ ID | | NO: 116) | | AVYYCAA | | NO: 147) |
| | NO: 101) | | | | (SEQ ID | | |
| | | | | | NO: 139) | | |
| F2215810 | EVQLLESGGG | GYTNRIKC | MGWFRQAPG | ISTGTGNTY | YADSVKGRFTI | DVRPDGFTCH | RGQGTLVTVS |
| 1 hSAL- | LVQPGGSLRLS | (SEQ ID NO: 73) | KEREEVAS | (SEQ ID NO: 5) | SQDKSKNTLY | YNS | S |
| BI-001 | CAAS | | (SEQ ID | | LQMNSLKAED | (SEQ ID NO: 84) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRIKC | MGWFRQAPG | ISTGTGNTY | YADSVKGRFTI | DVRPDGTTCH | RGQGTLVTVS |
| 2 hSAI- | LVQPGGSLRLS | (SEQ ID NO: 73) | KEREEVAS | (SEQ ID NO: 5) | SQDKSKNTLY | YNK | S |
| BI-002 | CAAS | | (SEQ ID | | LQMNSLKAED | (SEQ ID NO: 85) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRLKC | MGWFRQAPG | ISTGTGNAY | YADSVKGRFTI | DVRPDGTTCH | RGQGTLVTVS |
| 3 h5A1- | LVQPGGSLRLS | (SEQ ID NO: 4) | KEREEVAS | (SEQ ID NO: 95) | SQDKSKNTLY | YNK | S |
| BI-003 | CAAS | | (SEQ ID | | LQMNSLKAED | (SEQ ID NO: 85) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRIKC | MGWFRQAPG | ISTGTGNAY | YADSVKGRFTI | DVRPDGTTCH | RGQGTLVTVS |
| 4 h5A1- | LVQPGGSLRLS | (SEQ ID NO: 73) | KEREEVAS | (SEQ ID NO: 95) | SQDKSKNTLY | YNK | S |
| BI-004 | CAAS | | (SEQ ID | | LQMNSLKAED | (SEQ ID NO: 85) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRLKC | MGWFRQAPG | ISTGTGNTY | YADSVKGRFTI | DVRPDGFTCH | RGQGTLVTVS |
| 5 h5A1- | LVQPGGSLRLS | (SEQ ID NO: 4) | KEREEVAS | (SEQ ID NO: 5) | SQDKSKNTLY | YNK | S |
| BI-005 | CAAS | | (SEQ ID | | LQMNSLKAED | (SEQ ID NO: 86) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRIKC | MGWFRQAPG | ISTGTGNTY | YADSVKGRFTI | DVRPDGRTCH | RGQGTLVTVS |
| 6 hSAI- | LVQPGGSLRLS | (SEQ ID NO: 73) | KEREEVAS | (SEQ ID NO: 5) | SQDKSKNTLY | YNS | S |
| BI-006 | CAAS | | (SEQ ID | | LQMNSLKAED | (SEQ ID NO: 87) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRLKC | MGWFRQAPG | ISTGTGNAY | YADSVKGRFTI | DVRPDGRTCH | RGQGTLVTVS |
| 7 h5A1- | LVQPGGSLRLS | (SEQ ID NO: 4) | KEREEVAS | (SEQ ID NO: 95) | SQDKSKNTLY | YNS | S |
| BI-007 | CAAS | | (SEQ ID | | LQMNSLKAED | (SEQ ID NO: 87) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRIKC | MGWFRQAPG | ISTGTGNAY | YADSVKGRFTI | DVRPDGRTCH | RGQGTLVTVS |
| 8 hSAI- | LVQPGGSLRLS | (SEQ ID NO: 73) | KEREEVAS | (SEQ ID NO: 95) | SQDKSKNTLY | YNS | S |
| BI-008 | CAAS | | (SEQ ID | | LQMNSLKAED | (SEQ ID NO: 87) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215810 | EVQLLESGGG | GYTNRIKC | MGWFRQAPG | ISTGTGNSY | YADSVKGRFTI | DVRPDGRTCH | RGQGTLVTVS |
| 9 h5A1- | LVQPGGSLRLS | (SEQ ID NO: 73) | KEREEVAS | (SEQ ID NO: 96) | SQDKSKNTLY | YNK | S |
| BI-009 | CAAS | | (SEQ ID | | LQMNSLKAED | (SEQ ID NO: 88) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVOLLESGGG | GYTNRLKC | MGWFRQAPG | ISTGTGNTY | YADSVKGRFTI | DVRPDGRTCH | RGQGTLVTVS |
| 0h5A1- | LVQPGGSLRLS | (SEQ ID NO: 4) | KEREEVAS | (SEQ ID NO: 5) | SQDKSKNTLY | YNK | S |
| BI-010 | CAAS | | (SEQ ID | | LQMNSLKAED | (SEQ ID NO: 88) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRIKC | MGWFRQAPG | ISTGTGNTY | YADSVKGRFTI | DVRPFGTTCH | RGQGTLVTVS |
| 1 h5Al- | LVQPGGSLRLS | (SEQ ID NO: 73) | KEREEVAS | (SEQ ID NO: 5) | SQDKSKNTLY | YNS | S |
| BI-011 | CAAS | | (SEQ ID | | LQMNSLKAED | (SEQ ID NO: 89) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRLKC | MGWFRQAPG | ISTGTGNTY | YADSVKGRFTI | DVRPFGRTCH | RGQGTLVTVS |
| 2 hSAI- | LVQPGGSLRLS | (SEQ ID NO: 4) | KEREEVAS | (SEQ ID NO: 5) | SQDKSKNTLY | YNS | S |
| BI-012 | CAAS | | (SEQ ID | | LQMNSLKAED | (SEQ ID NO: 90) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRLKC | MGWFRQAPG | ISTGTGNTY | YADSVKGRFTI | DVRPFGTTCH | RGQGTLVTVS |
| 3 h5A1- | LVQPGGSLRLS | (SEQ ID NO: 4) | KEREEVAS | (SEQ ID NO: 5) | SQDKSKNTLY | YNK | S |
| BI-013 | CAAS | | (SEQ ID | | LQMNSLKAED | (SEQ ID NO: 91) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRIKC | MGWFRQAPG | ISTGTGNTY | YADSVKGRFTI | DVRPFGRTCH | RGQGTLVTVS |
| 4 hSAI- | LVQPGGSLRLS | (SEQ ID NO: 73) | KEREEVAS | (SEQ ID NO: 5) | SQDKSKNTLY | YNS | S |
| BI-014 | CAAS | | (SEQ ID | | LQMNSLKAED | (SEQ ID NO: 90) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRIKC | MGWFRQAPG | ISTGTGNTY | YADSVKGRFTI | DVRPFGTTCH | RGQGTLVTVS |
| 5 h5A1- | LVQPGGSLRLS | (SEQ ID NO: 73) | KEREEVAS | (SEQ ID NO: 5) | SQDKSKNTLY | YNK | S |
| BI-015 | CAAS | | (SEQ ID | | LQMNSLKAED | (SEQ ID NO: 91) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRLKC | MGWFRQAPG | ISTGTGNAY | YADSVKGRFTI | DVRPHGTTCH | RGQGTLVTVS |
| 6 h5A1- | LVQPGGSLRLS | (SEQ ID NO: 4) | KEREEVAS | (SEQ ID NO: 95) | SQDKSKNTLY | YNS | S |
| BI-016 | CAAS | | (SEQ ID | | LQMNSLKAED | (SEQ ID NO: 80) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRIKC | MGWFRQAPG | ISTGTGNAY | YADSVKGRFTI | DVRPHGTTCH | RGQGTLVTVS |
| 7 h5A1- | LVQPGGSLRLS | (SEQ ID NO: 73) | KEREEVAS | (SEQ ID NO: 95) | SQDKSKNTLY | YNS | S |
| BI-017 | CAAS | | (SEQ ID | | LQMNSLKAED | (SEQ ID NO: 80) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRLKC | MGWFRQAPG | ISTGTGNAY | YADSVKGRFTI | DVRPHGRTCH | RGQGTLVTVS |
| 8 h5A1- | LVQPGGSLRLS | (SEQ ID NO: 4) | KEREEVAS | (SEQ ID NO: 95) | SQDKSKNTLY | YNS | S |
| BI-018 | CAAS | | (SEQ ID | | LQMNSLKAED | (SEQ ID NO: 92) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215811 | EVQLLESGGG | GYTNRIKC | MGWFRQAPG | ISTGTGNAY | YADSVKGRFTI | DVRPHGRTCH | RGQGTLVTVS |
| 9 h5A1- | LVQPGGSLRLS | (SEQ ID NO: 73) | KEREEVAS | (SEQ ID NO: 95) | SQDKSKNTLY | YNS | S |
| BI-019 | CAAS | | (SEQ ID | | LQMNSLKAED | (SEQ ID NO: 92) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215812 | EVQLLESGGG | GYTNRLKC | MGWFRQAPG | ISTGTGNAY | YADSVKGRFTI | DVRPHGTTCH | RGQGTLVTVS |
| 0 hSAI- | LVQPGGSLRLS | (SEQ ID NO: 4) | KEREEVAS | (SEQ ID NO: 95) | SQDKSKNTLY | YNK | S |
| BI-020 | CAAS | | (SEQ ID | | LQMNSLKAED | (SEQ ID NO: 93) | (SEQ ID |
| | (SEQ ID | | NO: 111) | | TAVYYCAA | | NO: 147) |
| | NO: 101) | | | | (SEQ ID | | |
| | | | | | NO: 140) | | |
| F2215812 | EVQLLESGGG | GYTNRIKC | MGWFRQAPG | ISTGTGNAY | YADSVKGRFTI | DVRPHGTTCH | RGQGTLVTVS |
| 1 h5A1- | LVQPGGSLRLS | (SEQ ID NO: 73) | KEREEVAS | (SEQ ID NO: 95) | SQDKSKNTLY | YNK | S |
| BI-021 | CAAS | | (SEQ ID | | LQMNSLKAED | (SEQ ID NO: 93) | (SEQ ID |
| | (SEQ ID | | NO: 111) | | TAVYYCAA | | NO: 147) |
| | NO: 101) | | | | (SEQ ID | | |
| | | | | | NO: 140) | | |
| BI-HU9- | EVQLLESGGG | GYTNRLKC | MGWFRQAPG | ISTGTGNTY | YADSVKGRFTI | DVRPDGTTCH | RGQGTLVTVS |
| HK h5A1 | LVQPGGSLRLS | (SEQ ID NO: 4) | KERELVAS | (SEQ ID NO: 5) | SRDNSKNTLY | YNS (SEQ ID | S |
| | CAAS | | (SEQ ID | | LQMNSLKAED | NO: 6) | (SEQ ID |
| | (SEQ ID | | NO: 114) | | TAVYYCAA | | NO: 147) |
| | NO: 101) | | | | (SEQ ID | | |
| | | | | | NO: 121) | | |
| BI-HU9- | EVQLLESGGG | GYTNRLKC | MGWFRQAPG | ISTGTGNTY | YADSVKGRFTI | DVRPDGTTCH | RGQGTLVTVS |
| HK-1 | LVQPGGSLRLS | (SEQ ID NO: 4) | KEREEVAS | (SEQ ID NO: 5) | SQDKSKNTLY | YNS (SEQ ID | S |
| h5A1 | CAAS | | (SEQ ID | | LQMNSLKAED | NO: 6) | (SEQ ID |
| | (SEQ ID | | NO: 111) | | TAVYYCAA | | NO: 147) |
| | NO: 101) | | | | (SEQ ID | | |
| | | | | | NO: 140) | | |
| BI-HU9- | EVQLLESGGG | GYTNRLKC | MGWFRQAPG | ISTGTGNTY | YADSVKGRFTI | DVRPDGTTCH | WGQGTQVTV |
| VH9 | LVPRGGSLRLS | (SEQ ID NO: 4) | KEREEIAT | (SEQ ID NO: 5) | SRDNSRNTLY | YNS (SEQ ID | SS |
| h5A1 | CTAS | | (SEQ ID | | LQMKTLRAED | NO: 6) | (SEQ ID |
| | (SEQ ID NO: 99) | | NO: 112) | | TAVYYCAA | | NO: 146) |
| | | | | | (SEQ ID | | |

NO: 120)

| | | | | | | | |
|---|---|---|---|---|---|---|---|
| BI-HU9- | EVQLLESGGG | GYTNRLKC | MGWFRQAPG | ISTGTGNTY | YADSVKGRFTI | DVRPDGTTCH | WGQGTQVTV |
| VH9-1 | LVPRGGSLRLS | (SEQ ID NO: 4) | KEREEIAT | (SEQ ID NO: 5) | SQDKSRNTLY | YNS (SEQ ID | SS |
| h5A1 | CTAS | | (SEQ ID | | LQMKTLRAED | NO: 6) | (SEQ ID |
| | (SEQ ID NO: 99) | | NO: 112) | | TAVYYCAA | | NO: 146) |
| | | | | | (SEQ ID | | |
| | | | | | NO: 141) | | |
| BI-HU9- | EVQLLESGGG | GFTFRLKC | MGWFRQAPG | ISTGTGNTY | YADSVKGRFTI | DVRPDGTTCH | WGQGTTVTVS |
| VHH11 | LVQPGGSLRLS | (SEQ ID NO: 74) | KGLEEVAT | (SEQ ID NO: 5) | SRDNSKNTLY | YNS (SEQ ID | S |
| h5A1 Fc | CAAS | | (SEQ ID | | LQMNSLRAED | NO: 6) | (SEQ ID |
| | (SEQ ID | | NO: 117) | | TAVYYCAA | | NO: 149) |
| | NO: 101) | | | | (SEQ ID | | |
| | | | | | NO: 142) | | |
| BI-HU9- | EVQLLESGGG | GYTNRLKC | MGWFRQAPG | ISTGTGNTY | YADSVKGRFTI | DVRPDGTTCH | WGQGTTVTVS |
| VHH12 | LVQPGGSLRLS | (SEQ ID NO: 4) | KGLEEVAT | (SEQ ID NO: 5) | SRDNSKNTLY | YNS (SEQ ID | S |
| h5A1 Fc | CAAS | | (SEQ ID | | LQMNSLRAED | NO: 6) | (SEQ ID |
| | (SEQ ID | | NO: 117) | | TAVYYCAA | | NO: 149) |
| | NO: 101) | | | | (SEQ ID | | |
| | | | | | NO: 142) | | |
| BI-HU9- | EVQLLESGGG | GYTNRLKC | MGWFRQAPG | ISTGTGNTY | YADSVKGRFTI | DVRPDGTTCH | WGQGTTVTVS |
| VHH13 | LVQPGGSLRLS | (SEQ ID NO: 4) | KGLEEVAT | (SEQ ID NO: 5) | SQDKSKNTLY | YNS (SEQ ID | S |
| h5A1 | CAAS | | (SEQ ID | | LOMNSLRAED | NO: 6) | (SEQ ID |
| | (SEQ ID | | NO: 117) | | TAVYYCAA | | NO: 149) |
| | NO: 101) | | | | (SEQ ID | | |
| | | | | | NO: 143) | | |
| BI-HU9- | QVQLVESGGG | GFTFRLKC | MGWFRQAPG | ISTGTGNTY | YADSVKGRFTI | DVRPDGTTCH | WGQGTTVTVS |
| VHH21 | VVQPGGSLRL | (SEQ ID NO: 74) | KGLEEVAT | (SEQ ID NO: 5) | SRDNSKNTLY | YNS (SEQ ID | S |
| h5A1 | SCAAS | | (SEQ ID | | LQMNSLRAED | NO: 6) | (SEQ ID |
| | (SEQ ID | | NO: 117) | | TAVYYCAA | | NO: 149) |
| | NO: 109) | | | | (SEQ ID | | |
| | | | | | NO: 142) | | |
| BI-HU9- | QVQLVESGGG | GYTNRLKC | MGWFRQAPG | ISTGTGNTY | YADSVKGRFTI | DVRPDGTTCH | WGQGTTVTVS |
| VHH22 | VVQPGGSLRL | (SEQ ID NO: 4) | KGLEEVAT | (SEQ ID NO: 5) | SRDNSKNTLY | YNS (SEQ ID | S |
| h5A1 | SCAAS | | (SEQ ID | | LQMNSLRAED | NO: 6) | (SEQ ID |
| | (SEQ ID | | NO: 117) | | TAVYYCAA | | NO: 149) |
| | NO: 109) | | | | (SEQ ID | | |
| | | | | | NO: 142) | | |
| BI-HU9- | QVQLVESGGG | GYTNRLKC | MGWFRQAPG | ISTGTGNTY | YADSVKGRFTI | DVRPDGTTCH | WGQGTTVTVS |
| VHH23 | VVQPGGSLRL | (SEQ ID NO: 4) | KGLEEVAT | (SEQ ID NO: 5) | SQDKSKNTLY | YNS (SEQ ID | S |
| h5A1 | SCAAS | | (SEQ ID | | LQMNSLRAED | NO: 6) | (SEQ ID |
| | (SEQ ID | | NO: 117) | | TAVYYCAA | | NO: 149) |
| | NO: 109) | | | | (SEQ ID | | |
| | | | | | NO: 143) | | |
| BI-HU9- | EVQLVESGGG | GFTVRLKC | MGWFRQAPG | ISTGTGNTY | YADSVKGRFTI | DVRPDGTTCH | WGQGTTVTVS |
| VHH31 | LVQPGGSLRLS | (SEQ ID NO: 75) | KGLEEVAT | (SEQ ID NO: 5) | SRDNSKNTLY | YNS (SEQ ID | S |
| h5A1 | CAAS | | (SEQ ID | | LQMNSLRAED | NO: 6) | (SEQ ID |
| | (SEQ ID | | NO: 117) | | TAVYYCAA | | NO: 149) |
| | NO: 110) | | | | (SEQ ID | | |
| | | | | | NO: 142) | | |
| BI-HU9- | EVQLVESGGG | GYTNRLKC | MGWFRQAPG | ISTGTGNTY | YADSVKGRFTI | DVRPDGTTCH | WGQGTTVTVS |
| VHH32 | LVQPGGSLRLS | (SEQ ID NO: 4) | KGLEEVAT | (SEQ ID NO: 5) | SRDNSKNTLY | YNS (SEQ ID | S |
| h5A1 | CAAS | | (SEQ ID | | LQMNSLRAED | NO: 6) | (SEQ ID |
| | (SEQ ID | | NO: 117) | | TAVYYCAA | | NO: 149) |
| | NO: 110) | | | | (SEQ ID | | |
| | | | | | NO: 142) | | |
| BI-HU9- | EVQLVESGGG | GYTNRLKC | MGWFRQAPG | ISTGTGNTY | YADSVKGRFTI | DVRPDGTTCH | WGQGTTVTVS |
| VHH33 | LVQPGGSLRLS | (SEQ ID NO: 4) | KGLEEVAT | (SEQ ID NO: 5) | SQDKSKNTLY | YNS (SEQ ID | S |
| h5A1 | CAAS | | (SEQ ID | | LQMNSLRAED | NO: 6) | (SEQ ID |
| | (SEQ ID | | NO: 117) | | TAVYYCAA | | NO: 149) |
| | NO: 110) | | | | (SEQ ID | | |
| | | | | | NO: 143) | | |

TABLE 10A
Polypeptide 1 comprises an Fc domaine; Polypeptide 2 comprises an Fc domain; and Polypeptide 3
comprises the light chain, The VHH antibody may be included in Polypeptide 1, polypeptide 2
and/or polypeptide 3, and can utilize a linker between them. In this table the anti-CD3 domain
is on the N-terminal of the Fc. The “(VHH antibody)” can be selected from Table 7.

| | Polypeptide 1 | Polypeptide 2 | |
|---|---|---|---|
| Mono- | (VHH | | |
| clonal | antibody)EPKSADKTHTCPPCPAPELLGG | | |
| Fc | PSVFLFPPKPKDTLMISRTPEVTCVVVDVSH | | |
| | EDPEVKFNWYVDGVEVHNAKTKPREEQYN | | |
| | STYRVVSVLTVLHQDWLNGKEYKCKVSN | | |
| | KALPAPIEKTISKAKGQPREPQVYTLPPSRE | | |
| | EMTKNQVSLTCLVKGFYPSDIAVEWESNG | | |
| | QPENNYKTTPPVLDSDGSFFLYSKLTVDKS | | |
| | RWQQGNVFSCSVMHEALHNHYTQKSLSL | | |
| | SPGK (SEQ ID NO: 198) | | |
| Ac13 | EVOLVESGGGLVQPGGSLRLSCAASGFTFS | (VHH antibody) | QAVVTQEPSLTVSPGGTVTLTCRSS |
| | TYAMNWVRQAPGKGLEWVARIRSKYNN | EPKSSDKTHTCPPCPAPEAAGGPSVFLF | TGAVTTSNYANWVQQKPGQAPRGLI |
| | YATYYADSVKDRFTISRDDSKNTLYLQMN | PPKPKDTLMISRTPEVTCVVVDVSHEDP | GGTNKRAPGTPARFSGSLLGGKAAL |
| | SLRAEDTAVYYCARHGNFGNSYVSWFAY | EVKFNWYVDGVEVHNAKTKPREEQYNS | TLSGVQPEDEAEYYCALWYSNLWVF |
| | WGQGTMVTVSSASTKGPSVFPLAPSSKSTS | TYRVVSVLTVLHQDWLNGKEYKCKVS | GGGTKLTVLGQPKAAPSVTLFPPSS |
| | GGTAALGCLVKDYFPEPVTVSWNSGALTS | NKALPAPIEKTISKAKGQPREPQVYTLP | EELQANKATLVCLISDFYPGAVTVA |
| | GVHTFPAVLQSSGLYSLSSVVTVPSSSLGT | PSREEMTKNQVSLWCLVKGFYPSDIAVE | WKADSSPVKAGVETTTPSKQSNNKY |
| | QTYICNVNHKPSNTKVDKKVEPKSCDKTH | WESNGQPENNYKTTPPVLDSDGSFFLYS | AASSYLSLTPEQWKSHRSYSCQVTH |
| | TCPPCPAPEAAGGPSVFLFPPKPKDTLMISR | KLTVDKSRWQQGNVFSCSVMHEALHN | EGSTVEKTVAPTECSGGGGSGGGGS |
| | TPEVTCVVVDVSHEDPEVKFNWYVDGVE | HYTQKSLSLSPGK (SEQ ID | GGGGSGGGGS (VHH antibody) |
| | VHNAKTKPREEQYNSTYRVVSVLTVLHQ | NO: 199) | (SEQ ID NO: 200) |
| | DWLNGKEYKCKVSNKALPAPIEKTISKAK | | |
| | GQPREPQVYTLPPSREEMTKNQVSLSCAV | | |
| | KGFYPSDIAVEWESNGQPENNYKTTPPVL | | |
| | DSDGSFFLVSKLTVDKSRWQQGNVFSCSV | | |
| | MHEALHNRFTQKSLSLSPGK (SEQ ID | | |
| | NO: 153) | | |

TABLE 10B

| | DNA1 | DNA2 | DNA3 |
|---|---|---|---|
| Mono- | (DNA encoding VHH antibody) | | |
| clonal | GAGCCCAAATCTGCTGACAAAACTCAC | | |
| Fc | ACATGCCCACCGTGCCCAGCACCTGAA | | |
| | CTCCTGGGGGGACCGTCAGTCTTCCTCT | | |
| | TCCCCCCAAAACCCAAGGACACCCTCA | | |
| | TGATCTCCCGGACCCCCGAGGTCACAT | | |
| | GCGTGGTGGTGGACGTGAGCCACGAAG | | |
| | ACCCTGAGGTCAAGTTCAACTGGTACG | | |
| | TGGACGGCGTGGAGGTGCATAATGCCA | | |
| | AGACAAAGCCGCGGGAGGAGCAGTAC | | |
| | AACAGCACGTACCGTGTGGTCAGCGTC | | |
| | CTCACCGTCCTGCACCAGGACTGGCTG | | |
| | AATGGCAAGGAGTACAAGTGCAAGGTC | | |
| | TCCAACAAAGCCCTCCCAGCCCCCATC | | |
| | GAGAAAACCATCTCCAAAGCCAAAGGG | | |
| | CAGCCCCGAGAACCACAGGTGTACACC | | |
| | CTGCCCCCATCCCGGGAGGAGATGACC | | |
| | AAGAACCAGGTCAGCCTGACCTGCCTG | | |
| | GTCAAAGGCTTCTATCCCAGCGACATC | | |
| | GCCGTGGAGTGGGAGAGCAATGGGCA | | |
| | GCCGGAGAACAACTACAAGACCACGCC | | |
| | TCCCGTGCTGGACTCCGACGGCTOCTTC | | |
| | TTCCTCTACAGCAAGCTCACCGTGGAC | | |
| | AAGAGCAGGTGGCAGCAGGGGAACGT | | |
| | CTTCTCATGCTCCGTGATGCATGAGGCT | | |
| | CTGCACAACCACTACACGCAGAAGAGC | | |
| | CTCTCCCTGTCTCCGGGTAAA (SEQ | | |
| | ID NO: 201) | | |
| Ac13 | GAGGTGCAGCTGGTGGAGTCCGGAGGA | (DNA encoding VHH antibody) | CAGGCTGTGGTGACCCAGGAGCCCAGC |
| | GGACTGGTGCAGCCTGGAGGAAGCCTG | GAGCCCAAATCTAGCGACAAAACTCAC | CTGACCGTGAGCCCCGGAGGAACAGTG |
| | AGGCTGTCCTGCGCTGCTTCCGGATTTA | ACATGCCCACCGTGCCCAGCACCTGAA | ACCCTGACCTGCAGAAGCAGCACCGGC |
| | CATTTAGCACATATGCCATGAACTGGG | GCCGCCGGGGGACCGTCAGTCTTCCTCT | GCCGTGACAACCTCAAACTACGCCAAC |
| | TGAGGCAGGCCCCTGGAAAAGGTCTGG | TCCCCCCAAAACCCAAGGACACCCTCA | TGGGTGCAGCAGAAACCTGGACAGGCC |
| | AATGGGTGGCTAGGATTCGGTCCAAGT | TGATCTCCCGGACCCCCGAGGTCACAT | CCCAGAGGACTGATCGGCGGAACCAAC |
| | ACAACAATTACGCCACCTACTATGCCG | GCGTGGTGGTGGACGTGAGCCACGAAG | AAGAGAGCCCCCGGCACCCCCGCCAGA |
| | ACAGTGTCAAGGACAGGTTTACCATCT | ACCCTGAGGTCAAGTTCAACTGGTACG | TTCAGCGGAAGTCTGCTGGGCGGCAAA |
| | CCAGGGACGATTCAAAGAACACCCTGT | TGGACGGCGTGGAGGTGCATAATGCCA | GCCGCCCTGACCCTGAGCGGAGTGCAG |
| | ACCTGCAGATGAACTCCCTGAGGGCOG | AGACAAAGCCGCGGGAGGAGCAGTAC | CCTGAGGATGAAGCCGAGTACTACTGC |
| | AGGATACCGCTGTGTATTACTGTGCCCG | AACAGCACGTACCGTGTGGTCAGCGTC | GCTCTGTGGTACAGCAACCTGTGGGTG |
| | GCACGGAAATTTCGGGAACTCCTACGT | CTCACCGTCCTGCACCAGGACTGGCTG | TTCGGAGGAGGCACCAAGCTGACCGTG |
| | GTCCTGGTTCGCATACTGGGGCCAGGG | AATGGCAAGGAGTACAAGTGCAAGGTC | CTGGGTCAGCCCAAGGCTGCCCCCTCG |
| | AACAATGGTGACTGTGTCCTCCGCTAG | TCCAACAAAGCCCTCCCAGCCCCCATC | GTCACTCTGTTCCCGCCCTCCTCTGAG |
| | CACCAAGGGCCCATCGGTCTTCCCCCTG | GAGAAAACCATCTCCAAAGCCAAAGGG | GAGCTTCAAGCCAACAAGGCCACACTG |
| | GCACCCTCCTCCAAGAGCACCTCTGGG | CAGCCCCGAGAACCACAGGTGTACACC | GTGTGTCTCATAAGTGACTTCTACCCG |
| | GGCACAGCGGCCCTGGGCTGCCTGGTC | CTGCCCCCATCCCGGGAGGAGATGACC | GGAGCCGTGACAGTGGCCTGGAAGGCA |
| | AAGGACTACTTCCCCGAACCGGTGACG | AAGAACCAGGTCAGCCTGTGGTGCCTG | GATAGCAGCCCCGTCAAGGCGGGAGTG |
| | GTGTCGTGGAACTCAGGCGCCCTGACC | GTCAAAGGCTTCTATCCCAGCGACATC | GAGACCACCACACCCTCCAAACAAAGC |
| | AGCGGCGTGCACACCTTCCCGGCTGTC | GCCGTGGAGTGGGAGAGCAATGGGCA | AACAACAAGTACGCGGCCAGCAGCTAC |
| | CTACAGTCCTCAGGACTCTACTCCCTCA | GCCGGAGAACAACTACAAGACCACGCC | CTGAGCCTGACGCCTGAGCAGTGGAAG |
| | GCAGCGTGGTGACCGTGCCCTCCAGCA | TCCCGTGCTGGACTCCGACGGCTCCTTC | TCCCACAGGAGCTACAGTTGCCAGGTC |
| | GCTTGGGCACCCAGACCTACATCTGCA | TTCCTCTACAGCAAGCTCACCGTGGAC | ACGCATGAAGGGAGCACCGTGGAGAAG |
| | ACGTGAATCACAAGCCCAGCAACACCA | AAGAGCAGGTGGCAGCAGGGGAACGT | ACAGTGGCCCCTACAGAATGTTCAGGT |
| | AGGTGGACAAGAAAGTTGAGCCCAAAT | CTTCTCATGCTCCGTGATGCATGAGGCT | GGCGGAGGGTCTGGTGGTGGAGGATCA |
| | CTTGTGACAAAACTCACACATGCCCAC | CTGCACAACCACTACACGCAGAAGAGC | GGGGGTGGAGGTTCAGGAGGCGGGGGA |
| | CGTGCCCAGCACCTGAAGCOGCAGGGG | CTCTCCCTGTCTCCGGGTAAA (SEQ | AGT |
| | GACCGTCAGTCTTCCTCTTCCCCCCAAA | ID NO: 202) | (DNA encoding VHH antibody) |
| | ACCCAAGGACACCCTCATGATCTCCCG | | (SEQ ID NO: 203) |
| | GACCCCCGAGGTCACATGCGTGGTGGT | | |
| | GGACGTGAGCCACGAAGACCCTGAGGT | | |
| | CAAGTTCAACTGGTACGTGGACGGCGT | | |
| | GGAGGTGCATAATGCCAAGACAAAGCC | | |
| | GCGGGAGGAGCAGTACAACAGCACGT | | |
| | ACCGTGTGGTCAGCGTCCTCACCGTCCT | | |
| | GCACCAGGACTGGCTGAATGGCAAGGA | | |

GTACAAGTGCAAGGTCTCCAACAAAGC
CCTCCCAGCCCCCATCGAGAAAACCAT
CTCCAAAGCCAAAGGGCAGCCCCGAGA
ACCACAGGTGTACACCCTGCCCCCATC
CCGGGAGGAGATGACCAAGAACCAGG
TCAGCCTGAGCTGCGCGGTCAAAGGCT
TCTATCCCAGCGACATCGCCGTGGAGT
GGGAGAGCAATGGGCAGCCGGAGAAC
AACTACAAGACCACGCCTCCCGTGCTG
GACTCCGACGGCTCCTTCTTCCTCGTCA
GCAAGCTCACCGTGGACAAGAGCAGGT
GGCAGCAGGGGAACGTCTTCTCATGCT
CCGTGATGCATGAGGCTCTGCACAACA
GATTTACGCAGAAGAGCCTCTCCCTGTC
TCCGGGTAAA (SEQ ID NO: 171)

TABLE 11
The sequences, antibodies and fragments thereof (e.g., VHH, Fab regions, or scFv) in the references, publications,
patent documents, webpages contained in this table are hereby incorporated by reference as though fully set forth.

| Name | Alternative names | Description |
|---|---|---|
| Anti-CD3 | | |
| muromonab | OKT3 (Ortho | First monoclonal murine antibody to become available |
| | Kung T3) | for therapy in humans - mouse IgG2a is directed |
| | | against the CD3 epsilon chain of the CD3/TCR complex |
| Teplizumab | hOKT3γ1 (Ala- | CDRs granted onto human IgG backbone with two mutations |
| | Ala) and MGA031 | (L234/A, L235A) |
| Otelixizumab | ChAglyCD3, | chimeric and humanized IgG1 bears a single mutation |
| | TRX4, | in the γ1 Fc portion (N297/A) to avoid glycosylation |
| | GSK2136525 | and thus inhibit FcR binding |
| Visilizumab | Nuvion, HuM291 | humanized IgG2 antibody rendered non mitogenic by |
| | | two point mutations in its Fc region (V234/A, V237/A) |
| Foralumab | 28F11-AE; | entirely human anti-CD3 mAb whose Fc portion of this |
| | NI-0401 | human IgG1 was mutated (L234/A, V235E) such that the |
| | | mAb is non FcR binding in vitro and exhibits only minor |
| | | cytokine release |
| Bispecific | | |
| Blinatumomab | AMG 103 | linked scFv(s) againt CD19 on tumor cells and CD3 on |
| | | T cells; no Fc domain; Anti-CD3 comes from murine mAb: |
| | | L2K-07; published sequences encoding scFv addressing CD3ε |
| | | (diL2K, the de-immunized version of the mouse monoclonal |
| | | antibody L2K) |
| flotetuzumab | MGD006 | DART targeting CD3 and CD123 |
| Cibisatamab | | 1 binding site for CD3e chaim and 2 CEA binding sties |
| | | for cancer cells |
| Catumaxomab | | consists of mouse IgG2 and rat IgG2b and targets CD3 |
| | | on T cells and EpCAM on tumor cells |
| Mosunetuzumab | | CD20 × CD3 T-cell engaging bsAb |

All candidates shared the same CD3-specific singlechain antibody derived from mouse monoclonal antibody
L2K, which is distinct from monoclonal antibodies TR66 and OKT3. L2K was subjected to a procedure that
identifies human T helper cell epitopes and removes by point mutations MHC class II anchor amino acids
within potential epitopes (Jones et al., 2004). This resulted in “de-immunized” L2K (diL2K)
(sequence encoding scFv addressing CD3e is disclosed in K. Brischwein et al./Molecular Immunology 43 (2006) 1129-1143)

| TR66 | monoclonal anti-human CD3 epsilon antibody |
|---|---|
| PIT565 | Trispecific: CD19 × CD3 × CD2 |
| Four CD3 mAb-based binders: OKT3, | |
| L2K, h38E4.v1 and TRX4 | |
| OKT3 | |
| TRX4 | |
| h38E4.v1 | Binds N-terminal portion of CD3e extracellular domain |
| L2K | L2K, for instance shares over 90% sequence identity with |
| | OKT3 and recognizes the same epitope on CD3ε but differs |
| | in affinity by about 100-fold |
| Anti-CD16 | |
| 3G8 | monoclonal anti-human CD16 |
| U.S. Pat. No. 9,035,026 | |
| HRS-3/A9 | A9 is anti-CD16 mouse IgG1/λ Mab |
| Humanization of anti-CD16 Fab | Protein Engineering, Design and Selection, Volume 22, Issue |
| | 3, March 2009, Pages 175-188. the CDRs of the anti-CD16 mouse |
| | IgG1/λ MAb A9 were grafted onto human Ig sequences (CDRs were |
| | transplanted onto a human κ light chain + Affinity maturation |
| | via radon mutation in the humanized VH and Vκ domains ) |
| AFM13 | Tetravalent bispecific CD30/CD16 (binds CD16A on NK cell |
| | surface, thus activating and recruiting NK cells to CD30+ |
| | tumor cell) |
| GTB-3550 | Trispecific CD16/IL-15/CD33 (anti-CD16 nanobody, or 1st |
| | generation uses scFv anti-CD16) |
| IPH6501 | Antibody-based tetraspecific against NKp46, CD16a, CD122 |
| | or IL-2R, and CD20 |

AFM13: anti-CD16A binder is variable domains from human scFv clone LSIV21
Other human scFv anti-CD16A clones: LSIV14, LSIII49, LBIII6, LBIII5, LBIII3, 50NI

| Cite: Reusch et al., miAbs 6:3, 727-738, | |
|---|---|
| May/June 2014 | |
| Binders for V gamma 9, V delta 2 | |
| gene, V delta 1 gene segments in | |
| TCR | |
| LAVA-051 | Lava Therapeutics. bispecific gamma delta T cell engagers |
| | (bsTCEs): Vγ9Vδ2 T cell receptor occupancy of LAVA-051 |
| | increased with LAVA-051 dose increases and peripheral blood |
| | Vγ9Vδ2 T cells also expressed higher levels of activation |
| | markers after LAVA-051 dosing |
| | LAVA-051 consists of two humanized single domain antibodies |
| | (VHH) linked via a short five a.a. G-S linker, wherein one |
| | doamin antibody recognizes the Vδ2 chain of the Vγ9Vδ2 T cell |
| | receptor, and the other domain antibody is specific for CD1d. |
| LAVA-1207 | Binds: the Vδ2 chain of Vγ9Vδ2-T cells and prostate specific |
| | membrane antigen (PSMA) |

| | |
|---|---|
| Bruin et al., Clinical Immunology, y. | set of Vγ9Vδ2-T cell specific VHH (or nanobody) |
| 169, 128-138. August 2016 | |

Clones: 5C1, 5D3, 5E3, 5G3, 5F5, 5C7, 5D7, 5E7, 5C8, 5B11, 6A1, 6C1, 6H1, 6E3, 6G3, 6H3, 6C4, 6E4, 6H4, 6F6

| U.S. Pat. Nos. 10,501,540 & 11,384,145 | | |
|---|---|---|
| clone 7A5: the variable regions of the | | Construct: Vg9 TCR × Her2/neu; that is, (Her2)₂xVγ9 bispecific |
| Vg9gd T-cell-specific antibody ( | | antibody |
| GDX012 | GammaDelta | allogeneic, non-engineered, variable delta I (Vδ1) gamma-delta (γδ) T cell therapy |
| | Therapeutics | |
| See Pat. Appl. pub.: US20220403025; | | |
| US20220290101; | | |
| Anti-CD137 (4- | | |
| 1BB/TNFRSF9) | | |
| AGEN2373 | | by Agenus Inc; mAb IgG1; U.S. Pat. Nos. 11,098,117 & 11,242,385 |
| utomilumab | PF-05082566 | Humanized IgG2 mAb; binding site: cysteine-rich domain (CRD)s |
| | | III and IV |
| Urelumab | BMS-663513 | Human IgG4 mAb; binding site: CRD I; trial by BMS |
| CTX-471 | by Compass | fully human IgG4 mAb |
| | Therapeutics | |
| ADG106 | by Adagene Suzhou | fully human ligand-blocking, agonistic IgG4 |
| LVGN6051 | by Lyvgen | IgG1, improved FcγRIIB binding |
| | Biopharma/Merck | |
| | Sharp & Dohme | |
| ATOR-1017 | by Alligator | IgG4 |
| | Bioscience AB | |
| HOT-1030 | by Shanghai Huabo | IgG1, abrogated FcγRIIB binding |
| | Biopharmaceutical | |
| | (Huaota)/Eutilex | |
| EU101 | by | IgG |
| | Eutilex/Zhejiang | |
| | Huahai | |
| | Pharmaceutical | |
| YH004 | by Eucure | IgG1 |
| | (Beijing) | |
| | Biopharma | |
| ADG206 | by Adagene | IgG1, enhanced IgG1 binding, 4-1BB binding sites are masked |
| | Suzhou | |
| PE0116 | by Shanghai | IgG4 |
| | Hyamab Biotech | |
| FS120 | by F-Star | OX40/CD137 mAb² dual agonist bispecific antibody |
| | Therapeutics Inc. | |
| cinrebafusp alfa | PRS-343 | bispecific 4-1BB (Anticalin ®) × HER2 |
| RG7827 | RO 7122290 | FAP × 4-1BBL antibody fusion protein |
| INBRX-105 | ES101 | humanized, bispecific IgG1 targeting PD-L1 × 4-1BB |
| Acasunlimab | GEN 1046, BNT | Fc-silenced IgG1 bispecific PD-L1 × 4-1BB |
| | 311 | |
| MCLA-145 | | bispecific CD137 × PD-L1 |
| Englumafusp alfa | RG6076, | CD19 × CD137 |
| | RO7227166 | |
| Additional bi/multispecific in MAbs. | | |
| 2023: 15(1): 2167189 | | |
| Anti-CD28 | | |
| Theralizumab | TGN1412, CD28- | CYTOKINE storm failed human safety trial; humanised |
| | SuperMAB, | monoclonal antibody directed against the human CD28 |
| | >Renamed as | Trial for rheumatoid arthritis |
| | TAB08 | |
| Anti-DNAM-1 | | |
| (CD226 or PTA1) | | |
| LY3435151 | | agonistic anti-CD226; NCT04099277 by Eli Lilly; U.S. Pat. |
| | | No. 11,440,959 |
| Anti-NKp46 | | |
| (NCR1, CD335) | | |
| IPH6501 | | NKp46-Targeting Tetraspecific Antibody-Based Natural Killer |
| | | Cell Engager Therapeutic Armed with a Non-Alpha IL-2 Variant |
| | | (by Innate Pharma) |
| Trifunctional | | Targeting NKp46, CD16, and a tumor antigen (Cell 177, 1701-1713, |
| NKCE | | Jun. 13, 2019); U.S. Pat. No. 10,519,234 (by Innate Pharma) |
| | | Other Innate Pharma owned NKp46 binder patents: 11,001,629, |
| | | 11,377,492, and 11,267,897 |
| Anti-NKp30 | | |
| | Compass | US20200109195, US20200079867, particularly SEQ ID NOs: 14 and 18 |
| | Therapeutics | |
| Anti-NKG2D | | |
| | NCT05213195 by | US20200360437 by Asko (Suzhou) |
| | Zhejiang Univ. | |
| | | NCT05213195, NCT05247957, NCT04324996, NCT04623944, NCT03415100 |
| Anti-OX40 | | |
| (CD134; TNFRSF4) | | |
| INCAGN1949 | | human IgG1 |
| | | Anti-OX40 patents: U.S. Pat. Nos. 11,472,883, 11,447,557, |
| | | 11,359,028, 11,332,536, 11,136,404, 10,626,181, 10,259,882 |
| HFB301001 | | 2nd gen. fullly human IgG1 OX40 agonist antibody by HiFiBio |
| GSK3174998 | | anti-OX40 mAb |
| INBRX 106 | | hexavalent OX40 agonist, active ingredient: hexavalent IgG |
| | | antibody that targets the human OX40 receptor (TNFRSF4) |
| FS120 | by F-Star | OX40/CD137 mAb² dual agonist bispecific antibody |
| | Therapeutics Inc. | |
| Ivuxolimab | PF-04518600 or | humanized agonistic IgG2 monoclonal antibodies against OX40 |
| | PF 8600 | |
| BGB-A445 | | does not block the OX40-OX40L interaction; |
| MOXR0916 | by Genentech | humanized effector-competent agonist IgG1 mAb; completely |
| | | blocked OX40 binding to OX40L |
| BMS-986178 | | fully human IgG1 agonist mAb |
| Anti-ICOS (CD278, | | |
| AILIM, CVID1) | | |
| Izuralimab | XmAb23104 by | bispecific PD1 × ICOS |
| | Xencor | |
| MEDI-570 | by Astrazeneca | |
| BMS-986226 | | |
| KY1044 | by Kymab | fully human anti-ICOS IgG1 |
| feladilimab | GSK3359609, | humanized IgG4 anti-ICOS monoclonal |
| | INDUCE-1 | |
| vopratelimab | JTX-2011 by | |
| | Jounce | |
| | Therapeutics | |
| Acazicolcept | | ICOS × CD28 |
| Anti-CD2 | | |
| Siplizumab | TCD601, MEDI- | humanized monoclonal anti-CD2 IgG1 (Humanized form of |
| | 507, Hu-BTI-322 | BTI-322 below) |
| PIT565 | by Novartis | Trispecific CD19 × CD3 × CD2 (NCT05397496) |
| Alefacept | | fusion protein composed of the first extracellular domain |
| | | of LFA-3 (natural ligand of CD2) fused to the human IgG1 |
| | | hinge, CH2, and CH3 domains |
| BTI-322 | Lo-CD2a | rat anti-human CD2 IgG2b mAb |
| Anti-CD40L | | |
| (CD154) | | |
| ruplizumab | BG 9588, hu5c8 | humanized IgG1k |
| Dazodalibcp | HZN-4920 by | antagonistic |
| Dapirolizumab | CDP7657 | anti-CD40L antibody fragment with the Fc moiety substituted |
| pegol | | by polyethylene glycol |
| Tegoprubart | AT-1501 | humanized mAb |
| Frexalimab | SAR441344 | 2nd gen. mAb engineered to lack the domain that activated FcgRIIA |
| Lu AG22515 | APB-A1; by | differentiated anti-CD40L antibody |
| | Lundbeck | |
| Letolizumab | BMS 986004 | humanized mAb (a dimeric fusion protein composed of the C- |
| | | terminus of the domain antibody (dAb) BMS2h-572-633 targeting |
| | | CD40L linked to a modified Fc fragment of IgG1) |
| BIIB063 | | high affinity MAb, engineered to reduce Fc effector function |
| | | and avoid half antibody formation in vivo (IgG4P |
| Anti-CD40 | | |
| (TNFRSF5) | | |
| CDX-1140 | | fully human IgG2 agonistic mAb |
| Sotigalimab | APX005M | humanized IgG1 agonistic mAb |
| 2141-VII | | human anti-CD40 antibody with modified Fc domain (IgG1), |
| | | which may induce durable anti-tumor immunity |
| selicrelumab | RO7009789 by | human IgG2 agonistic mAb; CP-870,893 |
| | Roche, or CP- | |
| | 870893 | |
| dalnicastobart | LVGN7409 | humanized IgG1 mAb |
| Iscalimab | CFZ533 | fully human IGg1 mAb |
| Dacetuzumab | SGN-40 | humanized IgG1 form of S2C6 (a murine anti-human CD40) |
| BI 655064 | by Boehringer | humanised, nondepleting, antagonistic anti-CD40 |
| | Ingelheim | |
| Bleselumab | ASKP1240 | fully human IgG4 mAb |
| YH003 | | humanized agonistic CD40 IgG2 mAb |
| SL-172154 | SIRPα-Fc-CD40L | Bifunctional CD47 × CD40 |
| Giloralimab | ABBV-927 | agonistic mAb |
| Lucatumumab | CHIR 12.12 or | fully human mAb |
| | HCD122 | |
| Mitazalimab | ADC-1013 | agonistic human IgG1 mAb |
| GEN1042 | | agonistic bispecific CD40 × 4-1BB |
| FFP104 | PG102 | humanised |
| KPL-404 | | humanized IgG4 antibody engineered to bind CD40 |
| | | without triggering Fc effector functions |
| Cifurtilimab | SEA-CD40 | humanized IgG1 mAb |
| Ravagalimab | ABBV-323 | antagonistic |
| ABBV-428 | | Bispecific CD40 × MSLN |
| BMS-986090 | INX-189 | a dimeric anti-human CD40 VH antagonist domain antibody |
| | | formatted with a human IgG4 Fc |
| BSI-038 | | agonistic mAb |
| MEDI5083 | | by MedImmune, now Astrazeneca; hexameric CD40L-Fc fusion |
| RG6189 | | a FAP-targeted CD40 agonistic bispecific antibody |
| Inezetamab | AMG 994 | an immunoglobulin G1-scFv_L-lambda2 dimer |

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

As shown in FIG. 1, panel A depicts an exemplary bispecific TCE in format 11A whereby a CD3-specific Fab is fused to the N-terminus of a knob chain of a knob-in-hole (KIH) heterodimeric IgG1 Fc scaffold and two anti-TAA VHH are fused in tandem to the N-terminus of a hole chain of the KIH scaffold. Panel B is an exemplary bispecific TCE in format 13cA whereby a CD3-specific Fab is fused in one of its two polypeptide chains to the N-terminus of the knob chain of a KIH scaffold, a first TAA-specific VHH is fused to the N-terminus of a hole chain, and a second TAA-specific VHH is fused to the C-terminus of the light chain of the CD3-specific Fab. Panel C is an exemplary bispecific TCE in format Ac13 whereby a CD3-specific Fab is fused in one of its polypeptide chains to the N-terminus of a hole chain of a KIH scaffold, a first TAA-specific VHH is fused to the N-terminus of a knob chain, and a second TAA-specific VHH is fused to the C-terminus of the light chain of the CD3-specific Fab. Format Ac13 in panel B is a mirror image of 13cA depicted in panel B. Panels D and E represent control bispecific TCEs used for the examples below in formats 12A (panel D) and 1A (panel E), respectively. In both cases, the CD3-specific Fab is fused in one of its two polypeptide chains to the N-terminus of the knob chain of a KIH scaffold and a well described TAA specific scFV is fused to the N-terminus of the hole chain (formats 1A and 12A) or to both the N-terminus and C-terminus of the hole chain (format 12A). In some embodiments, the CD3-specific Fab can be a variant of clone of the 1F3 antibody, hu-1F3.

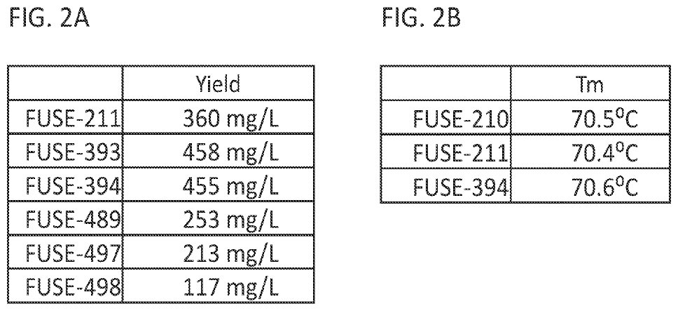

As shown in FIG. 2, tables are presented of the protein yields from the transient CHO cell transfections of exemplary TCEs (FIG. 2A) and their melting temperatures (FIG. 2B).

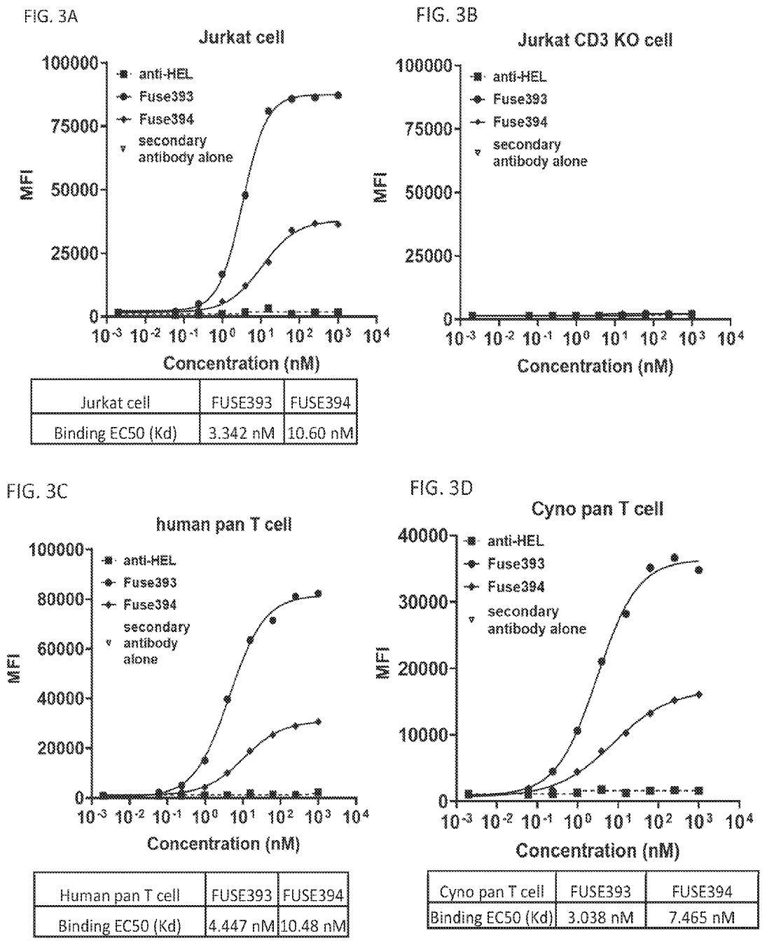

As shown in FIG. 3, the apparent binding affinity (EC₅₀) of TCE for human and non-human primate (NHP, Cynomolgus) CD3-expressing T cells were studied. We examined the magnitude by which ROR1-specific TCEs bound to cell surface expressed CD3 (selected TCEs, FUSE-393 and FUSE-394, are shown). Graphs in panels A to D are non-linear x-y plots illustrating the apparent binding affinities (EC50-binding) of FUSE-393 (format 11A incorporating the 5A1 ROR1-specific VHH) and FUSE-394 (format 13cA incorporating the 5A1 ROR1-specific VHH), for human CD3 on Jurkat T cells (panel A), human CD3 on CD3 knockout Jurkat T cells (panel B), NHP CD3 on cynomolgus pan-T cells (panel D), and human CD3 on T cells isolated from human PBMC (panel C). The CD3-specific Fab binding arm is presumed responsible for the observed binding. A Hen Egg Lysozyme (HEL)-specific antibody was used as the negative isotype control. Binding was measured via flow cytometry at various concentrations of test articles using a fluorescently labeled secondary antibody specific for the Fc domain of IgG1. The apparent affinity calculated for each test subject based on the data is shown under each graph. A less than 2-fold difference in the apparent binding affinity for human and for cynomolgus CD3 was observed for FUSE-393 and FUSE-394; and no appreciable non-specific binding was observed. Binding to mouse CD3 was not tested because the anti-CD3 clone, 1F3-3, incorporated into the TCE is a humanized variant of clone SP34, which was derived in mouse and is well described as not binding mouse CD3.

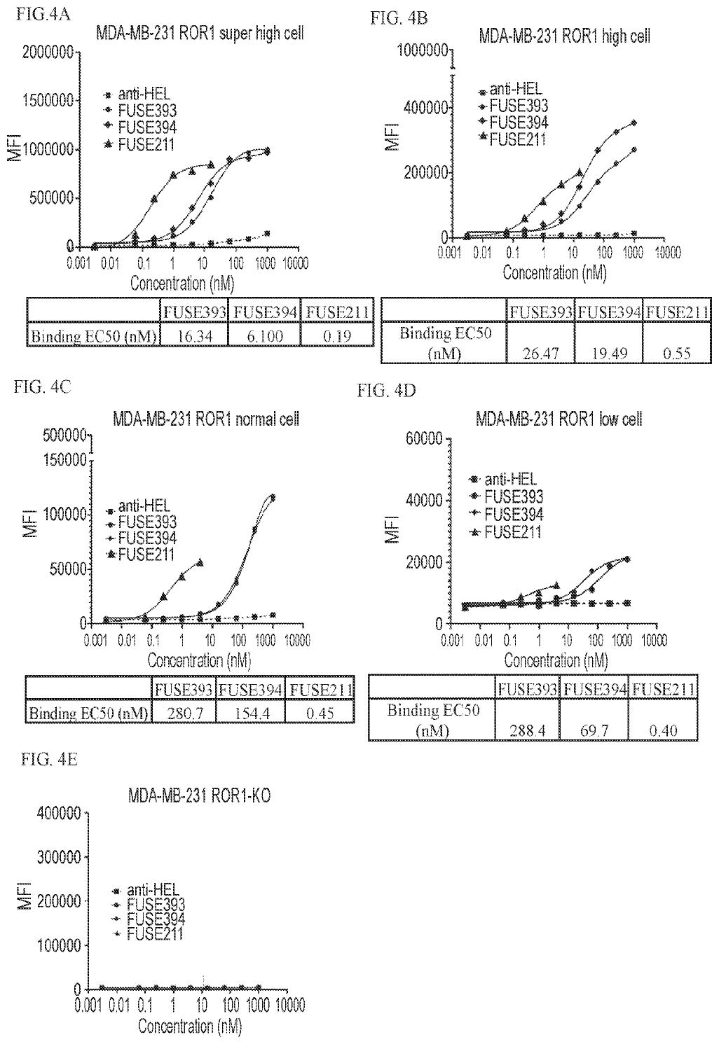

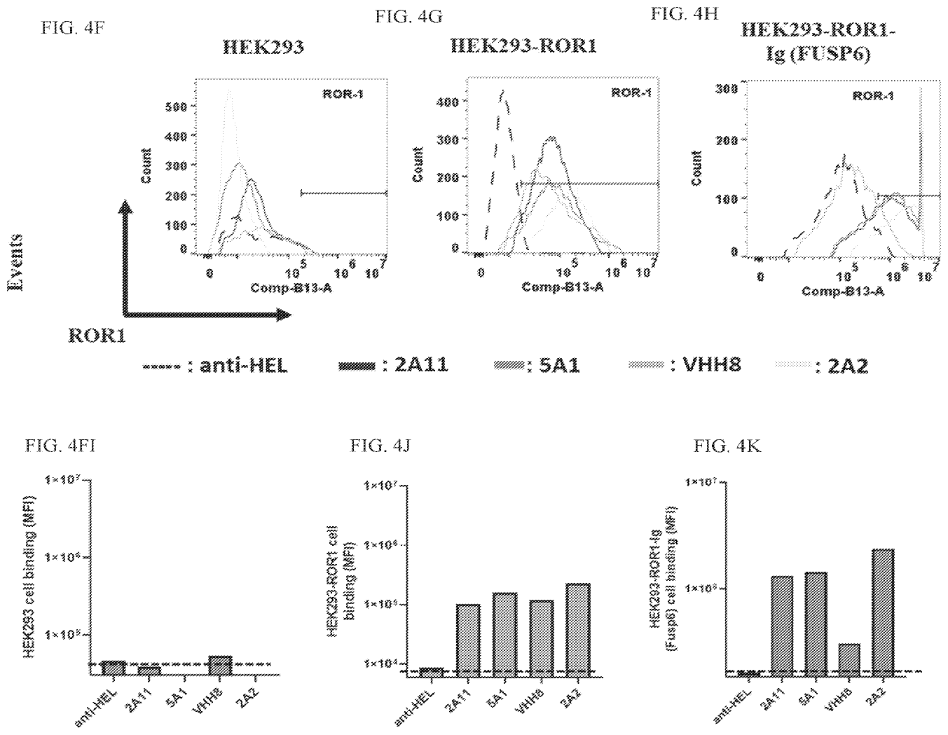

As shown in FIG. 4, the apparent binding affinity (EC₅₀) of TCEs for ROR1 at different cell surface densities of the target tumor cell was studied. Panels A-D are non-linear x-y plots illustrating the apparent binding affinities (EC50-binding) of FUSE-394 (i.e., the apparent affinity derived from the ROR1-specific VHH in format 13cA incorporating the ROR1-specific VHH having the 5A1 sequence) and FUSE-393 (format 11A incorporating the ROR1-specific VHH, 5A1) for human ROR1 expressed at different densities on the surface of engineered MDA-MB-231 cell variants. The cell surface densities are as follows: approximately 2,000,000 molecules per cell (panel A), approximately 500,000 molecules per cell (panel B), approximately 35,000 molecules per cell (panel C), and approximately 17,000 molecules per cell (panel D). In addition, a ROR1-knock out MDA-MB-231 cell line was also generated using CRISPR/Cas9, which served as a control for non-specific binding (panel E). A Hen Egg Lysozyme (HEL)-specific antibody was used as the negative isotype control. Binding was measured via flow cytometry at various concentrations of test articles using a fluorescently labeled secondary antibody specific for the Fc domain of IgG1. The apparent affinity calculated based on the data for each test article is shown under each graph. FUSE-394 was found to bind to ROR1 with a higher apparent binding affinity than FUSE-393, which ranged from approximately 4-fold to less than 2-fold greater depending on the cell surface density of ROR1. In general, the apparent affinity for both FUSE-393 and FUSE-394 correlated positively with cell surface density of ROR1. For FUSE-394, the apparent affinity ranged from approximately 100 nM at a low cell surface density to approximately 5 nM at a high cell surface density. For FUSE-393, the apparent affinity ranged from approximately 300 nM at a low cell surface density to approximately 15 nM at a high cell surface density. No appreciable non-specific binding was observed with either for the HEL-specific control with ROR1+ MDA-MB-231 cells or for the TCEs with MDA-MB-231 ROR1-knockout cells. No binding to mouse ROR1 was observed using mouse 4T1 cell engineered to stably express mouse ROR1 (data not shown). Qi et al. (see www.pubmed.ncbi.nlm.nih.gov/29844189/) previously reported that the ROR1 specific domain targeted by a CD3×ROR1 bispecific antibody or TCE can impact its potency. This includes the EC50 of pro-inflammatory cytokine release and EC50 of killing of ROR1 expressing tumor cells (but not necessarily maximum killing). The authors demonstrated that the closer the TCE's ROR1 binder's epitope is to the cell membrane, the more potent the TCE. The ROR1 ectodomain consists of three domains. The membrane proximal Kringle domain, the membrane distal Ig domain and the Frizzled domain in-between. Hence, the authors demonstrated that the Kringle domain specific binder, R11, when formatted as a CD3 bispecific antibody is more potent than a binder specific for the region between the Ig and Frizzled domain. Importantly, Gohil demonstrated that the same ROR1 binder (hF(1×1)) when formatted as a (scFV)2 or BiTE (short synapse) but not a symmetric bispecific antibody (larger synapse), mediated killing of ROR1+ tumor cells (see www.discovery.ucl.ac.uk/id/eprint/10042372/1/GOHIL_SH_DEPOSIT.pdf). This relationship between synaptic size and potency is thought to be the result of the magnitude of the signal transmitted via the TCR/CD3 complex, whereby the shorter the bridge or synapse between the T cell and targeted tumor, the stronger the activation signal. This is mediated, at least in part, by greater engagement of adhesion molecules exclusion of large inhibitory molecules such as CD45 from the smaller synapse. The strong activation signal, however, is associated with an important caveat. That is, that although the signal strength required to induce killing is lower than that required for cytokine release (see www.pnas.org/doi/10.1073/pnas.2334336100), very short synapses are unable to take advantage of this phenomenon to induce strong tumor cell killing with modulated or tuned cytokine release to reduce the risk of cytokine mediated toxicities such as Cytokine Release Syndrome (CRS) and neurotoxicity. While not wishing to be bound by any particular theory, we believe that although binding to the Kringle or Frizzled domain may maximize killing potency, it may be possible to dose higher into similar maximal killing by targeting the membrane distal Ig domain. FIGS. 4F-4H are overlayed flow cytometric histograms of binding of exemplary test articles to HEK-293 cells (negative control; FIG. 4F), HEK-293 cells transfected with full length human ROR1 (FIG. 4G) and HEK-293 cells transfected with the ROR1 Ig domain linked to the cell surface via the GPI linker from CD59 (FIG. 4H). Binding of 2A11 (black line), 5A1 (dark grey line) and VHH8 (medium grey line) were tested in TCE format 12A. The commercial ROR1 specific Ab reported to bind the ROR1-Ig domain 2A2 (light grey line), served as the positive control and anti-HEL (dotted black line) served as the negative control. To better visualize binding of each test article to the three cells lines, we plotted MFI on the y-axis for each test article for HEK-293 cells (negative control; FIG. 4I), HEK-293 cells transfected with full length human ROR1 (FIG. 4J) and HEK-293 cells transfected with the ROR1 Ig domain linked to the cell surface via the GPI linker from CD59 (FIG. 4K). We found that the ROR1 specific 2A11 and 5A1 VHHs bound to the ROR1 Ig domain but ROR1 specific VHH8 did not.

As shown in FIGS. 5A-5D, the binding affinities (KD) of TCEs for ROR1 were assessed. We examined the magnitude by which several TCEs and mutants thereof bind to recombinant ROR1. Kinetic binding graphs were generated via Bio-Layer Interferometry (BLI) using an Octet system. For each TCE, the biosensor tips were coated with FUSE proteins at 15 μg/ml and probed with either recombinant His tagged ROR1 at concentrations ranging from 100 pM to 10 μM. The parental TCEs tested were FUSE-211 (format 11A incorporating 2A11, a ROR1-specific VHH), FUSE-393 (format 11A incorporating 5A1, a ROR1-specific VHH), and FUSE-394 (format 13cA incorporating 5A1, a ROR1-specific VHH), shown in FIGS. 5A-5C, respectively. The binding affinity (KD), on rate (k-on) and off rate (k-dis), are summarized for ROR1 in FIG. 5D.

We further examined the impact of mutating the cysteine residues located in CDR1 and CDR3 of 2A11 VHH and 5A1 VHH. As such the parental 2A11 VHH and 5A1 VHH were expressed as VHH-human-Fc fusion proteins, FUSE-112 and FUSE-179, respectively. For the mutant 2A11 (FUSE-453) and mutant 5A1 (FUSE-454) the cysteine residues in both CDR1 and CDR3 were mutated to valines such that 2A11-CDR1 was changed from GSTYSANC (SEQ ID NO:1; FUSE-112) to GSTYSANV (SEQ ID NO:204; FUSE-453), 2A11-CDR3 was changed from AYGGSRCVYNY (SEQ ID NO:3; FUSE-112) to AYGGSRVVYNY (SEQ ID NO:205; FUSE-453), 5A1-CDR1 was changed from GYTNRLKC (SEQ ID NO: 4; FUSE-179) to GYTNRLKV (SEQ ID NO:206; FUSE-454) and 5A1-CDR3 was changed from DVRPDGTTCHYN (SEQ ID NO:6; FUSE-179) to DVRPDGTTVHYN (SEQ ID NO:207; FUSE-454). The binding affinity (KD), on rate (k-on) and off rate (k-dis) are summarized for ROR1 in FIGS. 5G and 5J. The binding curves for FUSE-112, FUSE-453, FUSE-179 and FUSE-454 are shown in FIGS. 5E, 5F, 5H, and 5I, respectively where FIG. 5E versus FIG. 5F compares 2A11 parental to the 2A11 mutant and FIG. 5H versus 5I compares 5A1 parental to the 5A1 mutant. Both FUSE-112 and FUSE-179 bound to ROR1 with KDs of about 4 nM, whereas binding of FUSE-453 and FUSE-454 to ROR1 was so weak that a reliable KD could not be calculated or was below the limit of calculation (BLC). We estimate the affinity of FUSE-453 and FUSE-454 for ROR1 is >100 fold lower than FUSE-112 and FUSE-454, respectively. As such, the cysteine residues within CDR1 and CDR3 of 2A11 VHH and 5A1 VHH are critical to the ROR1 specific paratopes.

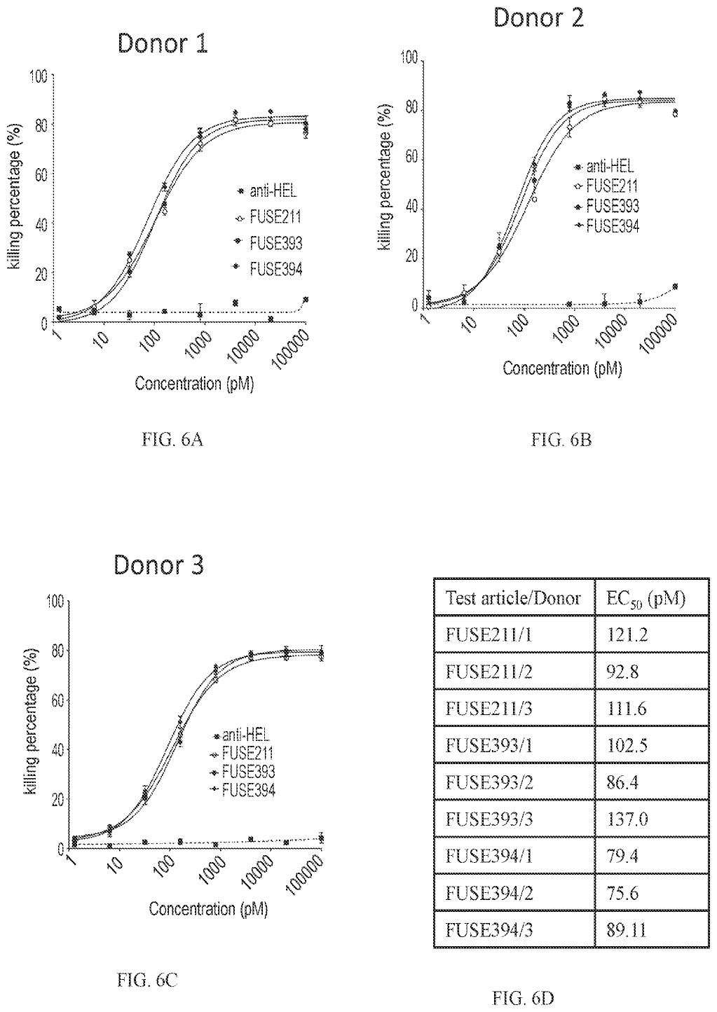

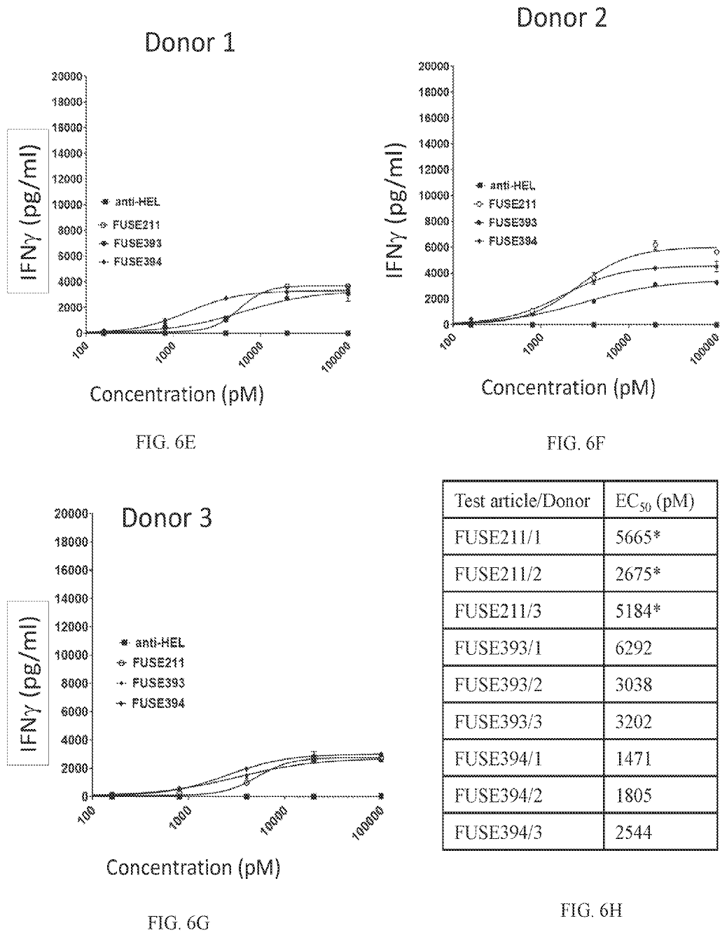

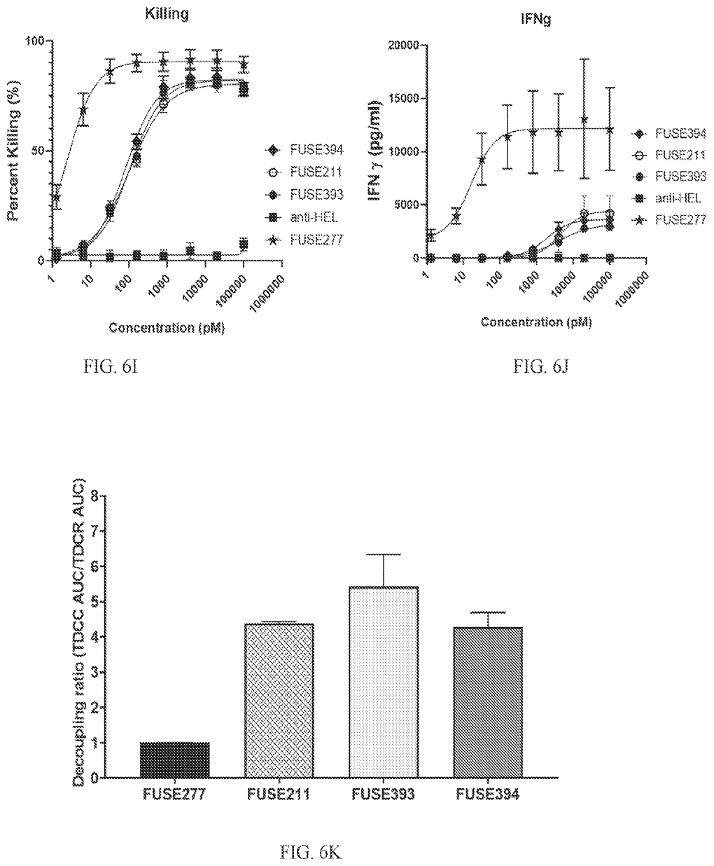

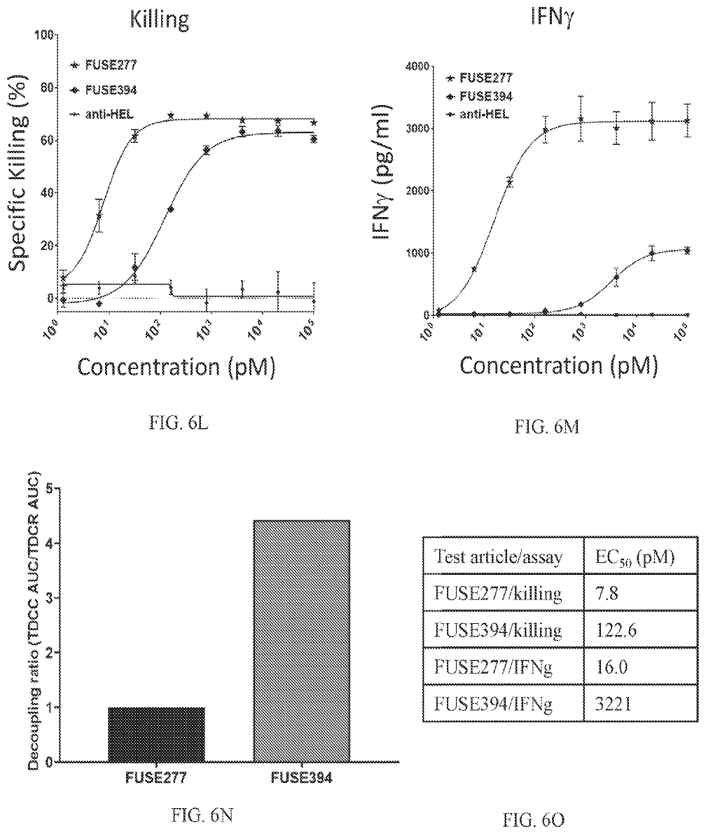

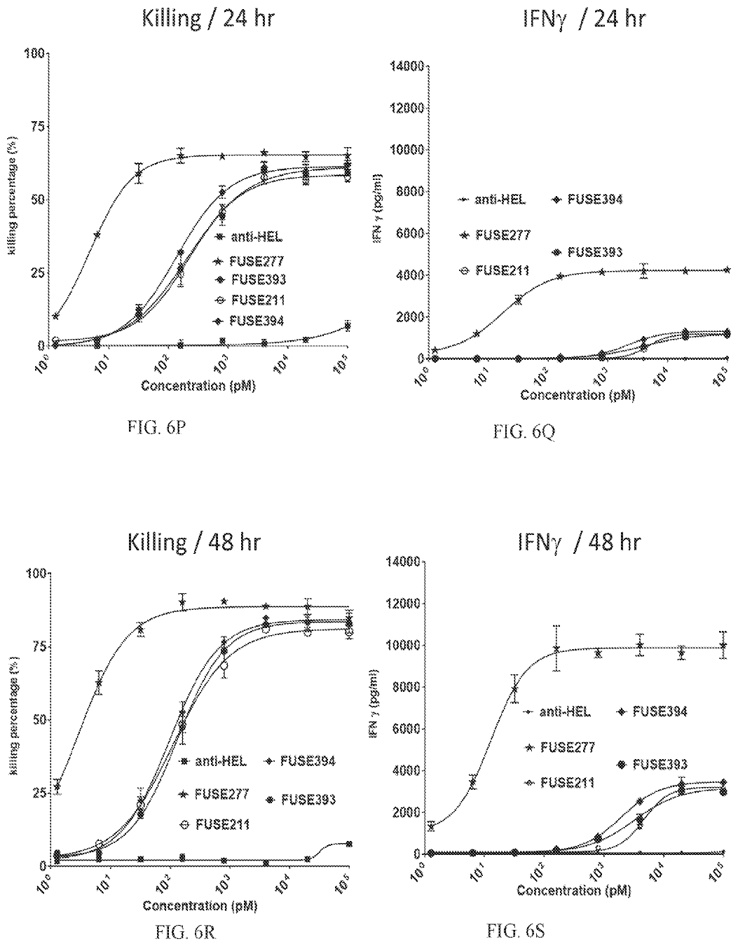

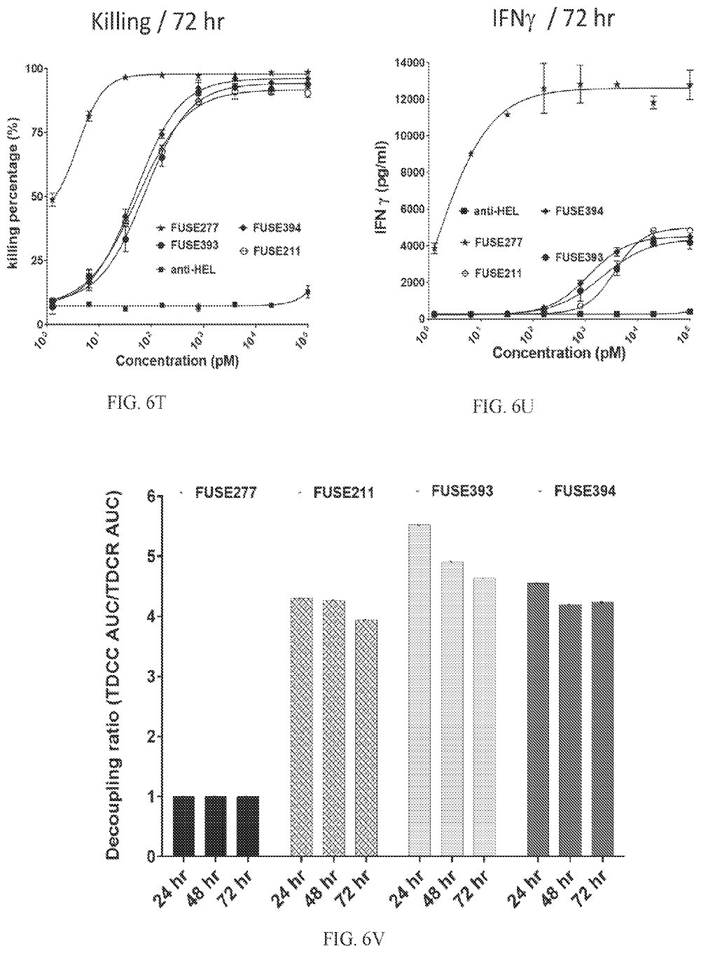

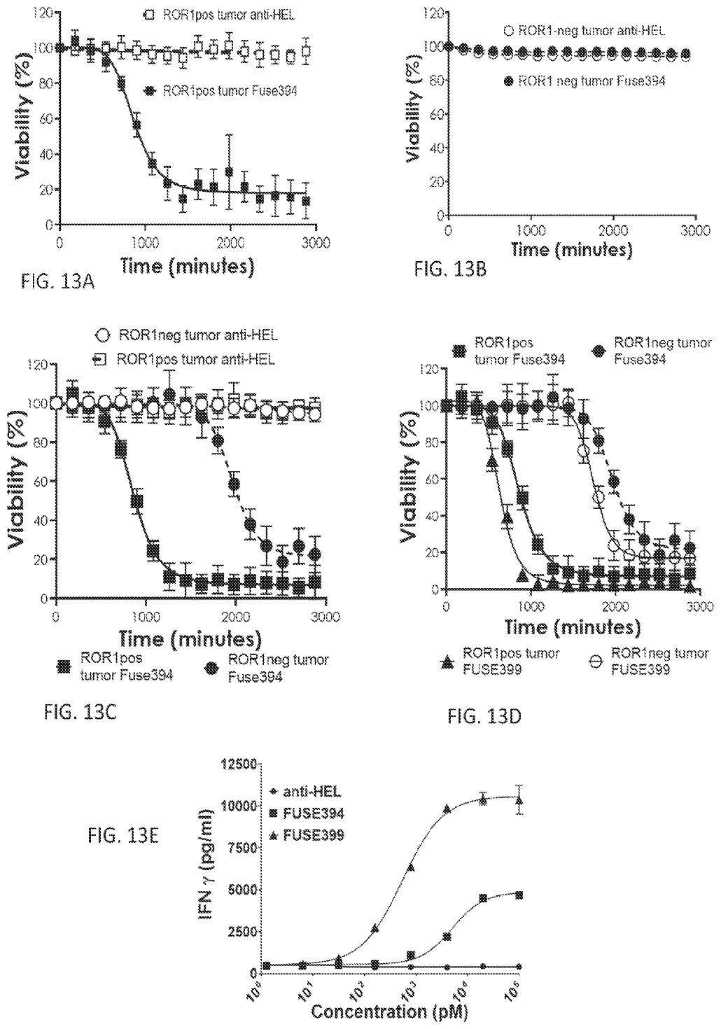

As shown in FIGS. 6A-6H, non-linear x-y plots illustrate the induction ROR1-dependent, human peripheral blood mononuclear cells (PBMC) release of IFNγ and cytotoxic activity (i.e. killing of ROR1+ MDA-MB-231 tumor cells) induced by fusion proteins FUSE-211 (format 11A incorporating 2A11, a ROR1-specific VHH), FUSE-394 (format 13cA incorporating 5A1, a ROR1-specific VHH), and FUSE-393 (format 11A incorporating 5A1, a ROR1-specific VHH). In FIGS. 6I and 6J, IFNγ release and cytotoxicity of the aforementioned TCE are compared to a highly potent TCE, FUSE-277 (format 12A incorporating R11, a ROR1 specific scFV) and the ratio of cytotoxicity to cytokine (IFNγ) release measured as the “decoupling ratio”. We examined the capacity of the aforementioned TCEs to induce killing of ROR1⁺ target cells, MDA-MB-231. Results from three exemplary PBMC donors are shown in FIGS. 6A, 6B and 6C. FIG. 6D shows the potency of killing defined as the EC₅₀-killing. We further examined the capacity of several TCEs to induce PBMC mediated IFNγ release in the presence of ROR1⁺ target cells, MDA-MB-231. Results from three exemplary PBMC donors are shown in FIGS. 6E, 6F and 6G. FIG. 6H shows the potency of cytokine release defined as the EC₅₀-IFNγ. For both readouts, a Hen Egg Lysozyme (HEL)-specific antibody was used as the negative isotype control. To measure cytotoxic potency and IFNγ release, freshly isolated human PBMCs were co-cultured for 48 hours with human ROR1⁺ MDA-MB-231 tumor cells engineered to stably express firefly luciferase and GFP. Supernatant was collected and IFNγ measured via ELISA. Luminescent activity induced by firefly luciferase in the presence of its substrate, D-Luciferin, was used as a measure of ROR1⁺ MDA-MB-231 cell viability. We observed ROR1-dependent cytotoxic potency in the pM range for FUSE-211, FUSE-393 and FUSE-394 across three sets of PBMCs isolated from different healthy control donor subjects. FUSE-394 was the most potent followed by FUSE-393 and FUSE-211, the latter two of which were not appreciably different. We also observed ROR1-dependent IFNγ release. Again, FUSE-394 was the most potent followed by FUSE-393 and FUSE-211. The values for IFNγ release for FUSE-211 at 100 nM were calculated based on the curve. For all the aforementioned TCEs, IFNγ release was weaker, both in EC50 and max release, than what would be expected from a traditional TCE such as a Bi-specific T-cell engager (BiTE) or Dual-Affinity Re-Targeting (DART) molecule. Both BiTES and DARTs contain small linkers between their CD3 binder and target antigen binder. As such, the synapse or bridge formed between T cells and tumor cells is generally short unless in the very rare circumstance that the target antigen is very large and the epitope is very membrane distal. This results in a stronger activation signal via the TCR/CD3 complex, which has been hypothesized to occur because more adhesion molecules are engaged, and CD45, a key inhibitory phosphatase, is excluded from the T cell/tumor synapse. In contrast, FUSE-211, FUSE-393 and FUSE-394 incorporate ROR1 specific VHH that bind the membrane distal ROR1 Ig domain and geometries selected for high killing efficacy (e.g., maximum killing) but reduced pro-inflammatory cytokine release (IFNγ used as example since early neutralization of IFNγ can reduce symptoms of CRS (see e.g., www.pubmed.nebi.nlm.nih.gov/8370401/)) to decrease the risk of toxicity. Of note, the expression of IFNγ following T cell activation correlates with that of IL-2, TNFα and IL-6 is well accepted. TNFα and IL-6 are also tightly linked to CRS. IL-2 has also been linked toxicity and could have implications to Treg activity, which constitutively express the high affinity IL-2 receptor, CD25. We therefore compared ROR1 dependent PBMC mediated IFNγ release and cytotoxicity of MDA-MB-231 associated with FUSE-211, FUSE-393 and FUSE-394 with a TCE that bridges CD3 to the membrane proximal Kringle domain of ROR1. The latter TCE, FUSE-277, binds to ROR1 via the well described ROR1 specific clone, R11. The non-linear x-y plot illustrating the killing of MDA-MB-231 by the average of three donor PBMCs is shown in FIG. 6I. The non-linear x-y plot illustrating MDA-MB-231 ROR1 dependent PBMC mediated IFNγ release (mean of three donors) is shown in FIG. 6J. For PBMC mediated cytotoxicity (FIG. 6I), we observed that the potency (EC50_{KILLING}) of FUSE-277 was approximately 2 pM, or 40-50 fold greater than FUSE-211, FUSE-393 and FUSE-394. Importantly, killing efficacy for FUSE-277 was only 6-10% greater than those observed for FUSE-211, FUSE-393 and FUSE-394 indicating that the latter TCE could be dosed to a nearly equivalent efficacy. Further, the extremely potent nature of FUSE-277 may result in difficulty identifying a sufficiently low dose in the clinic to prevent tumor lysis syndrome when tumor burden is high. For PBMC mediated IFNγ release (FIG. 6J), we observed that the potency (EC50_{IFNγ}) of FUSE-277 was approximately 5 pM, on average about 1000 fold greater than FUSE-211, FUSE-393 and FUSE-394. Perhaps more critical, the maximum release of IFNγ induced by FUSE-277 was on average approximately 3 fold greater than those observed for FUSE-211, FUSE-393 and FUSE-394. This data suggests one could dose FUSE-211, FUSE-393 and FUSE-394 into similar killing efficacy as FUSE-277 with a reduced risk of CRS. Of interest, although FUSE-211, FUSE-393 and FUSE-394 were observed to reach maximum killing efficacy at about 1 nM, IFNγ levels slowly increased from close to baseline at 1 nM to between 2.5-5 ng/ml at 20 nM. This gap represents about a 20,000 pM range, or “in-vitro therapeutic window”, in which the dose of FUSE-211, FUSE-393 and FUSE-394 can be titrated to achieve a dose that incorporates both maximizing killing and a safe/effective dose of pro-inflammatory cytokine, important for inflaming the tumor and facilitating bystander killing (see FIG. 13). Thus, the data suggests that it may be possible to dose FUSE-211, FUSE-393 and FUSE-394 to maximize efficacy but titrate the cytokine release to achieve tumor inflammation with a reduced risk of CRS. In contrast, no such “in-vitro therapeutic window” was observed with FUSE-277. To further quantitate the degree by which each TCE decoupled cytotoxicity from cytokine (IFNγ) release, we measured killing magnitude as the area under the curve (AUC) for cytotoxicity from FIG. 6I and IFNγ release magnitude as the area under the curve (AUC) for IFNγ from FIG. 6J. A “decoupling ratio”, defined as the magnitude of killing divided by the magnitude of IFNγ release (normalized to FUSE-277, for which an “in-vitro therapeutic window” was not observed) was then assigned to each TCE (FIG. 6K). The decoupling ratios for FUSE-277, FUSE-211, FUSE-393 and FUSE-394 were 1 (no decoupling), 4.4, 5.4 and 4.3, respectively. We wished to confirm that TCE induced tumor cell cytotoxicity and IFNγ release observed using PBMC as effectors was mediated by T cells. As such, we performed the same assay shown in FIGS. 61 and 6J but substituted purified Pan-T cells for PBMC. FIGS. 6L and 6M are non-linear x-y plots of specific killing or IFNγ release versus test article concentration, respectively. Both assays were run for 24 hours and unlike PBMC that were freshly isolated, Pan-T cells were cryo-preserved. In both cases, FUSE-394 was compared to the non-decoupling control TCE, FUSE-277. Anti-HEL was used as the negative control, for which non-specific activity was absent. A summary of potencies (EC50s) for ROR1 dependent cytotoxicity and IFNγ release is shown in FIG. 6O. Similar to that observed with PBMC, FUSE-277 was about 15 fold more potent (EC50_{KILLING}) than FUSE-394 but both reached similar efficacy readouts (maximum killing) and the potency of FUSE-394 remained in the low triple digit pM range (FIG. 6L). We consider such a potency more conducive for managing tumor lysis syndrome. In contract to ROR1 dependent tumor cell cytotoxicity, we observed about a 200 fold reduced potency related to T cell mediated IFNγ release induced by FUSE-394 compared to FUSE-277. Importantly, maximum IFNγ release associated with FUSE-394 plateaued at approximately 30% that of FUSE-277 (FIG. 6M) and the “in-vitro therapeutic window” calculated for FUSE-394 was about 20,000 pM. This resulted in a decoupling ratio for FUSE-394 that was between 4-5× greater than that for FUSE-277 (FIG. 6N). Although, the comparison was between PBMC and Pan-T, it is interesting to note that reducing the assay time period from 48 hours (PBMC) to 24 hours (T cells) led to only about a 25% drop maximum killing but 4-5 fold drop in maximum IFNγ release. To obtain further insight into the kinetics of cytotoxicity and cytokine release induced by (a) a non-decoupling TCE (FUSE-277) and (b) exemplary TCEs that decouple cytotoxicity from cytokine release (FUSE-211, FUSE-393 and FUSE-394), we performed target dependent cytotoxicity (TDCC) assays and target dependent cytokine release (TDCR) assays for 24 hours, 48 hours and 72 hours. MDA-MB-231 was used as the ROR1 expressing tumor cell, PBMC as effector cells and IFNγ as the exemplary proinflammatory cytokine. As seen in FIGS. 6P, 6R and 6T, FUSE-277 was about 50-100 fold more potent (EC50-killing) than FUSE-211, FUSE-393 and FUSE-394 but the maximum killing (efficacy) induced by the decoupled TCEs was able to reach values not appreciably different from FUSE-277. Killing potency induced by FUSE-211, FUSE-393 and FUSE-394 was within 2-3 fold of one another. As would be expected, maximum killing mediated by all test articles increased with time with values of approximately 70%, 85% and 95% at 24, 48 and 72 hours, respectively. Unlike ROR1 dependent killing, maximum cytokine release induced by FUSE-211, FUSE-393 and FUSE-394 was consistently about 3 fold less than FUSE-277 at each timepoint (FIGS. 6Q, 6S and 6U). Potency (EC50-IFNγ) associated with the three decoupled TCEs was as much as 10,000 fold weaker than FUSE-277, the “in-vitro therapeutic window” ranged from 10,000 to 20,000 pM and decoupling ratios (FIG. 6V) ranged from values of about 4 (FUSE-211 and 394) to between 4.5-5 (FUSE-393). Although there was no significant difference in decoupling ratios at different timepoints, a trend developed such that the decoupling ratio at 24 hours (the key safety timepoint) appeared to be the highest. As observed with cytotoxicity, potency associated with cytokine release varied only between 2-3 fold between FUSE-211, FUSE-393 and FUSE-394. Most importantly, the absolute values for maximum IFNγ release at 24 hours for the three decoupled TCEs did not exceed 1.5 ng/ml and could be titrated between about 200 μg/ml and 1.5 ng/ml while maintaining maximum killing. Matthys and colleagues (see www.pubmed.ncbi.nlm.nih.gov/8370401/) reported that early but not late neutralization of IFNγ was capable of reducing the symptoms of CRS. In fact, rebound levels of IFNγ at later timepoints that exceeded treatment with saline did not contribute to such symptoms. This suggests that initiating treatment of a patient with a dose of FUSE-211, FUSE-393 or FUSE-394 that induces maximum killing but minimal IFNγ should reduce the risk of CRS and the need for expensive interventions such neutralization of IFNγ, TNFα and/or IL-6 thus vastly improving patient compliance. Importantly, at later timepoints the dose of FUSE-211, FUSE-393 or FUSE-394 can be safely increased such that IFNγ levels accumulate to the point that secondary mechanisms of tumor cell killing that involve tumor inflammation and bystander killing mediated by death receptors are supported. The latter is explored in FIG. 13.

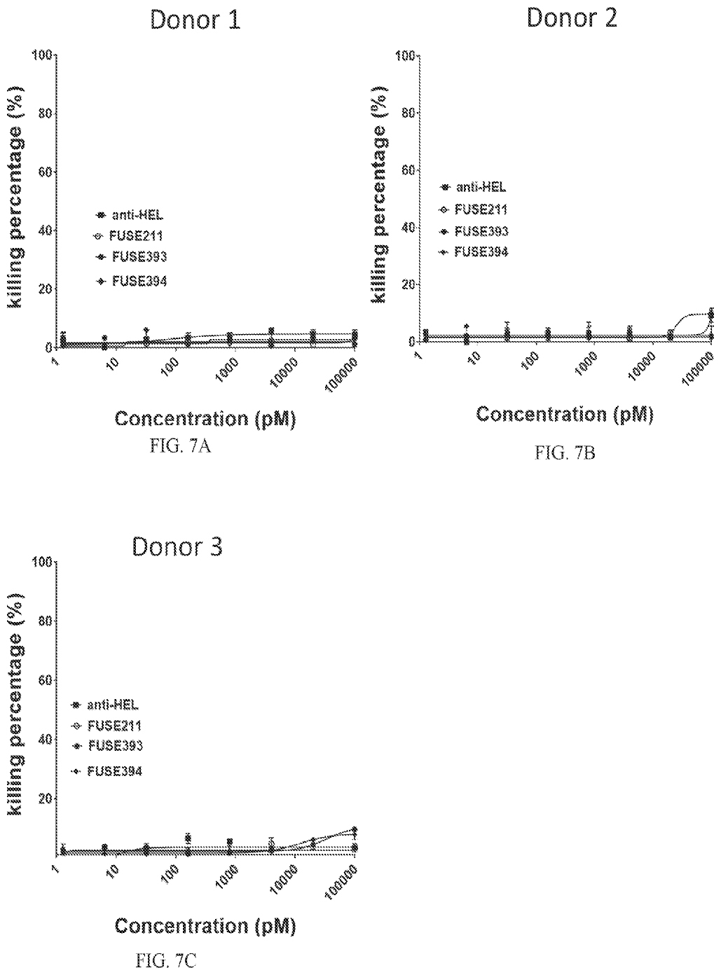

As shown in FIGS. 7A-7C, none-linear x-y plots illustrate the induction of human PBMC-mediated, ROR1-independent cytotoxic activity by FUSE-211 (format 11A incorporating the ROR1 specific VHH, 2A11), FUSE-394 (format 13cA incorporating the ROR1 specific VHH, 5A1), and FUSE-393 (format 11A incorporating the ROR1 specific VHH, 5A1). We examined the ROR1 specificity of several TCEs with reference to their induction of killing of target cells that do not express ROR1, i.e., T47-D cell line. Results from three exemplary PBMC donors are shown in panels A-C, respectively. A Hen Egg Lysozyme (HEL)-specific antibody was used as the negative isotype control. To measure non-specific cytotoxic potency, freshly isolated human PBMC were co-cultured for 48 hours with human ROR1-negative T-47D tumor cells engineered to stably express firefly luciferase and GFP. Luminescent activity induced by firefly luciferase in the presence of its substrate, D-Luciferin, was used as a measure of ROR1⁺ target viability. We observed very little to no appreciable cytotoxicity towards T-47D target cells. At the highest and supraphysiological concentration of 100 nM, non-specific cytotoxicity was observed towards <10% of the T-47D cells in donors 2 and 3 but not in donor 1. Further, such non-specific killing was highly variable between test articles including the anti-HEL isotype control, which was indicative of irrelevant artifacts likely resulting from the IgG1 backbone or perhaps NK cell-mediated antibody dependent cellular cytotoxicity.

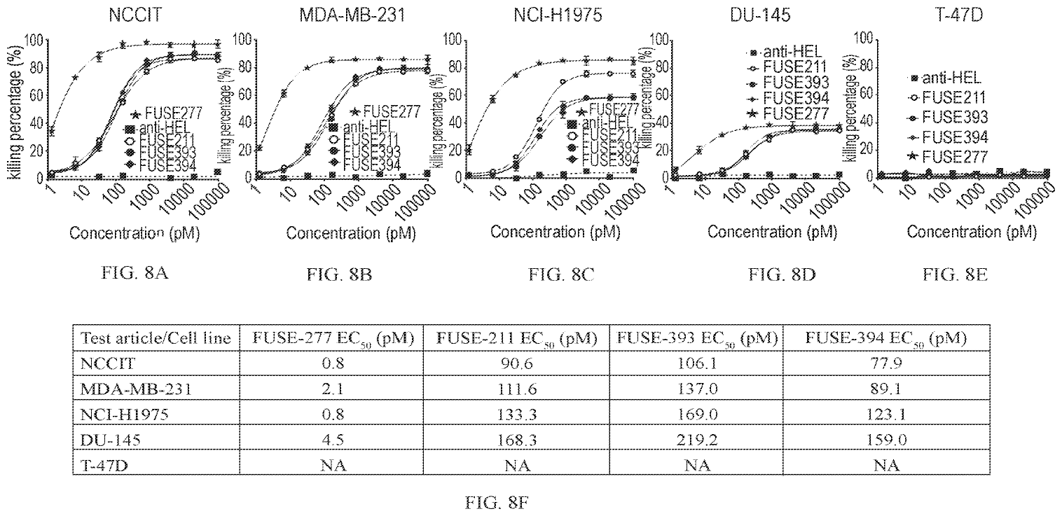

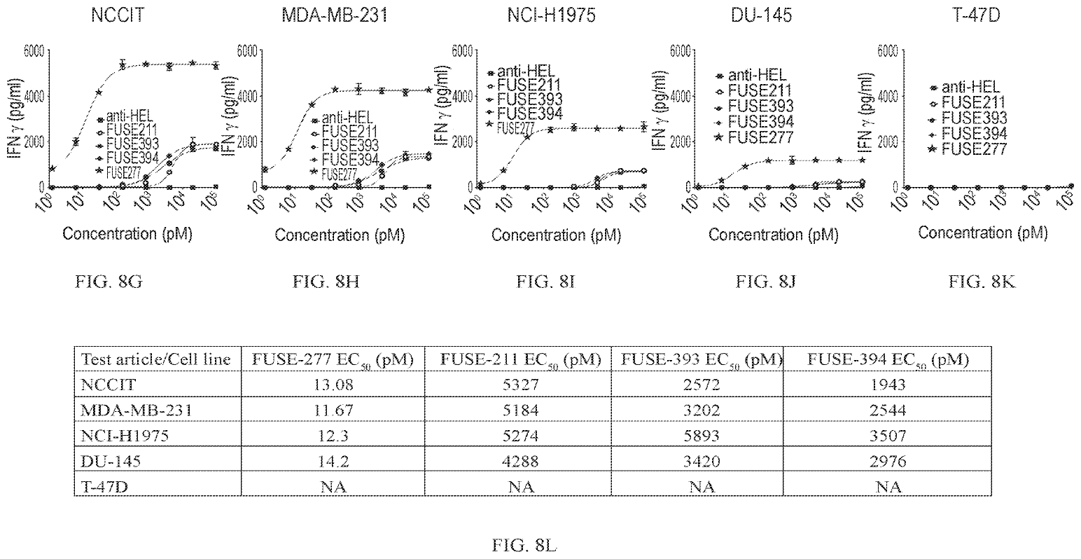

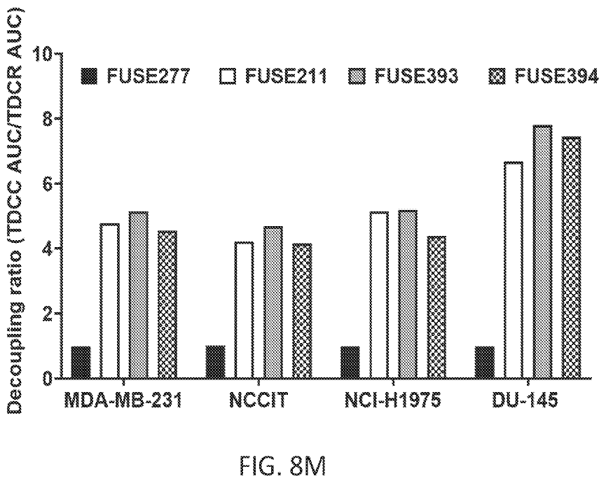

As shown in FIGS. 8A-8E, and 8G-8K, we provide non-linear x-y plots of dose contingent TCE induced human PBMC-mediated ROR1-dependent (a) cytotoxic activity, and (b) IFNγ release, respectively, when admixed with one of a series of tumor cells. The TCEs tested were the non-decoupling control TCE, FUSE-277, and the following TCEs that decouple cytotoxicity from cytokine release: FUSE-211, FUSE-393 and FUSE-394. From highest to lowest cell surface density of ROR1, the cell lines tested were NCCIT (FIGS. 8A, 8G), MDA-MB-231 (FIGS. 8B, 8H), NCI-H1975 (FIGS. 8C, 8I), DU-145 (FIGS. 8D, 8J) and ROR1-negative T-47D (FIGS. 8E and 8K). A Hen Egg Lysozyme (HEL)-specific antibody was used as the negative isotype control. We observed PBMC mediated killing of all ROR1⁺ cell lines and very little to no appreciable non-specific cytotoxicity towards the ROR1-negative target cells, i.e., T-47D cells. For each TCE, the potency of killing (EC₅₀-killing) was observed to weaken as the density of cell surface ROR1 decreased. As expected, the potency of FUSE-277 ranged between 70-100 fold greater than the 3 decoupled TCEs, which demonstrated very similar killing potencies across ROR1 cell surface density whereby the fold difference between any two was less than 2 fold. Further, we observed that maximum killing trended positively with ROR1 cell surface density on the target cells and importantly, with the exception of killing of NCI-1975, FUSE-211, FUSE-393 and FUSE-394 reached maximum killing values that were not appreciably different from FUSE-277. A summary table of EC₅₀-killing for each test article is shown in FIG. 8F. As previously observed when MDA-MB-231 tumor cells were employed as ROR1+ targets, we observed that compared to FUSE-277, for which no “in-vitro therapeutic window” was observed, the three decoupled TCEs induced PBMC mediated cytokine release that was associated with (a) 1000 to >10,000 fold reduced potency (EC₅₀-IFNγ), (b) maximum IFNγ values that were at least 3 fold lower and (c) “in-vitro therapeutic windows” of approximately 20,000 pM, (d) decoupling ratios (FIG. 8M) between 4-5 (NCCIT, MDA-MB-231 and NCI-H1975) and 6-7 (DU-145), and (e) potencies and maximum levels within 2 fold of each other across the entire cell line array. A summary table of EC₅₀-IFNγ for each test article is shown in FIG. 8L that includes the lack of non-specific activity observed with T-47D (FIG. 8K). The values for IFNγ release for FUSE-211 at 100 nM were calculated based on the curve. Like ROR1 dependent cytotoxicity, ROR1 dependent cytokine release trended positively with ROR1 cell surface density, which was measured as between 5,000-7,000 molecules per cell on DU-145 (prostate cancer cell line). This level is consistent with that thought to be expressed on most normal tissues that express ROR1, including gut epithelial cells and pancreatic islet cells (see www.pubmed.ncbi.nlm.nih.gov/27852699/) Importantly, unlike FUSE-277 that induced appreciable PBMC mediated IFNγ release, that induced by the three decoupled TCEs was negligible (≤250 pg/ml). This suggests that FUSE-211, FUSE-393 and FUSE-394 are able to “distinguish” between “normal” and malignant levels of ROR1 such that these TCE do not induce appreciable cytokine production when ROR1 density reaches levels at or below about 7,000 cell surface molecules per cell.

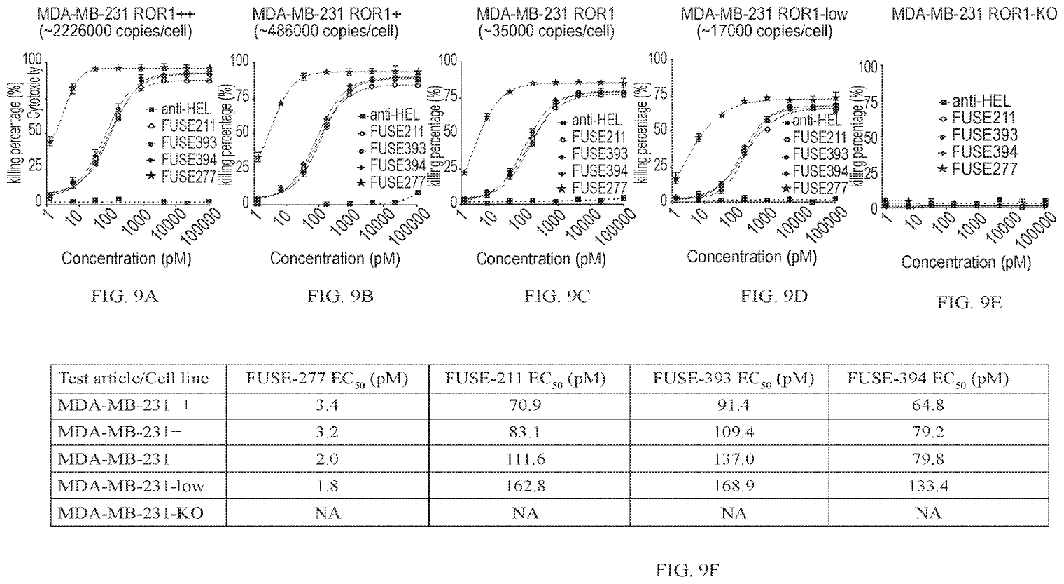

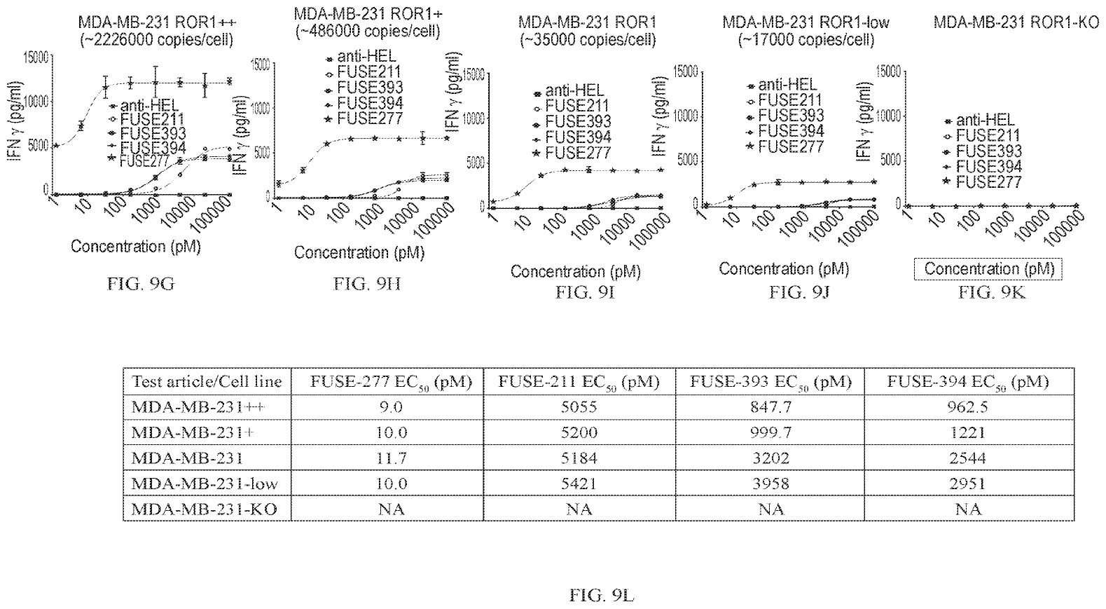

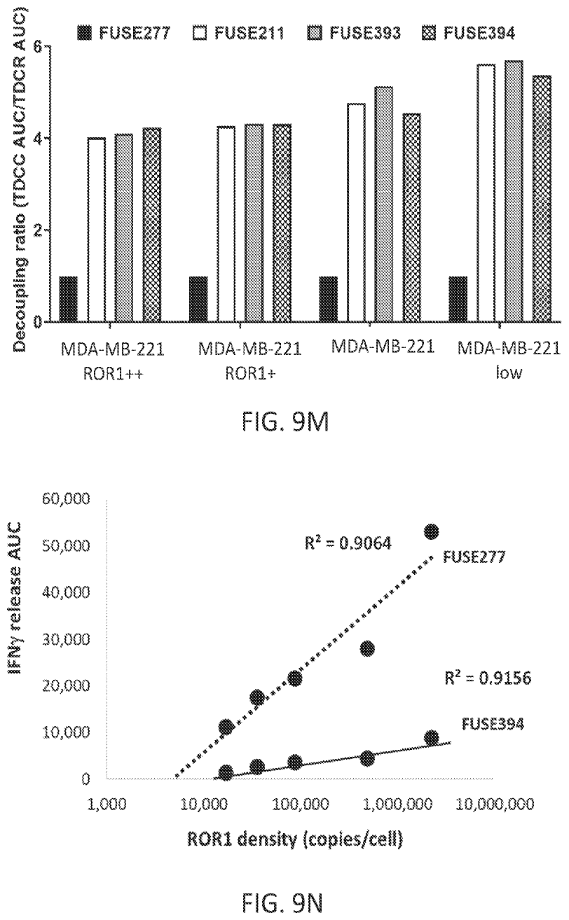

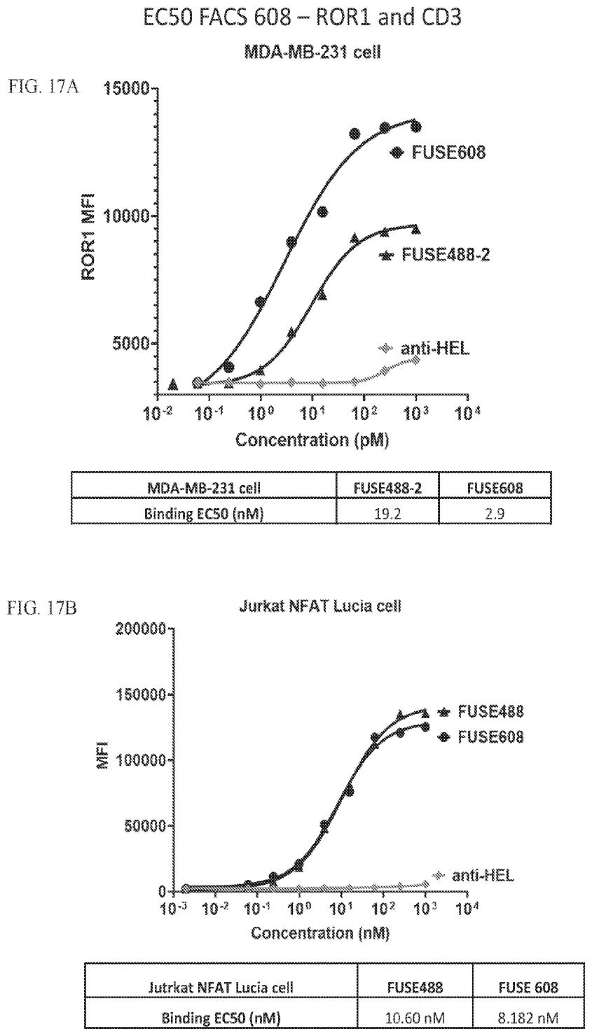

As shown in FIGS. 9A-9E, and 9G-9K, we provide non-linear x-y plots of dose contingent TCE induced human PBMC-mediated ROR1-dependent (a) cytotoxic activity, and (b) IFNγ release, respectively, when admixed with one of a series of tumor cells as described below. The TCEs tested were the non-decoupling control TCE, FUSE-277, and the following TCEs that decouple cytotoxicity from cytokine release: FUSE-211, FUSE-393 and FUSE-394. The target cells consisted of MDA-MB-231 cells engineered to stably express different cell surface densities of ROR1. The cell surface densities were as follows: ˜2,000,000 molecules per cell (MDA++; FIGS. 9A and 9G), ˜500,000 molecules per cell (MDA+; FIGS. 9B and 9H), ˜35,000 molecules per cell (MDA; FIGS. 9C and 9I), and 17,000 molecules per cell (MDA-low; FIGS. 9D and 9J). In addition, a ROR1-knock out, MDA-MB-231 cell line (MDA-KO; FIGS. 9F and 9K) was generated using CRISPR/Cas9, which served as a control and reflected ROR1-independent, non-specific cytotoxicity. A Hen Egg Lysozyme (HEL)-specific antibody was used as the negative isotype control. To measure cytotoxic potency and IFNγ release, freshly isolated human PBMCs were co-cultured for 48 hours with each of the MDA-MB-231 ROR1 density variants. We observed very little to no appreciable non-specific IFNγ release from PBMC nor cytotoxicity of the MDA-MB-231 ROR1-knockout target cells. For each TCE, the potency of killing (EC5-killing) and IFNγ release (EC5-IFNγ) were observed to weaken as the density of cell surface ROR1 decreased. As anticipated, the potency of FUSE-277 ranged between 70-100 fold greater than the 3 decoupled TCEs, which demonstrated very similar killing potencies across ROR1 cell surface density whereby the fold difference between any two was less than 2 fold. Further, we observed that maximum killing trended positively with ROR1 cell surface density on the target cells and importantly, FUSE-211, FUSE-393 and FUSE-394 reached maximum killing values that were not appreciably different from FUSE-277. A summary table of EC50-killing for each test article is shown in FIG. 9F. No “in-vitro therapeutic window” was observed with FUSE-277. In contrast, the observed “in-vitro therapeutic window” for the three decoupled TCEs was consistently about 20,000 pM. Further, PBMC mediated cytokine release induced by FUSE-211. FUSE-393 and FUSE-393 was associated with (a) 1000 to >10,000 fold reduced potency (EC₅₀-IFNγ), (b) maximum IFNγ values that were at least 3 fold lower and (c) decoupling ratios (FIG. 9M) between 4-5.5. Of note, a trend appeared such that decoupling ratios increased in value as the density of ROR1 on the cell surface of the MDA-MB-231 variants decreased. A summary table of EC₅₀-IFNγ for each test article is shown in FIG. 9L. The values for IFNγ release for FUSE-211 at 100 nM were calculated based on the curve. We calculated the area under the curve for IFNγ release termed the magnitude of IFNγ release and performed a regression analysis of such versus ROR1 cell surface density (FIG. 9N). Each dot in the regression analysis represents a different variant of MDA-MB-231 whereby the density of ROR1 increases from left to right. FUSE-277, the non-decoupling TCE control, was associated with an appreciably steeper slope than FUSE-394, the exemplary non-decoupling TCE shown. FUSE-211 and FUSE-393 were associated with similar results (data not shown). Importantly, FUSE-277 but not FUSE-394 was observed to be associated with IFNγ release at ROR1 densities below 10,000 cell surface molecules, discussed above as the likely demarcation between the expression of ROR1 on most normal versus malignant cells. Taken together with the data from FIG. F

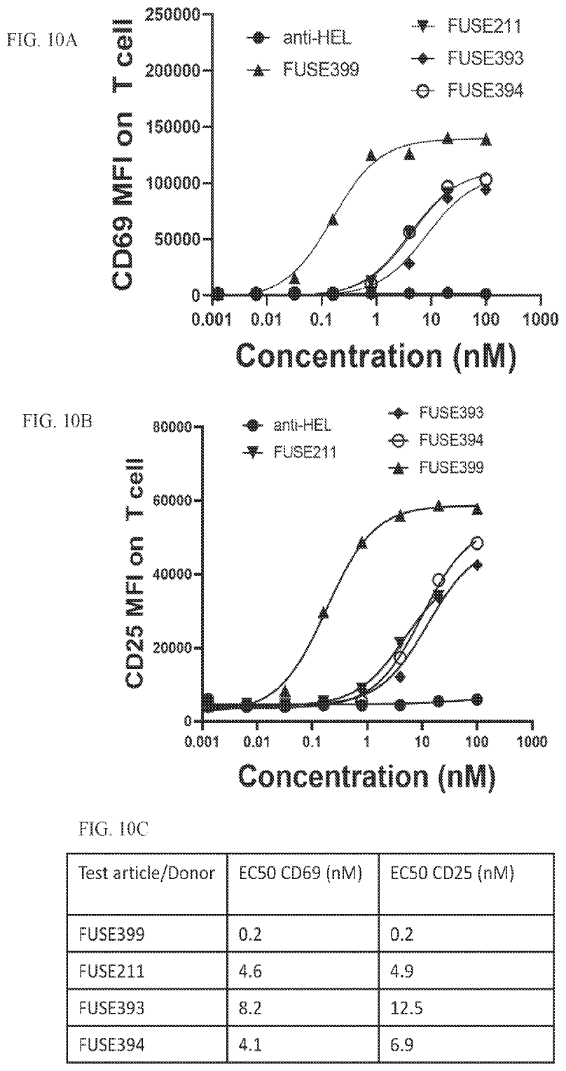

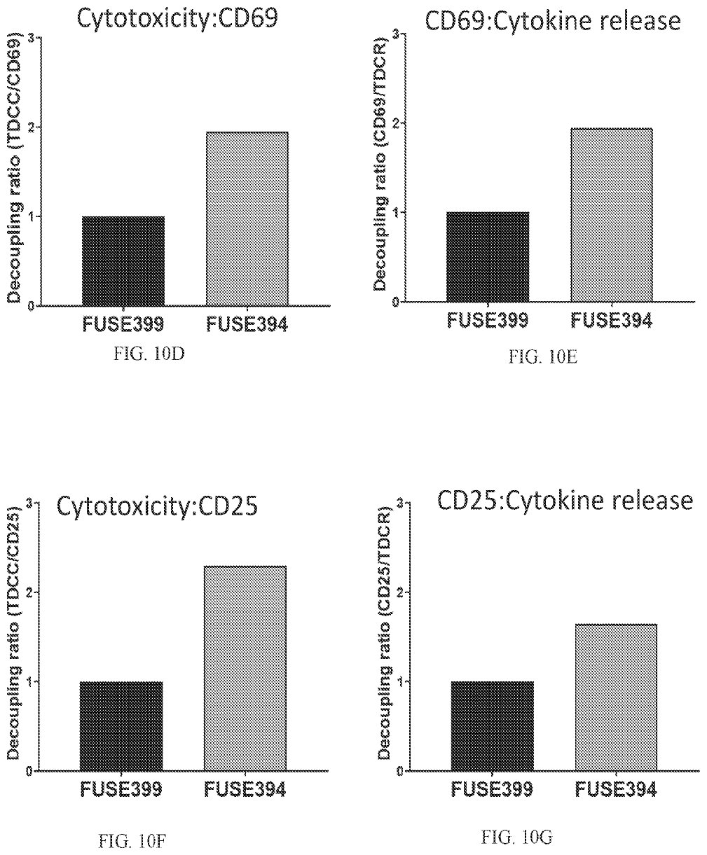

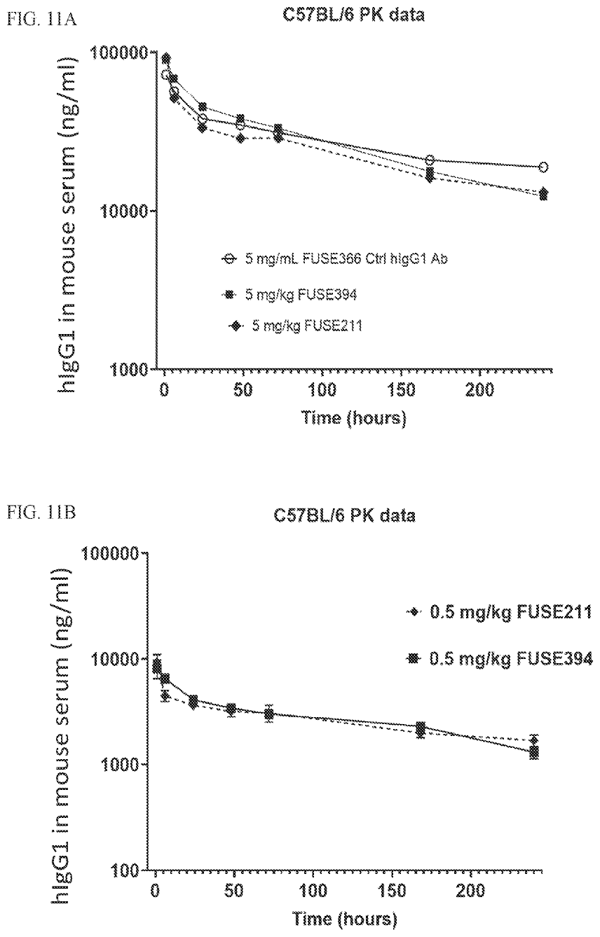

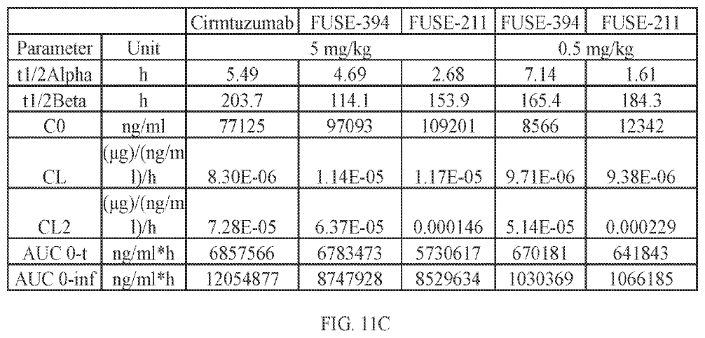

As shown in FIGS. 10A-10C, we examined the capacity of anti-ROR1 TCEs to induce activation of T cells when ligated to ROR1 on target cells (MDA-MB-231). Exemplary TCEs that decouple cytotoxicity from cytokine release, FUSE-211, FUSE-393 and FUSE-394 are shown relative to the non-decoupling control TCE, FUSE-399 (format 1A incorporating the R11 scFV specific for the ROR1 Kringle domain). PBMCs were mixed with MDA-MB-231 for 24 hours after which flow cytometry was used to assess the early and late activation markers, CD69 (FIG. 10A) and CD25 (FIG. 10B), respectively, on gated CD3+ T cells. Mean Fluorescence Intensity (MFI) for each of CD69 and CD25 is shown on the y-axis of each pot. Each x-axis is representative of a titration of test article ranging from 5 pM to 100 nM. A Hen Egg Lysozyme (HEL)-specific antibody was used as the negative isotype control (filled closed circles). We observed ROR1 dependent induction of CD69 and CD25 expression. For both activation markers, the potency by which expression was induced (EC50-CD69 and EC50-CD25) by FUSE-399 (filled triangles) was between 200-600 fold greater than that of FUSE-211 (inverted filled triangles), FUSE-394 (open circles) or FUSE-393 (filled diamonds). In all cases, the difference in EC50-CD69 and EC50-CD25 between each of the three decoupled TCEs was no greater than 2 fold and averaged about 10 nM. Non specific activation was not observed with the anti-HEL negative control. A summary table of potencies for induction of CD69 and CD25 by each TCE is shown in FIG. 10C. We had previously observed an average potency of ROR1 dependent cytotoxicity (EC50-killing) of between 75-150 pM for FUSE-211, FUSE-393 and FUSE-394. Thus, this data suggested that in a similar fashion to cytokine release, these three TCEs may also decouple cytotoxicity from induction of early and late activation markers. To address this possibility, we used FUSE-394 as the exemplary TCE that decouples cytotoxicity from cytokine release to calculate the area under the curve (AUC) for induction of CD69 (FIG. 10A) and induction of CD25 (FIG. 10B) and used these values for calculating the divisor of AUC-killing divided by AUC-69 (Cytotoxicity/CD69 decoupling ratio; FIG. 10D) or AUC-killing divided by AUC-CD25 (Cytotoxicity/CD25 decoupling ratio; FIG. 10F), normalized to FUSE-399 (set to a value of 1). We calculated that FUSE-394 decoupled cytotoxicity from CD69 and CD25 induction at the orders of 2 fold and 2.4 fold, respectively. Given that CD25 and IFNγ both reach peak expression between 24-48 hours after T cell activation, the decoupling of cytotoxicity from CD25 induction was not surprising. However, CD69, in part due to the protein being preformed in T cells, is rapidly expressed on the surface of activated T cells such that it can be observed in under 4 hours and peaks roughly 24 hours after TCR/CD3 stimulation. Thus, it was surprising that relative to FUSE-399, FUSE-394 decoupled cytotoxicity from CD69. Given that expression of CD69 has been linked to an exhaustion-like phenotype of tumor infiltrating T cells and reduced anti-tumor activity (see www.academic.oup.com/intimm/article/30/12/559/5067178), our data suggest that FUSE-394 (as well as FUSE-393 and FUSE-211) may be associated with a reduced risk of inducing T cell exhaustion and more improved anti-tumor activity compared to non-decoupled TCEs such as FUSE-399 (and FUSE-277). To our knowledge, we are the first to observe decoupling of cytotoxicity from induction of CD69 expression suggesting that release of perforin and cytotoxic proteases including Granzyme B may not always be associated with expression of CD69. Given that the ratio for decoupling cytotoxicity from induction of CD69 and CD25 was measured as between 2-2.4 for FUSE-394, that is about 2 fold lower than its capacity to decouple cytotoxicity from cytokine (IFNγ) release (average ratio for FUSE-394 was about 4; see above), while not wishing to be bound by any particular theory, we believe that FUSE-394 (as well as FUSE-393 and FUSE-211) likely decouple the induction of CD69 and CD25 expression from cytokine (IFNγ) release. This would be especially interesting for CD25 since, as stated above, the kinetics of CD25 and IFNγ expression after T cell activation are very similar. As shown in FIG. 10E and FIG. 10G, compared to FUSE-399, FUSE-394 decoupled the induction of CD69 and CD25 expression from cytokine (IFNγ) release by 2 fold and 1.8 fold, respectively. Overall, the data indicate that cytotoxicity associated with FUSE-394 is decoupled from both cytokine release and the induction of activation markers but that the latter is intermediate compared to the former such that induction of activation markers is further decoupled from cytokine release. FUSE-211 and FUSE-394 were not appreciably different whereas FUSE-393 was approximately 2-fold less potent. No non-specific activation was observed with the anti-HEL antibody.

As shown in FIGS. 11A-11C, we examined the single dose PK in wild type C57BL/6 mice of two TCEs, FUSE-211 (format 11A incorporating the ROR1 specific VHH, 2A11) and FUSE-394 (format 13cA incorporating the ROR1 specific VHH, 5A1). A recombinant version of the clinical stage ROR1-specific monoclonal antibody, Cirmzuzimab (also denoted as FUSE-366), generated from the published sequence, was used as the IgG antibody control described to have pharmacokinetics consistent with a wild type antibody. FIGS. 11A and 11B are x-y plots of the concentration of test article identified in mouse serum (FUSE-211, FUSE-394, or FUSE-366) on the y-axis versus time on the x-axis. In each case, mice received a single dose of a test article at either 5 mg/kg (FIG. 11A) or 0.5 mg/kg (FIG. 11B). Cirmzuzimab (also denoted as FUSE-366) was only included in the 5 mg/kg cohort. In all cases, mice were injected IV with one of the 3 test articles at time zero and serum collected at the several time points (in hours): 0, 1, 6, 24, 48, 72, 168, and 240, Three mice were sampled at each time point. After collection of serum, the concentration of each test article was quantitated using an ELISA specific for the Fc domain of human IgG. No appreciable difference was observed in the PK of FUSE-211 versus FUSE-366, and both exhibited very similar alpha distribution and beta elimination phases. Importantly, although the slope of the late beta elimination phase (>150 hours) at 5 mg/kg for the control antibody, FUSE-366, was gentler than that of FUSE-211 and FUSE-394 (FIG. 11A), the PK for both TCEs was not appreciably different from the control antibody between 0-150 hours. This indicates that both FUSE-211 and FUSE-394 have PK properties similar to a standard monoclonal antibody.

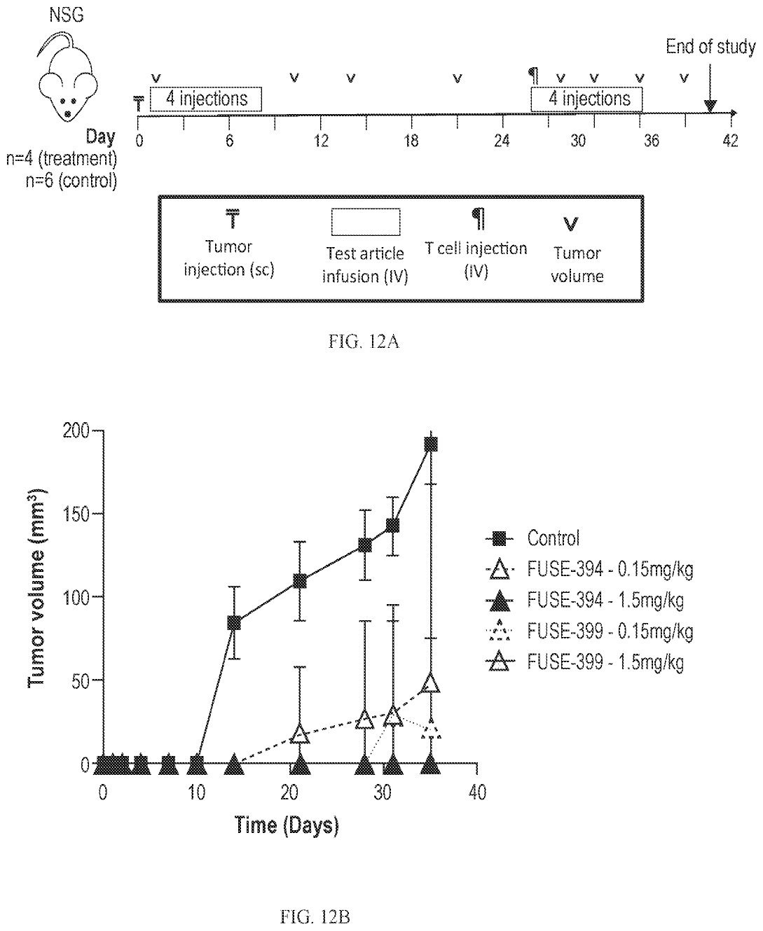

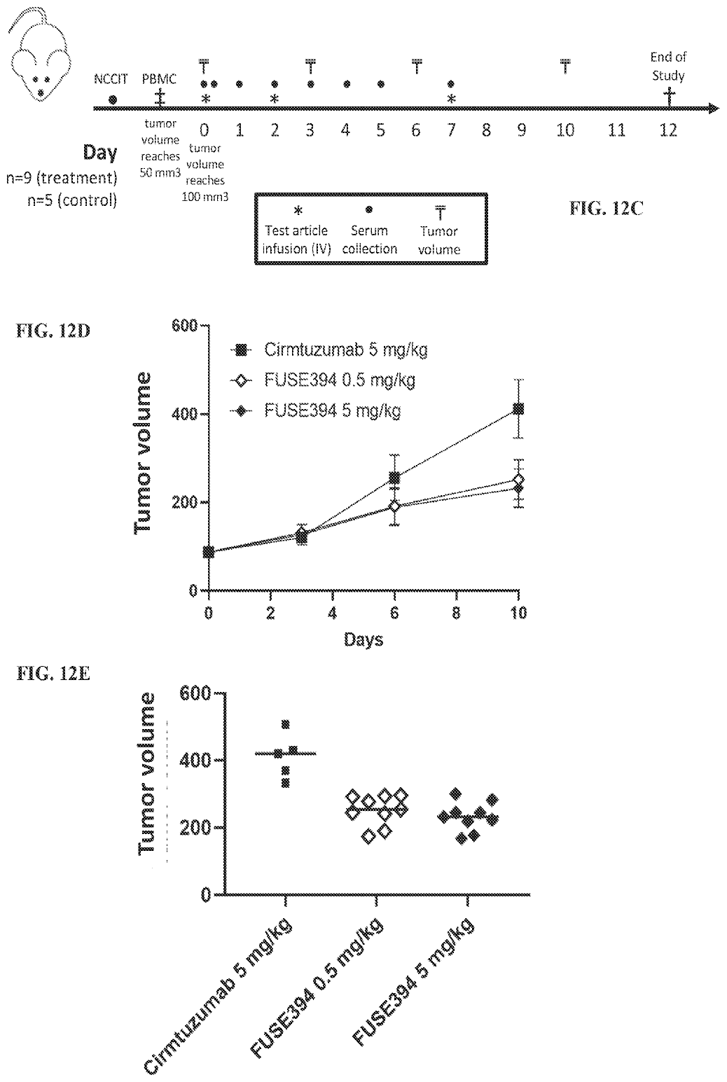

As shown in FIGS. 12A-12E, we examined the capacity of FUSE-394 and the non-decoupled control TCE, FUSE399, to mediate tumor growth inhibition in two humanized mouse xenograft models. In both cases, the highly aggressive human ROR1⁺ teratoma, NCCIT was used and injected subcutaneously. In the first model, NSG mice were injected with a mixture of NCCIT and expanded T cells such that the ratio of tumor to CD8+ T cells constituted 1:4. In an attempt to mimic the phenotype of human TIL that would reflect the “human condition” and not necessarily be expected to fully control tumor growth in the absence of circulating T cells, T cells were expanded for 12 days and recovered for an additional 3 days in IL-2 to mimic a progenitor exhausted phenotype, which would not be expected to be fully functional nor proliferate in-vivo in a hypoxic tumor mass. As such, these T cells did not display alloreactivity towards NCCIT in-vitro. A treatment regimen of four intravenous (IV) injections was initiated on Day 1 and thereafter the mice were monitored for “breakthrough” tumor growth. Due to concerns that matrigel may be toxic to TIL, the former was omitted from this study making tumors more difficult to measure, especially in the treated groups in which tumors growth, if present, was flat in nature and therefore subjective. On Day 21, we observed what appeared to be possible tumor formation in 2 mice that received the low dose of FUSE-394 (0.15 mg/kg), 2 mice that received the higher dose of FUSE-394 (1.5 mg/kg), 1 mouse that received the low dose of FUSE-399 (0.15 mg/kg) and 1 mouse that received the higher dose of FUSE-399 (1.5 mg/kg). We interpreted this finding as possible “breakthrough” tumors and therefore injected all the mice with 3.5 million expanded T cells and initiated a second round of treatment (4 IV injections) on Day 24. Such a dosing regimen resembled the “several weeks on, several weeks off” nature of TCE dosing often used in the clinic. At this point, circulating T cells could be recruited into any tumors that evaded TIL. By day 28, with the exception of one mouse in the group receiving the low dose of FUSE-394, all tumors shrunk to below 10 mm cubed. Thereafter, with the exception of the same mouse from the group receiving the low dose of FUSE-394 and one mouse receiving the low dose of FUSE-399, no tumors were visible in any of the treatment groups. In contrast, tumors grew in mice that were treated with PBS, all of which were visibly raised. In the second model, we investigated the capacity of FUSE-394 to control tumor growth of established NCCIT tumors growing rapidly. In this case, NSG mice in which both MHC-I and MHC-II were knocked out (NSG-MHC-I/II dKO; n=25) were implanted subcutaneously with 5 million ROR1⁺ NCCIT cells. When the tumor volume reached ˜100 mm³, mice were injected intravenously with 30 million freshly isolated PBMC from a healthy human donor. T cell frequency within the PBMC was ˜50%, of which a further 60% were CD4+ T cells and ˜3% were regulatory T cells. T cells did not expand nor reconstitute in our hands. This is because there are no major xeno-antigens present in these mice. As such, PBMC reconstitution in NSG-MHC-I/II dKO is highly donor dependent and is often not-successful. As such, this system best reflects the capacity of naïve circulating T cells to infiltrate a non-immunogenic highly aggressive tumor absent of TIL (“desert tumor”) in which the only agent inducing tumor reactivity is the TCE. Once the tumor volume reached ˜100 mm³, the mice were randomly divided into three groups; two groups of ten that would receive bolus IV injections of FUSE-394 at either 5 mg/kg or 0.5 mg/kg. A third group of 5 mice were designated to receive the control treatment, Cimmtuzumab, a ROR1 targeting antibody that blocks Wnt5-alpha signaling but has little to no effect in mouse xenograft tumor models. This allowed for the additional study of the impact of CD3/ROR1 bridging with FUSE-394 versus ROR1 binding alone with a well characterized clinical stage antibody. Mice received treatment (repeated IV bolus injection of a test article at either 5 mg/kg or 0.5 mg/kg) on days 0, 2, and 7. FIG. 12D is an x-y plot of tumor volume (y-axis) versus time in days (x-axis). Tumor growth inhibition (TGI) mediated by FUSE-394 was observed as early as 6 days after treatment and achieved a magnitude of >45% versus Cirmtuzumab after ten days (FIG. 12E). No appreciable difference between 0.5 mg/kg and 5 mg/kg was observed at day 10.

As shown in FIGS. 13A-13D, we investigated the kinetics by which FUSE-394 (format 13cA incorporating the ROR1 specific VHH, 5A1) induces T cell (PBMC derived T cells expanded for 10 days) mediated killing of ROR1-positive MDA-MB-231 tumor cells (FIG. 13A), ROR1-negative T-47D tumor cells (FIG. 13B) or the mixture of ROR1-positive MDA-MB-231 and ROR1-negative T-47D tumor cells (FIGS. 13C and 13D). Shown are non-linear x-y plots of tumor viability on the y-axis and time on the x-axis. To distinguish between ROR1-positive MDA-MB-231 tumor cells and ROR1-negative T-47D tumor cells, the former was engineered to express RFP and the latter to express GFP. As seen in FIG. 13A, in the presence of 800 pM FUSE-394 but not the anti-HEL negative control, T cells killed between ˜85% of ROR1-positive MDA-MB-231 with an IC50_{VIABILITY} of 14.4 hours. A full titration of TCE from 100 nM to 1.28 pM was performed. Similar maximum killing was observed at concentrations of FUSE-394 between 800 pM and 100 nM (data not shown). At 160 pM, 32 pM, 6.4 pM, and 1.28 pM of FUSE-395, maximum killing dropped to ˜58%, 47%, 25% and 16%, respectively (data not shown). When ROR1-negative T47-D tumor cells were co-cultured with T cells, FUSE-394 or anti-HEL, no appreciable loss in tumor cell viability was observed at 800 pM of test article (FIG. 13B) nor any concentration between 1.28 pM and 100 nM (data not shown). This indicates that FUSE-394 is specific for ROR1 and in the absence of ROR1-positive cells, T cells do not mediate killing of T47-D. Importantly and specific to FUSE-394 and not anti-HEL, when T cells were used as effectors in co-culture with a mixture of ROR1-positive MDA-MB-231 and ROR1-negative T-47D tumor cells, we observed killing of both types of tumor cells. The IC50_{VIABILITY} for ROR1-positive MDA-MB-231 was 14.3 hours and that for ROR1-negative T-47D tumor cells was 32.6 hours, which equates to an 18.3 hour delay between ROR1-positive and ROR1-negative tumor cells. This timing is consistent with the phenomenon referred to direct (early) and bystander (late) killing whereby MDA-MB-231 is killed primarily via Perforin/Granzyme B and T-47D is killed via several death receptor mediated pathways. These include Fas that are upregulated by IFNγ, which influences not only the expression of death receptors (e.g. TRAIL-R, TNFR, FAS) but also adhesion molecules such as ICAM-1 that help strengthen T cell-tumor synaptic junctions. The counter-receptors (e.g. FasL, TRAIL, TNFα, LFA-1) are induced by T cell activation, in this case mediated by cross-linking of the TCR/CD3 complex by the bridge formed by FUSE-394 between ROR1 and CD3s. TCE mediated bystander killing was previously described for EGFR specific BITES (see www.pubmed.ncbi.nlm.nih.gov/28837681/), which was observed to induce cytokine release and killing in a coupled fashion. The data suggests that the amount of IFNγ induced by FUSE-394, although safer than that associated non-decoupled TCEs, is still sufficient to mediate similar killing efficacy of tumors expressing heterogeneous levels of ROR1. Indeed, as seen in FIG. 13D, we observed no appreciable difference in maximum bystander killing (killing efficacy) induced by FUSE-394 and FUSE-399, that latter being a non decoupled TCE in format 1A incorporating an R11 scFV specific for the membrane proximal Kringle domain. As shown in FIG. 13E, after a 24 hour co-culture of T cells and MDA-MB-231, the potency and maximum IFNγ release induced by FUSE-394 is 100-300 fold lower and less than 50% that of FUSE-399. Importantly, at 800 pM of test article, while FUSE-399 was within 10% of its maximum cytokine release (˜EC90_{IFNγ}), FUSE-394 had reached only about its EC30_{IFNγ}. This provides further support for our view that compared to a standard non-decoupled TCE, the size of the window to safely tune cytokine production to maximize both efficacy and safety would be far larger with FUSE-394.

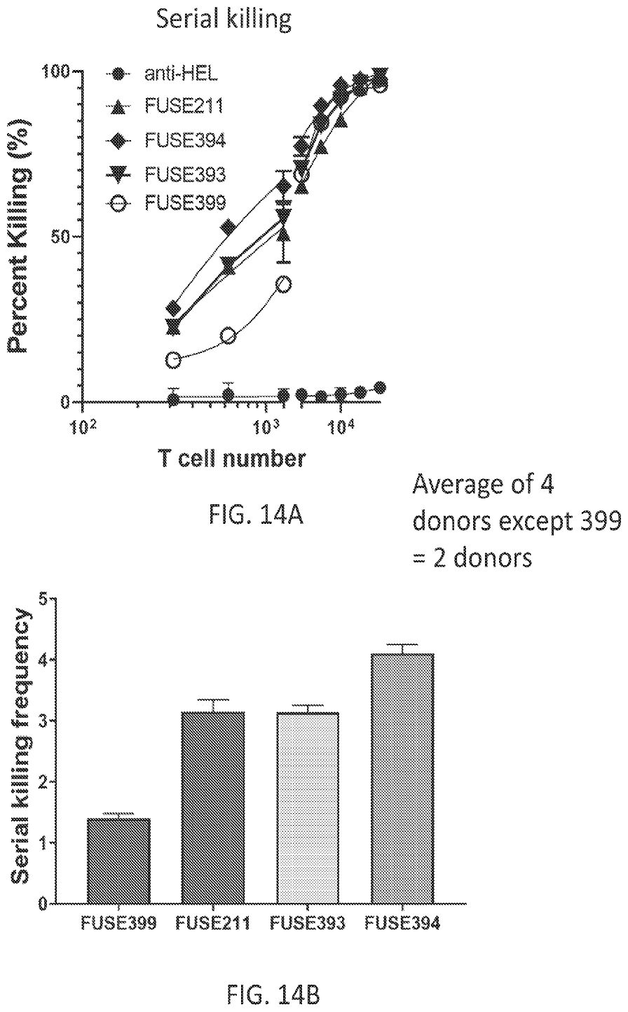

As shown in FIGS. 14A and 14B, we assessed the capacity of exemplary TCEs to induce T cell mediated killing of a constant number of ROR1+ tumor cells (MDA-MB-231) across a range of T cell numbers. This allows for the calculation of (a) the number of T cells needed by different TCEs to kill a given percentage of ROR1+ tumor cells (FIG. 14A) and (b) the average number of tumor cells killed per T cell, also termed “serial killing” (FIG. 14B). To generate the data, different numbers of activated CD8+ T cells ranging from 300 to 40,000 cells were mixed with 5000 MDA-MB-231 (ROR1+) tumor cells. Tumor cell viability was measured after 24 hours. The range of CD8+ T cell numbers added to the assay equated to effector to target ratios of 1:16, 1:8, 1:4, 1:2, 1:1, 2:1, 4:1 and 8:1. FUSE-399 (format 1A incorporating R1l scFV specific for the ROR1 Kringle domain) was used as the ROR1 specific non-decoupling TCE control, and was compared to FUSE-211 (format 11A incorporating 2A11, ROR1-Ig domain-specific VHH), FUSE-393 (format 11A incorporating 5A1, a ROR1-Ig domain-specific VHH) and FUSE-394 (format 13cA incorporating 5A1, a ROR1-Ig domain-specific VHH). At E:T ratios of 1:2 to 8:1 (FIG. 14A, right segment of split x-axis), we observed no appreciable difference in tumor cell killing between any of the test articles indicating that at E:T ratios consistent with hematological tumors, the decoupled TCEs (FUSE-211, FUSE-393 and FUSE-394) were not outperformed in terms of killing efficacy (maximum killing) by FUSE-399. At lower T cell numbers corresponding to between 300 and 1250 T cells (E:T of 1:16, 1:8 and 1:4), the decoupled TCEs were observed to induce greater T cell mediated ROR1 dependent tumor cell killing than FUSE-399, both in terms of potency and efficacy. FUSE-393 and FUSE-211 were associated with nearly identical killing curves with maximum killing about 0.3 fold weaker than FUSE-394. Compared to FUSE-399, FUSE-211, FUSE-393 and FUSE-394 were between 2.5-3 fold more potent and 2-2.5 fold more efficacious. For example, to kill approximately 25% of the tumor cells, FUSE-211, FUSE-393 and FUSE-394 required about 450, 450 and 300 T cells, respectively, whereas FUSE-399 required about 1100 T cells. At very low E:T ratios (e.g. 1:16 and 1:8), each T cell (on average) likely killed more than one tumor cell. For example, at an E:T of 1:16 (312 T cells added to the assay), FUSE-394 was associated with 28-29% killing, which equates to about 1450 tumor cells. As such, on average each of the 312 T cells killed about 4 tumor cells. The phenomenon of a single T cell killing more than one tumor cell is termed serial killing and is especially important in solid tumors where the number of T cells may be sparse with E:T ratios ranging from 1:2 to <1:8. FIG. 14B is a bar chart illustrating serial killing for each TCE, which is calculated as the mean number of tumor cells killed per T cell at an E:T of 1:16 and 1:8. In alignment with the overall cytotoxicity observed in FIG. 14A, FUSE-394 was observed to support the highest degree of serial killing at approximately 4.1 tumor cells/T cell. FUSE-211 and FUSE-393 supported slightly less serial killing between 3-3.2 tumor cells/T cell. Importantly, the non-decoupling TCE control, FUSE-399, only supported serial killing at the order of about 1.4 tumor cells/T cell. The mechanism of action for the observed difference likely stems from multiple factors that lead to a longer T cell/tumor residency time and/or T cell “over-activation” with FUSE-399 compared to FUSE-211, FUSE-393 and FUSE-394. For example, the shorter T cell/tumor synapse mediated by FUSE-399 versus FUSE-211, FUSE-393 and FUSE-394, will (a) result in a stronger activation signal that could result in more release and hence loss availability of Granzyme B and perforin for multiple kills and (b) likely lead to a tighter bridge between T cell and tumor due to engagement of a greater number and higher affinity adhesion molecules. All data reported are derived from the average of two donor T cells. We wished to confirm that TCE induced tumor cell cytotoxicity and IFNγ release observed using PBMC as effectors was mediated by T cells. As such, we performed the same assay shown in FIGS. 61 and 6J but substituted purified Pan-T cells for PBMC.

### Example 2

### Techniques and Procedures

The following techniques and procedures are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Human Peripheral Blood Mononuclear Cell (PBMC) Isolation

PBMCs were isolated from whole blood from healthy donors using Ficoll-Paque Plus medium. In brief, 35 mL of diluted whole blood (1 volume of whole vs 1 volume of PBS) was gently overlayed on the top of 15 mL Ficoll-Paque Plus medium without disturbing the interface in a 50-ml conical tube. After centrifuge for 40 min at 400×g at room temperature without brake, the buffy coat (interface layer between Ficoll and serum) was collected and diluted in 5 volumes of PBS. After centrifuge for 5 min at 500×g at room temperature, PBMCs were resuspended in PBS and washed once in PBS by centrifuging for 5 min at 500×g at room temperature. PBMCs were then resuspended in 5 mL of ACK lysis buffer and incubate for 5 minutes at room temperature to remove red blood cell residues. After 5-minute's incubation, 45 mL PBS was added to PBMCs and centrifuge for 15 min at 100×g at room temperature. At last, PBMCs were resuspended in culture medium (RPMI1640 with 10% FBS and 1% penicillin/streptomycin for cytotoxicity and IFN gamma release assay setting up.

### Human T Cell Isolation, Activation, and Recovery

PBMCs were isolated from whole blood from healthy donors following the procedure described above. T cell were isolated from fresh human PBMCs using EASAYSEP human T cell isolation kit from STEMCELL. In Brief, PBMCs were mixed with isolation antibody cocktail (provided with the kit) in Ca²⁺- and Mg²⁺-free PBS containing 2% FBS and 1 mM EDTA. After 5-minute incubation at room temperature, RapidSpheres™ bead (provided with the kit) was added to the mixture and then immediately assembled on EASY50 EASY SEP magnet from Stemcell. After 10-minute incubation at room temperature, cells in mixture solution were collected and assembled on EASY50 EASY SEP magnet from Stemcell again. After 5-minute incubation at room temperature, purified T cells in mixture solution were collected and pelleted by centrifugation. Purified T cells were frozen down and kept in liquid nitrogen for further usage.

To activate purified T cell, isolated T cells were resuspended in ImmunoCult™-XF T cell expansion medium from Stemcell. ImmunoCult™ human CD3/CD28 T cell activator from Stemcell and 100 U/mL human IL-2 from R&D System were added to the T cells to activate for 12 days. During the 12-days activation, fresh ImmunoCult™-XF T cell expansion medium with 100 U/mL human IL-2 was added to the cells whenever the cell density was above 1 million cells per milliliter. At the end of 12-day activation, activated T cells were frozen down and kept in liquid nitrogen.

To recover T cells, frozen activated T cells or T cells without activation were recovered from liquid nitrogen and thawed in 37° C. water bath. The recovered T cells were resuspended in RMPI1640 medium with 10% heat-inactivated FBS with 250 U/mL human IL-2. After 48-hour incubation at 37° C., T cells were ready for further usage.

### Human CD8+ T Cell Isolation, Activation, and Recovery

PBMCs were isolated from whole blood from healthy donors following the procedure described above. CD8+ T cell were isolated from fresh human PBMCs using EASAYSEP human CD8+ T cell isolation kit from STEMCELL. In Brief, PBMCs were mixed with isolation antibody cocktail (provided with the kit) in Ca²⁺- and Mg²⁺-free PBS containing 2% FBS and 1 mM EDTA. After 5-minute incubation at room temperature, RapidSpheres™ bead (provided with the kit) was added to the mixture and then immediately assembled on EASY50 EASY SEP magnet from Stemcell. After 10-minute incubation at room temperature, cells in mixture solution were collected and assembled on EASY50 EASY SEP magnet from Stemcell again. After 5-minute incubation at room temperature, purified CD8+ T cells in mixture solution were collected and pelleted by centrifugation. Purified CD8+ T cells were frozen down and kept in liquid nitrogen for further usage.

To activate CD8+ T cell, purified human CD8+ T cells were resuspended in ImmunoCult™-XF T cell expansion medium from Stemcell. ImmunoCult™ human CD3/CD28 T cell activator from Stemcell and 100 U/mL human IL-2 from R&D System were added to the T cells to activate for 12 days. During the 12-days activation, fresh ImmunoCult™-XF T cell expansion medium with 100 U/mL human IL-2 was added to the cells whenever the cell density was above 1 million cells per milliliter. At the end of 12-day activation, activated T cells were frozen down and kept in liquid nitrogen.

To recover CD8+ T cells, activated CD8+ T cells or CD8+ t cells were recovered from liquid nitrogen and thawed in 37° C. water bath. CD8+ T cells were resuspended in RMPI1640 medium with 10% heat-inactivated FBS with 250 U/mL human IL-2. After 48-hour incubation at 37° C., T cells were ready for further usage.

### Cytotoxicity Assay

On the day before assay setting up, selective antibiotics was removed from target cell lines. On the day of assay setting up, target cell lines were collected by brief TrypLE treatment and then washed with culture medium by centrifuge at 500×g for 5 min at room temperature. Target cell lines were then resuspended in culture medium to determine the viability by trypan blue exclusion on Cellometer. The viable cell density was adjusted to 50,000 cells/mL in culture media. 100 uL target cell suspension (5000 target cell) was carefully dispensed to each well of a 96-well black clear flat-bottom tissue culture plate using multichannel pipettor. The plate was then incubated for 4-5 hours in tissue culture incubator to make sure that the target cells have attached to the bottom of the 96-well plate.

Fresh PBMCs or recovered T cells were pelleted down by centrifuge for 5 min at 500×g at room temperature and resuspended in culture medium. The viability of cells was also determined via trypan blue exclusion. The viable PBMC density was adjusted to 3 million cells/mL in RPMI1640 medium with 10% FBS and 1% penicillin/streptomycin while the viable T cell density was adjusted to 0.5 million cells/mL in RPMI1640 medium with 10% FBS and 1% penicillin/streptomycin. After 4-5 hours' incubation, culture medium was carefully removed from the 96-well plates with target cells. 50 uL of 3 million cells/mL PBMCs suspension (150,000 PBMCs) or 50 uL of 0.5 million cells/mL PBMCs suspension (25,000 T cells) was added to the designated well in the 96-well plate with target cell, which would result in the E:T ratio of 30:1 for PBMC and 5:1 for T cells.

CD3×ROR1 T-cell-engagers (TCEs) were prepared and serially diluted (5-fold serial dilution) in RPMI1640 medium with 10% FBS and 1% penicillin/streptomycin ranging from 200 nM to 2.56 pM. 50 uL of prepared CD3×ROR1 TCEs at different concentrations was then added to the designated wells in the 96-well plate with PBMC and target cells and incubated for one, two or three days at 37° C. with 5% CO₂. At the end of incubation, the 96-well plates were centrifuged for 1 minute at 500×g to transfer 50 μL of supernatant to V-bottom storage plate using a multichannel pipettor for IFN gamma release assay. ONE-Glo Luciferase Assay solution was brought to room temperature. 50 uL of One-Glo solution was then added to the designated well and incubate for 2 min at room temperature. The bioluminescence was measured on a plate reader with preset Bio-luminance protocol. The killing percentage was calculated using the formula: killing percentage=100*(bioluminescence intensity of target cell alone−bioluminescence intensity of sample)/(bioluminescence intensity of target cell alone−bioluminescence intensity of PBMC alone).

### IFN Gamma Release Detection

IFN gamma release from cytotoxicity assay was measured using human IFN gamma ELISA detection kit. In brief, coating antibody provided with the kit was diluted to suggested concentration by following the protocol provided by the kit manufacturer. 100 ul of diluted coating antibody was added to the Nunc MaxiSorp flat-bottom 96-well plate, sealed plate and incubated at 4° C. overnight. The plates were then washed 4 times with Wash Buffer. To block non-specific binding and reduce background, 200 μL Assay Diluent A was added and incubated the plate at room temperature for 1 on a plate shaker (400 rpm). After blocking, the plates were washed 4 times with Wash Buffer. 100 μL/well of standards (prepared with culture medium) or samples were then added to the designated wells and incubated at room temperature for 2 hours on a plate shaker (400 rpm). After 2-hour incubation with samples or standards, the plates were washed 4 times with Wash Buffer and 100 μL of diluted Detection Antibody solution was added to incubate at room temperature for 1 hour on a plate shaker (400 rpm). After 1-hour incubation with detection antibody, the plates were washed 4 times with Wash Buffer and 100 μL of diluted Avidin-HRP solution was added to incubate at room temperature for 30 minutes on a plate shaker (400 rpm). After 30-minute incubation with Avidin-HRP, the plates were washed 5 times with Wash Buffer and 100 μL of freshly mixed TMB Substrate Solution was added to incubate at room temperature for 20 minutes in the dark. 100 μL of Stop Solution was then added to the well to stop the reaction. The absorbance at 450 nm was measured on a plate reader and the concentration of IFN gamma in each sample was backcalculated by using the standard curve generated with the absorbance of different concentration of standards.

### Decoupling Ratio Calculation

Cytotoxicity and IFN gamma release were plotted against the concentration of testing articles in GraphPad Prism. Area under the curve (AUC) for cytotoxicity and IFN gamma release was calculated using the AUC algorithm in Prism. The decoupling ratio was generated by dividing the AUC of cytotoxicity with the AUC of IFN gamma release for the same testing article. All decoupling ratios were normalized to certain non-decoupling testing article to compare the variation among different experiments.

### Binding Assay by Flow Cytometry

Adherent target cells were collected by brief TrypLE treatment while suspension cells were directly collected. Collected cells were washed once with PBS by centrifugation at 500×g for 5 minutes at room temperature and added to V-bottom 96-well plate (200,000-300,000 cells per well). To prepared Live/Dead fixable red, one vial of powder was dissolved into 50 uL of DMSO to make stock solution and then diluted 1 uL of stock Live/Dead fixable red in 1 mL of PBS to prepare the working solution. 100 uL of Live/Dead fixable red working buffer was then added to the cell in the V-bottom 96-well plate (200,000-300,000 cells per well) and incubated for 15 minutes at room temperature. After the 15-minute incubation, the cells were washed twice with PBS by centrifugation at 500 g for 5 minutes at room temperature. The cells were then cultured with antibodies at different concentrations in 100 uL of Biolegend staining buffer at 4° C. for 45 minutes. After the 45-minute incubation with primary antibody, the cells were washed with PBS by centrifugation at 500 g for 5 minutes at room temperature. The cells were then cultured with PE-Cy7-conjugated mouse-anti-human IgG secondary antibody in Biolegend staining buffer at 4° C. for 45 minutes. After the 45-minute incubation with secondary antibody, the cells were washed with PBS by centrifugation at 500 g for 5 minutes at room temperature and resuspended in 100 uL of BioLegend fixation buffer. The samples were then analyzed on Cytek flow cytometer. Data were analyzed with FlowJo and the gating strategy was followed: In brief, target cell population was gated by FSC and SSC. Single cells were selected from target cell population by FSC-A and FSA-H. Living single cells were gated from single cells by live/dead red dye low. The binding to target was determined by specific antibody staining and the gate was decided by the staining of isotype control of antibody on the same cell.

### HEK293 Cell Transfection and Binding Assay

HEK293 cells from ATCC were maintained in Eagle's Minimum Essential Medium with 10% heat-inactivated FBS. On one day before transfection, HEK293 cells were harvested and resuspended in Eagle's Minimum Essential Medium with 10% heat-inactivated FBS at the density of 0.4 million cells per milliliter. HEK293 cells were then added to T-75 flask by 20 mL of cell suspension per T-75 flask and incubated at 37° C. for 24 hours. On the day of transfection, 19 ug of DAN plasmid was mixed with 1.9 mL of Opti-MEM® I Reduced-Serum Medium from ThermoFisher and 57 uL of TransIT-293 Reagent from Mirus and incubated for 30 minutes at room temperature. After 30-minute incubation, DNA plasmid/TransIT-293 Reagent mixture was added to the T-75 flask with HEK293 cells drop-by-drop at different area of the flask. The T-75 flasks were gently rocked back-and-forth and from side-to-side to evenly distribute the TransIT-293 Reagent: DNA complexes.

After 48-hour incubation, cells were collected to check the expression of transfected target and the binding of testing antibodies to the transfected target by flow cytometry as described herein under Binding assay by flow cytometry. In brief, collected cells were washed once with PBS in V-bottom 96-well plate (200,000-300,000 cells per well) and resuspended in 100 uL of Live/Dead fixable red working buffer. After the 15-minute incubation, the cells were washed twice with PBS and cultured with antibodies at different concentrations in 100 uL of Biolegend staining buffer at 4° C. for 45 minutes. After the 45-minute incubation with primary antibody, the cells were washed with PBS and cultured with PE-Cy7-conjugated mouse-anti-human IgG secondary antibody in Biolegend staining buffer at 4° C. for 45 minutes. After the 45-minute incubation with secondary antibody, the cells were washed with PBS and resuspended in 100 uL of BioLegend fixation buffer. The samples were then analyzed on Cytek flow cytometer. Data were analyzed with FlowJo and the gating strategy was followed: In brief, target cell population was gated by FSC and SSC. Single cells were selected from target cell population by FSC-A and FSA-H. Living single cells were gated from single cells by live/dead red dye low. The binding to target was determined by specific antibody staining and the gate was decided by the staining of isotype control of antibody on the same cell.

### Binding Assay by Biolayer Interferometry

Antibodies were prepared in 1×PBS with 0.02% Tween-20 with the final concentration of 15 ug/ml. Human ROR1-His or CD3e-His protein were also prepared, and serial diluted (4-fold dilution ranging from 400 nM to 0.098 nM) in 1×PBS with 0.02% Tween-20. The biosensors were pre-moistened in 200 uL of 1×PBS with 0.02% Tween-20 for 10 minutes. Meanwhile, Octet BLI system (ForteBio) was prewarmed for 30 minutes and the flow rate was set to 1000 rpm. The biosensors (Anti-hIgG Fc Capture biosensor) were soaked in 250 uL of 1×PBS with 0.02% Tween-20 for 60 seconds at 30° C. to get an initial baseline reading. After the 60-second baseline reading, the biosensors were exposed to testing antibody for 300 seconds at 30° C. for the association between antibody and the biosensors (coupling antibody with biosensor). The biosensors with antibody were then exposed to human ROR1 or CD3e in 250 uL of 1×PBS with 0.02% Tween-20 at 30° C. for 300 seconds for the association reaction between antibody and human ROR1 or CD3e (association curve). After 300-second association reaction between antibody and antigen, the biosensors with antibody and antigen were then exposed to 250 uL of 1×PBS with 0.02% Tween-20 at 30° C. for 300 seconds for the dissociation reaction between antibody and antibody (dissociation curve). The binding affinity was calculated by the built-in data fitting algorithm.

### CD69 and CD25 Detection on T Cells

Following the experiment setting up for cytotoxicity assay, target cells were added to the wells (5,000 target cells per well) in 96-wells plate and incubated for 5 hours. After 5-hour incubation, cell culture medium was removed from the plate without touching the target cells and 50 uL of fresh PBMCs (150,000 PBMCs per well) were added to the wells with target cells (E:T ratio 0f 30:1). 50 μL of CD3×ROR1 TCEs were then added to the designated wells with 150,000 PBMCs and 5,000 target cells. After incubating at 37° C. overnight, PBMCs were collected and washed once with PBS by centrifugation at 500×g for 5 minutes at room temperature and added to V-bottom 96-well plate. To prepared Live/Dead fixable red, one vial of powder was dissolved into 50 uL of DMSO to make stock solution and then diluted 1 uL of stock Live/Dead fixable red in 1 mL of PBS to prepare the working solution. 100 uL of Live/Dead fixable red working buffer was then added to the cell in the V-bottom 96-well plate and incubated for 15 minutes at room temperature. After the 15-minute incubation, the cells were washed twice with PBS by centrifugation at 500 g for 5 minutes at room temperature. The cells were then cultured with antibodies (PE-Cy5-conjugated mouse anti-human TCRa/b antibody, PE-conjugated mouse anti-human CD19 antibody, BB515-conjugated mouse anti-human CD56 antibody, BB700-conjugated mouse anti-human CD14 antibody, PE-Cy7-conjugated mouse anti-human CD69 or CD25 antibody) in 100 uL of Biolegend staining buffer at 4° C. for 45 minutes. After the 45-minute incubation with antibody, the cells were washed with PBS by centrifugation at 500 g for 5 minutes at room temperature and resuspended in 100 uL of BioLegend fixation buffer. The samples were then analyzed on Cytek flow cytometer. Data were analyzed with FlowJo and the gating strategy was followed: In brief, target cell population was gated by FSC and SSC. Single cells were selected from target cell population by FSC-A and FSA-H. Living single cells were gated from single cells by live/dead red dye low. T cells were gated from living single cells by TCRa/b high. CD69 and CD25 expression was then gated on T cells with CD69 or CD25 high.

### CD107a Detection on T Cells

Following the experiment setting up for cytotoxicity assay, target cells were added to the wells (5,000 target cells per well) in 96-wells plate and incubated for 5 hours. After 5-hour incubation, cell culture medium was removed from the plate without touching the target cells and 50 uL of fresh PBMCs (150,000 PBMCs per well) were added to the wells with target cells (E:T ratio 0f 30:1). 50 uL of CD3×ROR1 TCEs were then added to the designated wells with 150,000 PBMCs and 5,000 target cells. After incubating at 37° C. for 5 hours, PE-Cy7-conjugated mouse anti-human CD107a antibody (40:1) and Monensin (1500:1) was added to the culture medium and incubated for 8 hours. PBMCs were then collected and washed once with PBS by centrifugation at 500×g for 5 minutes at room temperature and added to V-bottom 96-well plate. To prepared Live/Dead fixable red, one vial of powder was dissolved into 50 uL of DMSO to make stock solution and then diluted 1 uL of stock Live/Dead fixable red in 1 mL of PBS to prepare the working solution. 100 uL of Live/Dead fixable red working buffer was then added to the cell in the V-bottom 96-well plate and incubated for 15 minutes at room temperature. After the 15-minute incubation, the cells were washed twice with PBS by centrifugation at 500 g for 5 minutes at room temperature. The cells were then cultured with antibodies (PE-Cy5-conjugated mouse anti-human TCRa/b antibody, PE-conjugated mouse anti-human CD19 antibody, BB515-conjugated mouse anti-human CD56 antibody, BB700-conjugated mouse anti-human CD14 antibody) in 100 uL of Biolegend staining buffer at 4° C. for 45 minutes. After the 45-minute incubation with antibody, the cells were washed with PBS by centrifugation at 500 g for 5 minutes at room temperature and resuspended in 100 uL of BioLegend fixation buffer. The samples were then analyzed on Cytek flow cytometer. Data were analyzed with FlowJo and the gating strategy was followed: In brief, target cell population was gated by FSC and SSC. Single cells were selected from target cell population by FSC-A and FSA-H. Living single cells were gated from single cells by live/dead red dye low. T cells were gated from living single cells by TCRa/b high. CD107a was then gated on T cells with CD107a high.

### Bystander Killing Assay

On the day before assay setting up, selective antibiotics was removed from target cell lines (ROR1+ MDA-MB-231 RFP cell or ROR1-T-47D eGFP FLUC cell). On the day of assay setting up, target cell lines were collected by brief TrypLE treatment and then washed with culture medium by centrifuge at 500×g for 5 min at room temperature. Target cell lines were then resuspended in culture medium to determine the viability by trypan blue exclusion on Cellometer. The viable cell density was adjusted to 50,000 cells/mL in culture media. 100 uL. ROR1-target cell suspension (5000 cell) was carefully dispensed to the designated well of a 96-well black clear flat-bottom tissue culture plate using multichannel pipettor for the group with ROR1-target cell only. 100 uL ROR1+ target cell suspension (5000 cell) was dispensed to the designated well of the 96-well black clear flat-bottom tissue culture plate for the group with ROR1+ target cell only. For the group with both ROR1+ and ROR1-target cells, 100 uL ROR1-target cell suspension (5000 cell) and 100 uL ROR1+ target cell suspension (5000 cell) was added to the designated well of the 96-well black clear flat-bottom tissue culture plate. The plate was then incubated for 4-5 hours in tissue culture incubator to make sure that the target cells have attached to the bottom of the 96-well plate.

Recovered T cells were pelleted down by centrifuge for 5 min at 500×g at room temperature and resuspended in culture medium. The viable T cell density was adjusted to 0.5 million cells/mL in RPMI1640 medium with 10% FBS and 1% penicillin/streptomycin. After 4-5 hours' incubation, culture medium was carefully removed from 96-well plates with target cells. 50 uL of 0.5 million cells/mL T cell suspension (25,000 T cells) was added to the designated well in the 96-well plate with target cell, which would result in the E:T ratio of 5:1 for T cells.

CD3×ROR1 T-cell-engagers (TCEs) were prepared and serially diluted (5-fold serial dilution) in RPMI1640 medium with 10% FBS and 1% penicillin/streptomycin ranging from 200 nM to 2.56 pM. 50 uL of prepared CD3×ROR1 TCEs at different concentrations was then added to the designated wells in the 96-well plate with T cells and target cells and incubated in CELLINK CELLCYTE X at 37° C. with 5% CO₂. Cell number of ROR1+ MD-MB-231 RFP cell and ROR1-T-47D eGFP FLUC cell in each well was monitored every three hours for 72 hours. The change of target cell number in each well was plotted against time and the killing percentage of target cell was calculated by using target cell number at the time zero as the 100% base.

### Serial Killing Assay

On the day before assay setting up, selective antibiotics was removed from target cell lines. On the day of assay setting up, target cell lines were collected by brief TrypLE treatment and then washed with culture medium by centrifuge at 500×g for 5 min at room temperature. Target cell lines were then resuspended in culture medium to determine the viability by trypan blue exclusion on Cellometer. The viable cell density was adjusted to 200,000 cells/mL in culture media. A serial dilution of target cell (factor 2) was made from 200,000 cells/mL to 3,125 cells/mL (200,000 cells/mL, 100,000 cells/mL, 50,000 cells/mL, 25,000 cells/mL, 12,500 cells/mL, 6,250 cells/mL, 3,125 cells/mL, 0 cell/mL) with culture medium. 50 uL target cell suspension at different densities was carefully dispensed to designated standard control wells of a 96-well black clear flat-bottom tissue culture plate using multichannel pipettor, which yielded 10,000 cells, 5,000 cells, 2,500 cells, 1,250 cells, 625 cells, 313 cells, 156 cells, 0 cell per well as standard controls. For all testing article wells, 100 uL target cell suspension at the density of 50,000 cell/mL (5,000 cell per well) was carefully dispensed to designated wells. The plate with target cells were incubated at 37° C. for 4 hours.

After 48-hour recovery in 250 U/mL IL-2, activated CD8+ T cells were pelleted down by centrifuge for 5 min at 500×g at room temperature and resuspended in culture medium. The viability of cells was also determined via trypan blue exclusion. The viable recovered CD8+ T cells was adjusted to 800,000 cells/mL in culture medium. A serial dilution of activated CD8+ T cell (factor 2) was made from 800,000 cells/mL to 6,250 cells/mL (800,000 cells/mL, 400,000 cells/mL, 200,000 cells/mL, 100,000 cells/mL, 50,000 cells/mL, 25,000 cells/mL, 12,500 cells/mL, 6,250 cells/mL) with culture medium. 50 uL recovered activated CD8+ T cell suspension at different densities was carefully dispensed to designated wells with 5,000 target cells for testing articles or controls in a 96-well black clear flat-bottom tissue culture plate using multichannel pipettor, which yielded 40,000 cells (E:T ratio of 8:1), 20,000 cells (B: T ratio of 4:1), 10,000 cells (E:T ratio of 2:1), 5,000 cells (E:T ratio of 1:1), 2,500 cells (E:T ratio of 1:2), 1,250 cells (E:T ratio of 1:4), 625 cells (E:T ratio of 1:8), 313 cells (E:T ratio of 1:16) per well for all testing articles or controls.

Testing articles and proper negative controls were prepared in culture medium at the concentration of 1 nM. 100 uL of prepared testing articles of negative controls was added to the designated wells with 5,000 target cells and different number of activated CD8+ T cells. For the wells with different number of target cells as standard controls, 150 uL of culture medium was added to bring the total volume to 200 uL. The cells were incubated for one day at 37° C. with 5% CO₂. At the end of incubation, the 96-well plates were centrifuged for 1 minute at 500×g to transfer 150 μL of supernatant to V-bottom storage plate using a multichannel pipettor for further usage. ONE-Glo Luciferase Assay solution was brought to room temperature. 50 uL of One-Glo solution was then added to the designated well and incubate for 2 min at room temperature. The bioluminescence was measured on a plate reader with preset Bio-luminance protocol. To calculate the killing frequency, the bioluminescence intensity of target cell at different seeding numbers as standard controls were plotted against the cell numbers to generate a standard curve. The number of living target cell number in each testing sample wells were calculated with the standard curve by using the bioluminescence intensity of each testing sample well. The killed target cell number was calculated by subtracting the calculated living target cell number for each testing sample from 5000 (plating number of target cells). The killing frequency of target cells in each well was generated by dividing killed target cell number with the input CD8+ T cell number for the same well.

### Mouse Pharmacokinetics (PK) Study

Single dose pharmacokinetics (PK) for antibody-like protein was performed in 8-10-week-old wild type C57BL/6j mice obtained from the Jackson Laboratory. Each protein test article was prepared in sterile phosphate buffered saline (PBS) pH 7.4 at a concentration of 100 ug/50 uL (2 mg/ml) for a dose of 5 mg/kg or 10 ug/50 uL (0.2 mg/ml) for a dose of 0.5 mg/kg. At time “0”, 50 uL of protein was injected as an intravenous (IV) bolus into 9 mice per test article dose level such that 3 mice could be sampled per time points of 0, 1, 6, 24, 48, 72, 168, 240 hours and no single mouse was sampled more than twice. Peripheral blood was collected (approximately 60 uL) into pre-chilled (0-4° C.) lithium heparin tubes via the sublingual, retrobulbar or via tail vein route (tail snip) under inhalation anesthesia (isoflurane) prior to IV injection and at 1, 6, 24, 48, 72, 168, 240 hours. After blood collection, blood samples were centrifuged (2500 g/10 minutes at 4° C.) and plasma immediately stored at −80° C.

The concentration of each test article (FUSE protein with human IgG1 backbone) in mouse serum was quantified using human IgG SimpleStep ELISA Kit from Abcam. In brief, all reagents, working standards, and samples were brought to room temperature before the assay. All samples and standards were prepared in manufacturer-provided Sample Diluent NS. When the assay began, 50 μL of all samples or standard were added to the designated wells in the 96-well plate provided by the manufacturer. 50 μL of Antibody Cocktail were then added to the wells. After sealing with film, the plate was incubated for 40 minutes at room temperature on a plate shaker (400 rpm). After the 40-minutes incubation, the plate was washed three time with manufacturer-provided 1× Wash Buffer PT buffer and 100 μL of manufacturer-provided TMB Development Solution was then added to each well. After incubating for 5 minutes in the dark on a plate shaker (400 rpm), 100 μL of manufacturer-provided Stop Solution was added to the well. The plate was shaken on a plate shaker for 1 minute to measure the OD at 450 nm for each well on a plate reader. The concentration of testing article in each sample was backcalculated by using the standard curve generated with the absorbance of different concentration of standard human IgG.

### Mouse Xenograft Tumor Models in Immunocompromised Mice

In-vivo PK/PD (Pharmacodynamics) and efficacy were assessed using two human ROR1+ xenograft models in humanized immunocompromised mice. In the first model, NSG mice were injected with a mixture of 5 million NCCIT and expanded T cells such that the ratio of tumor to CD8+ T cells constituted 1:4 without matrigel. Such T cells were expanded for 12 days and recovered for an additional 3 days in IL-2 to mimic a progenitor exhausted phenotype. T cells from 2 healthy donors were assessed for alloreactivity via an in-vitro bioluminescent cytotoxicity and IFNγ release (as described above) assay using NCCIT engineered to constitutively express firefly luciferase. The donor observed to display minimal alloreactivity (defined as <15% maximum killing and <150 μg/ml maximum IFNγ release at an E:T of 1:3) towards NCCIT in-vitro (donor 2) was used in-vivo. Mice were either treated with PBS (n=4), FUSE-394 (0.15 mg/kg; n=6), FUSE-394 (1.5 mg/kg; n=6), FUSE-399 (0.15 mg/kg; n=6) or FUSE-399 (1.5 mg/kg; n=6) on day 1, 2, 4 and 7. In all cases the volume of treatment was 50 μL in pBS pH 7.4. Next, we examined mice for tumor growth. If tumor breakthrough was suspected, all mice were intravenously injected with 3.5 million expanded T cells and a second round of treatment initiated such that intravenous injections of test articles were given of days 0, 2, 4 and 6 following the adoptive transfer of T cells. Tumor volumes were measured every 3-5 days. Mice were sacrificed at the end of the study. In the second model, NSG mice in which both MHC-I and MHC-II were knocked out (NSG-MHC-I/II dKO; n=25) were implanted subcutaneously with 5 million ROR1+ NCCIT teratoma cells mixed with matrigel. When the tumor volume reached 50 mm cubed, mice were injected intravenously with 30 million freshly isolated PBMC from a healthy human donor. Once the tumor volume reached 100 mm cubed (“day 0”), the mice were randomly divided into three groups. Group 1 (n=5) received 5 mg/kg of the negative control treatment, Cirmtuzumab (FUSE-366), a ROR1 targeting antibody that blocks Wnt5-alpha signaling but has little to no effect in mouse xenograft tumor models. Groups 2 and 3 (n=10 each) received a selected TCE at either 5 mg/kg or 2 mg/kg, respectively. Treatment consisted of bolus IV injections of test article prepared in PBS pH 7.4 such that 50 μL was injected IV via the tail vein. For 5 mg/kg, 100 μg of test article was injected in 50 μL. For 0.5 mg/kg, 10 μg of test article was injected in 50 μL. Mice received treatment on days 0, 2, and 7. Tumor volume was measured every 4 days starting on day 0. Mice were sacrificed at the end of the study.

### Mouse Xenograft Tumor Model in Humanized Immunocompromised Mice

The capacity of the ROR1×CD3 bsAbs to mediate in-vivo tumor growth inhibition (TGI) was assessed using the human ROR1+ xenograft TNBC mouse tumor model, MDA-MB-231. Briefly, NSG (NOD-scid IL2Rgamma^{null}; Jackson Laboratory) mice were injected subcutaneously with 5 million MDA-MB-231 admixed in Matrigel Matrix (Corning) into the right dorsal flanks of the animals. On the same day, the mice were also injected intravenously with 5 million expanded human T cells. Expanded T cells for injection were selected from several healthy donors based on minimal alloreactivity towards MDA-MB-231. In short, T cells from two healthy donors were expanded using ImmunoCult™ human CD3/CD28 T cell activator reagent (Stemcell) as per the manufacturer's recommended protocol. Alloreactivity was examined using our in-vitro bioluminescent cytotoxicity and IFNγ release assays (as described above) and was defined as maximum killing equal to 15% or less and maximum IFNγ release equal to 300 ug/ml or less at an E:T of 5:1. When tumor volumes reached between 75-100 cubic millimeters, the mice were randomized into 5 groups. Group 1, consisting of 5 mice, received the mock treatment, PBS twice weekly. Groups 2-4 consisted of 10 mice each and received two weekly doses of FUSE608 at 0.03 mg/kg, 0.3 mg/kg or 3 mg/kg. Group 5 consisted of 7 mice and received two weekly doses of FUSE399 at 3 mg/kg. Tumor volumes were measured every 3-5 days and the survival of mice was also recorded. Mice were sacrificed at the end of the study.

### Example 3

A ROR1 Specific CD3 Bispecific T-Cell Engager Engineered for Solid Tumors with an Expanded Therapeutic Window

We engineered two ROR1 specific TCEs with properties to increase the size of their therapeutic windows using a unique ROR1 specific VHH from Camelus bactrianus (clone 5A1) that contains a non-canonical disulfide-bond between CDR1 and CDR3 and ROR1 specific valency that (1) decouples cytotoxicity from cytokine release and (2) facilitates the discrimination between ROR1 cell surface density on normal versus tumor cells.

When 5A1 was paired with an anti-CD3 Fab in two orientations that include a half-life extending Fc domain, we were able to establish a T cell/tumor synapse that resulted in sufficient activation to induce degranulation/tumor-killing at TCE concentrations of 10-30 pM (EC50˜150-200 nM). In contrast, expression of CD69, CD25 and IFNγ, the latter of which is directly upstream of CRS, was not observed until the concentration of TCEs reached ˜1 nM (EC50˜5-10 nM). Compared to the non-decoupling control TCE, 5A1 TCEs demonstrated 3.5-4.5 fold greater decoupling. In the context of JeKo1 CD19+/ROR1+ tumor targets, the 5A1 TCEs were 6-7.5 fold better decoupled compared to blinatumomab. We also observed an EC50-cytotoxicity for blinatumomab of below 100 fM, suggesting that the risk of both CRS and TLS are multiple folds higher than for 5A1 TCEs. Compared to the non-decoupling control TCE, the 5A1 TCEs mediated superior cytotoxicity/serial killing at an effector to target ratio (E:T) as low as 1:16, more consistent with solid-tumors. The 5A1 TCEs were also utilized ROR1 specific binding valency to discriminate between cell surface densities of ROR1 typical to tumor cells (>15 k molecules/cell) versus normal cells (generally <10,000 molecules/cell). The concentration of TCEs required for maximum IFNγ release and killing of tumor cells registered in the pM range, as opposed to the supraphysiologic nM/uM range that was insufficient to induce more than 25% and 35% of IFNγ release and cytotoxicity, respectively, on cells expressing <10,000 molecules of ROR1.

While not wishing to be bound by any particular theory, we believe that our ROR1 TCEs provide a superior therapeutic index and safety profile associated with reduced risks of CRS and TLS, the capacity to dose higher, avoid continuous infusion and potentially mediate better efficacy in solid tumors.

### Example 4

As shown in FIG. 1, the format(s) used for the CD3×ROR1 bispecific antibodies (bsAbs) are that of knobs into holes and contain modular subunits of the humanized CD3 specific fab 1F3-3 (or variants thereof) and the ROR1 specific VHHs, 5A1 or 2A11 or variants thereof. The ROR1 specific scFV, R11, which binds to the membrane proximal domain of ROR1 (termed the Kringle domain) was used to generate the non-decoupled benchmark bsAb, FUSE399.

As an example, Format Ac13 can consists of a 1F3-3 fab genetically fused to the N-terminus of the heavy chain hole and ROR1 specific binders genetically fused to the N-terminus of the heavy chain knob and C-terminus of the light chain.

### Example 5

As shown in the tables below, mouse B16-F10 melanoma (Tables 9A-9F) or LS174T human colorectal adenocarcinoma (Tables 9G-9H) cells genetically engineered to express human ROR1 at high and low densities, respectively, were stained for flow cytometric analysis with 100 nM of humanized variants of the ROR1 specific VHHs, 5A1 or 2A11 in either a VHH-Fc format or the Ac13 format shown in FIG. 1. Cells were analyzed on a flow cytometer after the primary test article was fluorescently tagged with a goat anti human IgG-Fc specific PE secondary antibody. The positive control used was PE conjugated anti-human ROR1 clone 2A2. Negative controls included a PE labeled mouse IgG Ab, the secondary antibody only, the combination of a Hen Egg Lysozyme (HEL) specific antibody plus goat anti human IgG-Fc specific PE and Fluorescence Minus One (FMO). Other controls included the non-humanized version of 5A1-Fc (FUSE179), 2A11-Fc (FUSE112) and 5A1-Ac13 (FUSE488). Humanized variants were generated using computational design (FUSE489, FUSE497-498, FUSE563, FUSE569-570, and BI-HU9-VHH11-33), a yeast display library in which mutations were inserted into the Vernier zone residues of human framework indicated in GenBank accession number M99660 (FUSE524-525, FUSE589, FUSE597-598, FUSE537, FUSE661-662, BI-HU9-HK and BI-HU9-HK-1), and a second yeast display library in which mutations were inserted into the Vernier zone residues of human framework indicated in GenBank accession number AWH66715 and amino acid position 103 within CDR3 (FUSE559, FUSE590-596, BI-HU9-VH9-1, FUSE654-655 and FUSE661-662. Affinity matured variants of FUSE559 were also generated using computational design (F22158101-21; Table 9H).

TABLE 9A
Single concentration FACS screen

| B16-F10-ROR1-H | MFI |
|---|---|
| FMO | 5.96 |
| mIgG-PE | 6.1 |
| anti-human ROR1(clone 2A2)-PE | 35240 |
| 2^{nd} Anti-human IgG-PE antibody | 5.67 |
| 100 nM anti-HEL4-hIgG1 | 9.86 |
| 100 nM FUSE-179-2 (5A1 Fc) | 4220 |
| 100 nM FUSE-488 (5A1-Ac13) | 3909 |
| 100 nM FUSE-489 (h5A1-Ac13) | 1055 |
| 100 nM FUSE-497 (h5A1-Ac13) | 811 |
| 100 nM FUSE-498 (h5A1-Ac13) | 1363 |
| 100 nM FUSE-524 (h5A1-HK1_Fc) | 6473 |
| 100 nM FUSE 525 (h5A1-HK1v Fc) | 5.84 |

TABLE 9B
Single concentration FACS screen

| B16-F10-ROR1-H | MFI |
|---|---|
| FMO | 4.07 |
| mIgG-PE | 4.36 |
| anti-human ROR1(clone 2A2)-PE | 19948 |
| 2nd Anti-human IgG-PE antibody | 15.5 |
| 100 nM anti-HEL4-hIgG1 | 12.1 |
| 100 nM FUSE-179-2-AKTA | 3732 |
| 100 nM FUSE-559 (h5A1-FA9-HK1 Fc) | 6082 |
| 100 nM FUSE-563 (h5A1-FA11 Fc) | 388 |
| 100 nM FUSE-569 (h5A1-FA17 Fc) | 590 |
| 100 nM FUSE-570 (h5A1-FA18 Fc) | 583 |

TABLE 9C
Single concentration FACS screen

| B16-F10-ROR1-H | MFI |
|---|---|
| FMO | 4.32 |
| mIgG-PE | 4.31 |
| anti-human ROR1(clone 2A2)-PE | 38704 |
| 2nd Anti-human IgG-PE antibody | 4.45 |
| 100 nM anti-HEL4-hIgG1 | 6.85 |
| 100 nM FUSE-179-2 (5A1 Fc) | 8479 |
| 100 nM FUSE-112-2 (2A11 Fc) | 11934 |
| 100 nM FUSE-597 (h5A1-HK2 Fc) | 10088 |
| 100 nM FUSE-598 (h5A1-HK6 Fc) | 9555 |

TABLE 9D
Single concentration FACS screen

| B16-F10-ROR1-H | MFI |
|---|---|
| FMO | 2.14 |
| mIgG-PE | 2.08 |
| anti-human ROR1(clone 2A2)-PE | 21570 |
| 2^{nd} Anti-human IgG-PE antibody | 3.01 |
| 100 nM anti-HEL4-hIgG1 | 3.86 |
| 100 nM FUSE537 (h2A11-HK1_Fc) | 862 |

TABLE 9E
Single concentration FACS screen

| B16-F10-ROR1-H | MFI |
|---|---|
| FMO | 6.48 |
| mIgG-PE | 6.53 |
| anti-human ROR1(clone 2A2)-PE | 27104 |
| 2nd Anti-human IgG-PE antibody | 9.92 |
| 100 nM anti-hCD47 | 9.77 |
| 100 nM anti-HEL4-hIgG1 | 13.3 |
| 100 nM FUSE-179-2 (5A1 Fc) | 4475 |
| 100 nM FUSE-112-2 (2A11 Fc) | 7840 |
| 100 nM FUSE-589 (h2A11-HK5 Fc) | 6951 |
| 100 nM FUSE-590 (h5A1-FA9-HK2_Fc) | 7329 |
| 100 nM FUSE-591 (h5A1-FA9-HK3_Fc) | 7768 |
| 100 nM FUSE-592 (h5A1-FA9-HK4_Fc) | 7539 |
| 100 nM FUSE-593 (h5A1-FA9-HK5_Fc) | 7525 |
| 100 nM FUSE-594 (h5A1-FA9-HK6_Fc) | 7911 |
| 100 nM FUSE-595 (h5A1-FA9-HK7_Fc) | 7180 |
| 100 nM FUSE-596 (h5A1-FA9-HK8 Fc) | 6256 |

TABLE 9F
Single concentration FACS screen

| B16-F10-ROR1-H | MFI |
|---|---|
| FMO | 356 |
| mIgG-PE | 94.6 |
| anti-human ROR1(clone 2A2)-PE | 416000 |
| 2nd Anti-human IgG-PE antibody | 125 |
| 100 nM anti-HEL4-hIgG1 | 1135 |
| 100 nM FUSE-179-2 (5A1 Fc) | 93806 |
| 100 nM FUSE-112-2 (2A11 Fc) | 207782 |
| 100 nM FUSE-559 (h5A1-FA9-HK1 Fc) | 198683 |
| 100 nM FUSE-560 (h2A11-HK2 Fc) | 159403 |
| 100 nM FUSE-561(h2A11-HK3 Fc) | 157168 |
| 100 nM FUSE-562 (h2A11-HK4 Fc) | 155016 |
| 100 nM (BI-HU9-VHH11 h5A1 Fc) | 14127 |
| 100 nM (BI-HU9-VHH12 h5A1 Fc) | 21000 |
| 100 nM (BI-HU9-VHH13 h5A1 Fc) | 21114 |
| 100 nM (BI-HU9-VHH21 h5A1 Fc) | 15601 |
| 100 nM (BI-HU9-VHH22 h5A1 Fc) | 22260 |
| 100 nM (BI-HU9-VHH23 h5A1 Fc) | 23588 |
| 100 nM (BI-HU9-VHH31 h5A1 Fc) | 17939 |
| 100 nM (BI-HU9-VHH32 h5A1 Fc) | 20060 |
| 100 nM (BI-HU9-VHH33 h5A1 Fc) | 21956 |
| 100 nM (BI-HU9-VH9 h5A1 Fc) | 14450 |
| 100 nM (BI-HU9-VH9-1 h5A1 Fc) | 16607 |
| 100 nM (BI-HU9-HK h5A1 Fc) | 133 |
| 100 nM (BI-HU9-HK-1 h5A1 Fc) | 135858 |

TABLE 9G
Single concentration FACS screen

| LS174T-ROR1-L | MFI |
|---|---|
| FMO | 22.3 |
| 2nd Anti-human IgG-PE antibody | 22.6 |
| mIgG-PE | 23.2 |
| anti-human ROR1(clone 2A2)-PE | 7956 |
| 100 nM Human IgG1 | 30.8 |
| 100 nM FUSE-179-2 (5A1 Fc) | 836 |
| 100 nM FUSE-559 (h5A1-FA9-HK1 Fc) | 3120 |
| 100 nM FUSE-654 - h5A1-FA9-HK6-R19K Fc | 4558 |
| 100 nM FUSE-655 - h5A1-FA9-HK6-N84S Fc | 4010 |
| 100 nM FUSE-112-2 (2A11) | 4336 |
| 100 nM FUSE-661- h2A11-HK2-3-N84S Fc | 4881 |
| 100 nM FUSE-662 - h2A11-HK2-16-N84S Fc | 4351 |

TABLE 9H
Single concentration FACS screen

| LS174T-ROR1-L | MFI |
|---|---|
| FMO | 58.4 |
| mIgG-PE | 54 |
| anti-human ROR1(clone 2A2)-PE | 36206 |
| 2nd Anti-human IgG-PE antibody | 45.3 |
| 100 nM Human IgG1 | 316 |
| 100 nM FUSE-179-2 (5A1 Fc) | 3058 |
| 100 nM FUSE-559 (h5A1-FA9-HK1 Fc) | 24426 |
| 100 nM F22158101 (h5A1-BI-001 Fc) | 19719 |
| 100 nM F22158102 (h5A1-BI-002 Fc) | 20481 |
| 100 nM F22158103 (h5A1-BI-003 Fc) | 15800 |
| 100 nM F22158104 (h5A1-BI-004 Fc) | 28869 |
| 100 nM F22158105 (h5A1-BI-005 Fc) | 22268 |
| 100 nM F22158106 (h5A1-BI-006 Fc) | 17400 |
| 100 nM F22158107 (h5A1-BI-007 Fc) | 11471 |
| 100 nM F22158108 (h5A1-BI-008 Fc) | 25086 |
| 100 nM F22158109 (h5A1-BI-009 Fc) | 34983 |
| 100 nM F221581010 (h5A1-BI-010 Fc) | 18659 |
| 100 nM F221581011 (h5A1-BI-011 Fc) | 19368 |
| 100 nM F221581012 (h5A1-BI-012 Fc) | 14200 |
| 100 nM F221581013 (h5A1-BI-013 Fc) | 23838 |
| 100 nM F221581014 (h5A1-BI-014 Fc) | 28696 |
| 100 nM F221581015 (h5A1-BI-015 Fc) | 29280 |
| 100 nM F221581016 (h5A1-BI-016 Fc) | 14976 |
| 100 nM F221581017 (h5A1-BI-017 Fc) | 28569 |
| 100 nM F221581018 (h5A1-BI-018 Fc) | 17492 |
| 100 nM F221581019 (h5A1-BI-019 Fc) | 33815 |
| 100 nM F221581020 (h5A1-BI-020 Fc) | 26535 |
| 100 nM F221581021 (h5A1-BI-021 Fc) | 35788 |

### Example 6

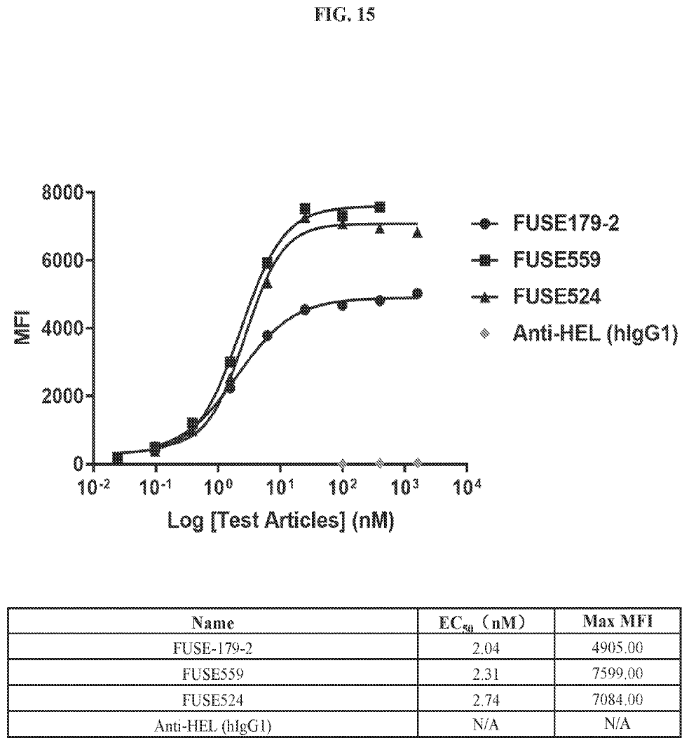

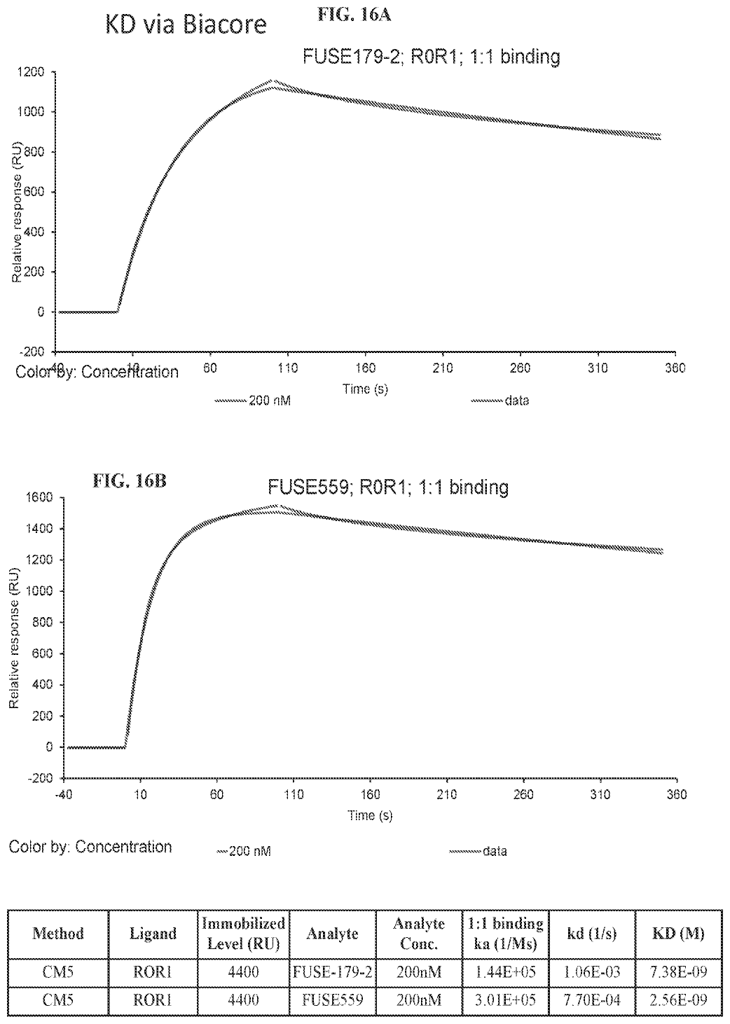

As shown in FIG. 15 the apparent binding affinity (EC50-binding) of the ROR1 specific VHH-Fc incorporating either the non humanized VHH clone 5A1 (FUSE179; black circle), the humanized clone 5A1-FA9-HK1 (FUSE559; black square) or the humanized clone 5A1-HK1 (FUSE524; black triangle) was calculated. Shown is a non-linear x-y plot of binding to B16-F10 cell engineered to express human ROR1 relative to the concentration of test article used for staining. A Hen Egg Lysozyme (HEL)-specific antibody was used as the negative isotype control. Binding was measured via flow cytometry using a fluorescently labeled secondary antibody specific for the Fc domain of IgG1. A summary table of EC50s and maximum MFI is shown beneath under the graph.

### Binding Assay by Surface Plasmon Resonance (SPR)

Protein test article binding to recombinant human ROR1 (Acro Biosystems, RO1-H522y) was detected on a BIAcore 8K (Cytiva) biosensor system using an SPR assay. Briefly, a carboxymethylated CM5 sensor chip was activated with 1:1 mixture of 0.4 M N-ethyl-N-(3-dimethylaminopropyl) carbodiimide and 0.1 M′ N-hydroxysuccinimide. Recombinant human ROR1 in 10 mM NaOAc (pH5) was then immobilized on the sensor chip by amine-coupling according to the manufacturer's instructions to yield the stable RU of 4400. Unreacted sites were blocked with 1M ethanolamine/HCl (pH 8). Control flow cells (blank) were activated and blocked in the absence of ROR1. Flow cells were routinely equilibrated with running buffer (PBS, 0.005% surfactant P20). Test article proteins used for assessment of binding to recombinant human ROR1 binding were diluted in HBS-EP running buffer and injected into the sensor chip at different concentrations. Kinetic and affinity data of ROR1/test article binding were obtained at a flow rate of 30 μL/min, with a set association time of 100 sec and a dissociation time of 200 sec. Data from duplicate assays were modeled for binding equilibrium and dot plots for 200 nM of protein test article was calculated. Association constant (ka), dissociation constant (kd) and binding affinity (KD=kd/ka) were generated from integrated software in the BIAcore 8K system.

### PD-1, TIGIT, and CD69 Detection on T Cells

Following the experiment setting up for cytotoxicity assay, target cells were added to the wells (5,000 target cells per well) in 96-wells plate and incubated for 5 hours. After 5-hour incubation, cell culture medium was removed from the plate without touching the target cells and 50 uL of fresh PBMCs (150,000 PBMCs per well) were added to the wells with target cells (E:T ratio 0f 30:1), 50 uL of CD3×ROR1 TCEs were then added to the designated wells with 150,000 PBMCs and 5,000 target cells. After incubating at 37° C. for 24, 48 or 72 hours, PBMCs were collected and washed once with PBS by centrifugation at 500×g for 5 minutes at room temperature and added to V-bottom 96-well plate. To prepare Live/Dead fixable red, one vial of powder was dissolved into 50 uL of DMSO to make stock solution and then diluted 1 uL of stock Live/Dead fixable red in 1 mL of PBS to prepare the working solution. 100 uL of Live/Dead fixable red working buffer was then added to the cell in the V-bottom 96-well plate and incubated for 15 minutes at room temperature. After the 15-minute incubation, the cells were washed twice with PBS by centrifugation at 500 g for 5 minutes at room temperature. The cells were then cultured with antibodies (PE-Cy5-conjugated mouse anti-human TCRa/b antibody, PE-conjugated mouse anti-human CD19 antibody, BB515-conjugated mouse anti-human CD56 antibody, BB700-conjugated mouse anti-human CD14 antibody, PE-Cy7-conjugated mouse anti-human PD-1, TIGIT or CD69 antibody) in 100 uL of Biolegend staining buffer at 4° C. for 45 minutes. After the 45-minute incubation with antibody, the cells were washed with PBS by centrifugation at 500 g for 5 minutes at room temperature and resuspended in 100 uL of BioLegend fixation buffer. The samples were then analyzed on Cytek flow cytometer. Data were analyzed with FlowJo and the gating strategy was followed: In brief, target cell population was gated by FSC and SSC. Single cells were selected from target cell population by FSC-A and FSA-H. Living single cells were gated from single cells by live/dead red dye low. T cells were gated from living single cells by TCRa/b high. PD-1, TIGIT and CD69 expression was then gated on T cells with PD-1, TIGIT or CD69 high.

### Example 7

As shown in FIG. 16, the affinity (KD) of the ROR1 specific VHH-Fc incorporating either the non humanized VHH clone 5A1 (FUSE179; FIG. 16A) or the humanized clone 5A1-FA9-HK1 (FUSE559; FIG. 16B) was calculated. Shown are sensorgrams of surface plasmon resonance (SPR) data generated via Biacore analysis. A summary table of binding affinity (KD), on rate (k-a) and off rate (kd) is shown below FIG. 16B.

### Example 8

As shown in FIG. 17, we assessed the apparent binding affinity (EC50-binding) of the two bispecific antibodies (bsAbs), FUSE488 (humanized CD3 clone 1F3.3×ROR1 VHH 5A1) and FUSE608 (humanized CD3 clone 1F3.3×humanized clone 5A1-FA9-HK1) for human ROR1 (FIG. 17A) and human CD3 (FIG. 17B).

FIG. 17A is a non-linear x-y plot depicting binding of FUSE488 (black triangle) or FUSE608 (black circle) to the human TNBC ROR1+ MDA-MB-231 cell line at various concentrations of test articles. FUSE608 demonstrated appreciably greater apparent affinity for ROR1 than FUSE608. This may be attributed to the “optimization” of the Vernier zone residues as well as the amino acid substitution in CDR3 of 5A1-5A9-HK1, D103H.

FIG. 17B is a non-linear x-y plot depicting binding of FUSE488 (black triangle) or FUSE608 (black circle) to the human lymphoma CD3+ Jurkat cell line (which has been engineered to express Lucia luciferase downstream of an NEAT promoter) at various concentrations of test articles. Given that both FUSE488 and FUSE608 use the same CD3 binder and incorporate the format, it is not surprising that we observed no appreciable difference in apparent binding for CD3 between the two bsAbs.

An Hen Egg Lysozyme (HEL)-specific antibody was used as the negative isotype control. Binding was measured via flow cytometry using a fluorescently labeled secondary antibody specific for the Fc domain of IgG1.

A summary table of EC50s and maximum MFI is shown beneath each graph.

### Example 9

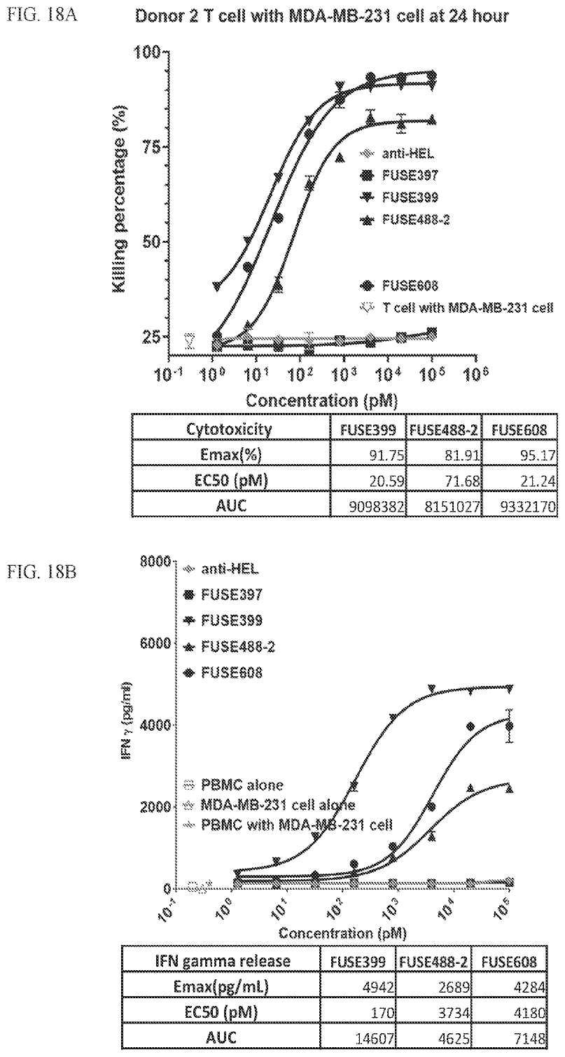

As shown in FIG. 18, we examined the capacity of several ROR1×CD3 specific bsAbs to induce (1; FIG. 18A) T cell mediated killing of ROR1+ MDA-MB-231 tumor cells and (2; FIG. 18B) T cell mediated release of IFNγ when co-mixed with ROR1+ MDA-MB-231 tumor cells. Both assays were read out at 24 hours. The test articles examined were (a) the non decoupled ROR1×1F3-3 benchmark bsAb, FUSE399 (non humanized Kringle domain specific scFV, R11, x humanized 1F3-3 bsAb), (b) FUSE488 (non humanized 5A1×1F3-3 bsAb in format Ac13) and (c) FUSE608 (humanized CD3 clone 1F3-3×humanized clone 5A1-FA9-HK1). The negative controls tested were non targeted 1F3-3 control, FUSE397 and the human IgG1 anti-HEL isotype control antibody. The assay system utilized expanded T cells purified from healthy donors.

Shown are exemplary non-linear x-y plots of tumor cell killing (FIG. 18A) and IFNγ release (FIG. 18B) whereby the rank order of both potency (EC50) and efficacy (Emax) were observed to as FUSE399 (black reverse triangle)>FUSE608 (black circle)>FUSE488 (black triangle). Importantly, the difference in the potency/efficacy of tumor cell killing between FUSE608 and FUSE399 was not appreciable. In contrast, FUSE608 was about 25× less potent at IFNγ release than FUSE399 indicating that a concentration range of FUSE608 was observed in which maximum tumor cell killing can be achieved without concomitant maximum release of IFNγ. This property associated with FUSE608, which may be termed “decoupling of cytotoxicity from cytokine release” might reduce the risk of toxic side effects, such as Cytokine Release Syndrome (CRS), associated with CD3 bsAbs, without sacrificing tumor cell killing. An appreciable concentration range with similar properties was not observed for FUSE399, which was specifically chosen as the “non-decoupled benchmark”.

Little to no activity was observed in either assay with FUSE397 (black square) nor the anti-HEL (gray diamond) negative control test article. Summary tables of EC50, Emax and AUC for each test article are shown below each graph.

### Example 10

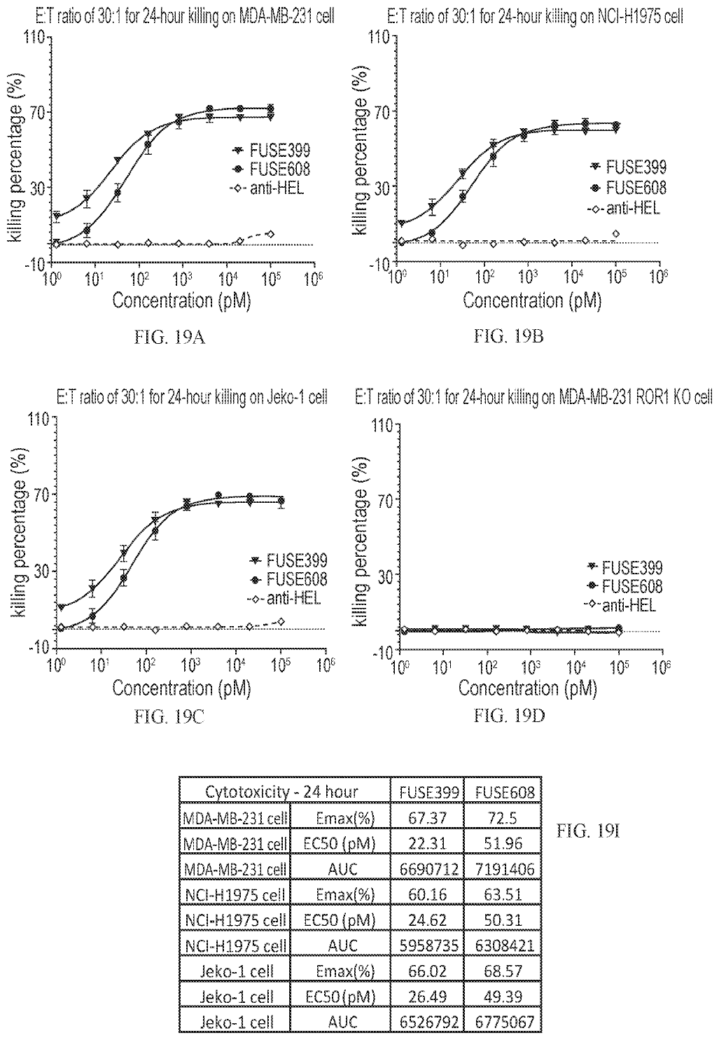

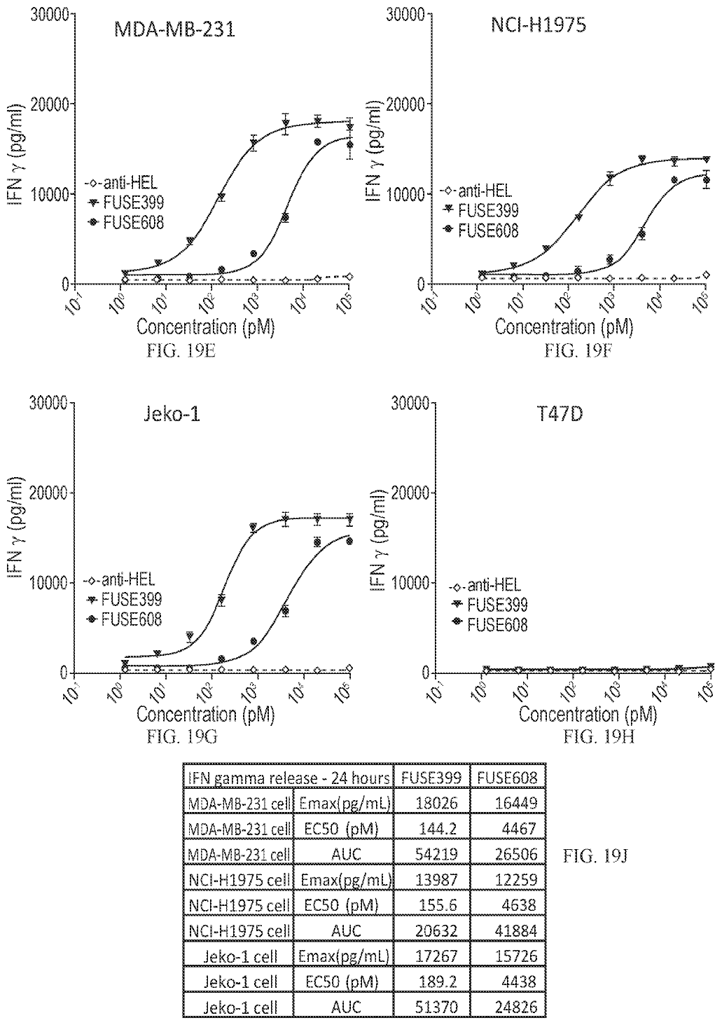

As shown in FIG. 19, we examined the capacity of several ROR1×CD3 specific bsAbs to induce (1; FIGS. 19A-19D) PBMC mediated killing of four different tumor cell lines and (2; FIGS. 19E-19H), PBMC mediated release of IFNγ when co-mixed with the aforementioned tumor cells. Both assays were read out at 24 hours. The test articles examined were (a) the non decoupled ROR1×1F3-3 benchmark bsAb, FUSE399 (non humanized Kringle domain specific scFV, R11, x humanized 1F3-3 bsAb) and (b) FUSE608 (humanized CD3 clone 1F3-3×humanized clone 5A1-FA9-HK1). The human IgG1 anti-HEL antibody served as the negative isotype control. The assay system utilized PBMC purified from healthy donors and the following different tumor cell lines: (1) the ROR1+ triple negative breast cancer (TNBC), MDA-MB-231 (FIG. 19A, 19E), the ROR1+ non-small cell lung cancer (NSCLC) NCI-H1975 (FIG. 19B, 19F), ROR1+ mantle cell lymphoma, JeKo-1 (FIG. 19C, 19G), and ROR1 negative breast cancer, T-47D (FIG. 19D, 19H). The rank order cell surface expression of ROR1 was as follows: JeKo-1>MDA-MB-231>NCI-H1973>T-47D. As shown in the exemplary non-linear x-y plots labelled as FIGS. 19A-19D, we observed that FUSE608 (black circle) induced potent (EC50) and efficacious (Emax) PBMC mediated ROR1 dependent tumor cell killing within 3 fold the EC50 and within 5% the Emax attributed to the benchmark FUSE399 (black reverse triangle). No non-specific killing was associated with the anti-HEL control nor was any observed with FUSE399 or FUSE608 in the context of the ROR1 negative target, T-47D. In contrast, as shown in FIGS. 19E-19H, FUSE608 was between 25-30× less potent at IFNγ release than FUSE399 across the three ROR1 positive tumor cell lines examined. This result indicated that a concentration range of FUSE608 in which maximum tumor cell killing can be achieved without concomitant maximum release of IFNγ is not a singular observation related to MDA-MB-231 but can rather be extended to all the ROR1+ tumor cell lines tested. No appreciable concentration range with similar properties was observed for FUSE399. Further, No non-specific IFNγ release was associated with the anti-HEL control nor was any observed with FUSE399 or FUSE608 in the context of the ROR1 negative target, T-47D.

Summary tables of EC50, Emax and AUC for each test article are shown below FIGS. 19A-D and FIGS. 19E-H (FIGS. 19I and 19J).

### Example 11

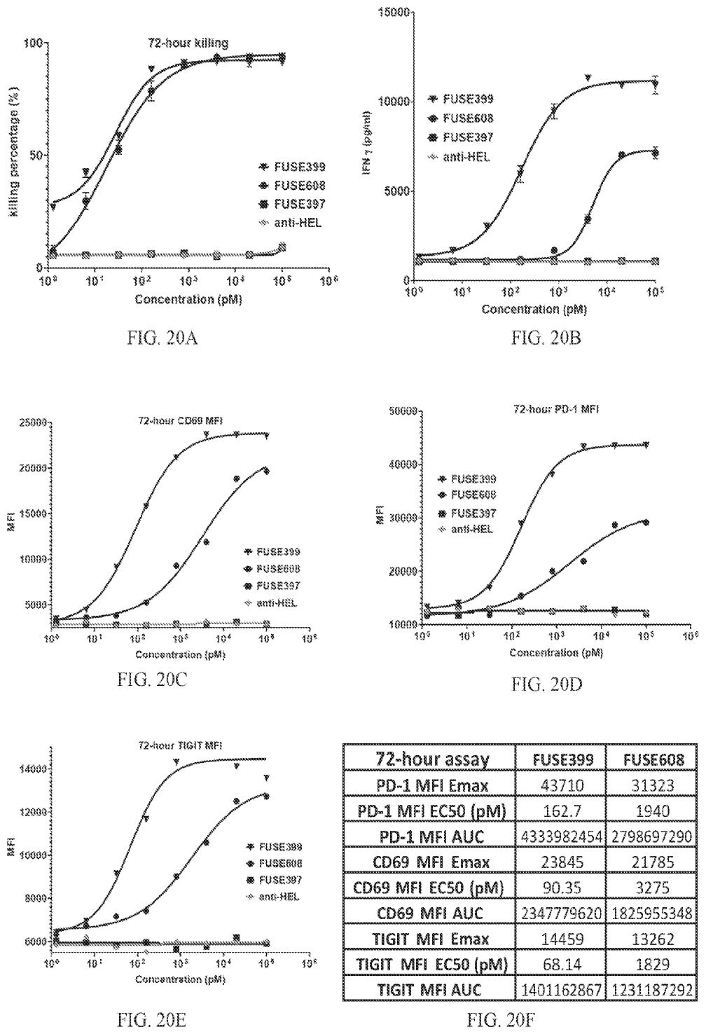

As shown in FIG. 20, we examined the capacity of several ROR1×CD3 specific bsAbs to induce (1; FIG. 20A) PBMC mediated killing of ROR1+ MDA-MB-231 tumor cells and (2; FIG. 20B), PBMC mediated release of IFNγ when co-mixed with ROR1+ MDA-MB-231 tumor cells, (3. FIG. 20C) upregulation of cell surface CD69 expression on T cells within PBMC when co-mixed with ROR1+ MDA-MB-231 tumor cells, (4. FIG. 20D) upregulation of cell surface PD-1 expression on T cells within PBMC when co-mixed with ROR1+ MDA-MB-231 tumor cells, and (5. FIG. 20E) upregulation of cell surface TIGIT expression on T cells within PBMC when co-mixed with ROR1+ MDA-MB-231 tumor cells. All readouts were assessed at 72 hours. The test articles examined were (a) the non decoupled ROR1×1F3-3 benchmark bsAb, FUSE399 (non humanized Kringle domain specific scFV, R11, ×humanized 1F3-3 bsAb) and (b) FUSE608 (humanized CD3 clone 1F3-3×humanized clone 5A1-FA9-HK1). The human IgG1 anti-HEL antibody served as the negative isotype control. We observed that the potency (EC50) and efficacy (Emax) of tumor cell killing was not appreciably different between FUSE608 (black circle) and FUSE399 (black reverse triangle). Importantly, in a similar fashion to the “decoupling of cytotoxicity from cytokine (IFNγ) release” observed for FUSE608 but not the non-decoupled benchmark CD3 bsAb, FUSE399, (see FIG. 20B) we observed that upregulation of cell surface CD69, PD-1 and TIGIT on T cells within the PBMC co-mixed with MDA-MB-231 and test article was also decoupled from tumor mediated killing. That is, FUSE608 was about 10×, 30× and 27× less potent at inducing the upregulation of CD69, PD-1 and TIGIT, respectively, compared to FUSE399. These results indicate that a concentration range of FUSE608 was observed in which maximum tumor cell killing can be achieved without concomitant maximum upregulation of CD69, PD-1 and TIGIT. An appreciable concentration range with similar properties was not observed for FUSE399. This may be of physiological significance because CD69 (pubmed.ncbi.nlm.nih.gov/30085193/), PD-1 (www.ncbi.nlm.nih.gov/pmc/articles/PMC5341794/) and TIGIT (jitc.bmj.com/content/8/2/e000957) have all been reported to be markers of T cell (and NK cell) “exhaustion” and the treatment of tumor bearing mice with antagonist antibodies specific for each protein has been reported to mediate tumor growth inhibition.

No non-specific tumor cell killing, IFNγ release nor induction of CD69, PD-1 or TIGIT was associated with the anti-HEL negative control isotype control antibody.

Summary tables of EC50, Emax and AUC for each test article are shown following FIGS. 8A-8E (FIG. 20F)

### Example 12

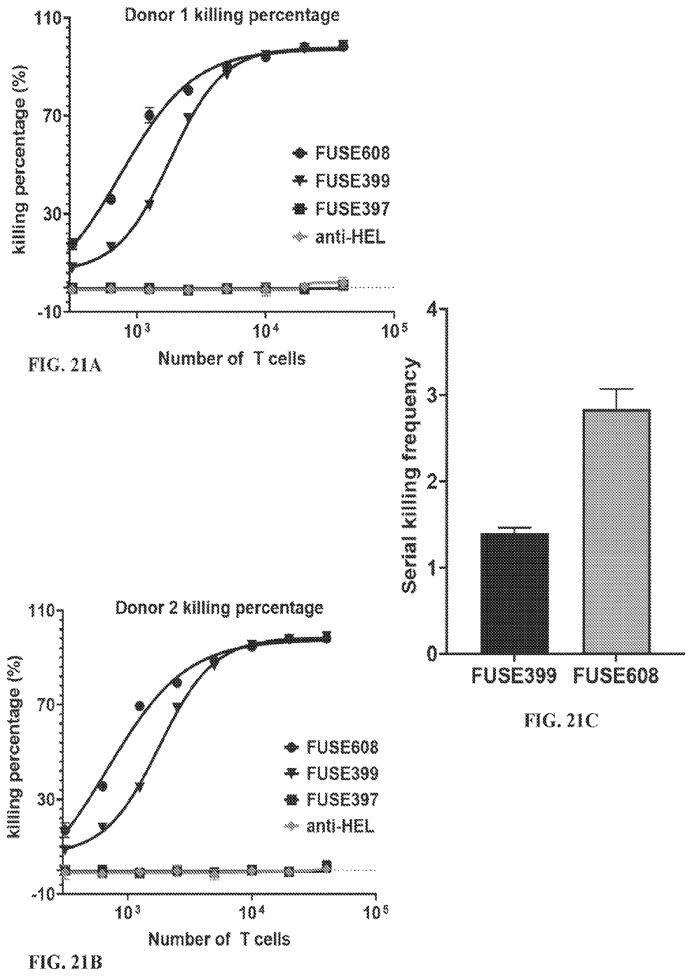

As shown in FIG. 21, we examined the capacity of FUSE399 (black reverse triangle) and FUSE608 (black circle) to induce CD8+ T cell mediated killing of a constant number (5000 cells) of ROR1+ MDA-MB-231 cells across a range of CD8+ T cell numbers from 313 to 40,000. This allows for the calculation of (a) the number of T cells needed for a given ROR1×CD3 bsAb to induce T cell mediating killing of a given amount of ROR1+ tumor cells (FIGS. 21A and 21B) and (b) the average number of tumor cells killed per T cell, also termed “serial killing” (FIG. 21C). Tumor cell viability was measured after 24 hours of co-culture. Effector to target ratios of 1:16, 1:8, 1:4, 1:2, 1:1, 2:1, 4:1 and 8:1 were analyzed. FUSE397 (black square) and anti-HEL (gray diamond) were used as the negative control test articles. Two distinct healthy donor derived expanded CD8+ T cells were tested. Donor 1 and 2 are shown in FIGS. 21A and 21B, respectfully. At E:T ratios of 1:2 to 8:1 no significant difference in tumor cell killing efficacy (Emax) was observed. At lower E:T ratios, FUSE608 was observed to induce greater T cell mediated ROR1 dependent tumor cell killing than FUSE399. For example, to kill approximately 30% of the tumor cells, FUSE399 required about 1100 T cells, whereas FUSE608 required about 450 T cells. At very low E:T ratios (e.g. 1:16 and 1:8), each T cell, on average, killed more than one tumor cell. For example, at an E:T ratio of 1:16 (312 T cells added to the assay), FUSE608 was associated with 18% killing, which equates to approximately 900 tumor cells or about 3 tumor cells killed per T cell per 24 hours.

FIG. 21C is a bar chart illustrating the average serial killing associated with FUSE399 and FUSE608 derived from the T cells from each individual donor shown in FIG. 21A and FIG. 21B. The value is reported as the number of tumor cells killed per T cell over a 24 hour period. It was calculated as the average value from the E:T ratios of 1:16 and 1:8. FUSE608 supports serial killing at approximately 3 tumor cells/T cell, whereas this number is less than 1.5 tumor cells/T cell for FUSE399. The phenomenon whereby a single T cell kills more than one tumor cell is especially important in solid tumors, where the number of T cells may be sparse with E:T ratios ranging from 1:2 to <1:8.

### Example 13

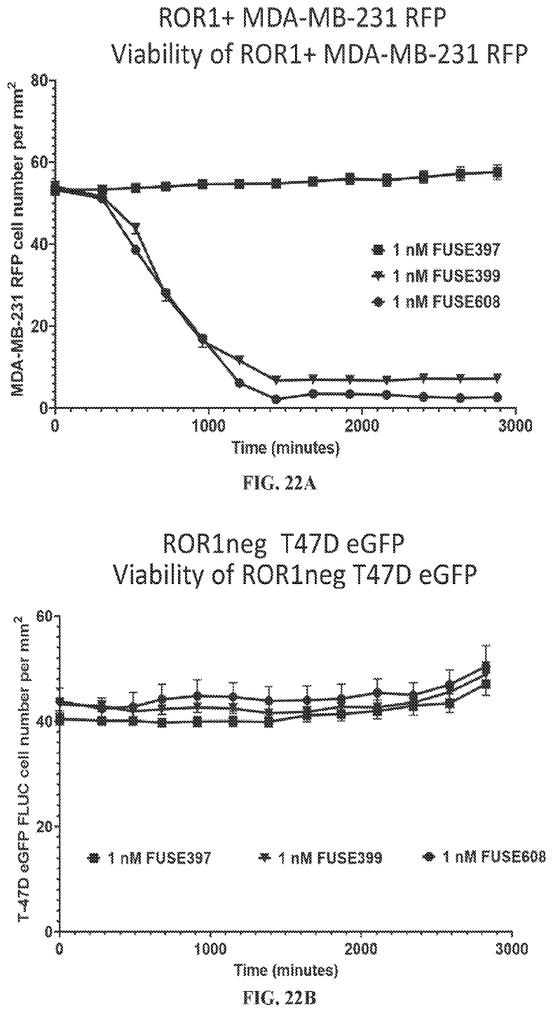

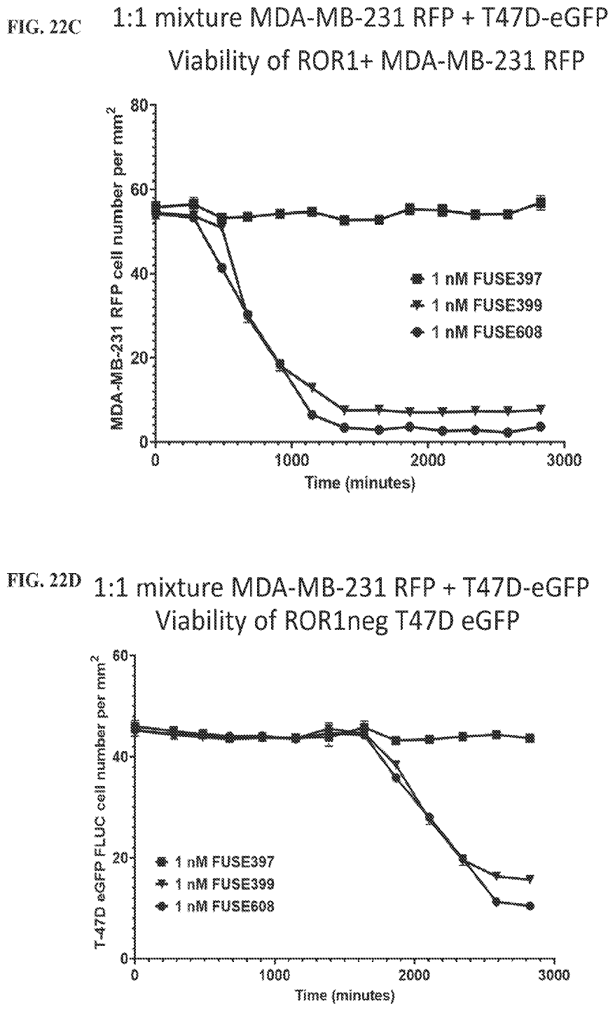

As shown in FIG. 22, we addressed whether FUSE608 induced bystander killing of ROR1 negative tumors co-mixed with ROR1 positive tumors. Bystander killing has been reported to be induced by death receptors, impacted by adhesion molecules and that the upregulation of both is dependent on IFNγ (journals.plos.org/plosone/article?id=10.1371/journal.pone.0183390). Therefore, assessment of this parameter is important for a CD3 bsAb such as FUSE608 that has been engineered for decoupling of cytotoxicity from cytokine release. A query is whether FUSE608 induce sufficient IFNγ release to support bystander killing.

As such, we examined the kinetics by which FUSE608 (black circle) and FUSE399 (black reverse triangle; the non-decoupled benchmark ROR1×CD3 bsAb) induced expanded T cell mediated killing of ROR1+ MDA-MB-231 tumor cells (FIG. 22A), ROR1-negative T-47D tumor cells (FIG. 22B) or a 1:1 mixture of both cells (FIGS. 22C-22D). Shown are x-y plots of tumor viability on the y-axis and time on the x-axis. To distinguish between ROR1 positive MDA-MB-231 and ROR1 negative T-47D cells, the former were engineered to express RFP and the latter to express eGFP. FUSE397 (black square) was used as the negative control.

As seen in FIG. 22A and consistent with the results from our previous killing assays, in the presence of expanded T cells and MDA-MB-231 RFP alone, both FUSE399 and FUSE608 (but not the control test article, FUSE397) induced strong T cell mediated killing of ROR1+ MDA-MB-231 RFP with potencies (IC50_{VIABILITY}) and Imax values that were not appreciably different from one another. Also consistent with our previous observations, when ROR1-negative T-47D eGFP cells were co-cultured with expanded T cells, neither FUSE608 nor FUSE399 (or the control test article, FUSE397) induced loss in T-47D eGFP viability (FIG. 22B), indicating that the killing observed with MDA-MB-231 RFP was ROR1 dependent.

To examine bystander killing, expanded T cells were used as effector cells in co-culture with a 1:1 mixture of ROR1-positive MDA-MB-231 RFP and ROR1-negative T-47D cGFP. Test articles were tested at multiple concentrations. Shown is the 1 nM concentration because this level was previously observed to be the most consistent concentration at which both FUSE608 and FUSE399 induced similar maximum killing of MDA-MB-231 but dramatically differed in IFNγ release. That is, at 1 nM, FUSE399 reached maximum IFNγ release but the release of IFNγ by FUSE608 was below its EC50 value and therefore well within the decoupled portion of its curve.

Here, we observed that FUSE608 was as efficient at inducing direct killing of ROR1-positive MDA-MB-231 RFP (FIG. 22C) and bystander killing of ROR1-negative T-47D eGFP (FIG. 22D) as FUSE399. This indicates that although FUSE608 can be dosed for lower IFNγ release than FUSE399 while maintaining maximum killing of ROR1+ tumors, FUSE608 still induces sufficient IFNγ to mediate bystander killing. No loss in either tumor cell viability was observed with the negative control test article, FUSE397.

Killing of ROR1-positive MDA-MB-231 RFP (FIG. 22D) when admixed with T-47D eGFP occurred with similar kinetics as that observed without co-mixture of T-47D eGFP cells (see FIG. 22A). Such “direct killing” has been previously shown to be perforin/granzyme mediated (journals.plos.org/plosone/article?id=10.1371/journal.pone.0183390). In contrast, bystander killing of ROR1-negative T-47D eGFP only occured when co-mixed with MDA-MB-231 and is delayed with an

IC50_{VIABILITY} of about 36 hours (i.e., approximately 22 hours after perforin/granzyme mediated killing of MDA-MB-231 RFP). This timing is consistent with a previous report describing CD3 bsAb induced IFNγ dependent bystander killing (journals.plos.org/plosone/article?id=10.1371/journal.pone.0183390).

### Example 14

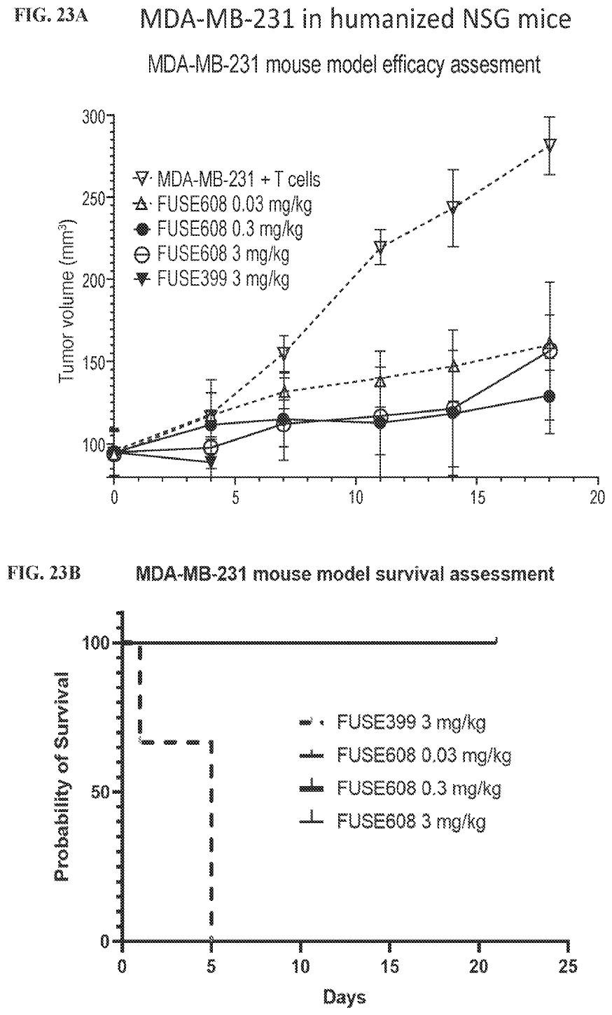

As shown in FIG. 23, we examined the capacity of FUSE608 and the non-decoupled benchmark ROR1×CD3 bsAb, FUSE399, to mediate tumor growth inhibition (TGI) in a mouse tumor xenograft model. NSG mice were subcutaneously implanted with the human ROR1+ TNBC cell line, MDA-MB-231 and intravenously reconstituted with expanded human T cells from a healthy human donor. T cells from several human donors were first screened to identity those with minimal “background” alloreactivity against MDA-MB-231 but which induced potent ROR1 dependent tumor cell killing when co-mixed with FUSE399 or FUSE608 in a similar fashion to that observed in FIG. 18.

When the tumor volume reached ˜100 mm3, mice were treated as such: group 1 (gray reverse open triangle) received PBS, group 2 (open circle) received 3 mg/kg of FUSE608, group 3 (black closed circle) received 0.3 mg/kg of FUSE608, group 4 (gray closed triangle) received 0.03 mg/kg of FUSE608 and group 5 (black reverse triangle) received 3 mg/kg of FUSE399. Tumor volume and survival as a function of time are shown in FIGS. 23A and 23B, respectively. Test articles were administered intraperitoneally twice a week.

We observed highly appreciable TGI from FUSE608 with dose levels of 0.3 mg/kg and 3 mg/kg associated with slightly better efficacy than 0.03 mg/kg.

Importantly, all mice treated with FUSE608 survived whereas all mice treated with FUSE399 died within the first 5 days after the first treatment from toxicity consistent with CRS.

### Example 15

### Humanization Libraries of V_{H}H Clones 5A1 and 2A11

Many tumors, such as lung or breast cancer, show increased expression of ROR1. ROR1, which is normally only expressed during embryogenesis except for hematogones, plays a crucial role in cell proliferation, migration, and survival. Two ROR1 targeting VHH variants (2A11 and 5A1) were obtained from a Camelus dromedarius immunization campaign and contained a putative non-canonical disulfide bridge between CDR1 and CDR3, which is present in about 30% of all V_{H}Hs of HCAbs from Camelus dromedarius. The position of an additional disulfide bridge depends on the camelid species. In Camelus dromedarius, for example, it is located between the CDR1 and CDR3 loops, whereas in llama V_{H}Hs, the CDR3 loop is linked to the CDR2 loop. The two V_{H}H clones have a 71.3% sequence identity (FIG. 1F). Despite their many desirable properties, the non-human origin of V_{H}H could lead to the development of undesirable immune responses upon therapeutic administration. Thus, to broaden the range of therapeutic applications, humanization is a promising method. The first comprehensive report describing the humanization of V_{H}Hs was published by Vincke et al. in 2009. In their study, they proposed two different general methods for the humanization of V_{H}Hs: CDR grafting and resurfacing (Veneering). In the grafting approach, the CDRs of a V_{H}H are grafted onto another human or humanized framework (16A). In the Veneering strategy, residues in the framework regions of the camelid V_{H}H are substituted to mimic the sequence of human V_{H}s.

The putative CDR loops were determined based on a statistical comparison of V_{H}Hs by Melarkode Vattekatte et al. In the context of humanization, undesirable effects such as affinity attenuation or impaired protein stability may occur due to the modulation of framework regions. Therefore, so-called Vernier residues are of great importance for specificity and affinity. They underlie the CDRs and can potentially impact the conformation of the CDR loops. It has been shown that back-mutations of Vernier residues to the non-human amino acid can restore the original conformation of CDR loops.

For each clone, eight putative Vernier positions underlying the CDR loops were proposed based on the work of Vincke et al., Soler et al., and Sang et al., as well as on the comparison with the crystal structure of a similar VHH. The nearest human framework (FR), which is IGHV3-23*01 in both cases, was identified by utilizing Ig-blast (www.ncbi.nlm.nih.gov/igblast/; accessed 1 Jun. 2022). For clone 2A11, the sequence identity is 71.1%, and for 5A1, it is 64.9%. To identify which human amino acids should be allowed besides the camelid one at each Vernier position, the five human frameworks with the highest sequence identity were chosen as templates. A characteristic difference between human V_{H} and camelid V_{H}H is the replacement of conserved hydrophobic amino acids in the FR2 (V42F; G49E; L50R; W52G). Conserved key residues in FR2 were not altered, except for glutamate to leucine at position 52, as this increased VHH stability in the past.

Further substitutions with potential beneficial effects for the VHH properties were made. For example, a reduced aggregation could be observed for the Q1E mutation in framework region 1 (FR1) and the exchange to a hydrophobic residue at position 5 residue has been shown to improve stability. In FR4, the exchange of tryptophan to arginine can improve solubility and reduce aggregation by masking hydrophobic residues. Thus, 16 residues outside from the Vernier residues were replaced in the 5A1 humanization library. In the 2A11 humanization library, seven such residues were altered. The minimal guaranteed identity to the human germline gene IGHV3-23 with this library approach is 75.3% with consideration of the CDR regions and 87.6% without these regions.

For the generation of yeast surface display libraries, the protocol of Benatuil et al. was utilized. Inserts were obtained via a PCR assembly reaction with primers containing degenerated codons. They were designed to allow the selected human amino acids and the camelid residue at the Vernier positions (FIG. 15). Yeast Surface Display takes advantage of eukaryotic post-translational modification machinery. Folding in the oxidizing environment of the endoplasmatic reticulum (ER) and glycosylation enhances the presentation of natively proteins. Furthermore, chaperones and foldases assist in the presentation of correctly folded, more complex proteins. This also allows the surface presentation of multidomain proteins containing multiple disulfide bridges. This makes YSD an efficient method for screening antibody libraries. The function of displayed proteins can be rapidly and quantitatively determined by using fluorescence activated cell sorting (FACS). FACS-based screening over several rounds was used to find functional variants.

### Simultaneous Vernier-Optimization and Affinity Maturation Based on FUSE497

The computational humanized clone FUSE497, showed improved expression properties compared to the parental 5A1 clone. Therefore, this clone was used as template for an additional humanization campaign. The FUSE497-based library (FIG. 16) allows for the optimization of Vernier residues, the potential increasement of the proportion of human residues and affinity maturation by randomization of a position in CDR3 with a degenerated NNS codon. New putative Vernier residues were introduced in the positions marked in orange compared to the previous libraries. On the one hand, this allows back mutations at potential Vernier positions and on the other hand, it enables an increase in sequence homology with the human germline gene IGHV-3-23*01 compared to the template FUSE497. They were identified as described above.

The insert for the library was generated with degenerated primers, as described in FIG. 5. Twelve different PCRs with varying NNS codons in the CDR3 region were performed and mixed in a stochiometric ratio.

### Yeast Library Generation

Yeast library generation was performed according to the protocol published by Benatuli et al. in 2010. 50 mL of YPD medium were inoculated with S. cerevisiae EBY100 and incubated overnight at 30° C. and 200 rpm. The next day, 1 L YPD Medium was inoculated with the pre-culture to a starting OD₆₀₀ of 0.3 and incubated at 30° C. and 200 rpm until an OD₆₀₀ of 1.6 was reached. The cells were sedimented by centrifugation at 4000 rpm for 3 min. After washing twice with ice-cold water and once with ice-cold electroporation buffer (1 M sorbitol; 1 mM CaCl₂), cells were collected in 250 mL resuspension buffer (0.1 M LiAc; 10 mM DTT) and incubated at 30° C. and 200 rpm for 30 min. Finally, the cells were washed four times with ice-cold electroporation buffer, collected in 10 mL electroporation buffer, and kept on ice until electroporation. For each electroporation, to 400 μL of the electrocompetent EBY100 solution, 12 ug insert and 4 ug restricted vector (pCT digested by Bamh I and Nhe I) was added. After transfer to an ice-cold electroporation cuvette, the mixture was incubated for 5 min on ice. Afterward, cells were electroporated at 200 Ω, 2.5 kV, 25 μF, transferred to 8 mL of a 1:1 mixture of 1 M Sorbitol: YPD, and incubated at 30° C. and 200 rpm for 1 h. To determine the library size, a dilution series was prepared up to a dilution of 10-9. Dilution 10⁻⁴ to 10⁻⁹ were plated on selective SD agar plates. After an incubation of 3 days at 30° C., the colonies were calculated to determine the number of transformed cells. The rest of the regenerated cells were transferred to 1 L of selective SD medium (5.4 g/L Na₂HPO₄; 8.6 g/L NaH₂PO₄; 20 g/L glucose; 5 g/L ammonium sulfate; 1.7 g/L yeast nitrogen base; 5 g/L Bacto Casamino Acids (−Trp)) and incubated for 2 d at 30° C. and 200 rpm.

### Induction of Gene Expression for YSD

Surface presentation of V_{H}Hs was mediated via Aga2p (FIG. 18). Gene expression of those linked proteins was controlled by the Gall promoter. Therefore, expression of the Aga2p fusion protein is induced by galactose in the culture medium. In order to start to display gene expression, cells were grown in SG medium (5.4 g/L Na₂HPO₄; 8.6 g/L NaH₂PO₄; 20 g/L galactose; 5 g/L ammonium sulfate; 1.7 g/L yeast nitrogen base; 5 g/L Bacto Casamino Acids (−Trp)) or a mixture of 4:1 SG: SD medium at 30° C. and 200 rpm for 12 to 48 h.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention. While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A polypeptide, comprising:
a polypeptide having a complementarity-determining region (CDR) 1, a polypeptide having a CDR2, and a polypeptide having a CDR3 selected from Table 8A or Table 8B, wherein CDR1, CDR2 and CDR3 are selected from the same row in Table 8A or Table 8B; OR
a variant of the polypeptide having the polypeptide having the CDR1, the polypeptide having the CDR2, and the polypeptide having the CDR3, wherein the variant of the polypeptide having the CDR1 comprises one or more deletions, additions or substitutions of an amino acid residue in the polypeptide having the CDR1, wherein the variant of the polypeptide having the CDR2 comprises one or more deletions, additions or substitutions of an amino acid residue in the polypeptide having the CDR2, and wherein the variant of the polypeptide having the CDR3 comprises one or more deletions, additions or substitutions of an amino acid residue in the polypeptide having the CDR3, wherein CDR1, CDR2 and CDR3 are selected from the same row in Table 8A or Table 8B.

2. The polypeptide of claim 1, wherein the polypeptide comprises:
a polypeptide having a CDR1 of SEQ ID NO:4, a polypeptide having a CDR2 of SEQ ID NO: 77, and a polypeptide having a CDR3 of SEQ ID NO:6; OR
a polypeptide having a CDR1 of SEQ ID NO:73, a polypeptide having a CDR2 of SEQ ID NO: 77, and a polypeptide having a CDR3 of SEQ ID NO:84; OR
a polypeptide having a CDR1 of SEQ ID NO:73, a polypeptide having a CDR2 of SEQ ID NO: 77, and a polypeptide having a CDR3 of SEQ ID NO:85; OR
a polypeptide having a CDR1 of SEQ ID NO:73, a polypeptide having a CDR2 of SEQ ID NO: 5, and a polypeptide having a CDR3 of SEQ ID NO:84; OR
a polypeptide having a CDR1 of SEQ ID NO:73, a polypeptide having a CDR2 of SEQ ID NO: 5, and a polypeptide having a CDR3 of SEQ ID NO:85.

3. The polypeptide of claim 1, further comprising framework region (FWR) 1, framework region 2, framework region 3, and framework region 4 selected from Table 8B, and wherein FWR1, FWR2, FWR3, and FWR4 are selected from the same row in Table 8B.

4. The polypeptide of claim 1,
wherein the polypeptide is selected from Table 7, or a variant of the polypeptide selected from Table 7,
wherein the variant comprises one or more deletions, additions or substitutions of an amino acid residues of the polypeptide, or
wherein the variant is at least 95% identical to the polypeptide selected from Table 7.

5. The polypeptide of claim 4, wherein the variant comprises up to 5 deletions, additions or substitutions of an amino acid residues of the polypeptide.

6. (canceled)

7. The polypeptide of claim 1, wherein the polypeptide comprises a polypeptide having SEQ ID NO: 16, SEQ ID NO:39, SEQ ID NO:40, or SEQ ID NO:60.

8. A polypeptide, comprising:
a polypeptide having SEQ ID NO:1 (complementarity-determining region (CDR) 1 of 2A11), a polypeptide having SEQ ID NO:2 (CDR2 of 2A11), a polypeptide having SEQ ID NO:3 (CDR3 of 2A11), or a combination thereof; OR
a variant of the polypeptide having SEQ ID NO:1 (CDR1 of 2A11), a variant of the polypeptide having SEQ ID NO:2 (CDR2 of 2A11), a variant of the polypeptide having SEQ ID NO:3 (CDR3 of 2A11), or a combination thereof, wherein the variant of the polypeptide having SEQ ID NO:1 comprises one or more deletions, additions or substitutions of an amino acid residue in the polypeptide having SEQ ID NO:1, wherein the variant of the polypeptide having SEQ ID NO:2 comprises one or more deletions, additions or substitutions of an amino acid residue in the polypeptide having SEQ ID NO:2, wherein the variant of the polypeptide having SEQ ID NO:3 comprises one or more deletions, additions or substitutions of an amino acid residue in the polypeptide having SEQ ID NO:3, and
wherein:
the variant of the polypeptide having SEQ ID NO: 1 and the variant of the polypeptide having SEQ ID NO:3 do not replace cysteine residues in the polypeptide having SEQ ID NO:1 and the polypeptide having SEQ ID NO:3, or
the variant of the polypeptide having SEQ ID NO: 1 and the variant of the polypeptide having SEQ ID NO:3 replaces one or both of the cysteine residues in the polypeptide having SEQ ID NO:1 and/or one or both of the cysteine residues in the polypeptide having SEQ ID NO:3 with an amino acid that contains a cross-linking functional group.

9. The polypeptide of claim 1, wherein the polypeptide comprises a polypeptide having SEQ ID NO: 7 (2A11—QVQLQESGGGSVPAGGSLRLSCAASGSTYSANCMGWFRQAPGKEREEVASMSIRSGRTYYSDSVK GRFTISQDGSKNTLYLQLNSLKAEDTALYYCAAAYGGSRCVYNYRGQGTQVTVSS).

10. A polypeptide, comprising:
a polypeptide having SEQ ID NO:4 (CDR1 of 5A1), a polypeptide having SEQ ID NO:5 (CDR2 of 5A1), a polypeptide having SEQ ID NO:6 (CDR3 of 5A1), or a combination thereof, OR
a variant of the polypeptide having SEQ ID NO:4 (CDR1 of 5A1), a variant of the polypeptide having SEQ ID NO:5 (CDR2 of 5A1), a variant of the polypeptide having SEQ ID NO:6 (CDR3 of 2A11), or a combination thereof, wherein the variant of the polypeptide having SEQ ID NO:4 comprises one or more deletions, additions or substitutions of an amino acid residue in the polypeptide having SEQ ID NO:4, wherein the variant of the polypeptide having SEQ ID NO:5 comprises one or more deletions, additions or substitutions of an amino acid residue in the polypeptide having SEQ ID NO:5, and wherein the variant of the polypeptide having SEQ ID NO:6 comprises one or more deletions, additions or substitutions of an amino acid residue in the polypeptide having SEQ ID NO:6, and
wherein:
the variant of the polypeptide having SEQ ID NO:4 and the variant of the polypeptide having SEQ ID NO: 6 do not replace cysteine residues in SEQ ID NO:4 and SEQ ID NO:6, or
the variant of the polypeptide having SEQ ID NO:4 and the variant of the polypeptide having SEQ ID NO:6 replaces one or both of the cysteine residues in the polypeptide having SEQ ID NO:4 and/or one or both of the cysteine residues in the polypeptide having SEQ ID NO:6 with an amino acid that contains a cross-linking functional group.

11. The polypeptide of claim 10, wherein
the polypeptide comprises a polypeptide having SEQ ID NO:8 (5A1—QVQLQESGGGSVQAGGSLKLSCTASGYTNRLKCMGWFRQAPGKEREEIATISTGTGNTYYADSVK GRFTFSQDKVKNTVYLQMNTLKPDDTGMYYCAADVRPDGTTCHYNSGGQGTQVTVSS); OR
the polypeptide comprises a polypeptide having:
SEQ ID NO: 11
(5A1-H1-EVQLLESGGGLVQPGGSLRLSCAASGYTNRLKCMGWFRQAPGKERELASISTGTGNTYYA
DSVKGRFTISRDNSKNTLYLQMNSLKAEDTAVYYCAADVRPDGTTCHYNSRGQGTLVTVS
S),
SEQ ID NO: 12
(5A1-H2-QVQLQESGGGLVQPGGSLRLSCTASGYTNRLKCMGWVRQAPGKEREEVATISTGTGNTYY
ADSVKGRFTISRDNSKNTLYLQMNSLRAEDTAMYYCAADVRPDGTTCHYNSGGQGTQVT
VSS),
SEQ ID NO: 13
(5A1-H3-(EVQLLESGGGLVQPGGSLRLSCAASGYTNRLKCMGWFRQAPGKEREEVSTISTGTGNTYY
ADSVKGRFTISQDKSKNTLYLRMNSLRAEDTALYYCAADVRPDGTTCHYNSGGQGTQVTV
SS),
SEQ ID NO: 14
(5A1-H4-(EVQLLESGGGLVPRGGSLRLSCTASGYTNRLKCMGWFRQAPGKEREEIATISTGTGNTYYA
DSVKGRFTISRDNSRNTLYLQMKTLRAEDTAVYYCAADVRPDGTTCHYNSWGQGTQVTV
SS),
or
SEQ ID NO: 15
(5A1-H5-EVQLVESGGGLVQPGGSLRLSCTASGYTNRLKCMGWFRQAPGKEREEIATISTGTGNTYYA
DSVKGRFTFSRDNSKNTLYLQMNSLRAEDTAVYYCAADVRPDGTTCHYNSGGQGTQVTV
SS).

12. A polynucleotide encoding a polypeptide claim 1.

13. The polynucleotide of claim 12, wherein the polynucleotide comprises a polynucleotide having:
SEQ ID NO: 9
(2A11-CAGGTCCAACTCCAAGAGAGCGGCGGCGGCTCCGTCCCAGCTGGAGGATCACTCAGACTCAGC
TGCGCCGCCAGCGGCTCCACCTACAGCGCTAACTGCATGGGCTGGTTCAGACAAGCCCCCGGC
AAAGAGAGAGAAGAGGTGGCTTCCATGTCAATCAGAAGCGGCCGTACCTACTACAGTGATTCC
GTGAAAGGCAGATTCACAATCAGCCAGGACGGAAGCAAGAACACCCTGTACCTGCAGCTGAAC
AGCCTGAAAGCCGAGGACACCGCCCTGTACTACTGCGCCGCCGCCTACGGGGGCTCTAGGTGT
GTGTACAACTACAGAGGCCAGGGCACACAAGTCACCGTCTCTAGC),
OR
SEQ ID NO: 10
(5A1-CAGGTCCAGCTCCAGGAAAGCGGCGGCGGCTCCGTCCAGGCAGGAGGAAGTCTCAAACTCTCC
TGCACAGCCTCCGGCTACACCAACAGACTCAAATGCATGGGCTGGTTCAGACAGGCACCCGGA
AAAGAGAGGGAAGAGATCGCTACCATCTCCACCGGCACCGGCAACACCTACTACGCCGACTCC
GTCAAAGGCAGGTTCACATTCAGCCAGGACAAGGTGAAGAACACAGTGTACCTGCAGATGAAC
ACACTGAAACCCGACGACACAGGCATGTACTACTGCGCCGCCGACGTTAGGCCCGATGGAACC
ACCTGCCACTACAACTCCGGAGGACAGGGAACCCAGGTCACCGTGAGCTCC).

14. The polynucleotide of claim 12, wherein the polynucleotide comprises a polynucleotide encoding a polypeptide selected from Table 7.

15. A vector comprising a polynucleotide of claim 12 or an isolated cell comprising a polynucleotide of claim 12.

16. (canceled)

17. A protein comprising a polypeptide of claim 1, and a fragment crystallizable region (Fc) of an antibody.

18. A multispecific antibody construct, comprising:
one or more first polypeptides, each independently selected from claim 1; and
a second polypeptide capable of binding an activation receptor and/or a costimulatory receptor expressed on an immune cell,
wherein optionally the activation receptor comprises cluster of differentiation (CD) 3, CD16, γ9 TCR, δ2 TCR or δ1 TCR, and the co-stimulatory/co-activation receptor comprises cluster of differentiation (CD) 137, CD28, DNAM-1, NKp46, NKG2D, NKp30, CD2, ICOS, OX40, CD40L, or CD40.

19. The multispecific antibody of claim 18, further comprising a fragment crystallizable region (Fc) of an antibody or a human serum albumin (HSA).

20. The multispecific antibody of claim 18, wherein the multispecific antibody comprises two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide having SEQ ID NO:7 or 8.

21. The multispecific antibody of claim 18, wherein the multispecific antibody comprises two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide selected from Table 7.

22. The multispecific antibody of claim 18, wherein the multispecific antibody comprises two or more VHH domains, or two or more single-chain variable fragments (scFv), capable of binding a tumor-associated antigen (TAA), wherein the two or more VHH domains are each independently the one or more first polypeptides, wherein the one or more first polypeptides comprise a polypeptide having a sequence as set forth in SEQ ID NO: 16, 39, 40 and 60.

23. The multispecific antibody of claim 18, wherein the multispecific antibody further comprises one or more linkers, and at least one of the linkers is between the Fc or the HSA and at least one of the first polypeptide, between the Fc or the HSA and the second polypeptide, between any two of the more first polypeptides, or between at least one of the one or more first polypeptides and the second polypeptide.

24. The multispecific antibody of claim 18, wherein the multispecific antibody is bispecific antibody.

25. A method of killing cancer cells or treating cancer in a subject in need thereof, comprising: administering a multispecific antibody of claim 18 to the subject in need thereof.

26. The method of claim 25, wherein the cancer cells express receptor tyrosine kinase-like orphan receptor 1 (ROR1).

27. The method of claim 25, wherein the cancer cells are cancer cells of the lung, bronchus, non-Hodgkin lymphoma, leukemia, pancreas, breast, prostate, colon, rectum, bladder, skin, kidney, mouth, tongue, pharynx, ovary, oral cavity, head and neck, thyroid, myeloid leukemia, mantle cell lymphoma, multiple myeloma, or combinations thereof.

28. (canceled)

29. (canceled)

30. (canceled)


SEQUENCE LISTING (208 sequences)

SEQ ID NO: 1 - protein, 8 aa, synthetic construct
GSTYSANC

SEQ ID NO: 2 - protein, 9 aa, synthetic construct
MSIRSGRTY

SEQ ID NO: 3 - protein, 11 aa, synthetic construct
AYGGSRCVYNY

SEQ ID NO: 4 - protein, 8 aa, synthetic construct
GYTNRLKC

SEQ ID NO: 5 - protein, 9 aa, synthetic construct
ISTGTGNTY

SEQ ID NO: 6 - protein, 13 aa, synthetic construct
DVRPDGTTCHYNS

SEQ ID NO: 7 - protein, 120 aa, Camelus bactrianus
QVQLQESGGGSVPAGGSLRLSCAASGSTYSANCMGWFRQAPGKEREEVASMSIRSGRTYYSDSVKGRFTISQDGSKNTLYLQLNSLKAEDTALYYCAAAYGGSRCVYNYRGQGTQVTVSS

SEQ ID NO: 8 - protein, 122 aa, Camelus bactrianus
QVQLQESGGGSVQAGGSLKLSCTASGYTNRLKCMGWFRQAPGKEREEIATISTGTGNTYYADSVKGRFTFSQDKVKNTVYLQMNTLKPDDTGMYYCAADVRPDGTTCHYNSGGQGTQVTVSS

SEQ ID NO: 9 - DNA, 360 bp, synthetic construct
  source 1..360: mol_type = other DNA
caggtccaactccaagagagcggcggcggctccgtcccagctggaggatcactcagactcagctgcgccgccagcggctccacctacagcgctaactgcatgggctggttcagacaagcccccggcaaagagagagaagaggtggcttccatgtcaatcagaagcggccgtacctactacagtgattccgtgaaaggcagattcacaatcagccaggacggaagcaagaacaccctgtacctgcagctgaacagcctgaaagccgaggacaccgccctgtactactgcgccgccgcctacgggggctctaggtgtgtgtacaactacagaggccagggcacacaagtcaccgtctctagc

SEQ ID NO: 10 - DNA, 366 bp, synthetic construct
  source 1..366: mol_type = other DNA
caggtccagctccaggaaagcggcggcggctccgtccaggcaggaggaagtctcaaactctcctgcacagcctccggctacaccaacagactcaaatgcatgggctggttcagacaggcacccggaaaagagagggaagagatcgctaccatctccaccggcaccggcaacacctactacgccgactccgtcaaaggcaggttcacattcagccaggacaaggtgaagaacacagtgtacctgcagatgaacacactgaaacccgacgacacaggcatgtactactgcgccgccgacgttaggcccgatggaaccacctgccactacaactccggaggacagggaacccaggtcaccgtgagctcc

SEQ ID NO: 11 - protein, 121 aa, synthetic construct
EVQLLESGGGLVQPGGSLRLSCAASGYTNRLKCMGWFRQAPGKERELASISTGTGNTYYADSVKGRFTISRDNSKNTLYLQMNSLKAEDTAVYYCAADVRPDGTTCHYNSRGQGTLVTVSS

SEQ ID NO: 12 - protein, 122 aa, synthetic construct
QVQLQESGGGLVQPGGSLRLSCTASGYTNRLKCMGWVRQAPGKEREEVATISTGTGNTYYADSVKGRFTISRDNSKNTLYLQMNSLRAEDTAMYYCAADVRPDGTTCHYNSGGQGTQVTVSS

SEQ ID NO: 13 - protein, 122 aa, synthetic construct
EVQLLESGGGLVQPGGSLRLSCAASGYTNRLKCMGWFRQAPGKEREEVSTISTGTGNTYYADSVKGRFTISQDKSKNTLYLRMNSLRAEDTALYYCAADVRPDGTTCHYNSGGQGTQVTVSS

SEQ ID NO: 14 - protein, 122 aa, synthetic construct
EVQLLESGGGLVPRGGSLRLSCTASGYTNRLKCMGWFRQAPGKEREEIATISTGTGNTYYADSVKGRFTISRDNSRNTLYLQMKTLRAEDTAVYYCAADVRPDGTTCHYNSWGQGTQVTVSS

SEQ ID NO: 15 - protein, 122 aa, synthetic construct
EVQLVESGGGLVQPGGSLRLSCTASGYTNRLKCMGWFRQAPGKEREEIATISTGTGNTYYADSVKGRFTFSRDNSKNTLYLQMNSLRAEDTAVYYCAADVRPDGTTCHYNSGGQGTQVTVSS

SEQ ID NO: 16 - protein, 122 aa, synthetic construct
EVQLLESGGGLVQPGGSLRLSCAASGYTNRLKCMGWFRQAPGKERELVASISTGTGNTYYADSVKGRFTISRDNSKNTLYLQMNSLKAEDTAVYYCAADVRPDGTTCHYNSRGQGTLVTVSS

SEQ ID NO: 17 - protein, 119 aa, synthetic construct
EVQLLESGGGLVQPGGSLRLSCAASGSTYSANCMGWFRQAPGKERELASMSIRSGRTYYADSVKGRFTISRDNSKNTLYLQMNSLKAEDTAVYYCAAAYGGSRCVYNYRGQGTLVTVSS

SEQ ID NO: 18 - protein, 122 aa, synthetic construct
EVQLLESGGGLVQPGGSLRLSCTASGYTNRLKCMGWFRQAPGKEREEIASISTGTGNTYYADSVKGRFTISRDKSRNTLYLQMNTLRAEDTAVYYCAADVRPHGTTCHYNSRGQGTQVTVSS

SEQ ID NO: 19 - protein, 119 aa, synthetic construct
EVQLLESGGGLVQPGGSLRLSCAASGSTYSANCMGWFRQAPGKERELASMSIRSGRTYYADSVKGRFTISRDSSKNTLYLQMNSLKAEDTAVYYCAAAYGGSRCVYNYRGQGTLVTVSS

SEQ ID NO: 20 - protein, 119 aa, synthetic construct
EVQLLESGGGLVQPGGSLRLSCAASGSTYSANCMGWFRQAPGKERELASMSIRSGRTYYADSVKGRFTISRDSSKNTLYLQMNSLRAEDTAVYYCAAAYGGSRCVYNYRGQGTLVTVSS

SEQ ID NO: 21 - protein, 119 aa, synthetic construct
EVQLLESGGGLVQPGGSLRLSCAASGSTYSANCMGWFRQAPGKERELASMSIRSGRTYYSDSVKGRFTISRDGSKNTLYLQMNSLKAEDTAVYYCAAAYGGSRCVYNYRGQGTLVTVSS

SEQ ID NO: 22 - protein, 122 aa, synthetic construct
EVQLLESGGGLVQPGGSLKLSCTASGYTNRLKCMGWFRQAPGKEREEIATISTGTGNTYYADSVKGRFTISQDRSKNTLYLQMNSLRAEDTAVYYCAADVRPDGTTCHYNSWGQGTLVTVSS

SEQ ID NO: 23 - protein, 121 aa, synthetic construct
QVQLVESGGGLVQPGGSLRLSCSASGYTNRLKCMGWFRQAPGKERELASISTGTGNTYYADSVKGRFTISRDNSKNTLYLQMNSLRADDTAVYYCAADVRPDGTTCHYNSWGQGTQVTVSS

SEQ ID NO: 24 - protein, 122 aa, synthetic construct
QVQLVESGGGLVQPGGSLRLSCSASGYTNRLKCMGWFRQAPGKEREEIATISSGTGNTYYADSVKGRFTFSRDNSKNTLYLQMNSLRAEDTGMYYCAADVRPDGTTCHYNSGGQGTQVTVSS

SEQ ID NO: 25 - protein, 119 aa, synthetic construct
EVQLLESGGGLVQPGGSLRLSCAASGSTYSANCMGWFRQAPGKERELASMSIRSGRTYYADSVKGRFTISQDGSKNTLYLQMNSLKAEDTAVYYCAAAYGGSRCVYNYRGQGTLVTVSS

SEQ ID NO: 26 - protein, 122 aa, synthetic construct
EVQLLESGGGLVQRGGSLRLSCTASGYTNRLKCMGWFRQAPGKEREEIASISTGTGNTYYSDSVKGRFTISRDKSRNTLYLQMKSLRAEDTAVYYCAADVRPRGTTCHYNSRGQGTQVTVSS

SEQ ID NO: 27 - protein, 122 aa, synthetic construct
EVQLLESGGGLVPRGGSLRLSCAASGYTNRLKCMGWFRQAPGKEREEIATISTGTGNTYYADSVKGRFTISQDKSRNTLYLQMNSLKAEDTAVYYCAADVRPKGTTCHYNKRGQGTQVTVSS

SEQ ID NO: 28 - protein, 122 aa, synthetic construct
EVQLLESGGGLVPRGGSLRLSCAASGYTNRLKCMGWFRQAPGKEREEVASISTGTGNTYYADSVKGRFTISQDKSRNTLYLQLNSLRAEDTAVYYCAADVRPRGTTCHYNSRGQGTQVTVSS

SEQ ID NO: 29 - protein, 122 aa, synthetic construct
EVQLLESGGGLVQRGGSLRLSCAASGYTNRLKCMGWFRQAPGKEREEIASISTGTGNTYYSDSVKGRFTISRDKSRNTLYLQLNSLKAEDTAVYYCAADVRPKGTTCHYNSRGQGTQVTVSS

SEQ ID NO: 30 - protein, 122 aa, synthetic construct
EVQLLESGGGLVPRGGSLRLSCTASGYTNRLKCMGWFRQAPGKEREEIASISTGTGNTYYADSVKGRFTISQDKSRNTLYLQMNSLKAEDTAVYYCAADVRPRGTTCHYNSRGQGTQVTVSS

SEQ ID NO: 31 - protein, 122 aa, synthetic construct
EVQLLESGGGLVQRGGSLRLSCTASGYTNRLKCMGWFRQAPGKEREEIATISTGTGNTYYADSVKGRFTISRDKSRNTLYLQMKSLKAEDTAVYYCAADVRPKGTTCHYNSRGQGTQVTVSS

SEQ ID NO: 32 - protein, 122 aa, synthetic construct
EVQLLESGGGLVPRGGSLRLSCTASGYTNRLKCMGWFRQAPGKEREEIASISTGTGNTYYSDSVKGRFTISRDKSRNTLYLKLKSLRAEDTAVYYCAADVRPDGTTCHYNSRGQGTQVTVSS

SEQ ID NO: 33 - protein, 121 aa, synthetic construct
EVQLLESGGGLVQPGGSLRLSCAASGYTNRLKCMGWFRQAPGKERELASISTGTGNTYYADSVKGRFTISRDGSKNTLYLQMNSLRAEDTAVYYCAADVRPDGTTCHYNSRGQGTLVTVSS

SEQ ID NO: 34 - protein, 121 aa, synthetic construct
EVQLLESGGGLVQPGGSLRLSCAASGYTNRLKCMGWFRQAPGKERELASISTGTGNTYYADSVKGRFTISQDGSKNTLYLQMNSLRAEDTAVYYCAADVRPDGTTCHYNSRGQGTLVTVSS

SEQ ID NO: 35 - protein, 122 aa, synthetic construct
EVQLLESGGGLVPRGGSLKLSCTASGYTNRLKCMGWFRQAPGKEREEIASISTGTGNTYYADSVKGRFTISQDKSRNTLYLQMNSLKAEDTAVYYCAADVRPRGTTCHYNSRGQGTQVTVSS

SEQ ID NO: 36 - protein, 122 aa, synthetic construct
EVQLLESGGGLVPRGGSLRLSCTASGYTNRLKCMGWFRQAPGKEREEIASISTGTGNTYYADSVKGRFTISQDKSRNTLYLQMSSLKAEDTAVYYCAADVRPRGTTCHYNSRGQGTQVTVSS

SEQ ID NO: 37 - protein, 119 aa, synthetic construct
EVQLLESGGGLVQPGGSLRLSCAASGSQRSANCMGWFRQAPGKDRELASMSIRSGRTYYADSVKGRFTISRDSSKNTLYLQMSSLKAEDTAVYYCAAAYGGSRCVYNYRGQGTLVTVSS

SEQ ID NO: 38 - protein, 119 aa, synthetic construct
EVQLLESGGGLVQPGGSLRLSCAASGVGYSANCMGWFRQAPGKDRELASMSIRSGRTYYADSVKGRFTISRDSSKNTLYLQMSSLKAEDTAVYYCAAAYGGSRCVYNYRGQGTLVTVSS

SEQ ID NO: 39 - protein, 122 aa, synthetic construct
EVQLLESGGGLVQPGGSLRLSCAASGYTNRIKCMGWFRQAPGKEREEVASISTGTGNTYYADSVKGRFTISQDKSKNTLYLQMNSLKAEDTAVYYCAADVRPDGFTCHYNSRGQGTLVTVSS

SEQ ID NO: 40 - protein, 122 aa, synthetic construct
EVQLLESGGGLVQPGGSLRLSCAASGYTNRIKCMGWFRQAPGKEREEVASISTGTGNTYYADSVKGRFTISQDKSKNTLYLQMNSLKAEDTAVYYCAADVRPDGTTCHYNKRGQGTLVTVSS

SEQ ID NO: 41 - protein, 122 aa, synthetic construct
EVQLLESGGGLVQPGGSLRLSCAASGYTNRLKCMGWFRQAPGKEREEVASISTGTGNAYYADSVKGRFTISQDKSKNTLYLQMNSLKAEDTAVYYCAADVRPDGTTCHYNKRGQGTLVTVSS

SEQ ID NO: 42 - protein, 122 aa, synthetic construct
EVQLLESGGGLVQPGGSLRLSCAASGYTNRIKCMGWFRQAPGKEREEVASISTGTGNAYYADSVKGRFTISQDKSKNTLYLQMNSLKAEDTAVYYCAADVRPDGTTCHYNKRGQGTLVTVSS

SEQ ID NO: 43 - protein, 122 aa, synthetic construct
EVQLLESGGGLVQPGGSLRLSCAASGYTNRLKCMGWFRQAPGKEREEVASISTGTGNTYYADSVKGRFTISQDKSKNTLYLQMNSLKAEDTAVYYCAADVRPDGFTCHYNKRGQGTLVTVSS

SEQ ID NO: 44 - protein, 122 aa, synthetic construct
EVQLLESGGGLVQPGGSLRLSCAASGYTNRIKCMGWFRQAPGKEREEVASISTGTGNTYYADSVKGRFTISQDKSKNTLYLQMNSLKAEDTAVYYCAADVRPDGRTCHYNSRGQGTLVTVSS

SEQ ID NO: 45 - protein, 122 aa, synthetic construct
EVQLLESGGGLVQPGGSLRLSCAASGYTNRLKCMGWFRQAPGKEREEVASISTGTGNAYYADSVKGRFTISQDKSKNTLYLQMNSLKAEDTAVYYCAADVRPDGRTCHYNSRGQGTLVTVSS

SEQ ID NO: 46 - protein, 122 aa, synthetic construct
EVQLLESGGGLVQPGGSLRLSCAASGYTNRIKCMGWFRQAPGKEREEVASISTGTGNAYYADSVKGRFTISQDKSKNTLYLQMNSLKAEDTAVYYCAADVRPDGRTCHYNSRGQGTLVTVSS

SEQ ID NO: 47 - protein, 122 aa, synthetic construct
EVQLLESGGGLVQPGGSLRLSCAASGYTNRIKCMGWFRQAPGKEREEVASISTGTGNSYYADSVKGRFTISQDKSKNTLYLQMNSLKAEDTAVYYCAADVRPDGRTCHYNKRGQGTLVTVSS

SEQ ID NO: 48 - protein, 122 aa, synthetic construct
EVQLLESGGGLVQPGGSLRLSCAASGYTNRLKCMGWFRQAPGKEREEVASISTGTGNTYYADSVKGRFTISQDKSKNTLYLQMNSLKAEDTAVYYCAADVRPDGRTCHYNKRGQGTLVTVSS

SEQ ID NO: 49 - protein, 122 aa, synthetic construct
EVQLLESGGGLVQPGGSLRLSCAASGYTNRIKCMGWFRQAPGKEREEVASISTGTGNTYYADSVKGRFTISQDKSKNTLYLQMNSLKAEDTAVYYCAADVRPFGTTCHYNSRGQGTLVTVSS

SEQ ID NO: 50 - protein, 122 aa, synthetic construct
EVQLLESGGGLVQPGGSLRLSCAASGYTNRLKCMGWFRQAPGKEREEVASISTGTGNTYYADSVKGRFTISQDKSKNTLYLQMNSLKAEDTAVYYCAADVRPFGRTCHYNSRGQGTLVTVSS

SEQ ID NO: 51 - protein, 122 aa, synthetic construct
EVQLLESGGGLVQPGGSLRLSCAASGYTNRLKCMGWFRQAPGKEREEVASISTGTGNTYYADSVKGRFTISQDKSKNTLYLQMNSLKAEDTAVYYCAADVRPFGTTCHYNKRGQGTLVTVSS

SEQ ID NO: 52 - protein, 122 aa, synthetic construct
EVQLLESGGGLVQPGGSLRLSCAASGYTNRIKCMGWFRQAPGKEREEVASISTGTGNTYYADSVKGRFTISQDKSKNTLYLQMNSLKAEDTAVYYCAADVRPFGRTCHYNSRGQGTLVTVSS

SEQ ID NO: 53 - protein, 122 aa, synthetic construct
EVQLLESGGGLVQPGGSLRLSCAASGYTNRIKCMGWFRQAPGKEREEVASISTGTGNTYYADSVKGRFTISQDKSKNTLYLQMNSLKAEDTAVYYCAADVRPFGTTCHYNKRGQGTLVTVSS

SEQ ID NO: 54 - protein, 122 aa, synthetic construct
EVQLLESGGGLVQPGGSLRLSCAASGYTNRLKCMGWFRQAPGKEREEVASISTGTGNAYYADSVKGRFTISQDKSKNTLYLQMNSLKAEDTAVYYCAADVRPHGTTCHYNSRGQGTLVTVSS

SEQ ID NO: 55 - protein, 122 aa, synthetic construct
EVQLLESGGGLVQPGGSLRLSCAASGYTNRIKCMGWFRQAPGKEREEVASISTGTGNAYYADSVKGRFTISQDKSKNTLYLQMNSLKAEDTAVYYCAADVRPHGTTCHYNSRGQGTLVTVSS

SEQ ID NO: 56 - protein, 122 aa, synthetic construct
EVQLLESGGGLVQPGGSLRLSCAASGYTNRLKCMGWFRQAPGKEREEVASISTGTGNAYYADSVKGRFTISQDKSKNTLYLQMNSLKAEDTAVYYCAADVRPHGRTCHYNSRGQGTLVTVSS

SEQ ID NO: 57 - protein, 122 aa, synthetic construct
EVQLLESGGGLVQPGGSLRLSCAASGYTNRIKCMGWFRQAPGKEREEVASISTGTGNAYYADSVKGRFTISQDKSKNTLYLQMNSLKAEDTAVYYCAADVRPHGRTCHYNSRGQGTLVTVSS

SEQ ID NO: 58 - protein, 122 aa, synthetic construct
EVQLLESGGGLVQPGGSLRLSCAASGYTNRLKCMGWFRQAPGKEREEVASISTGTGNAYYADSVKGRFTISQDKSKNTLYLQMNSLKAEDTAVYYCAADVRPHGTTCHYNKRGQGTLVTVSS

SEQ ID NO: 59 - protein, 122 aa, synthetic construct
EVQLLESGGGLVQPGGSLRLSCAASGYTNRIKCMGWFRQAPGKEREEVASISTGTGNAYYADSVKGRFTISQDKSKNTLYLQMNSLKAEDTAVYYCAADVRPHGTTCHYNKRGQGTLVTVSS

SEQ ID NO: 60 - protein, 122 aa, synthetic construct
EVQLLESGGGLVQPGGSLRLSCAASGYTNRLKCMGWFRQAPGKEREEVASISTGTGNTYYADSVKGRFTISQDKSKNTLYLQMNSLKAEDTAVYYCAADVRPDGTTCHYNSRGQGTLVTVSS

SEQ ID NO: 61 - protein, 122 aa, synthetic construct
EVQLLESGGGLVPRGGSLRLSCTASGYTNRLKCMGWFRQAPGKEREEIATISTGTGNTYYADSVKGRFTISQDKSRNTLYLQMKTLRAEDTAVYYCAADVRPDGTTCHYNSWGQGTQVTVSS

SEQ ID NO: 62 - protein, 122 aa, synthetic construct
EVQLLESGGGLVQPGGSLRLSCAASGFTFRLKCMGWFRQAPGKGLEEVATISTGTGNTYYADSVKGRFTISRDNSKNTLYLQMNSLRAEDTAVYYCAADVRPDGTTCHYNSWGQGTTVTVSS

SEQ ID NO: 63 - protein, 122 aa, synthetic construct
EVQLLESGGGLVQPGGSLRLSCAASGYTNRLKCMGWFRQAPGKGLEEVATISTGTGNTYYADSVKGRFTISRDNSKNTLYLQMNSLRAEDTAVYYCAADVRPDGTTCHYNSWGQGTTVTVSS

SEQ ID NO: 64 - protein, 122 aa, synthetic construct
EVQLLESGGGLVQPGGSLRLSCAASGYTNRLKCMGWFRQAPGKGLEEVATISTGTGNTYYADSVKGRFTISQDKSKNTLYLQMNSLRAEDTAVYYCAADVRPDGTTCHYNSWGQGTTVTVSS

SEQ ID NO: 65 - protein, 122 aa, synthetic construct
QVQLVESGGGVVQPGGSLRLSCAASGFTFRLKCMGWFRQAPGKGLEEVATISTGTGNTYYADSVKGRFTISRDNSKNTLYLQMNSLRAEDTAVYYCAADVRPDGTTCHYNSWGQGTTVTVSS

SEQ ID NO: 66 - protein, 122 aa, synthetic construct
QVQLVESGGGVVQPGGSLRLSCAASGYTNRLKCMGWFRQAPGKGLEEVATISTGTGNTYYADSVKGRFTISRDNSKNTLYLQMNSLRAEDTAVYYCAADVRPDGTTCHYNSWGQGTTVTVSS

SEQ ID NO: 67 - protein, 122 aa, synthetic construct
QVQLVESGGGVVQPGGSLRLSCAASGYTNRLKCMGWFRQAPGKGLEEVATISTGTGNTYYADSVKGRFTISQDKSKNTLYLQMNSLRAEDTAVYYCAADVRPDGTTCHYNSWGQGTTVTVSS

SEQ ID NO: 68 - protein, 122 aa, synthetic construct
EVQLVESGGGLVQPGGSLRLSCAASGFTVRLKCMGWFRQAPGKGLEEVATISTGTGNTYYADSVKGRFTISRDNSKNTLYLQMNSLRAEDTAVYYCAADVRPDGTTCHYNSWGQGTTVTVSS

SEQ ID NO: 69 - protein, 122 aa, synthetic construct
EVQLVESGGGLVQPGGSLRLSCAASGYTNRLKCMGWFRQAPGKGLEEVATISTGTGNTYYADSVKGRFTISRDNSKNTLYLQMNSLRAEDTAVYYCAADVRPDGTTCHYNSWGQGTTVTVSS

SEQ ID NO: 70 - protein, 122 aa, synthetic construct
EVQLVESGGGLVQPGGSLRLSCAASGYTNRLKCMGWFRQAPGKGLEEVATISTGTGNTYYADSVKGRFTISQDKSKNTLYLQMNSLRAEDTAVYYCAADVRPDGTTCHYNSWGQGTTVTVSS

SEQ ID NO: 71 - protein, 8 aa, synthetic construct
GSQRSANC

SEQ ID NO: 72 - protein, 8 aa, synthetic construct
GVGYSANC

SEQ ID NO: 73 - protein, 8 aa, synthetic construct
GYTNRIKC

SEQ ID NO: 74 - protein, 8 aa, synthetic construct
GFTFRLKC

SEQ ID NO: 75 - protein, 8 aa, synthetic construct
GFTVRLKC

SEQ ID NO: 76 - protein, 6 aa, synthetic construct
SIRSGR

SEQ ID NO: 77 - protein, 6 aa, synthetic construct
STGTGN

SEQ ID NO: 78 - protein, 5 aa, synthetic construct
IRSGR

SEQ ID NO: 79 - protein, 6 aa, synthetic construct
SSGTGN

SEQ ID NO: 80 - protein, 13 aa, synthetic construct
DVRPHGTTCHYNS

SEQ ID NO: 81 - protein, 13 aa, synthetic construct
DVRPRGTTCHYNS

SEQ ID NO: 82 - protein, 13 aa, synthetic construct
DVRPKGTTCHYNK

SEQ ID NO: 83 - protein, 13 aa, synthetic construct
DVRPKGTTCHYNS

SEQ ID NO: 84 - protein, 13 aa, synthetic construct
DVRPDGFTCHYNS

SEQ ID NO: 85 - protein, 13 aa, synthetic construct
DVRPDGTTCHYNK

SEQ ID NO: 86 - protein, 13 aa, synthetic construct
DVRPDGFTCHYNK

SEQ ID NO: 87 - protein, 13 aa, synthetic construct
DVRPDGRTCHYNS

SEQ ID NO: 88 - protein, 13 aa, synthetic construct
DVRPDGRTCHYNK

SEQ ID NO: 89 - protein, 13 aa, synthetic construct
DVRPFGTTCHYNS

SEQ ID NO: 90 - protein, 13 aa, synthetic construct
DVRPFGRTCHYNS

SEQ ID NO: 91 - protein, 13 aa, synthetic construct
DVRPFGTTCHYNK

SEQ ID NO: 92 - protein, 13 aa, synthetic construct
DVRPHGRTCHYNS

SEQ ID NO: 93 - protein, 13 aa, synthetic construct
DVRPHGTTCHYNK

SEQ ID NO: 94 - protein, 9 aa, synthetic construct
ISSGTGNTY

SEQ ID NO: 95 - protein, 9 aa, synthetic construct
ISTGTGNAY

SEQ ID NO: 96 - protein, 9 aa, synthetic construct
ISTGTGNSY

SEQ ID NO: 97 - protein, 25 aa, synthetic construct
QVQLQESGGGSVPAGGSLRLSCAAS

SEQ ID NO: 98 - protein, 25 aa, synthetic construct
QVQLQESGGGSVQAGGSLKLSCTAS

SEQ ID NO: 99 - protein, 25 aa, synthetic construct
EVQLLESGGGLVPRGGSLRLSCTAS

SEQ ID NO: 100 - protein, 25 aa, synthetic construct
EVQLVESGGGLVQPGGSLRLSCTAS

SEQ ID NO: 101 - protein, 25 aa, synthetic construct
EVQLLESGGGLVQPGGSLRLSCAAS

SEQ ID NO: 102 - protein, 25 aa, synthetic construct
EVQLLESGGGLVQPGGSLRLSCTAS

SEQ ID NO: 103 - protein, 25 aa, synthetic construct
EVQLLESGGGLVQPGGSLKLSCTAS

SEQ ID NO: 104 - protein, 25 aa, synthetic construct
QVQLVESGGGLVQPGGSLRLSCSAS

SEQ ID NO: 105 - protein, 25 aa, synthetic construct
EVQLLESGGGLVQRGGSLRLSCTAS

SEQ ID NO: 106 - protein, 25 aa, synthetic construct
EVQLLESGGGLVPRGGSLRLSCAAS

SEQ ID NO: 107 - protein, 25 aa, synthetic construct
EVQLLESGGGLVQRGGSLRLSCAAS

SEQ ID NO: 108 - protein, 25 aa, synthetic construct
EVQLLESGGGLVPRGGSLKLSCTAS

SEQ ID NO: 109 - protein, 25 aa, synthetic construct
QVQLVESGGGVVQPGGSLRLSCAAS

SEQ ID NO: 110 - protein, 25 aa, synthetic construct [sequence omitted]

SEQ ID NO: 111 - protein, 17 aa, synthetic construct
MGWFRQAPGKEREEVAS

SEQ ID NO: 112 - protein, 17 aa, synthetic construct
MGWFRQAPGKEREEIAT

SEQ ID NO: 113 - protein, 16 aa, synthetic construct
MGWFRQAPGKERELAS

SEQ ID NO: 114 - protein, 17 aa, synthetic construct
MGWFRQAPGKERELVAS

SEQ ID NO: 115 - protein, 17 aa, synthetic construct
MGWFRQAPGKEREEIAS

SEQ ID NO: 116 - protein, 16 aa, synthetic construct
MGWFRQAPGKDRELAS

SEQ ID NO: 117 - protein, 17 aa, synthetic construct
MGWFRQAPGKGLEEVAT

SEQ ID NO: 118 - protein, 39 aa, synthetic construct
YSDSVKGRFTISQDGSKNTLYLQLNSLKAEDTALYYCAA

SEQ ID NO: 119 - protein, 39 aa, synthetic construct
YADSVKGRFTFSQDKVKNTVYLQMNTLKPDDTGMYYCAA

SEQ ID NO: 120 - protein, 39 aa, synthetic construct
YADSVKGRFTISRDNSRNTLYLQMKTLRAEDTAVYYCAA

SEQ ID NO: 121 - protein, 39 aa, synthetic construct
YADSVKGRFTISRDNSKNTLYLQMNSLKAEDTAVYYCAA

SEQ ID NO: 122 - protein, 39 aa, synthetic construct
YADSVKGRFTISRDKSRNTLYLQMNTLRAEDTAVYYCAA

SEQ ID NO: 123 - protein, 39 aa, synthetic construct
YADSVKGRFTISRDSSKNTLYLQMNSLKAEDTAVYYCAA

SEQ ID NO: 124 - protein, 39 aa, synthetic construct
YADSVKGRFTISRDSSKNTLYLQMNSLRAEDTAVYYCAA

SEQ ID NO: 125 - protein, 39 aa, synthetic construct
YSDSVKGRFTISRDGSKNTLYLQMNSLKAEDTAVYYCAA

SEQ ID NO: 126 - protein, 39 aa, synthetic construct
YADSVKGRFTISQDRSKNTLYLQMNSLRAEDTAVYYCAA

SEQ ID NO: 127 - protein, 39 aa, synthetic construct
YADSVKGRFTISRDNSKNTLYLQMNSLRADDTAVYYCAA

SEQ ID NO: 128 - protein, 39 aa, synthetic construct
YADSVKGRFTFSRDNSKNTLYLQMNSLRAEDTGMYYCAA

SEQ ID NO: 129 - protein, 39 aa, synthetic construct
YADSVKGRFTISQDGSKNTLYLQMNSLKAEDTAVYYCAA

SEQ ID NO: 130 - protein, 39 aa, synthetic construct
YSDSVKGRFTISRDKSRNTLYLQMKSLRAEDTAVYYCAA

SEQ ID NO: 131 - protein, 39 aa, synthetic construct
YADSVKGRFTISQDKSRNTLYLQMNSLKAEDTAVYYCAA

SEQ ID NO: 132 - protein, 39 aa, synthetic construct
YADSVKGRFTISQDKSRNTLYLQLNSLRAEDTAVYYCAA

SEQ ID NO: 133 - protein, 39 aa, synthetic construct
YSDSVKGRFTISRDKSRNTLYLQLNSLKAEDTAVYYCAA

SEQ ID NO: 134 - protein, 39 aa, synthetic construct
YADSVKGRFTISRDKSRNTLYLQMKSLKAEDTAVYYCAA

SEQ ID NO: 135 - protein, 39 aa, synthetic construct
YSDSVKGRFTISRDKSRNTLYLKLKSLRAEDTAVYYCAA

SEQ ID NO: 136 - protein, 39 aa, synthetic construct
YADSVKGRFTISRDGSKNTLYLQMNSLRAEDTAVYYCAA

SEQ ID NO: 137 - protein, 39 aa, synthetic construct
YADSVKGRFTISQDGSKNTLYLQMNSLRAEDTAVYYCAA

SEQ ID NO: 138 - protein, 39 aa, synthetic construct
YADSVKGRFTISQDKSRNTLYLQMSSLKAEDTAVYYCAA

SEQ ID NO: 139 - protein, 39 aa, synthetic construct
YADSVKGRFTISRDSSKNTLYLQMSSLKAEDTAVYYCAA

SEQ ID NO: 140 - protein, 39 aa, synthetic construct
YADSVKGRFTISQDKSKNTLYLQMNSLKAEDTAVYYCAA

SEQ ID NO: 141 - protein, 39 aa, synthetic construct
YADSVKGRFTISQDKSRNTLYLQMKTLRAEDTAVYYCAA

SEQ ID NO: 142 - protein, 39 aa, synthetic construct
YADSVKGRFTISRDNSKNTLYLQMNSLRAEDTAVYYCAA

SEQ ID NO: 143 - protein, 39 aa, synthetic construct
YADSVKGRFTISQDKSKNTLYLQMNSLRAEDTAVYYCAA

SEQ ID NO: 144 - protein, 11 aa, synthetic construct
RGQGTQVTVSS

SEQ ID NO: 145 - protein, 11 aa, synthetic construct
GGQGTQVTVSS

SEQ ID NO: 146 - protein, 11 aa, synthetic construct
WGQGTQVTVSS

SEQ ID NO: 147 - protein, 11 aa, synthetic construct
RGQGTLVTVSS

SEQ ID NO: 148 - protein, 11 aa, synthetic construct
WGQGTLVTVSS

SEQ ID NO: 149 - protein, 11 aa, synthetic construct
WGQGTTVTVSS

SEQ ID NO: 150 - protein, 4 aa, synthetic construct
GGGS

SEQ ID NO: 151 - protein, 5 aa, synthetic construct
  UNSURE 5: note = Q or A or E or S
GGGGX

SEQ ID NO: 152 - protein, 5 aa, synthetic construct
GGGGS

SEQ ID NO: 153 - protein, 455 aa, synthetic construct
EVQLVESGGGLVQPGGSLRLSCAASGFTFSTYAMNWVRQAPGKGLEWVARIRSKYNNYATYYADSVKDRFTISRDDSKNTLYLQMNSLRAEDTAVYYCARHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 154 - protein, 455 aa, synthetic construct
EVQLVESGGGLVQPGGSLRLSCAASGFTFSTYAMNWVRQAPGKGLEWVARIRSKYNNYATYYADSVKDRFTISRDDSKNTLYLQMNSLRAEDTAVYYCARHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 215 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 156 - protein, 357 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSQVQLQESGGGSVQAGGSLKLSCTASGYTNRLKCMGWFRQAPGKEREEIATISTGTGNTYYADSVKGRFTFSQDKVKNTVYLQMNTLKPDDTGMYYCAADVRPDGTTCHYNSGGQGTQVTVSS

SEQ ID NO: 157 - protein, 487 aa, synthetic construct
QVQLQESGGGSVPAGGSLRLSCAASGSTYSANCMGWFRQAPGKEREEVASMSIRSGRTYYSDSVKGRFTISQDGSKNTLYLQLNSLKAEDTALYYCAAAYGGSRCVYNYRGQGTQVTVSSGGGGSGGGGSGGGGSQVQLQESGGGSVPAGGSLRLSCAASGSTYSANCMGWFRQAPGKEREEVASMSIRSGRTYYSDSVKGRFTISQDGSKNTLYLQLNSLKAEDTALYYCAAAYGGSRCVYNYRGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 158 - protein, 491 aa, synthetic construct
QVQLQESGGGSVQAGGSLKLSCTASGYTNRLKCMGWFRQAPGKEREEIATISTGTGNTYYADSVKGRFTFSQDKVKNTVYLQMNTLKPDDTGMYYCAADVRPDGTTCHYNSGGQGTQVTVSSGGGGSGGGGSGGGGSQVQLQESGGGSVQAGGSLKLSCTASGYTNRLKCMGWFRQAPGKEREEIATISTGTGNTYYADSVKGRFTFSQDKVKNTVYLQMNTLKPDDTGMYYCAADVRPDGTTCHYNSGGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 159 - protein, 354 aa, synthetic construct
QVQLQESGGGSVQAGGSLKLSCTASGYTNRLKCMGWFRQAPGKEREEIATISTGTGNTYYADSVKGRFTFSQDKVKNTVYLQMNTLKPDDTGMYYCAADVRPDGTTCHYNSGGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 160 - protein, 478 aa, synthetic construct
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVKGGGGSGGGGSGGGGSGGGGSQSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYCDFNIWGPGTLVTISSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 161 - protein, 354 aa, synthetic construct
QVQLQESGGGLVQPGGSLRLSCTASGYTNRLKCMGWVRQAPGKEREEVATISTGTGNTYYADSVKGRFTISRDNSKNTLYLQMNSLRAEDTAMYYCAADVRPDGTTCHYNSGGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 357 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSQVQLQESGGGLVQPGGSLRLSCTASGYTNRLKCMGWVRQAPGKEREEVATISTGTGNTYYADSVKGRFTISRDNSKNTLYLQMNSLRAEDTAMYYCAADVRPDGTTCHYNSGGQGTQVTVSS

SEQ ID NO: 163 - protein, 354 aa, synthetic construct
EVQLLESGGGLVQPGGSLRLSCAASGYTNRLKCMGWFRQAPGKEREEVSTISTGTGNTYYADSVKGRFTISQDKSKNTLYLRMNSLRAEDTALYYCAADVRPDGTTCHYNSGGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 357 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSEVQLLESGGGLVQPGGSLRLSCAASGYTNRLKCMGWFRQAPGKEREEVSTISTGTGNTYYADSVKGRFTISQDKSKNTLYLRMNSLRAEDTALYYCAADVRPDGTTCHYNSGGQGTQVTVSS

SEQ ID NO: 165 - protein, 354 aa, synthetic construct
EVQLLESGGGLVPRGGSLRLSCTASGYTNRLKCMGWFRQAPGKEREEIATISTGTGNTYYADSVKGRFTISRDNSRNTLYLQMKTLRAEDTAVYYCAADVRPDGTTCHYNSWGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 357 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSEVQLLESGGGLVPRGGSLRLSCTASGYTNRLKCMGWFRQAPGKEREEIATISTGTGNTYYADSVKGRFTISRDNSRNTLYLQMKTLRAEDTAVYYCAADVRPDGTTCHYNSWGQGTQVTVSS

SEQ ID NO: 167 - protein, 354 aa, synthetic construct
EVQLVESGGGLVQPGGSLRLSCTASGYTNRLKCMGWFRQAPGKEREEIATISTGTGNTYYADSVKGRFTFSRDNSKNTLYLQMNSLRAEDTAVYYCAADVRPDGTTCHYNSGGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 357 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSEVQLVESGGGLVQPGGSLRLSCTASGYTNRLKCMGWFRQAPGKEREEIATISTGTGNTYYADSVKGRFTFSRDNSKNTLYLQMNSLRAEDTAVYYCAADVRPDGTTCHYNSGGQGTQVTVSS

SEQ ID NO: 169 - protein, 353 aa, synthetic construct
EVQLLESGGGLVQPGGSLRLSCAASGYTNRLKCMGWFRQAPGKERELASISTGTGNTYYADSVKGRFTISRDNSKNTLYLQMNSLKAEDTAVYYCAADVRPDGTTCHYNSRGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 356 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSEVQLLESGGGLVQPGGSLRLSCAASGYTNRLKCMGWFRQAPGKERELASISTGTGNTYYADSVKGRFTISRDNSKNTLYLQMNSLKAEDTAVYYCAADVRPDGTTCHYNSRGQGTLVTVSS

SEQ ID NO: 171 - DNA, 1365 bp, synthetic construct
  source 1..1365: mol_type = other DNA
gaggtgcagctggtggagtccggaggaggactggtgcagcctggaggaagcctgaggctgtcctgcgctgcttccggatttacatttagcacatatgccatgaactgggtgaggcaggcccctggaaaaggtctggaatgggtggctaggattcggtccaagtacaacaattacgccacctactatgccgacagtgtcaaggacaggtttaccatctccagggacgattcaaagaacaccctgtacctgcagatgaactccctgagggccgaggataccgctgtgtattactgtgcccggcacggaaatttcgggaactcctacgtgtcctggttcgcatactggggccagggaacaatggtgactgtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaacagatttacgcagaagagcctctccctgtctccgggtaaa

SEQ ID NO: 172 - DNA, 1365 bp, synthetic construct
  source 1..1365: mol_type = other DNA
gaggtgcagctggtggagagcggggggggactggtgcagccaggaggaagcctgagactgagctgtgccgccagcgggtttacatttagcacatatgccatgaactgggtgagacaggcccccggaaaaggactggagtgggtggctagaatcagaagcaagtacaacaactacgccacctactacgccgacagtgtgaaggacagattcaccattagcagagatgatagcaagaacacactgtacctgcagatgaatagtctgagagccgaggacacagccgtgtactactgtgccaggcacggaaacttcgggaacagctacgtgagctggttcgcctactggggccagggcaccatggtgaccgtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 173 - DNA, 645 bp, synthetic construct
  source 1..645: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttca

SEQ ID NO: 174 - DNA, 1071 bp, synthetic construct
  source 1..1071: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttcaggtggcggagggtctggtggtggaggatcagggggtggaggttcaggaggcgggggaagtcaggtgcagctgcaggaatcaggcggaggaagcgtgcaggctggaggatctctgaagctgagctgcacagccagcggctacacaaacagactgaagtgcatgggctggttcagacaggcccccggaaaagagagagaggagattgctacaattagcacaggaaccggcaacacctactacgccgacagtgtgaaaggcagattcacattcagccaggacaaagtgaagaacaccgtgtacctgcagatgaacaccctgaagcccgacgacaccggaatgtactactgcgccgccgacgtgagacccgatggaacaacatgccactacaacagcggaggacagggaacccaggtgaccgtgtctagc

SEQ ID NO: 175 - DNA, 1461 bp, synthetic construct
  source 1..1461: mol_type = other DNA
caggtgcagctgcaggagagcggcggcggatcagtgccagccggaggaagcctgagactgagctgcgccgccagcggcagcacatacagcgccaactgcatgggctggttcagacaggctcccggaaaagaaagagaagaagtggccagcatgagcatcagaagcggccggacctactacagtgatagtgtgaagggcagattcaccattagccaggacggcagcaagaacacactgtacctgcagctgaacagcctgaaagccgaagacaccgccctgtactactgcgccgccgcctacggcggatctaggtgtgtgtacaactacagaggccagggcacccaggtgaccgtgagcagcggtggtggcggatcaggtgggggaggctctggtggaggcggtagtcaggtgcagctgcaggagagcggcggcggatcagtgccagccggaggaagcctgagactgagctgcgccgccagcggcagcacatacagcgccaactgcatgggctggttcagacaggctcccggaaaagaaagagaagaagtggccagcatgagcatcagaagcggccggacctactacagtgatagtgtgaagggcagattcaccattagccaggacggcagcaagaacacactgtacctgcagctgaacagcctgaaagccgaagacaccgccctgtactactgcgccgccgcctacggcggatctaggtgtgtgtacaactacagaggccagggcacccaggtgaccgtgagcagcgagcccaaatctagcgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 176 - DNA, 1473 bp, synthetic construct
  source 1..1473: mol_type = other DNA
caggtgcagctgcaggagagcggcggcggatcagtgcaggccggaggaagcctgaagctgagctgcacagccagcggctacacaaacagactgaaatgcatgggctggttcagacaggcccccggaaaagagagagaagagatcgccaccatcagcaccggcacaggaaacacatactacgccgacagcgtgaaaggcagattcaccttcagccaggacaaagtgaagaacaccgtgtacctgcagatgaacacactgaagcccgacgacaccggcatgtactactgcgccgccgacgtgaggcctgacggaacaacctgccactacaacagcggaggacagggaacccaggtgaccgtgtccagcggtggtggcggatcaggtgggggaggctctggtggaggcggtagtcaggtgcagctgcaggagagcggcggcggatcagtgcaggccggaggaagcctgaagctgagctgcacagccagcggctacacaaacagactgaaatgcatgggctggttcagacaggcccccggaaaagagagagaagagatcgccaccatcagcaccggcacaggaaacacatactacgccgacagcgtgaaaggcagattcaccttcagccaggacaaagtgaagaacaccgtgtacctgcagatgaacacactgaagcccgacgacaccggcatgtactactgcgccgccgacgtgaggcctgacggaacaacctgccactacaacagcggaggacagggaacccaggtgaccgtgtccagcgagcccaaatctagcgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 177 - DNA, 1062 bp, synthetic construct
  source 1..1062: mol_type = other DNA
caggtgcagctgcaggagtccggcggcggaagcgtgcaggcaggaggaagcctgaagctgagctgcacagcctccggctacacaaacaggctgaaatgcatgggctggttcaggcaggcacccggcaaagagagagaggagattgctacaatcagcaccggaaccggaaatacctactacgccgactccgtgaaaggcaggttcacattcagccaggacaaagtgaagaacaccgtgtacctgcagatgaacaccctgaagcccgacgacaccggcatgtactactgcgccgccgacgtgaggcccgatggaaccacctgccactacaactccggaggacagggaacccaggtgaccgtgtcctccgagcccaaatctagcgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 178 - DNA, 1434 bp, synthetic construct
  source 1..1434: mol_type = other DNA
gagctggtgatgacacagacacccagcagcacctccggcgccgtgggaggaacagtgacaattaactgtcaggccagccagagcattgatagcaacctggcctggttccagcagaagcccggccagccccccaccctgctgatctacagagctagtaacctggccagcggagtgcccagcagattttccggaagcagaagcggcacagaatacaccctgaccatctccggcgtgcagagagaagacgccgccacctactactgcctgggcggggtgggcaatgtgagttacagaacatcctttggcggaggaaccgaggtggtggtgaagggtggcggagggtctggtggtggaggatcagggggtggaggttcaggaggcgggggaagtcagagcgtgaaggagagcgagggcgacctggtgacccccgctggaaacctgaccctgacctgcacagccagcggcagcgacatcaacgactaccccatcagctgggtgagacaggcccccggaaagggactggaatggattggattcattaacagcggaggaagcacctggtacgccagctgggtgaaaggaagatttaccatcagccgcaccagcaccactgtggacctgaaaatgacaagcctgactaccgacgacacagccacctacttctgcgccagaggctatagcacctactactgcgactttaacatctggggccccggcaccctggtgaccattagctccgagcccaaatctagcgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 179 - DNA, 1062 bp, synthetic construct
  source 1..1062: mol_type = other DNA
caggtccagctccaggaaagcggcggcggcctcgtgcagcctggaggaagtttgaggctctcctgcacagcctccggctacaccaacagactcaaatgcatgggctgggtcagacaggcccccggaaaagaaagggaggaggtggctactatttccaccggaacaggaaacacctactacgccgacagtgtgaagggcaggttcaccatctccagggacaattctaagaacacactgtacttgcagatgaacagcttgagggccgaggacaccgccatgtactactgcgccgccgacgtgaggcccgacggaacaacctgccactacaacagcggaggccagggaacccaggtgaccgtgtcctccgagcccaaatctagcgacaaaactcacacatgcccaccgtgcccagcacctgaagccgccgggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 180 - DNA, 1071 bp, synthetic construct
  source 1..1071: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttcaggtggcggagggtctggtggtggaggatcagggggtggaggttcaggaggcgggggaagtcaggtccagctccaggaaagcggcggcggcctcgtgcagcctggaggaagtttgaggctctcctgcacagcctccggctacaccaacagactcaaatgcatgggctgggtcagacaggcccccggaaaagaaagggaggaggtggctactatttccaccggaacaggaaacacctactacgccgacagtgtgaagggcaggttcaccatctccagggacaattctaagaacacactgtacttgcagatgaacagcttgagggccgaggacaccgccatgtactactgcgccgccgacgtgaggcccgacggaacaacctgccactacaacagcggaggccagggaacccaggtgaccgtgtcctcc

SEQ ID NO: 181 - DNA, 1062 bp, synthetic construct
  source 1..1062: mol_type = other DNA
gaggtgcagttgttggagagcgggggggggctggtgcagccaggaggaagtctgaggttgtcctgcgcagcatccggatatacaaacaggttgaagtgtatggggtggtttaggcaggcacctggcaaggagagggaggaggtgagtacaatcagcaccggaaccggaaacacctattacgccgacagtgtcaagggcaggttcacaatctcccaggacaagagcaagaacacactgtacctgaggatgaacagcctgagggcagaagatactgctctgtattattgcgccgccgacgttaggcccgatggaacaacctgccactacaactccggaggacagggaacccaggtcaccgtgagctccgagcccaaatctagcgacaaaactcacacatgcccaccgtgcccagcacctgaagccgccgggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 182 - DNA, 1071 bp, synthetic construct
  source 1..1071: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttcaggtggcggagggtctggtggtggaggatcagggggtggaggttcaggaggcgggggaagtgaggtgcagttgttggagagcgggggggggctggtgcagccaggaggaagtctgaggttgtcctgcgcagcatccggatatacaaacaggttgaagtgtatggggtggtttaggcaggcacctggcaaggagagggaggaggtgagtacaatcagcaccggaaccggaaacacctattacgccgacagtgtcaagggcaggttcacaatctcccaggacaagagcaagaacacactgtacctgaggatgaacagcctgagggcagaagatactgctctgtattattgcgccgccgacgttaggcccgatggaacaacctgccactacaactccggaggacagggaacccaggtcaccgtgagctcc

SEQ ID NO: 183 - DNA, 1062 bp, synthetic construct
  source 1..1062: mol_type = other DNA
gaggtgcagttgttggagagcgggggggggctggtgcccagaggaggaagtttgaggttgtcctgcacagcaagcgggtacacaaacaggctgaagtgtatggggtggtttaggcaggcacctggcaaggagagggaggagatcgcaacaatcagcacaggaacaggcaacacctactatgccgacagtgtgaagggcaggttcacaatctcccgtgacaacagcaggaacaccctgtacctgcagatgaaaactttacgtgccgaagatactgctgtgtactattgcgccgccgacgttaggcccgatggaacaacctgccactacaactcctggggacagggaacccaggtcaccgtgagctccgagcccaaatctagcgacaaaactcacacatgcccaccgtgcccagcacctgaagccgccgggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 184 - DNA, 1071 bp, synthetic construct
  source 1..1071: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttcaggtggcggagggtctggtggtggaggatcagggggtggaggttcaggaggcgggggaagtgaggtgcagttgttggagagcgggggggggctggtgcccagaggaggaagtttgaggttgtcctgcacagcaagcgggtacacaaacaggctgaagtgtatggggtggtttaggcaggcacctggcaaggagagggaggagatcgcaacaatcagcacaggaacaggcaacacctactatgccgacagtgtgaagggcaggttcacaatctcccgtgacaacagcaggaacaccctgtacctgcagatgaaaactttacgtgccgaagatactgctgtgtactattgcgccgccgacgttaggcccgatggaacaacctgccactacaactcctggggacagggaacccaggtcaccgtgagctcc

SEQ ID NO: 185 - DNA, 1062 bp, synthetic construct
  source 1..1062: mol_type = other DNA
gaggtgcagttggtggagagcgggggggggctggtgcagcctggaggaagtttgaggttgagctgtacagcaagcgggtacacaaacaggttgaagtgtatggggtggttcaggcaggcacctggaaaggagagggaggagatcgcaaccatttccacaggaacaggcaacacctactacgccgacagtgtgaagggcaggttcacattctccagggacaactccaagaacacactgtacttgcagatgaacagcctgagggccgaagatactgctgtgtactactgcgccgccgacgttaggcccgacggaacaacatgccactacaactccggtggccagggcacccaggtcaccgtgtcctccgagcccaaatctagcgacaaaactcacacatgcccaccgtgcccagcacctgaagccgccgggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 186 - DNA, 1071 bp, synthetic construct
  source 1..1071: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttcaggtggcggagggtctggtggtggaggatcagggggtggaggttcaggaggcgggggaagtgaggtgcagttggtggagagcgggggggggctggtgcagcctggaggaagtttgaggttgagctgtacagcaagcgggtacacaaacaggttgaagtgtatggggtggttcaggcaggcacctggaaaggagagggaggagatcgcaaccatttccacaggaacaggcaacacctactacgccgacagtgtgaagggcaggttcacattctccagggacaactccaagaacacactgtacttgcagatgaacagcctgagggccgaagatactgctgtgtactactgcgccgccgacgttaggcccgacggaacaacatgccactacaactccggtggccagggcacccaggtcaccgtgtcctcc

SEQ ID NO: 187 - DNA, 1059 bp, synthetic construct
  source 1..1059: mol_type = other DNA
gaggtgcagctgctggagtccggaggaggactggtgcagcctggaggaagcctgaggctgtcctgcgctgcttccggatataccaacaggctgaagtgtatgggatggtttaggcaggcccccggaaaagagagggagctggcttccatcagcaccggaactggaaacacctactacgccgactccgtgaagggaaggttcacaatctccagagacaacagcaagaacacactctacctccagatgaactccctgaaagccgaggacaccgccgtgtattactgcgctgctgacgtgaggcccgatggaaccacctgccactataacagccggggacagggaaccctggtcaccgtgtcctccgagcctaaatcctccgacaaaacacacacatgccccccctgccctgcacctgaggctgctggaggaccttccgtgtttctgttcccccctaaacccaaggacaccctcatgatttcccgcacccccgaagtgacctgcgtggtggtggacgtgtctcatgaagaccccgaggtgaagttcaattggtacgtggacggcgtggaggtgcacaatgcaaaaaccaaacccagggaggagcagtataactccacatatagggtggtgtccgtgctgaccgtgctgcaccaggactggctgaatggaaaggagtacaaatgcaaggtgtccaacaaagcactgccagcccccattgagaaaaccatctccaaagccaagggccagcccagagaaccacaggtgtacacactgcccccctcaagagaagaaatgaccaagaaccaggtgagcctctggtgcctcgtgaagggattctacccctccgacatcgccgtggaatgggaatcaaacggccagcctgagaacaactacaaaaccaccccccccgtgctcgactcagatggatcttttttcctgtattccaagctgactgtggacaagtctaggtggcagcagggcaacgtcttcagctgtagcgtgatgcacgaggccctgcataaccactacacccagaagagtctgtccctgagccccggaaag

SEQ ID NO: 188 - DNA, 1068 bp, synthetic construct
  source 1..1068: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttcaggtggcggagggtctggtggtggaggatccgggggaggaggttcaggaggcgggggatccgaggtgcagctgctggagtccggaggaggactggtgcagcctggaggaagcctgaggctgtcctgcgctgcttccggatataccaacaggctgaagtgtatgggatggtttaggcaggcccccggaaaagagagggagctggcttccatcagcaccggaactggaaacacctactacgccgactccgtgaagggaaggttcacaatctccagagacaacagcaagaacacactctacctccagatgaactccctgaaagccgaggacaccgccgtgtattactgcgctgctgacgtgaggcccgatggaaccacctgccactataacagccggggacagggaaccctggtcaccgtgtcctcc

SEQ ID NO: 189 - protein, 120 aa, Camelus bactrianus
QVQLQESGGGSVPAGGSLRLSCAASGSTYSANVMGWFRQAPGKEREEVASMSIRSGRTYYSDSVKGRFTISQDGSKNTLYLQLNSLKAEDTALYYCAAAYGGSRVVYNYRGQGTQVTVSS

SEQ ID NO: 190 - protein, 122 aa, Camelus bactrianus
QVQLQESGGGSVQAGGSLKLSCTASGYTNRLKVMGWFRQAPGKEREEIATISTGTGNTYYADSVKGRFTFSQDKVKNTVYLQMNTLKPDDTGMYYCAADVRPDGTTVHYNSGGQGTQVTVSS

SEQ ID NO: 191 - DNA, 363 bp, synthetic construct
  source 1..363: mol_type = other DNA
gaggtgcagctgctggagtccggaggaggactggtgcagcctggaggaagcctgaggctgtcctgcgctgcttccggatataccaacaggctgaagtgtatgggatggtttaggcaggcccccggaaaagagagggagctggcttccatcagcaccggaactggaaacacctactacgccgactccgtgaagggaaggttcacaatctccagagacaacagcaagaacacactctacctccagatgaactccctgaaagccgaggacaccgccgtgtattactgcgctgctgacgtgaggcccgatggaaccacctgccactataacagccggggacagggaaccctggtcaccgtgtcctcc

SEQ ID NO: 192 - DNA, 366 bp, synthetic construct
  source 1..366: mol_type = other DNA
caggtccagctccaggaaagcggcggcggcctcgtgcagcctggaggaagtttgaggctctcctgcacagcctccggctacaccaacagactcaaatgcatgggctgggtcagacaggcccccggaaaagaaagggaggaggtggctactatttccaccggaacaggaaacacctactacgccgacagtgtgaagggcaggttcaccatctccagggacaattctaagaacacactgtacttgcagatgaacagcttgagggccgaggacaccgccatgtactactgcgccgccgacgtgaggcccgacggaacaacctgccactacaacagcggaggccagggaacccaggtgaccgtgtcctcc

SEQ ID NO: 193 - DNA, 366 bp, synthetic construct
  source 1..366: mol_type = other DNA
gaggtgcagttgttggagagcgggggggggctggtgcagccaggaggaagtctgaggttgtcctgcgcagcatccggatatacaaacaggttgaagtgtatggggtggtttaggcaggcacctggcaaggagagggaggaggtgagtacaatcagcaccggaaccggaaacacctattacgccgacagtgtcaagggcaggttcacaatctcccaggacaagagcaagaacacactgtacctgaggatgaacagcctgagggcagaagatactgctctgtattattgcgccgccgacgttaggcccgatggaacaacctgccactacaactccggaggacagggaacccaggtcaccgtgagctcc

SEQ ID NO: 194 - DNA, 366 bp, synthetic construct
  source 1..366: mol_type = other DNA
gaggtgcagttgttggagagcgggggggggctggtgcccagaggaggaagtttgaggttgtcctgcacagcaagcgggtacacaaacaggctgaagtgtatggggtggtttaggcaggcacctggcaaggagagggaggagatcgcaacaatcagcacaggaacaggcaacacctactatgccgacagtgtgaagggcaggttcacaatctcccgtgacaacagcaggaacaccctgtacctgcagatgaaaactttacgtgccgaagatactgctgtgtactattgcgccgccgacgttaggcccgatggaacaacctgccactacaactcctggggacagggaacccaggtcaccgtgagctcc

SEQ ID NO: 195 - DNA, 366 bp, synthetic construct
  source 1..366: mol_type = other DNA
gaggtgcagttggtggagagcgggggggggctggtgcagcctggaggaagtttgaggttgagctgtacagcaagcgggtacacaaacaggttgaagtgtatggggtggttcaggcaggcacctggaaaggagagggaggagatcgcaaccatttccacaggaacaggcaacacctactacgccgacagtgtgaagggcaggttcacattctccagggacaactccaagaacacactgtacttgcagatgaacagcctgagggccgaagatactgctgtgtactactgcgccgccgacgttaggcccgacggaacaacatgccactacaactccggtggccagggcacccaggtcaccgtgtcctcc

SEQ ID NO: 196 - DNA, 360 bp, synthetic construct
  source 1..360: mol_type = other DNA
caggtgcagctgcaggagtccggaggaggaagcgtgcctgctggaggaagcctgaggctgtcctgcgctgcttccggaagcacctattccgctaacgtcatgggctggtttaggcaggcccctggaaaagagagggaggaagtggctagtatgagcatcaggagcggcaggacctactactctgactccgtcaagggaagattcacaatcagccaggacggaagcaagaacacactgtacctgcagctgaactctctgaaggccgaggacaccgcactgtactactgcgctgctgcttacggcggttccagagtggtgtacaactacaggggccagggaacacaggtcaccgtgtcctcc

SEQ ID NO: 197 - DNA, 366 bp, synthetic construct
  source 1..366: mol_type = other DNA
caggtgcagctgcaggagtccggaggaggaagcgtgcaggctggaggaagcctgaaactctcctgcacagcctccggatatacaaacaggctcaaagtgatgggctggttcaggcaggcccctggaaaagaaagggaggagattgcaacaatctccaccggcacaggaaacacatactacgccgactctgtcaagggccggttcacattctcccaggacaaggtgaagaacacagtgtacctccagatgaacaccctgaagcccgacgacacaggcatgtactactgcgcagccgacgtgaggcctgatggaaccaccgtgcattacaacagcggaggccagggaacacaggtgacagtgagttcc

SEQ ID NO: 198 - protein, 232 aa, synthetic construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 235 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGS

SEQ ID NO: 201 - DNA, 696 bp, synthetic construct
  source 1..696: mol_type = other DNA
gagcccaaatctgctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 202 - DNA, 696 bp, synthetic construct
  source 1..696: mol_type = other DNA
gagcccaaatctagcgacaaaactcacacatgcccaccgtgcccagcacctgaagccgccgggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 203 - DNA, 705 bp, synthetic construct
  source 1..705: mol_type = other DNA
caggctgtggtgacccaggagcccagcctgaccgtgagccccggaggaacagtgaccctgacctgcagaagcagcaccggcgccgtgacaacctcaaactacgccaactgggtgcagcagaaacctggacaggcccccagaggactgatcggcggaaccaacaagagagcccccggcacccccgccagattcagcggaagtctgctgggcggcaaagccgccctgaccctgagcggagtgcagcctgaggatgaagccgagtactactgcgctctgtggtacagcaacctgtgggtgttcggaggaggcaccaagctgaccgtgctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaggagctacagttgccaggtcacgcatgaagggagcaccgtggagaagacagtggcccctacagaatgttcaggtggcggagggtctggtggtggaggatcagggggtggaggttcaggaggcgggggaagt

SEQ ID NO: 204 - protein, 8 aa, synthetic construct
GSTYSANV

SEQ ID NO: 205 - protein, 11 aa, synthetic construct
AYGGSRVVYNY

SEQ ID NO: 206 - protein, 8 aa, synthetic construct
GYTNRLKV

SEQ ID NO: 207 - protein, 12 aa, synthetic construct
DVRPDGTTVHYN

SEQ ID NO: 208 - protein, 223 aa, synthetic construct
EVQLVESGGGLVQPGGSLRLSCAASGFTFSTYAMNWVRQAPGKGLEWVARIRSKYNNYATYYADSVKDRFTISRDDSKNTLYLQMNSLRAEDTAVYYCARHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV